SEQUENCE LISTING (2 sequences)

SEQ ID NO: 1 - protein, 213 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 452 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0248937 A9
(43) Pub. Date: Aug. 27, 2026

(54) SELF-STABILIZING LINKER CONJUGATES

(71) Applicant: BEONE MEDICINES I GMBH, Basel (CH)

(72) Inventors: Charng-Sheng Tsai, Beijing (CN); Mei-Hsuan Tsai, Beijing (CN); Xiaodong Wei, Beijing (CN); Wei Luo, Beijing (CN); Liming Wu, Beijing (CN)

(73) Assignee: BEONE MEDICINES I GMBH, Basel (CH)

(21) Appl. No.: 19/292,082

(22) Filed: Aug. 6, 2025

Related U.S. Application Data
(63) Continuation of application No. PCT/CN2024/076657, filed on Feb. 7, 2024.

(30) Foreign Application Priority Data
Feb. 9, 2023 (WO) PCT/CN2023/075152

(51) Int. Cl. A61K 47/68

(52) U.S. Cl. CPC A61K 47/68037; A61K 47/68031; A61K 47/6849

(57) ABSTRACT

The present disclosure provides antibody drug conjugate platforms comprising a self-stabilizing linker assembly component, and antibody drug conjugates comprising platform-derived linker-payloads and antibodies or antigen-binding fragments thereof. In some embodiments, an antibody drug conjugate is of the following formula:

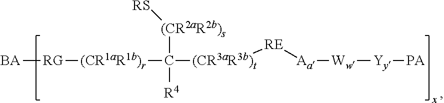

or a pharmaceutically acceptable salt, tautomer, solvate, or stereoisomer thereof, wherein values for the variables (e.g., BA, RG, R^{1a}, R^{1b}, r, RS, R^{2a}, R^{2b}, s, R⁴, R^{3a}, R^{3b}, t, RE, A, a′, W, w′, Y, y′, PA, x) are as described herein.

### 1. CROSS REFERENCE TO RELATED APPLICATIONS

This application is a continuation of International Application No. PCT/CN2024/076657, filed on Feb. 7, 2024, which claims priority to International Application No. PCT/CN2023/075152, filed on Feb. 9, 2023, the disclosure of each of which is hereby incorporated by reference in its entirety.

### 2. REFERENCE TO ELECTRONIC SEQUENCE LISTING

The application contains a Sequence Listing which has been submitted electronically in .XML format and is hereby incorporated by reference in its entirety. Said .XML copy, created on Feb. 5, 2024, is named “01368-0069-00PCT.xml” and is 3,423 bytes in size.

### 3. FIELD

Provided herein are antibody drug conjugate platforms and antibody drug conjugates (ADCs) comprising the platforms and an antibody, or antigen-binding fragment thereof, as well as uses of the ADC platforms and ADCs.

### 4. BACKGROUND

The antibody-drug conjugate (ADC) field has made significant progress with the advancement of many ADCs in the clinic. The linker component of ADCs is one important feature in developing optimized therapeutic agents that are highly active at well-tolerated doses. The electrophilic maleimide functional group has proven useful in the preparation of ADCs due to its high degree of specificity for reacting with thiol groups and the fast thiol addition kinetics under gentle conditions.

As has been noted by multiple investigators in the bioconjugate field, the thio-substituted product of the reaction between the electrophilic maleimide functional group and a free thiol of an antibody is subject to slow elimination, thus reversing the reaction.

When this reversible reaction occurs in a purified preparation of an ADC, the reaction is largely undetectable because the maleimide and thiol that are regenerated through the elimination process simply react again, thus reforming the intact conjugate. However, when other thiols are present, the net effect can be the transfer of the maleimide from the antibody of the ADC onto any other available thiol. This process has been documented to occur in plasma, in which the maleimide of an ADC transfers to cysteine 34 of serum albumin (Alley et al., Bioconjugate Chem. 2008, 19, 759-765). This process has also been reported when an ADC is incubated in the presence of excess cysteine or glutathione (Jununtula et al., Nature Biotech, 2012). The present disclosure is directed to, inter alia, bioconjugates that do not undergo this transfer reaction.

### 5. SUMMARY

Provided herein are antibody drug conjugate platforms and antibody drug conjugates (ADCs). Also provided are uses of the ADC platforms to prepare ADCs.

In some embodiments, provided herein are ADC compounds of Formula (I):

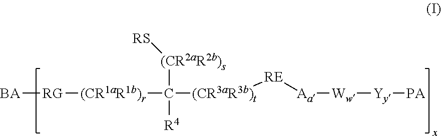

or a pharmaceutically acceptable salt, tautomer, solvate, or stereoisomer thereof, wherein values for the variables (e.g., BA, RG, RS, RE, PA, R^{1a}, R^{1b}, R^{2a}, R^{2b}, R^{3a}, R^{3b}, R⁴, A, W, Y, r, s, t, a′, w′, y′, x) are as described herein.

In some embodiments, the platform is linker-payload compound of Formula (II):

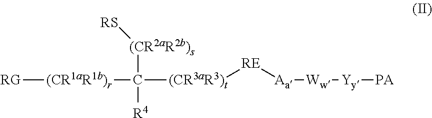

or a pharmaceutically acceptable salt, tautomer, solvate, or stereoisomer thereof, wherein values for the variables (e.g., RG, RS, RE, PA, R^{1a}, R^{1b}, R^{2a}, R^{2b}, R^{3a}, R^{3b}, R⁴, A, W, Y, r, s, t, a′, w′, y′) are as described herein.

In some embodiments, the platform is a linker compound of Formula (III):

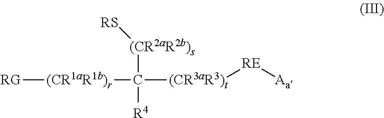

or a pharmaceutically acceptable salt, tautomer, solvate, or stereoisomer thereof, wherein values for the variables (e.g., RG, RS, RE, R^{1a}, R^{1b}, R^{2a}, R^{2b}, R^{3a}, R^{3b}, R⁴, A, r, s, t, a′) are as described herein.

Additional objects and advantages will be set forth in part in the description that follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate embodiments and together with the description serve to explain the principles described herein.

### 6. BRIEF DESCRIPTION OF THE DRAWINGS

FIGS. 1A-IL depict conjugator-antibody conjugate maleimide hydrolysis (ring opening) kinetics curves.

FIGS. 2A-2D depict stability data of conjugator-antibody conjugate 3-3 in pH 7.4 and 8.0 buffer with and without GSH.

FIGS. 3A-3D depict stability data of conjugator-antibody conjugate 3-4 in pH 7.4 and 8.0 buffer with and without GSH.

FIGS. 4A-4D depict stability data of conjugator-antibody conjugate 3-6 in pH 7.4 and 8.0 buffer with and without GSH.

FIGS. 5A-5D depict stability data of conjugator-antibody conjugate 3-7 in pH 7.4 and 8.0 buffer with and without GSH.

FIGS. 6A-6D depict stability data of conjugator-antibody conjugate 3-8 in pH 7.4 and 8.0 buffer with and without GSH.

FIGS. 7A-7D depict stability data of conjugator-antibody conjugate 3-9 in pH 7.4 and 8.0 buffer with and without GSH.

FIGS. 8A-8D depict stability data of conjugator-antibody conjugate 3-10 in pH 7.4 and 8.0 buffer with and without GSH.

FIGS. 9A-9D depict stability data of conjugator-antibody conjugate 3-11 in pH 7.4 and 8.0 buffer with and without GSH.

FIGS. 10A-10D depict stability data of conjugator-antibody conjugate 3-12 in pH 7.4 and 8.0 buffer with and without GSH.

FIGS. 11A-11D depict stability data of conjugator-antibody conjugate 3-13 in pH 7.4 and 8.0 buffer with and without GSH.

FIGS. 12A-12D depict stability data of conjugator-antibody conjugate 3-14 in pH 7.4 and 8.0 buffer with and without GSH.

FIGS. 13A-13B depict stability data of conjugator-antibody conjugate 3-3 in pH 5.5 histidine buffer for 168 h.

FIGS. 14A-14B depict stability data of conjugator-antibody conjugate 3-4 in pH 5.5 histidine buffer for 168 h.

FIGS. 15A-15B depict stability data of conjugator-antibody conjugate 3-6 in pH 5.5 histidine buffer for 168 h.

FIGS. 16A-16B depict stability data of conjugator-antibody conjugate 3-7 in pH 5.5 histidine buffer for 168 h.

FIGS. 17A-17D depict stability data of conjugator-antibody conjugate 3-8 in pH 5.5 histidine buffer for 168 h.

FIGS. 18A-18B depict stability data of conjugator-antibody conjugate 3-9 in pH 5.5 histidine buffer for 168 h.

FIGS. 19A-19B depict stability data of conjugator-antibody conjugate 3-10 in pH 5.5 histidine buffer for 168 h.

FIGS. 20A-20B depict stability data of conjugator-antibody conjugate 3-11 in pH 5.5 histidine buffer for 168 h.

FIGS. 21A-21B depict stability data of conjugator-antibody conjugate 3-12 in pH 5.5 histidine buffer for 168 h.

FIGS. 22A-22B depict stability data of conjugator-antibody conjugate 3-13 in pH 5.5 histidine buffer for 168 h.

FIGS. 23A-23B depict stability data of conjugator-antibody conjugate 3-14 in pH 5.5 histidine buffer for 168 h.

FIGS. 24A-24D depict stability data of ADC 4-6 in pH 7.4 or 8.0 GSH buffer.

FIGS. 25A-25D depict stability data of ADC 4-10 in pH 7.4 or 8.0 GSH buffer.

FIGS. 26A-26D depict stability data of ADC 4-11 in pH 7.4 or 8.0 GSH buffer.

FIGS. 27A-27D depict stability data of ADC 4-12 in pH 7.4 or 8.0 GSH buffer.

FIGS. 28A-28D depict stability data of ADC 4-13 in pH 7.4 or 8.0 GSH buffer.

FIGS. 29A-29D depict stability data of ADC 4-14 in pH 7.4 or 8.0 GSH buffer.

FIGS. 30A-30D depict stability data of ADC 4-16 in pH 7.4 or 8.0 GSH buffer.

FIGS. 31A-31D depict stability data of ADC 4-6 in formulation buffer.

FIGS. 32A-32D depict stability data of ADC 4-10 in formulation buffer.

FIGS. 33A-33B depict stability data of ADC 4-11 in formulation buffer.

FIGS. 34A-34B depict stability data of ADC 4-12 in formulation buffer.

FIGS. 35A-35B depict stability data of ADC 4-13 in formulation buffer.

FIGS. 36A-36B depict stability data of ADC 4-14 in formulation buffer.

FIGS. 37A-37B depict stability data of ADC 4-16 in formulation buffer.

FIG. 38 depicts ADC direct killing activity on a B7H3 high-expression cell line (H1650).

FIG. 39 depicts ADC direct killing activity on a B7H3 low-expression cell line (Capan-1).

FIG. 40 depicts ADC direct killing activity on a B7H3(−) cell line (MB-453).

FIG. 41 depicts ADC direct killing activity on a B7H3 high-expression cell line (H1650).

FIG. 42 depicts ADC direct killing activity on a B7H3 low-expression cell line (Capan-1).

FIG. 43 depicts ADC direct killing activity on a B7H3(−) cell line (MB-453).

FIG. 44 is a line graph showing ADC anti-tumor activity in an H1650 model of lung cancer.

FIG. 45 is a line graph showing dose-dependent ADC anti-tumor activity in an H1650 model of lung cancer.

FIG. 46 is a line graph showing payload release rate from ADCs in mouse plasma.

FIG. 47 is a line graph showing payload release rate from ADCs in human plasma.

FIG. 48 is a line graph showing the PK profile of ADCs.

### 7. DETAILED DESCRIPTION

Provided herein are antibody drug conjugates (ADCs) and covalent linkers and linker-payloads (platforms) for making ADCs. The ADCs may be used to treat a disease or disorder, such as cancer, such as by providing a composition comprising an ADC. The presently disclosed ADCs are more stable than known ADCs.

Some ADCs, such as those using interchain cysteine conjugation with a maleimide-based linker-payload, are known to undergo deconjugation under physiological conditions due to a retro-maleimide reaction. ADCs employing self-hydrolyzing maleimides have shown improved stability and pharmacological properties. In one example, conjugator-antibody conjugate 3-3 (see Table 3 below) underwent maleimide hydrolysis under pH 9.0 in one to three days. However, under such harsh conditions, post-translational modifications (e.g., oxidation) and deamination may occur on some antibodies, which may result in changes to physical properties, such as hydrophobicity, charge, and secondary and/or tertiary structure, and may lower the thermodynamic or kinetic barrier to unfold. Such changes may predispose an ADC to aggregation and other chemical modifications, which can alter the binding affinity, half-life, and efficacy of the ADC.

The presently disclosed conjugator-antibody conjugates can readily undergo maleimide hydrolysis under mild conditions (e.g., pH 7.0). The presently disclosed conjugators are not only conjugated with an antibody under conventional conditions (e.g., pH 6.5-7.0) but also maleimide hydrolysis can occur under conjugation conditions. Buffer exchange into a basic buffer for hydrolysis is not required. Adding a quenching reagent can be sufficient to stop the conjugation reaction. The presently disclosed conjugates can be subjected to buffer exchange into formulation buffer after maleimide hydrolysis is completed by monitoring via reduced LCMS.

### 7.1. Definitions

In the present disclosure, the following terms have the following meanings unless indicated otherwise. Unless defined otherwise, all technical and scientific terms used herein have the same meaning as is commonly understood by one of ordinary skill in the art to which this disclosure pertains. In the event that there is a plurality of definitions for a term provided herein, these Definitions prevail unless stated otherwise.

The term “antibody” herein is used in the broadest sense and specifically covers intact monoclonal antibodies, polyclonal antibodies, monospecific antibodies, multispecific antibodies (e.g., bispecific antibodies), and antibody fragments that exhibit the desired biological activity. An intact antibody has primarily two regions: a variable region and a constant region. The variable region binds to and interacts with a target antigen. The variable region includes a complementary determining region (CDR) that recognizes and binds to a specific binding site on a particular antigen. The constant region may be recognized by and interact with the immune system (see, e.g., Janeway et al., 2001, Immuno. Biology, 5th Ed., Garland Publishing, New York). An antibody can be of any type (e.g., IgG, IgE, IgM, IgD, and IgA), class (e.g., IgG1, IgG2, IgG3, IgG4, IgA1, and IgA2) or subclass. The antibody can be derived from any suitable species. In some embodiments, the antibody is of human or murine origin. An antibody can be, for example, human, humanized, or chimeric.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” is not to be construed as requiring production of the antibody by any particular method.

An “intact antibody” is one that comprises an antigen-binding variable region as well as a light chain constant domain (CL) and heavy chain constant domains, CH1, CH2, CH3, and CH4, as appropriate for the antibody class. The constant domains may be native sequence constant domains (e.g., human native sequence constant domains) or amino acid sequence variant thereof.

An “antibody fragment” comprises a portion of an intact antibody, comprising the antigen-binding or variable region thereof. Examples of antibody fragments include Fab, Fab′, F(ab′)₂, and Fv fragments, diabodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragment of any of the above which immunospecifically binds to a target antigen (e.g., a cancer cell antigen, a viral antigen or a microbial antigen).

An “antigen” is an entity to which an antibody specifically binds.

The terms “specific binding” and “specifically binds” mean that the antibody or antibody derivative will bind, in a highly selective manner, to its corresponding target antigen and not with the multitude of other antigens. Typically, the antibody or antibody derivative binds with an affinity of at least about 1×10⁻⁷ M, 10⁻⁸ M, 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely related antigen.

The term “inhibit” or “inhibition of” means to reduce by a measurable amount, or to prevent entirely.

The term “therapeutically effective amount” refers to an amount of a drug effective to treat a disease or disorder in a mammal. In the case of cancer, the therapeutically effective amount of a drug may reduce the number of cancer cells; reduce the tumor size; inhibit (i.e., slow to some extent or stop) cancer cell infiltration into peripheral organs; inhibit (i.e., slow to some extent or stop) tumor metastasis; inhibit, to some extent, tumor growth; and/or relieve to some extent one or more of the symptoms associated with the cancer. To the extent the drug may inhibit growth and/or kill existing cancer cells, it may be cytostatic and/or cytotoxic. For cancer therapy, efficacy can, for example, be measured by assessing the time to disease progression (TTP) and/or determining the response rate (RR).

The term “substantial” or “substantially” refers to a majority, i.e. >50% of a population, of a mixture or a sample, such as more than 50%, 55%, 60%, 65%, 70%, 75%, 80%, 85%, 90%, 91%, 92%, 93%, 94%, 95%, 96%, 97%, 98%, or 99% of a population.

The terms “intracellularly cleaved” and “intracellular cleavage” refer to a metabolic process or reaction inside a cell on a ligand drug conjugate (e.g., an antibody drug conjugate (ADC)), whereby the covalent attachment, e.g., the linker, between the drug moiety (D) and the ligand unit (e.g., an antibody (BA or Ab)) is broken, resulting in the free drug, or another metabolite of the conjugate dissociated from the antibody inside the cell. The cleaved moieties of the drug-linker-ligand conjugate are thus intracellular metabolites.

The term “cytotoxic activity” refers to a cell-killing, a cytostatic or an anti-proliferative effect of a drug-linker-ligand conjugate compound or an intracellular metabolite of a drug-linker-ligand conjugate. Cytotoxic activity may be expressed as the IC50 value, which is the concentration (molar or mass) per unit volume at which half the cells survive.

The term “cytotoxic agent” as used herein refers to a substance that inhibits the function of cells and/or causes destruction of cells. The term is intended to include radioactive isotopes (e.g., 211At, 131I, 125I, 90Y, 186Re, 188Re, 153Sm, 212Bi, 32P, 60C, and radioactive isotopes of Lu), chemotherapeutic agents, and toxins such as small molecule toxins or enzymatically active toxins of bacterial, fungal, plant, or animal origin, including synthetic analogs and derivatives thereof.

The terms “cancer” and “cancerous” refer to or describe the physiological condition or disorder in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer); lung cancer including small-cell lung cancer, non-small cell lung cancer (“NSCLC”), adenocarcinoma of the lung, and squamous carcinoma of the lung; cancer of the peritoneum; hepatocellular cancer; gastric or stomach cancer including gastrointestinal cancer; pancreatic cancer; glioblastoma; cervical cancer; ovarian cancer; liver cancer; bladder cancer; hepatoma; breast cancer; colon cancer; rectal cancer; colorectal cancer; endometrial or uterine carcinoma; salivary gland carcinoma; kidney or renal cancer; prostate cancer; vulval cancer; thyroid cancer; hepatic carcinoma; anal carcinoma; penile carcinoma; as well as head and neck cancer.

An “autoimmune disease” herein is a disease or disorder arising from and directed against an individual's own tissues or proteins.

Examples of a “patient” include, but are not limited to, mammals such as a human, rat, mouse, guinea pig, monkey, pig, goat, cow, horse, dog, or cat, and birds or fowl. In an embodiment, the patient is a human.

The terms “treat” or “treatment,” unless otherwise indicated by context, refer to therapeutic treatment and prophylactic measures to prevent relapse, wherein the object is to inhibit or slow down (lessen) an undesired physiological change or disorder, such as the development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder.

In the context of cancer, the term “treating” includes any or all of inhibiting growth of tumor cells, cancer cells, or of a tumor, inhibiting replication of tumor cells or cancer cells, lessening of overall tumor burden or decreasing the number of cancerous cells, and ameliorating one or more symptoms associated with the disease.

In the context of an autoimmune disease, the term “treating” includes any or all of inhibiting replication of cells associated with an autoimmune disease state including, but not limited to, cells that produce an autoimmune antibody, lessening the autoimmune-antibody burden, and ameliorating one or more symptoms of an autoimmune disease.

As used herein, and in the specification and the accompanying claims, the indefinite articles “a” and “an” and the definite article “the” include the plural as well as single referents, unless the context clearly indicates otherwise.

As used herein, and unless otherwise specified, the terms “about” and “approximately,” when used in connection with amounts, or weight percentage of ingredients of a composition, mean an amount or weight percent that is recognized by one of ordinary skill in the art to provide a pharmacological effect equivalent to that obtained from the specified amount or weight percent. In certain embodiments, the terms “about” and “approximately,” when used in this context, contemplate an amount or weight percent within 30%, within 20%, within 15%, within 10%, or within 5%, of the specified amount or weight percent.

As used herein, and unless otherwise specified, the terms “about” and “approximately,” when used in connection with a numeric value or range of values that is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to +0.2° 20 while still describing the particular XRPD peak.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 carbon atoms. Representative alkyl groups include-methyl, -ethyl, -n-propyl, -n-butyl, -n-pentyl, and n-hexyl; saturated branched alkyls include-isopropyl, -sec-butyl, -isobutyl, -tert-butyl, -isopentyl, 2-methylpentyl, 3-methylpentyl, 4-methylpentyl, 2,3-dimethylbutyl and the like. Examples of unsaturated alkyl groups include, but are not limited to, vinyl, allyl, CH═CH(CH₃), —CH═C(CH₃)₂, —C(CH₃)═CH₂, —C(CH₃)═CH(CH₃), C(CH₂CH₃)═CH₂, C≡CH, —C≡C(CH₃), —C≡C(CH₂CH₃), —CH₂C≡CH, —CH₂C≡C(CH₃), and CH₂C≡C(CH₂CH₃), among others. An alkyl group can be substituted or unsubstituted. In certain embodiments, when the alkyl groups described herein are said to be “substituted,” they may be substituted with any substituent or substituents as those found in the compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂; or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₈)alkenyls include-vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, 3-octenyl and the like. The double bond of an alkenyl group can be unconjugated or conjugated to another unsaturated group. An alkenyl group can be unsubstituted or substituted.

A “cycloalkyl” group is a saturated or a partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, and hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6 to 14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or 6 to 10 atoms in the ring portions of the groups. Suitable heteroatoms include oxygen, sulfur, and nitrogen. In certain embodiments, the heteroaryl ring system is monocyclic or bicyclic. Non-limiting examples include, but are not limited to, groups such as pyrrolyl, pyrazolyl, imidazolyl, triazolyl, tetrazolyl, oxazolyl, isoxazolyl, thiazolyl, pyrrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3H-imidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated, and saturated ring systems, such as, for example, imidazolyl, imidazolinyl, and imidazolidinyl groups. The term “heterocyclyl” includes fused ring species, including those comprising fused aromatic and non-aromatic groups, such as, for example, benzotriazolyl, 2,3-dihydrobenzo[1,4]dioxinyl, and benzo[1,3]dioxolyl. The term also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridin-2(3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula -alkyl-aryl, wherein alkyl and aryl are defined above. Substituted aralkyl groups may be substituted at the alkyl, the aryl, or both the alkyl and the aryl portions of the group. Representative aralkyl groups include, but are not limited to, benzyl and phenethyl groups and fused (cycloalkylaryl)alkyl groups such as 4-ethyl-indanyl.

A “heterocyclylalkyl” group is a radical of the formula -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocyclylalkyl groups include, but are not limited to, 4-ethyl-morpholinyl, 4-propylmorpholinyl, furan-2-yl methyl, furan-3-yl methyl, pyrdine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

A “hydroxyalkyl” group is an alkyl group as described above substituted with one or more hydroxy groups.

An “alkoxy” group is O(alkyl), wherein alkyl is defined above.

An “alkoxyalkyl” group is (alkyl) O(alkyl), wherein alkyl is defined above.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to, ethynyl, propynyl, and butynyl.

As used herein, “haloalkyl” refers to alkyl, as defined above, wherein the alkyl includes at least one substituent selected from a halogen, for example, fluorine (F), chlorine (Cl), bromine (Br), or iodine (I). Examples of haloalkyl include, but are not limited to, —CF₃, —CH₂CF₃, —CCl₂F, and —CCl₃.

As used herein, “haloalkoxy” refers to alkoxy, as defined above, wherein the alkoxy includes at least one substituent selected from a halogen, e.g., F, Cl, Br, or I.

As used herein, “arylalkyl” refers to a monovalent moiety that is a radical of an alkyl compound, wherein the alkyl compound is substituted with an aromatic substituent, i.e., the aromatic compound includes a single bond to an alkyl group and wherein the radical is localized on the alkyl group. An arylalkyl group bonds to the illustrated chemical structure via the alkyl group. An arylalkyl can be represented by the structure, e.g., B—CH₂—, B—CH₂—CH₂—, B—CH₂—CH₂—CH₂—, B—CH₂—CH₂—CH₂—CH₂—, B—CH(CH₃)—CH₂—CH₂—, B—CH₂—CH(CH₃)—CH₂—, wherein B is an aromatic moiety, e.g., phenyl. Arylalkyl is optionally substituted, i.e., the aryl group and/or the alkyl group, can be substituted as disclosed herein. Examples of arylalkyl include, but are not limited to, benzyl.

As used herein, “alkylaryl” refers to a monovalent moiety that is a radical of an aryl compound, wherein the aryl compound is substituted with an alkyl substituent, i.e., the aryl compound includes a single bond to an alkyl group and wherein the radical is localized on the aryl group. An alkylaryl group bonds to the illustrated chemical structure via the aryl group. An alkylaryl can be represented by the structure, e.g., —B—CH₃, —B—CH₂—CH₃, —B—CH₂—CH₂—CH₃, —B—CH₂—CH₂—CH₂—CH₃, —B—CH(CH₃)—CH₂—CH₃, —B—CH₂—CH(CH₃)—CH₃, wherein B is an aromatic moiety, e.g., phenyl. Alkylaryl is optionally substituted, i.e., the aryl group and/or the alkyl group, can be substituted as disclosed herein. Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g., C₆H₅—O—, for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy, and 6 to 10 ring carbon atoms, i.e., C₆₋₁₀ aryloxy. Examples of aryloxy moieties include, but are not limited to phenoxy, naphthoxy, and anthroxy.

An “amino” group is a radical of the formula NH₂.

A “hydroxyl amine” group is a radical of the formula N(R^{#})OH or NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula —N(R^{#})O-alkyl or —NHO-alkyl, wherein R^{#} is as defined above.

An “aralkoxyamine” group is a radical of the formula N(R^{#})O-aryl or NHOaryl, wherein R^{#} is as defined above.

An “alkylamine” group is a radical of the formula NHalkyl or N(alkyl)₂, wherein each alkyl is independently as defined above.

An “aminocarbonyl” group is a radical of the formula —C(═O)N(R^{#})₂, —C(═O)NH(R^{#}), or C(═O)NH₂, wherein each R^{#} is as defined above.

An “acylamino” group is a radical of the formula NHC(═O)(R^{#}) or N(alkyl)C(═O)(R^{#}), wherein each alkyl and R″ are independently as defined above.

An “O(alkyl)aminocarbonyl” group is a radical of the formula —O(alkyl)C(═O)N(R^{#})₂, —O(alkyl)C(═O)NH(R^{#}), or —O(alkyl)C(═O)NH₂, wherein each R^{#} is independently as defined above.

An “N-oxide” group is a radical of the formula —N⁺—O⁻.

A “carboxy” group is a radical of the formula C(═O)OH.

A “ketone” group is a radical of the formula C(═O)(R^{#}), wherein R^{#} is as defined above.

An “aldehyde” group is a radical of the formula —CH(═O).

An “ester” group is a radical of the formula C(═O)O(R^{#}) or OC(═O)(R^{#}), wherein R^{#} is as defined above.

A “urea” group is a radical of the formula —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or NHC(═O)NH₂^{#}, wherein each alkyl and R^{#} are independently as defined above.

An “imine” group is a radical of the formula —N═C(R^{#})₂ or —C(R^{#})═N(R^{#}), wherein each R^{#} is independently as defined above.

An “imide” group is a radical of the formula —C(═O)N(R#)C(═O)(R^{#}) or N((C═O)(R^{#}))₂, wherein each R″ is independently as defined above.

A “urethane” group is a radical of the formula —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})—NH, or —NHC(R^{#})—NH, wherein each R^{#} is independently as defined above.

A “guanidine” group is a radical of the formula —N(R^{#})C(═N(R^{#}))N(R^{#})₂, —NHC(═N(R^{#})N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#})NH₂, —NHC(═NH)NH(R^{#}), —NHC(═NH)NH₂, —N═C(N(R^{#})₂)₂, —N═C(NH(R^{#}))₂, or —N═C(NH₂)₂, wherein each R^{#} is independently as defined above.

An “enamine” group is a radical of the formula —N(R^{#})C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R^{#})₂, —C(R^{#})═C(R^{#})(N(R^{#})₂), C(R^{#})═C(R^{#})(NH(R^{#})) or —C(R^{#})═C(R^{#})(NH₂), wherein each R^{#} is independently as defined above.

An “oxime” group is a radical of the formula —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula —N(R^{#})N(R^{#})₂, —NHN(R^{#})₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula —C(═N—N(R^{#})₂)(R^{#})₂, —C(═NNH(R^{#}))(R^{#})₂, —C(═N—NH₂)(R^{#})₂, —N(R^{#})(N═C(R^{#})₂), or —NH(N═C(R^{#})₂), wherein each R^{#} is independently as defined above.

An “azide” group is a radical of the formula —N₃.

An “isocyanate” group is a radical of the formula N═C═O.

An “isothiocyanate” group is a radical of the formula N═C═S.

A “cyanate” group is a radical of the formula OCN.

A “thiocyanate” group is a radical of the formula SCN.

A “thioether” group is a radical of the formula —S(R^{#}), wherein R^{#} is as defined above.

A “thiocarbonyl” group is a radical of the formula —C(═S)(R^{#}), wherein R^{#} is as defined above.

A “sulfinyl” group is a radical of the formula —S(═O)(R^{#}), wherein R^{#} is as defined above.

A “sulfone” group is a radical of the formula —S(═O)₂ (R^{#}), wherein R^{#} is as defined above.

A “sulfonylamino” group is a radical of the formula —NHSO₂(R^{#}) or —N(alkyl)SO₂(R^{#}), wherein each alkyl and R^{#} are defined above.

A “sulfonamide” group is a radical of the formula —S(═O)₂N(R^{#})₂, —S(═O)₂NH(R^{#}), or —S(═O)₂NH₂, wherein each R^{#} is independently as defined above.

A “phosphonate” group is a radical of the formula —P(═O)(O(R^{#}))₂, —P(═O)(OH)₂, —OP(═O)(O(R^{#}))(R^{#}), or —OP(═O)(OH)(R^{#}), wherein each R″ is independently as defined above.

A “phosphine” group is a radical of the formula —P(R^{#})₂, wherein each R^{#} is independently as defined above.

When the groups described herein, with the exception of alkyl groups, are said to be “substituted,” they may be substituted with any appropriate substituent or substituents. Illustrative examples of substituents are those found in the compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether, imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid or base and an organic acid or base.

As used herein and unless otherwise indicated, the term “solvate” means a compound, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a compound, or a salt thereof, that further includes a stoichiometric or non-stoichiometric amount of water bound by non-covalent intermolecular forces.

As used herein and unless otherwise indicated, the term “prodrug” means a compound derivative that can hydrolyze, oxidize, or otherwise react under biological conditions (in vitro or in vivo) to provide an active compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters may be formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (WileyInterscience, New York, 1981); Wilen, S. H., et al., Tetrahedron 33:2725 (1977); Eliel, E. L., Stereochemistry of Carbon Compounds (McGrawHill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted that the compounds can include E and Z isomers, or a mixture thereof, and cis and trans isomers, or a mixture thereof. In certain embodiments, the compounds are isolated as either the cis or trans isomer. In other embodiments, the compounds are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in an aqueous solution, pyrazoles may exhibit the following isomeric forms, which are referred to as tautomers of each other:

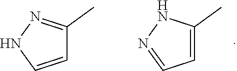

As readily understood by one skilled in the art, a wide variety of functional groups and other structures may exhibit tautomerism and all tautomers of the compounds are within the scope of the present disclosure.

It should also be noted the compounds can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

It should be noted that if there is a discrepancy between a depicted structure and a name for that structure, the depicted structure is to be accorded more weight.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “sugar” or “sugar group” or “sugar residue” refers to a carbohydrate moiety which may comprise 3-carbon (those) units, 4-carbon (tetrose) units, 5-carbon (pentose) units, 6-carbon (hexose) units, 7-carbon (heptose) units, or combinations thereof, and may be a monosaccharide, a disaccharide, a trisaccharide, a tetrasaccharide, a pentasaccharide, an oligosaccharide, or any other polysaccharide. In some instances, a “sugar” or “sugar group” or “sugar residue” comprises furanoses (e.g., ribofuranose, fructofuranose) or pyranoses (e.g., glucopyranose, galactopyranose), or a combination thereof. In some instances, a “sugar” or “sugar group” or “sugar residue” comprises aldoses or ketoses, or a combination thereof. Non-limiting examples of monosaccharides include ribose, deoxyribose, xylose, arabinose, glucose, mannose, galactose, and fructose. Non-limiting examples of disaccharides include sucrose, maltose, lactose, lactulose, and trehalose. Other “sugars” or “sugar groups” or “sugar residues” include polysaccharides and/or oligosaccharides, including, but not limited to, amylose, amylopectin, glycogen, inulin, and cellulose. In some instances, a “sugar” or “sugar group” or “sugar residue” is an amino-sugar. In some instances, a “sugar” or “sugar group” or “sugar residue” is a glucamine residue (1-amino-1-deoxy-D-glucitol) linked to the rest of molecule via its amino group to form an amide linkage with the rest of the molecule (i.e., a glucamide).

Certain groups, moieties, substituents, and atoms are depicted with a wiggly line, e.g., that intersects a bond or bonds, to indicate the atom through which the groups, moieties, substituents, atoms are bonded. For example, a phenyl group that is substituted with a propyl group depicted as:

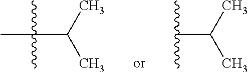

has the following structure:

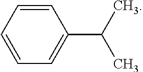

Illustrations showing substituents bonded to a non-cyclic group through a bond between two atoms are meant to indicate, unless specified otherwise, that the substituent may be bonded to either atom of the bond through which the substituent bond passes, according to techniques set forth herein or which are known in the field to which the instant disclosure pertains. Thus, for example,

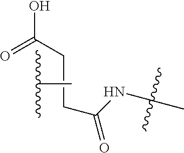

encompasses

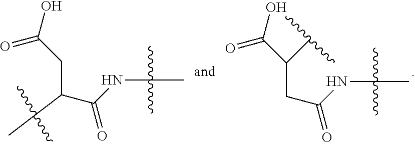 [SMILES: *NC(=O)C(*)CC(=O)O ; *NC(=O)CC(*)C(=O)O]

As used herein, “binding agent” refers to any molecule, e.g., antibody, capable of binding with specificity to a given binding partner, e.g., antigen.

As used herein, the term “amino acid” refers to an organic compound that contains amino (—NH₂) and carboxyl (—COOH) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, beta-alanine, ornithine, and citrulline.

As used herein “peptide”, in its various grammatical forms, is defined in its broadest sense to refer to a compound of two or more subunit amino acids, amino acid analogs, or other peptidomimetics. The subunits may be linked by peptide bonds or by other bonds, for example, ester, ether, and the like. As used herein, the term “amino acid” refers to either natural and/or unnatural, proteinogenic or non-proteinogenic, or synthetic amino acids, including glycine and both the D and L optical isomers, and amino acid analogs and peptidomimetics. If the peptide chain is short, e.g., two, three or more amino acids, it is commonly called an oligopeptide. If the peptide chain is longer, the peptide is typically called a polypeptide or a protein. Full-length proteins, analogs, mutants, and fragments thereof are encompassed by the definition. The terms also include post-expression modifications of the polypeptide, for example, glycosylation, acetylation, phosphorylation, and the like. Furthermore, as ionizable amino and carboxyl groups are present in the molecule, a particular peptide may be obtained as an acidic or basic salt, or in neutral form. A peptide may be obtained directly from the source organism or may be recombinantly or synthetically produced.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

As used herein, the term “anti-HER2 antibody” refers to an antibody selectively binding to the HER2 receptor, e.g., trastuzumab (Herceptin). In one embodiment, trastuzumab can be made and used as described in U.S. Pat. Nos. 6,407,213 and 5,821,337, the entire disclosures of which are incorporated herein by reference.

As used herein, the term “anti-HER3 antibody” refers to an antibody selectively binding to the HER3 receptor, e.g., patritumab. In one embodiment, patritumab can be made and used as described in U.S. Pat. No. 7,705,130, the entire disclosure of which is incorporated herein by reference.

As used herein, the term “anti-PTK7 antibody” refers to an antibody selectively binding to the PTK7 receptor, e.g., cofetuzumab. In one embodiment, cofetuzumab can be made and used as described in U.S. Pat. No. 9,777,070, the entire disclosure of which is incorporated herein by reference.

As used herein, the term “ifinatamab” refers to an antibody selectively binding to the B7H3 receptor. In one embodiment, ifinatamab can be made and used as described in U.S. Pat. No. 10,117,952 or WO2022102695, the entire disclosures of which are incorporated herein by reference.

As used herein, the term “cell-killing activity” refers to the activity that decreases or reduces the cell viability of the tested cell line.

In the claims that follow and in the preceding description, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments.

### 7.2. Conjugates

In embodiments, a conjugate, or a pharmaceutically acceptable salt, tautomer, solvate, or stereoisomer thereof, includes a protein linked to at least one payload or payload residue (also referred to herein as a Drug unit) and linked to at least one hydrophilic moiety via a covalent linker. The covalent linker is bonded directly or indirectly to each of the protein, the payload residue, and the hydrophilic moiety. In some embodiments, the protein is a binding agent, such as an antibody or antigen binding fragment thereof.

In some embodiments, the protein is bonded directly to a covalent linker, such as a linker set forth herein. In such cases, the binding agent is one bond position away from the covalent linker. The covalent linker may also be bonded directly to a payload residue such that the covalent linker is one bond position away from a payload residue. The payload may be any payload set forth herein. In some embodiments, the covalent linker is also bonded directly to a hydrophilic moiety such that the covalent linker is one bond position away from a hydrophilic moiety. The hydrophilic moiety may be any hydrophilic moiety (HG) set forth herein.

In some embodiments, the binding agent is bonded indirectly to a covalent linker such that the binding agent is more than one bond position away from the covalent linker. In such cases, the binding agent is bonded through another moiety to the covalent linker. For example, the binding agent may be bonded to a maleimide group which is bonded to a polyethylene glycol group which is bonded to the covalent linker.

In some examples, the covalent linker is also bonded indirectly to a payload residue such that the covalent linker is more than one bond position away from a payload residue. The covalent linker is bonded through another moiety to the payload. For example, the covalent linker may be bonded to a dipeptide, such as but not limited to Val-Ala or Val-Cit, which may be bonded to PAB which may be bonded to the payload residue.

In some embodiments, the covalent linker is bonded indirectly to a hydrophilic moiety such that the covalent linker is more than one bond position away from a hydrophilic moiety. The covalent linker is bonded through another moiety to the hydrophilic moiety.

### 7.2.1. Aspect 1

Provided herein are ADCs, e.g., for use in therapy, such as cancer therapy.

One embodiment is an ADC compound of Formula (I):

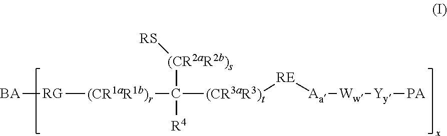

or a pharmaceutically acceptable salt, tautomer, solvate, or stereoisomer thereof,
BA is a binding agent selected from a humanized, chimeric, or human antibody or an antigen binding fragment thereof;
RG is a reactive group residue; RS is an open-ring stabilizing group;
RE is an open-ring enhancer;
each of R^{1a} and R^{1b} is, independently, H; substituted or unsubstituted C₁₋₄ alkyl; substituted or unsubstituted C₃₋₅ cycloalkyl; or R^{1a} and R^{1b} together with the atom to which they are attached form a substituted or unsubstituted C₃₋₅ cycloalkyl;
each of R^{2a} and R^{2b} is, independently, H; substituted or unsubstituted C₁₋₄ alkyl; substituted or unsubstituted C₃₋₅ cycloalkyl; or R^{2a} and R^{2b} together with the atom to which they are attached form a substituted or unsubstituted C₃₋₅ cycloalkyl;
each of R^{3a} and R^{3b} is, independently, H; substituted or unsubstituted C₁₋₄ alkyl; substituted or unsubstituted C₃₋₅ cycloalkyl; or R^{3a} and R^{3b} together with the atom to which they are attached form a substituted or unsubstituted C₃₋₅ cycloalkyl;
R⁴ is H; substituted or unsubstituted C₁₋₄ alkyl; or substituted or unsubstituted C₃₋₅ cycloalkyl;
each of r, s, and tis, independently, 0, 1, or 2;
A is a Stretcher unit residue;
subscript a′ is 0 or 1;
W is a Cleavable unit;
subscript w′ is 0 or 1;
Y is a Spacer unit;
subscript y′ is 0 or 1;
PA is a payload residue; and
subscript x is from 1 to 15.

In one embodiment, subscript x is from 1 to 12. In one embodiment, subscript x is from 1 to 10. In one embodiment, subscript x is from 2 to 10. In one embodiment, subscript x is from 3 to 10. In one embodiment, subscript x is from 4 to 10. In one embodiment, subscript x is from 4 to 9. In one embodiment, subscript x is from 4 to 8.

In some embodiments, each of Ria, R^{1b}, R^{2a}, R^{2b}, R^{3a}, R^{3b}, and R⁴ is, independently, H.

In some embodiments, RS is —NR^{5a}R^{5b}, wherein each of R^{5a} and R^{5b} is, independently, H, or substituted or unsubstituted C₁₋₄ alkyl.

In some embodiments, RS is an amino (—NH₂) group or —N(CH₃)₂.

In some embodiments, RS is an amino group.

In some embodiments, RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—; and R⁶ is H or substituted or unsubstituted C₁₋₄ alkyl.

In some embodiments, R⁶ is H, methyl, ethyl, or isopropyl.

In some embodiments, RE is —OC(═O)NR⁶—.

In some embodiments, RE is —OC(═O)NH—.

In some embodiments, RG is

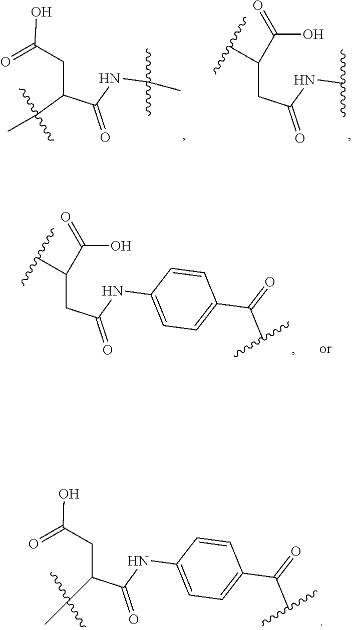 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1]

In some embodiments, RG is

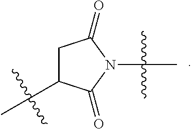 [SMILES: *C1CC(=O)N(*)C1=O]

### -(Succinimid-3-yl-N)—, or

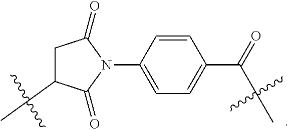 [SMILES: *C(=O)c1ccc(N2C(=O)CC(*)C2=O)cc1]

In some embodiments, r is 0.

In some embodiments, s is 1.

In some embodiments, t is 1 or 2.

In some embodiments, r is 0, s is 1, and t is 1 or 2.

In some embodiments, A is a bond, —(CH₂)ₙ—C(═O)—, —CH₂—C(═O)—NH—(CH₂)ₙ—C(═O)—, —(CH₂CH₂O)—CH₂CH₂—C(═O)—, —CH [—(CH₂)ₙ—COOH]—C(═O)—, —CH₂—C(═O)—NH—(CH₂)ₙ—C(═O)—NH—(CH₂)—C(═O)—, or —C(═O)—(CH₂)ₙ—C(═O)—, wherein each n independently represents an integer of 1, 2, 3, 4, or 5.

In some embodiments, W_{w′} is one of the following formulas:

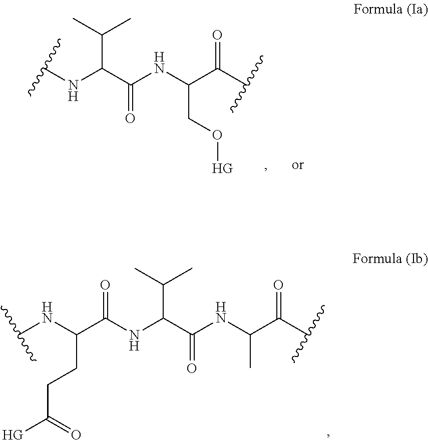

and HG is a hydrophilic moiety or hydrogen.

In some embodiments, HG is a saccharide, phosphate ester, sulfate ester, a phosphodiester, or a phosphonate.

In some embodiments, HG is a saccharide, and the saccharide is β-D-galactose, N-acetyl-P-D-galactosamine, N-acetyl-a-D-galactosamine, N-acetyl-P-D-glucosamine, β-D-glucuronic acid, a-L-iduronic acid, a-D-galactose, a-D-glucose, β-D-glucose, a-D-mannose, β-D-mannose, a-L-fucose, β-D-xylose, a neuraminic acid, sulfate, phosphate, carboxyl, amino, or an O-acetyl modification thereof. In some embodiments, HG is selected from β-D-galactose and β-D-glucuronic acid.

In some embodiments, HG is

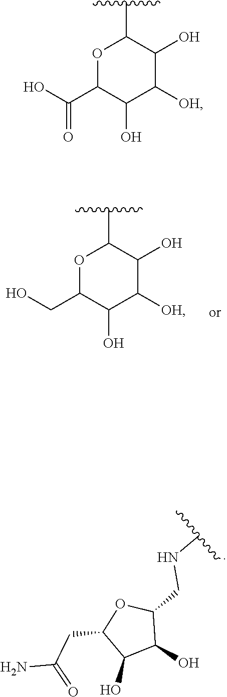 [SMILES: *C1OC(C(=O)O)C(O)C(O)C1O ; *C1OC(CO)C(O)C(O)C1O ; "or" ; *NC[C@H]1O[C@@H](CC(N)=O)[C@H](O)[C@@H]1O]

In some embodiments, Y_{y′} is a PAB unit.

In some embodiments, the Stretcher unit (-A-) is present and extends the framework of the covalent linker to provide more distance between a self-stabilizing linker assembly and a Drug unit (payload or payload residue). The self-stabilizing linker assembly may include the components of Formula (I) other than the binding agent (BA), Stretcher unit (A), Cleavable unit (W), Spacer unit (Y), and payload residue (PA). In embodiments, the self-stabilizing linker includes the reactive group residue (RG), open-ring stabilizing group (RS), open-ring enhancer (RE), and the carbon atoms and R groups connecting RG, RS, and/or RE. A Stretcher unit may link the self-stabilizing linker assembly to the Cleavable unit when the Cleavable unit is present, the self-stabilizing linker assembly to the Spacer unit when the Cleavable unit is absent and the Spacer unit is present, or the self-stabilizing linker assembly to the Drug unit when both the Cleavable unit and the Spacer unit are absent. A Stretcher unit may attach to more than one Cleavable unit, Spacer unit, and/or Drug unit. The self-stabilizing linker assembly, Cleavable unit, Spacer unit, and Drug unit may be any self-stabilizing linker assembly, Cleavable unit, Spacer unit, and Drug unit, respectively, described herein.

The Stretcher unit may alter the physiochemical properties of the Drug-Linker depending on the components of the Stretcher unit. In some implementations, the Stretcher unit may increase the solubility of the Drug-Linker and may include one or more solubility-enhancing groups such as ionic groups or water-soluble polymers. Water-soluble polymers may be soluble in water at room temperature and may include poly(ethylene)glycol groups as well as other polymers such as polyethyleneimines.

A Stretcher unit may comprise one or more stretcher groups. Examples of stretcher groups include, for example, —NH—C₁₋₁₀alkylene-, —NH—C₁₋₁₀ alkylene-NH—C(O)—C₁₋₁₀ alkylene-, —NH—C₁₋₁₀alkylene-C(O)—NH—C₁₋₁₀alkylene-, —NH—(CH₂CH₂O)ᵤ—, —NH—(CH₂CH₂O), —CH₂—, —NH—(CH₂CH₂NH)ᵤ—(CH₂)ᵤ—NH—(CH₂CH₂NH)ᵤ—(CH₂)ᵤ—NH—C(O)—(CH₂)ᵤ—NH—(C₃-C₈ carbocyclo)-, —NH-(arylene)-, and —NH—(C₃-C₈ heterocyclo-)—, wherein each u is independently 1-10.

In some embodiments, the Cleavable unit (—W_{w′}—) is present and may link the self-stabilizing linker assembly to the Spacer unit when the Spacer unit is present or link the self-stabilizing linker assembly to the Drug unit when the Spacer unit is absent. The linkage from the self-stabilizing linker assembly to the Spacer unit or to the Drug unit can be directly from the self-stabilizing linker assembly when the Stretcher unit is absent or via the Stretcher unit if the Stretcher unit is present.

In some embodiments, the Cleavable unit is directly conjugated to the self-stabilizing linker assembly on one end and to the Drug unit on the other end. In some embodiments, the Cleavable unit is directly conjugated to the Stretcher unit on one end and to the Drug unit on the other end. In further embodiments, the Cleavable unit is directly conjugated to the Stretcher unit on one end and to the Spacer unit on the other end. In still further embodiments, the Cleavable unit is directly conjugated to the self-stabilizing linker assembly on one end and to the Spacer unit on the other end. In embodiments, the Stretcher Unit and/or Spacer unit may be absent.

The Cleavable unit may form a cleavable bond with a Drug unit (PA) or a Spacer unit. Reactive groups for forming cleavable bonds can include, for example, sulfhydryl groups to form disulfide bonds; aldehyde, ketone, or hydrazine groups to form hydrazone bonds; carboxylic or amino groups to form peptide bonds; and carboxylic or hydroxy groups to form ester bonds.

The Cleavable unit may include a disulfide-containing linker cleavable through disulfide exchange, an acid-labile linker at acidic pH, or a linker cleavable by an enzyme such as a hydrolase, peptidase, esterase, or glucoronidase. The Cleavable unit may include one or more cleavage sites.

In some embodiments, the Cleavable unit includes one or more amino acids, such as 1 to 12. The Cleavable unit can include, for example, a monopeptide, dipeptide, tripeptide, tetrapeptide, pentapeptide, hexapeptide, heptapeptide, octapeptide, nonapeptide, decapeptide, undecapeptide, or dodecapeptide unit.

Each amino acid may be a natural or unnatural amino acid, and/or a D- or L-isomer of the same, provided that a cleavable bond is available. In some embodiments, the Cleavable unit includes only natural amino acids. Each amino acid may be a proteinogenic or non-proteinogenic amino acid.

In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, β-alanine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof. In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, and selenocysteine. In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, and valine.

In some embodiments, each amino acid is independently selected from an L isomer of the following natural amino acids: alanine, arginine, aspartic acid, asparagine, histidine, glycine, glutamic acid, glutamine, phenylalanine, lysine, leucine, serine, tyrosine, threonine, isoleucine, tryptophan and valine. In some embodiments, each amino acid is a D isomer of the following natural amino acids: alanine, arginine, aspartic acid, asparagine, histidine, glycine, glutamic acid, glutamine, phenylalanine, lysine, leucine, serine, tyrosine, threonine, isoleucine, tryptophan and valine.

In embodiments, the Cleavable unit is the dipeptide-Val-Cit-, -Phe-Lys-, or -Val-Ala.

In some embodiments, the Cleavable unit includes one or two terminal amino acids and is linked to the Drug unit and/or Spacer unit through functional units present in a terminal amino acid, e.g., its carboxylic acid or amino termini.

In some embodiments, the bond between the Cleavable unit and the Drug unit can be enzymatically cleaved by one or more enzymes, including a tumor-associated protease, to liberate the Drug unit (-PA), which may be protonated in vivo upon release to provide a Drug (PA).

Useful Cleavable units may be designed to optimize their selectivity for enzymatic cleavage by a particular enzyme, such as a tumor-associated protease. In one embodiment, a linkage between the Cleavable unit and the Drug unit or Spacer unit is one for which cleavage is catalyzed by cathepsin B, C, and/or D, or a plasmin protease.

In some embodiments, the Spacer unit (-Y_{y′}-) is present and extends the framework of the covalent linker. A Spacer unit may link a Cleavable unit to the Drug unit or a Stretcher unit to the Drug unit or a self-stabilizing linker assembly to a Drug unit. The Spacer unit may include one or more self-immolative or non-self-immolative groups. In some embodiments, the Spacer unit includes one or more self-immolative groups. In this context, the term “self-immolative group” refers to a bifunctional chemical moiety that is capable of covalently linking together two spaced chemical moieties into a normally stable tripartite molecule. The self-immolative group will spontaneously separate from the second chemical moiety if its bond to the first moiety is cleaved. In other embodiments, the Spacer unit is not self-immolative. In such embodiments, part or all of the Spacer unit remains attached to the Drug unit.

In some embodiments, -Y_{y′}- is a self-immolative group and is linked to a Cleavable unit via a methylene carbon atom of the self-immolative group, and linked directly to the Drug unit via a carbonate, carbamate, or ether group.

In some embodiments, -Y_{y′}- is a p-aminobenzyl alcohol (PAB) unit (e.g., —NH—(C₆H₄)—CH₂—O—C(═O)—. The phenylene portion of the PAB unit is optionally substituted with —C₁-C₈alkyl, —O—(C₁-C₈ alkyl), -halogen, -nitro, or -cyano.

In another embodiment, -Y_{y′}- is a carbonate group.

Other examples of self-immolative groups include, but are not limited to, aromatic compounds that are electronically similar to the PAB unit such as 2-aminoimidazol-5-methanol derivatives (see, e.g., Hay et al., 1999, Bioorg. Med. Chem. Lett. 9:2237) and ortho- or para-aminobenzylacetals. Suitable Spacer units include those that undergo cyclization upon amide bond hydrolysis, such as substituted and unsubstituted 4-aminobutyric acid amides (see, e.g., Rodrigues et al., 1995, Chemistry Biology 2:223), appropriately substituted bicyclo[2.2.1] and bicyclo[2.2.2] ring systems (see, e.g., Storm et al., 1972, J. Amer. Chem. Soc. 94:5815), and 2-aminophenylpropionic acid amides (see, e.g., Amsberry et al., 1990, J. Org. Chem. 55:5867). Elimination of amine-containing drugs that are substituted at the α-position of glycine (see, e.g., Kingsbury et al., 1984, J. Med. Chem. 27:1447) are also examples of suitable self-immolative groups.

Other suitable Spacer units are disclosed in U.S. Publication No. 2005-0238649, the disclosure of which is incorporated by reference herein.

Suitable Stretcher units, Cleavable units, and Spacer units for use with the presently disclosed linkers, platforms, and ADCs care described in WO 2004/010957, WO 2007/038658, WO 2005/112919, U.S. Pat. Nos. 6,214,345, 7,659,241, 7,498,298, 7,968,687, and 8,163,888, and U.S. Publication Nos. 2009-0111756, 2009-0018086, and 2009-0274713, each of which is incorporated herein by reference in its entirety and for all purposes.

### Binding Agents

Provided herein are binding agents (BA), e.g., for use in an ADC described herein.

Compounds of Formula (I) may include any BA described herein.

In some embodiments, BA is an antibody or antigen binding fragment thereof, e.g., a humanized, chimeric, or human antibody or an antigen binding fragment thereof.

In some embodiments, the antibody or antigen binding fragment thereof specifically binds human B7H3. In some embodiments, the antibody or antigen binding fragment thereof is ifinatamab.

In some embodiments, the antibody or antigen binding fragment thereof specifically binds PTK7. In some embodiments, the antibody or antigen binding fragment thereof is cofetuzumab.

In some embodiments, the antibody or antigen binding fragment thereof specifically binds HER3. In some embodiments, the antibody or antigen binding fragment thereof is patritumab.

In some embodiments, the antibody or antigen binding fragment thereof specifically binds HER2. In some embodiments, the antibody or antigen binding fragment thereof is trastuzumab.

In some embodiments, the antibody or antigen-binding fragment thereof is a monoclonal antibody, a chimeric antibody, a humanized antibody, a human engineered antibody, a single chain antibody (scFv), a Fab fragment, a Fab′ fragment, or a F(ab′)2 fragment.

In some embodiments, the antibody or antigen-binding fragment thereof has antibody dependent cellular cytotoxicity (ADCC) or complement dependent cytotoxicity (CDC).

In some embodiments, the Fc domain is an IgG1 with reduced effector function.

### Payloads

Provided herein are payloads (PA), e.g., for use in a platform and/or ADC described herein.

Compounds of Formula (I) may include any PA described herein.

In some embodiments, each PA is independently a cytotoxic agent.

In some embodiments, each PA is independently selected from the group consisting of DXd, 7-ethyl-10-hydroxy-camptothecin (SN-38), and monomethyl auristatin E (MMAE).

In some embodiments, each PA independently is a compound of formula (VI):

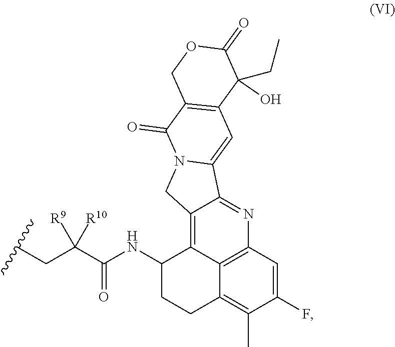

and each of R⁹ and R¹⁰ is independently hydrogen, halogen, or substituted or unsubstituted C₁₋₄ alkyl.

In some embodiments, each PA independently is

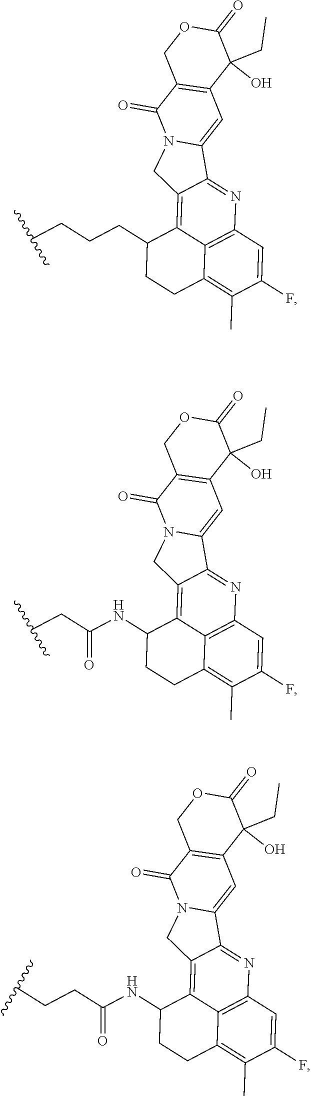 [SMILES: *CCCC1CCc2c(C)c(F)cc3nc4c(c1c23)Cn1c-4cc2c(c1=O)COC(=O)C2(O)CC ; *CC(=O)NC1CCc2c(C)c(F)cc3nc4c(c1c23)Cn1c-4cc2c(c1=O)COC(=O)C2(O)CC ; *CCC(=O)NC1CCc2c(C)c(F)cc3nc4c(c1c23)Cn1c-4cc2c(c1=O)COC(=O)C2(O)CC]

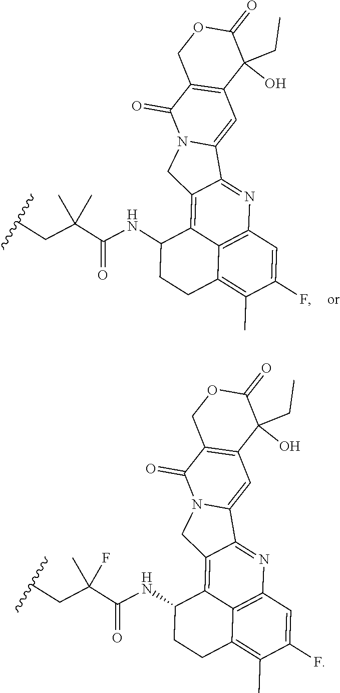

In some embodiments, the ADC has the following formula:

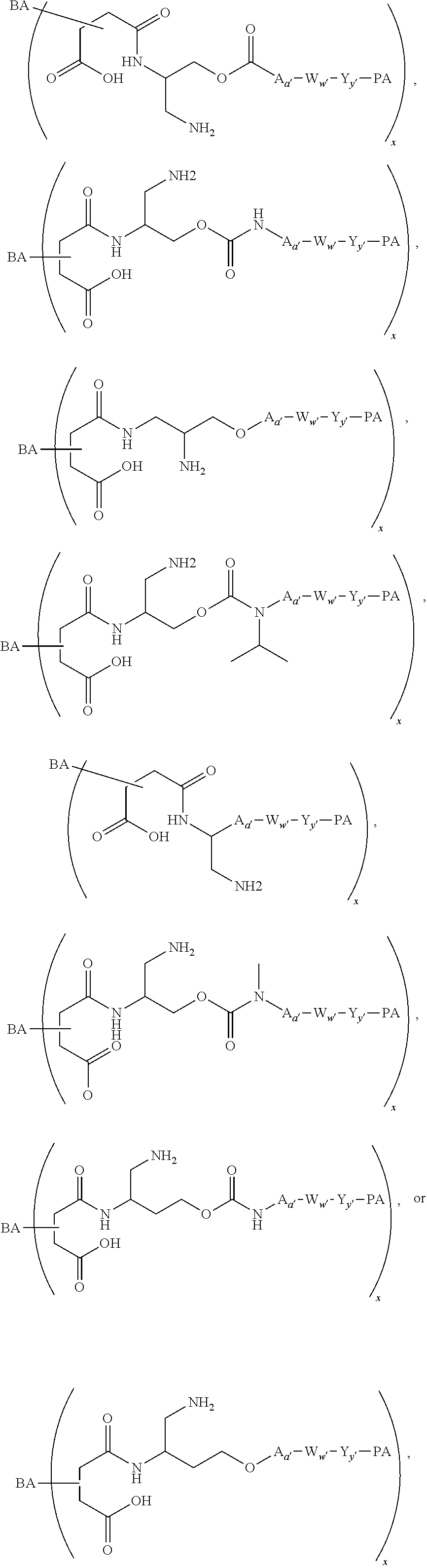

wherein values for the variables (e.g., BA, PA, A, W, Y, a′, w′, y′, x) are as described above.

In some embodiments, the ADC has the following formula:

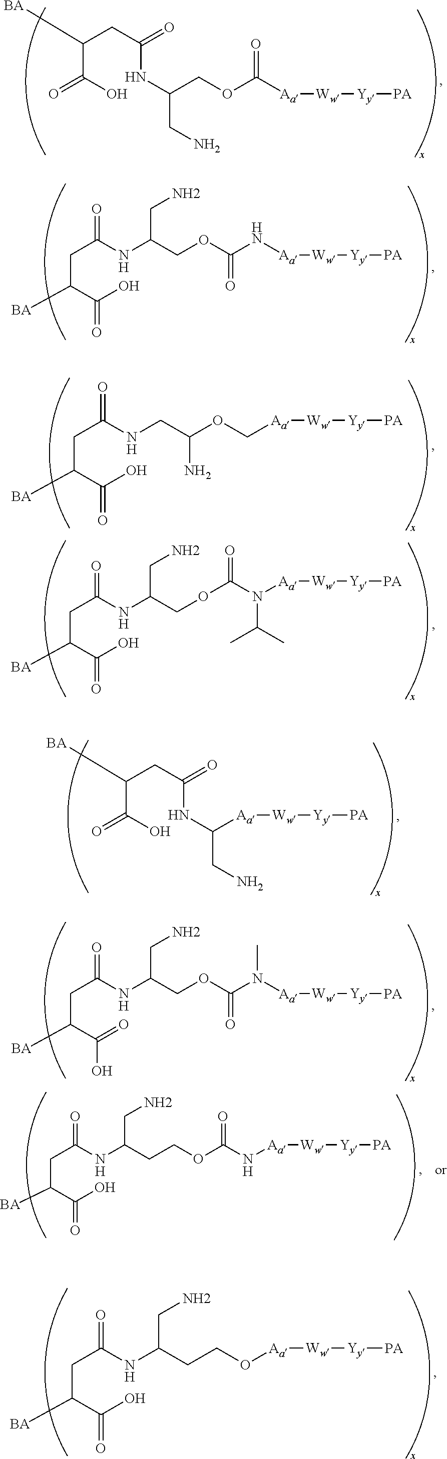

wherein values for the variables (e.g., BA, PA, A, W, Y, a′, w′, y′, x) are as described above.

### 7.2.2. Aspect 2

In some embodiments, the ADC compound is represented by one of the following formulas, or a pharmaceutically acceptable salt, solvate, and/or stereoisomer thereof:

| No. | Structure |
|---|---|
| ADC4-7o′ | 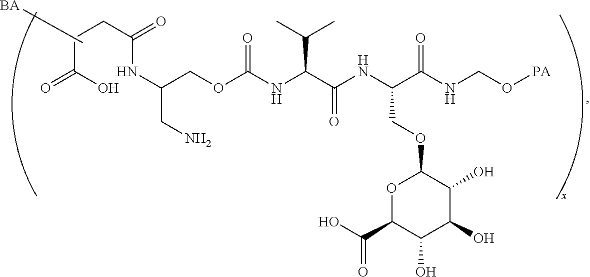 |
| ADC4-8o′ | 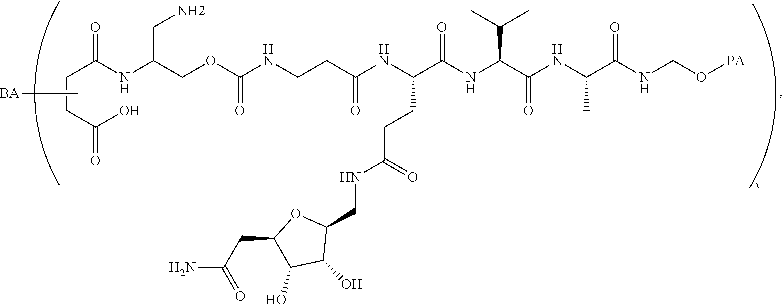 |
| ADC4-9o′ | 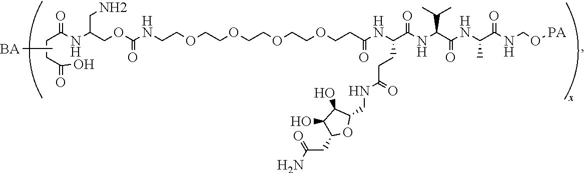 |
| ADC4-10o′ | 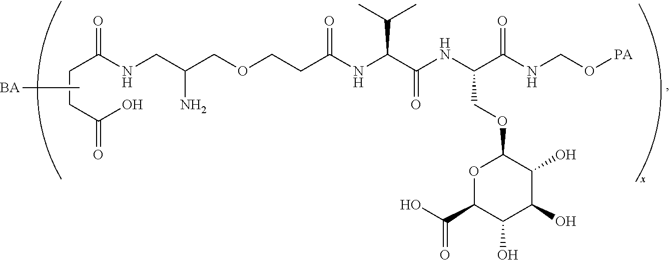 |
| ADC4-11o′ | 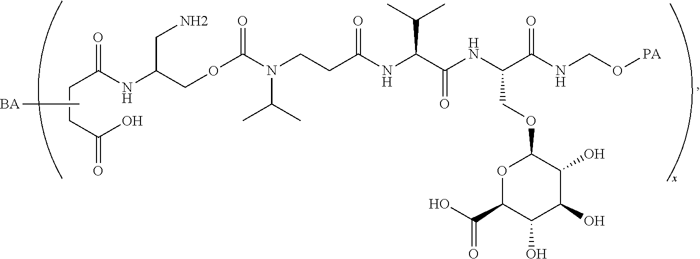 |
| ADC4-12o′ | 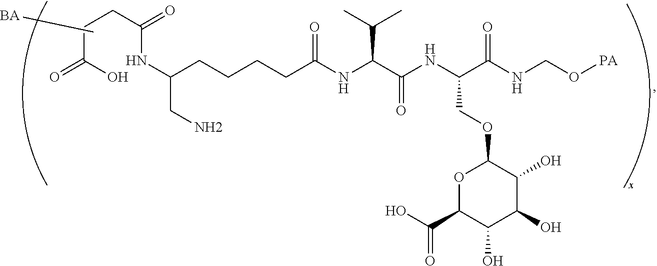 |
| ADC4-13o′ | 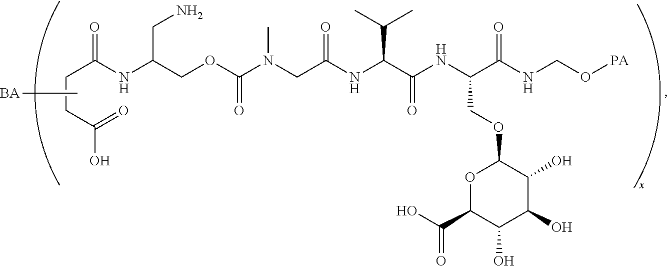 |
| ADC4-14o′ | 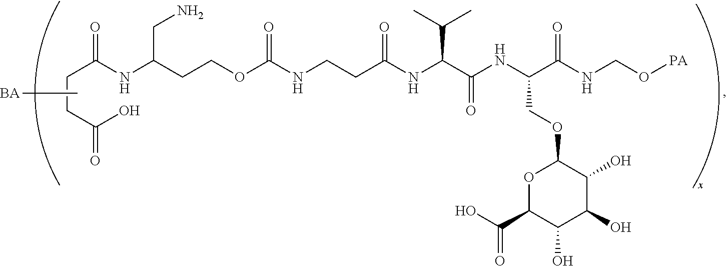 |
| ADC4-16o′ | 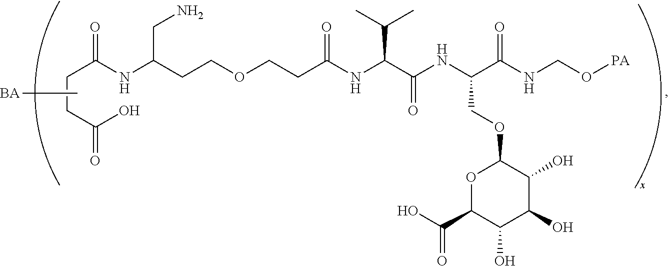 |

wherein BA is a humanized, chimeric, or human antibody or an antigen binding fragment thereof, subscript x is from 1 to 15, and PA is as described above in Aspect 1. Alternative values for BA are as set forth herein, e.g., with respect to Aspect 1. Alternative values for variable subscript x are as set forth herein, e.g., with respect to compounds of Formula (I).

In some embodiments, the ADC compound is represented by one of the following formulas, or a pharmaceutically acceptable salt, solvate, and/or stereoisomer thereof:

| No. | Structure |
|---|---|
| ADC4-7o′ | 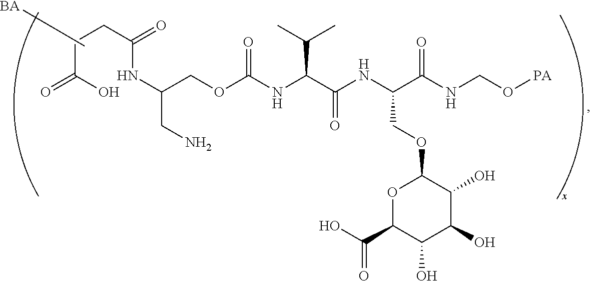 |
| ADC4-8o′ | 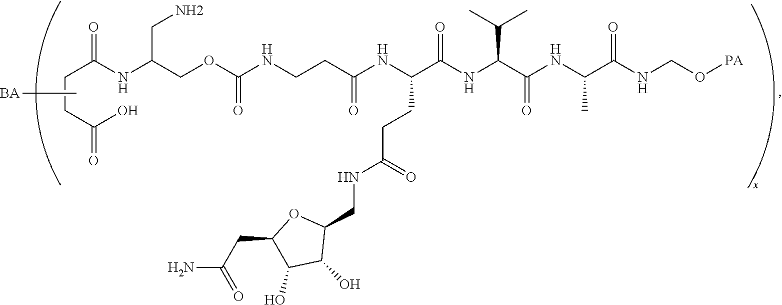 |
| ADC4-9o′ | 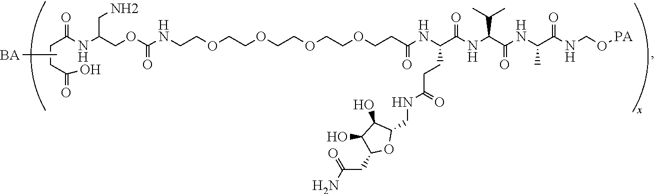 |
| ADC4-10o′ | 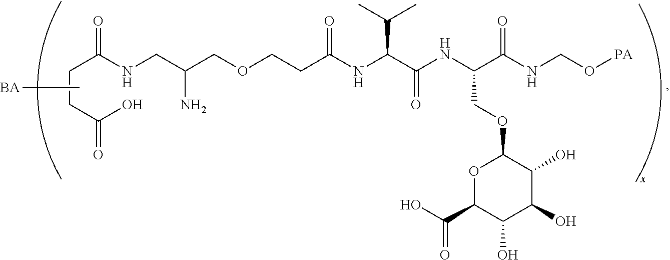 |
| ADC4-11o′ | 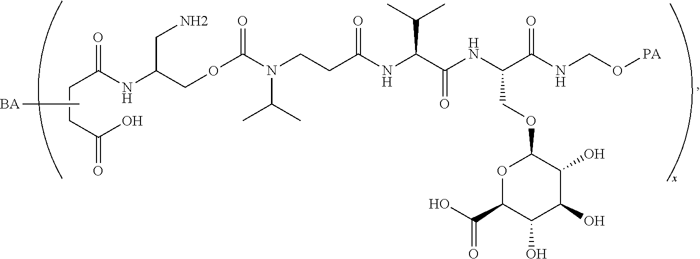 |
| ADC4-12o′ | 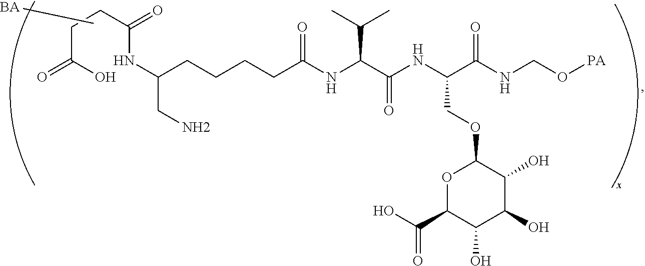 |
| ADC4-13o′ | 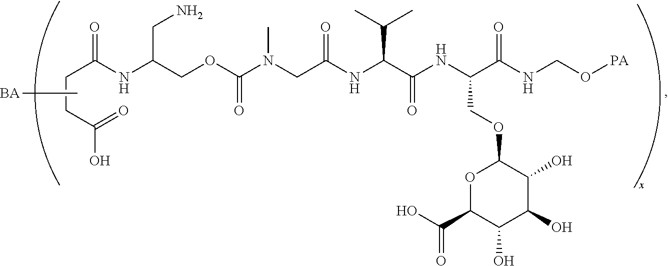 |
| ADC4-14o′ | 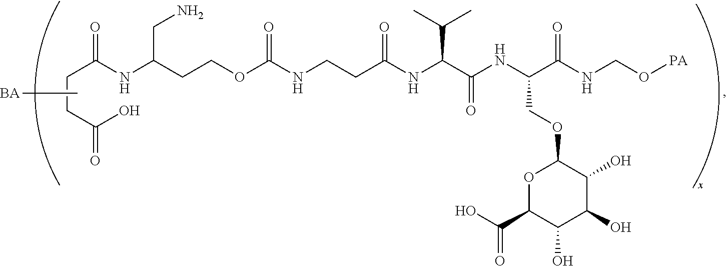 |
| ADC4-16o′ | 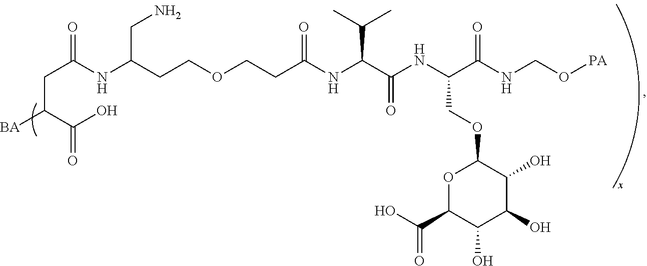 |
| ADC4-7′ | 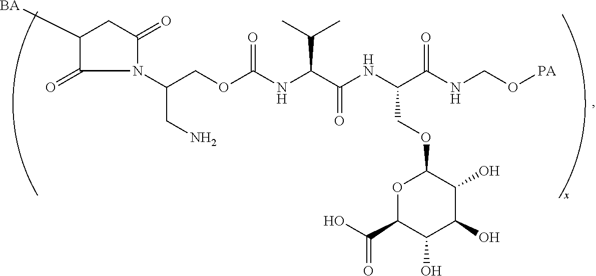 |
| ADC4-8′ | 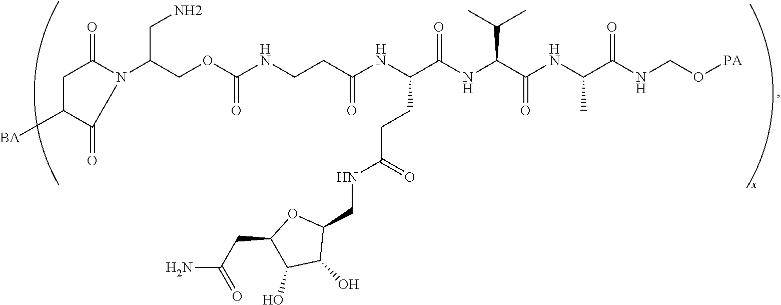 |
| ADC4-9′ | 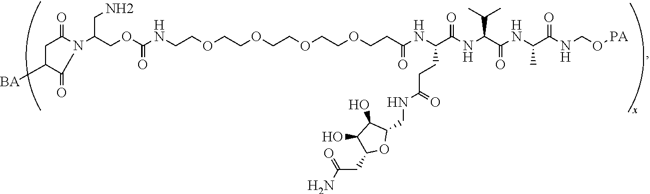 |
| ADC4-10′ | 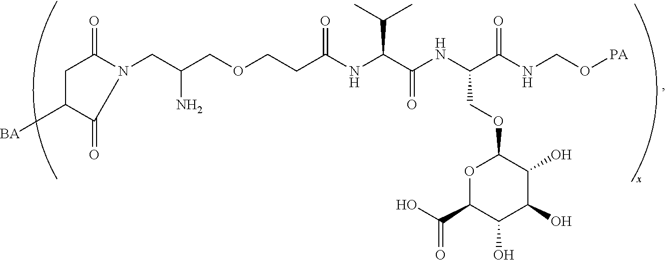 |
| ADC4-11′ | 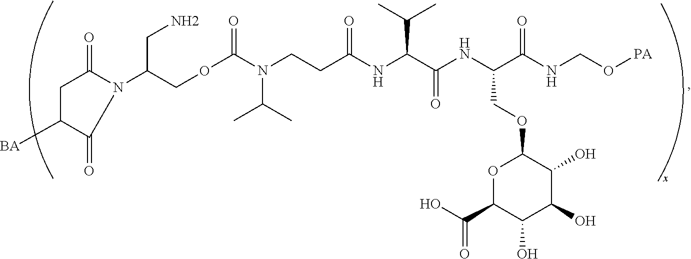 |
| ADC4-12′ | 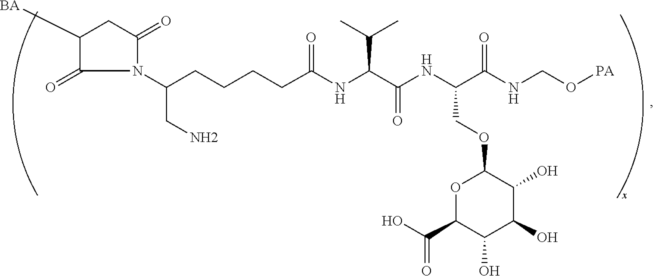 |
| ADC4-13′ | 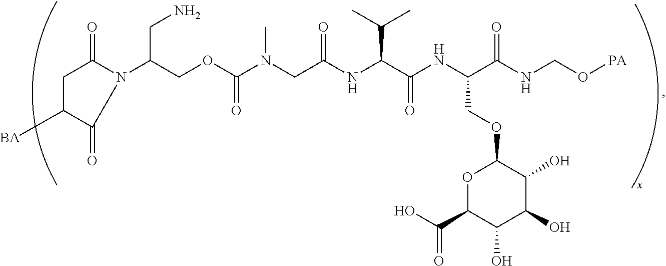 |
| ADC4-14′ | 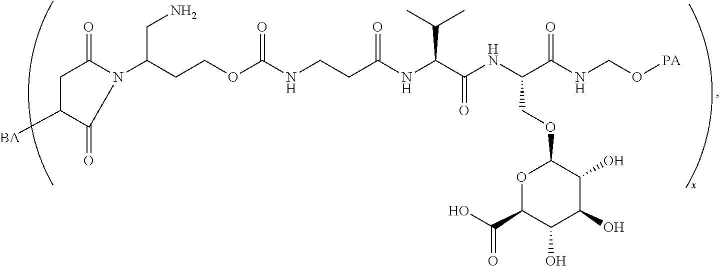 |
| | or |
| ADC4-16′ | 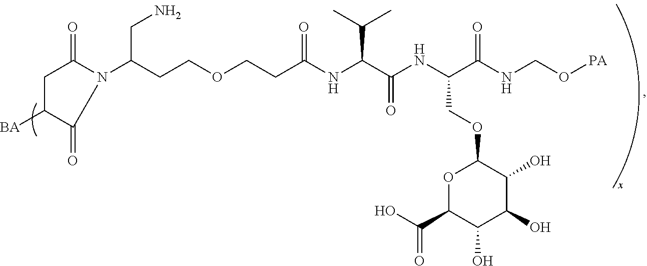 |

wherein BA is a humanized, chimeric, or human antibody or an antigen binding fragment thereof, subscript x is from 1 to 15, and PA is as described above in Aspect 1. Alternative values for BA are as set forth herein, e.g., with respect to Aspect 1. Alternative values for variable subscript x are as set forth herein, e.g., with respect to compounds of Formula (I).

### 7.2.3. Aspect 3

In some embodiments, the ADC compound is represented by one of the following formulas, or a pharmaceutically acceptable salt, solvate, and/or stereoisomer thereof:

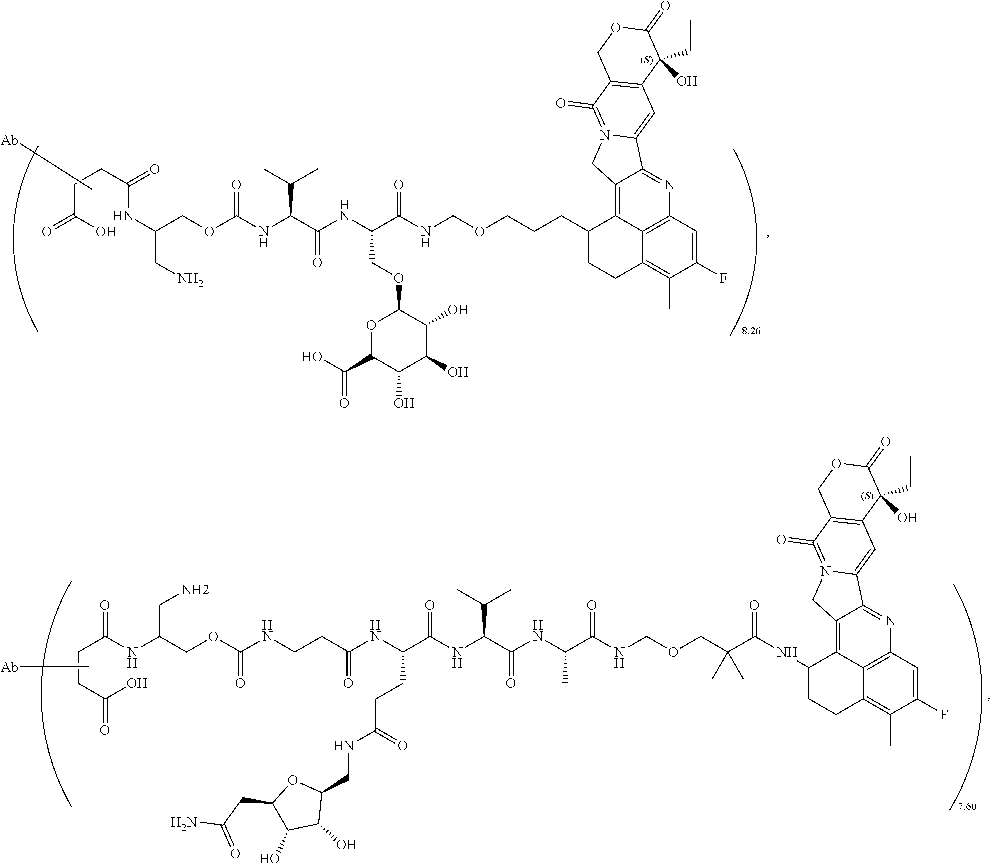

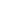

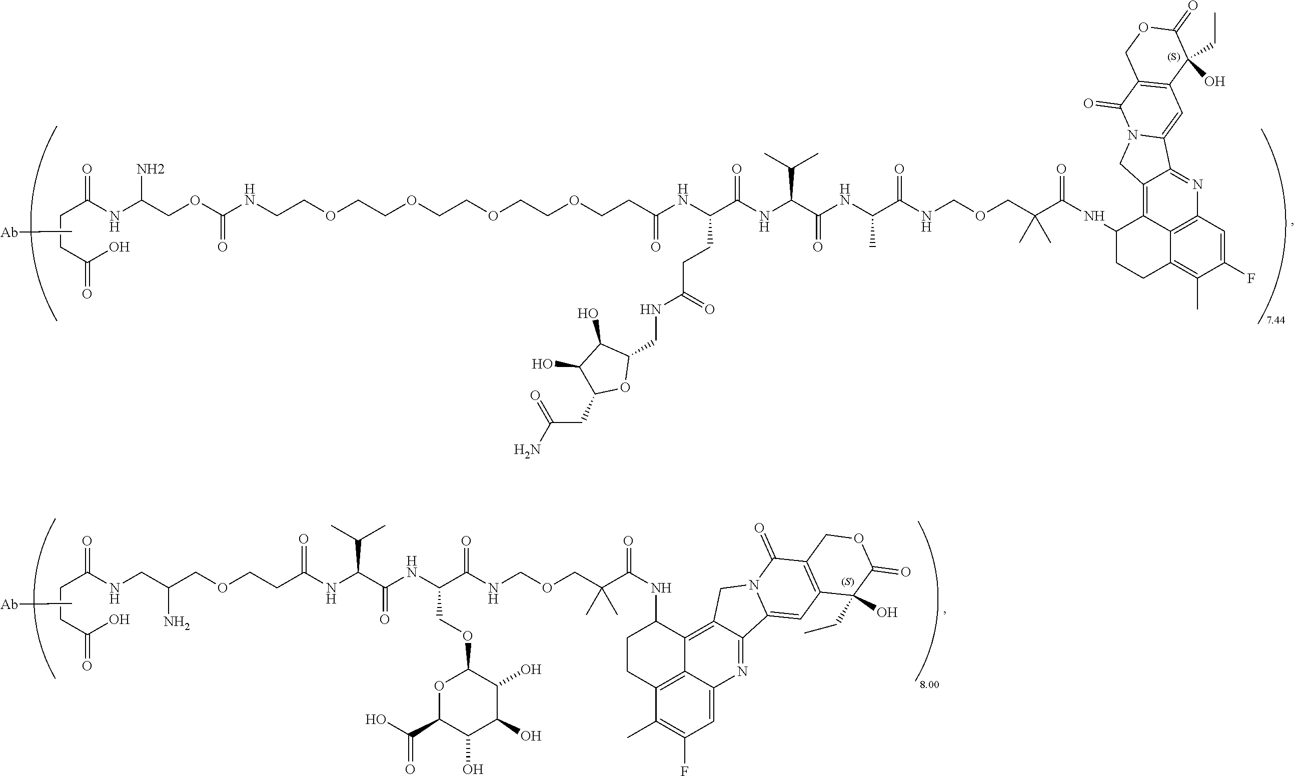

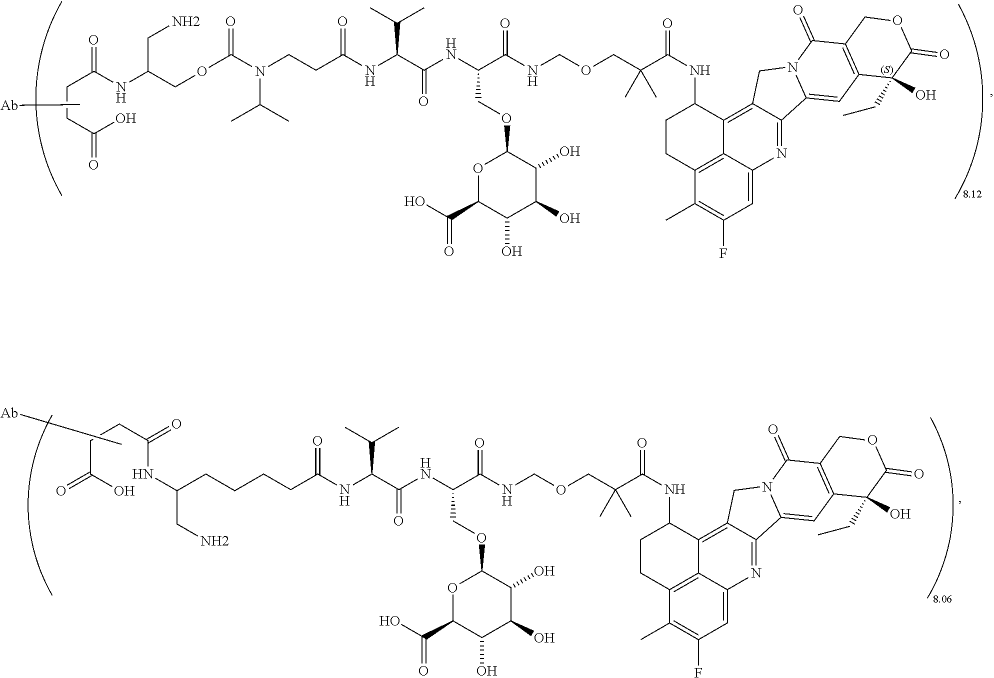

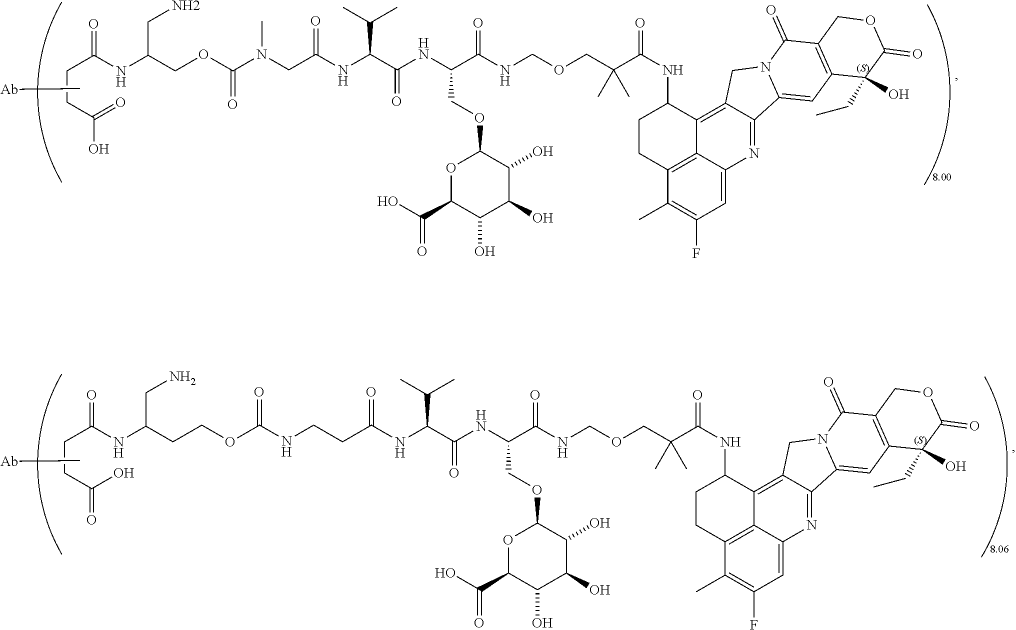

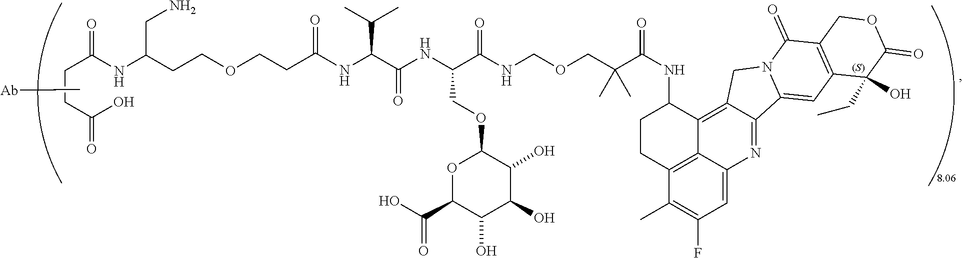

wherein Ab is a humanized, chimeric, or human antibody or an antigen binding fragment thereof.

In some embodiments, Ab is ifinatamab.

In some embodiments, the ADC compound is represented by one of the following formulas, or a pharmaceutically acceptable salt, solvate, and/or stereoisomer thereof:

| No. | Structure |
|---|---|
| ADC4-7o | 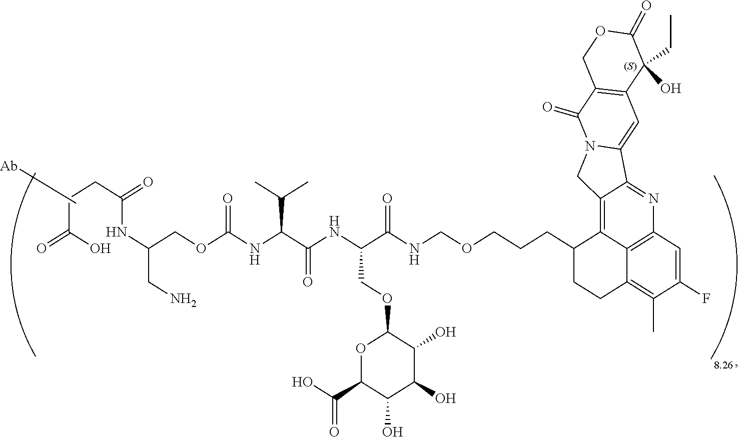 |
| ADC4-8o | 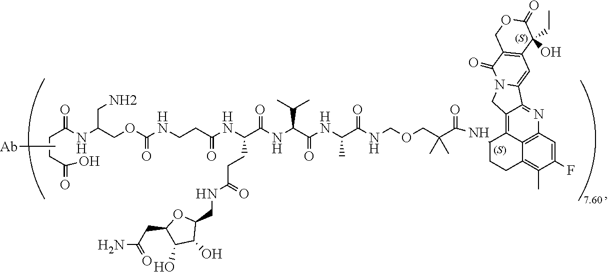 |
| ADC4-9o | 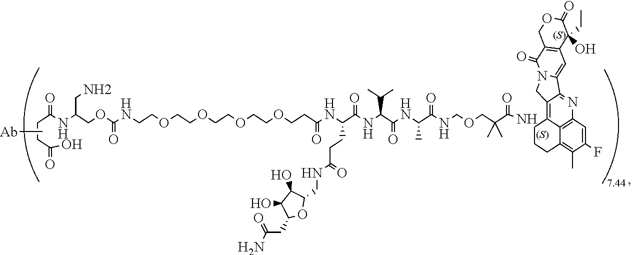 |
| ADC4-10o | 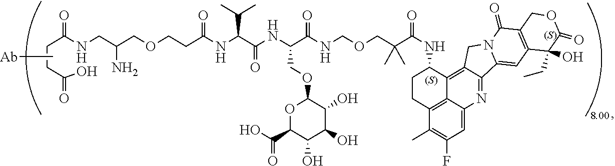 |
| ADC4-11o | 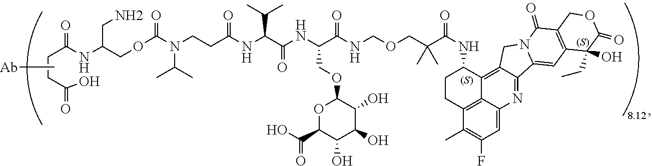 |
| ADC4-12o | 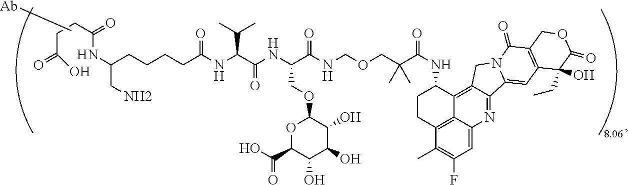 |
| ADC4-13o | 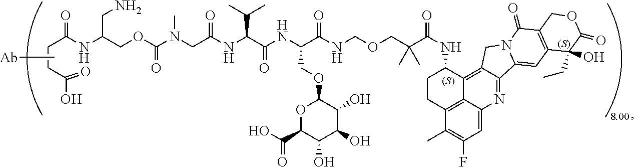 |
| ADC4-14o | 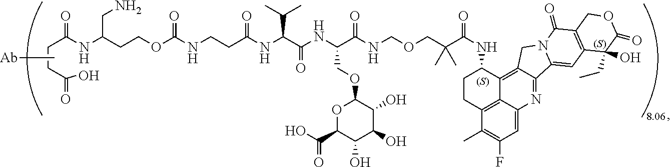 |
| | or |
| ADC4-16o | 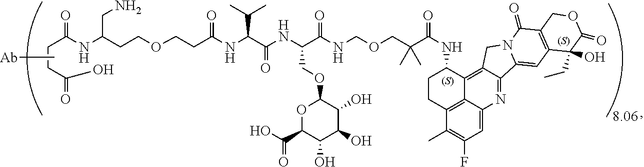 |

wherein Ab is a humanized, chimeric, or human antibody or an antigen binding fragment thereof.

In some embodiments, Ab is ifinatamab.

In some embodiments, the ADC compound is represented by one of the following formulas, or a pharmaceutically acceptable salt, solvate, and/or stereoisomer thereof:

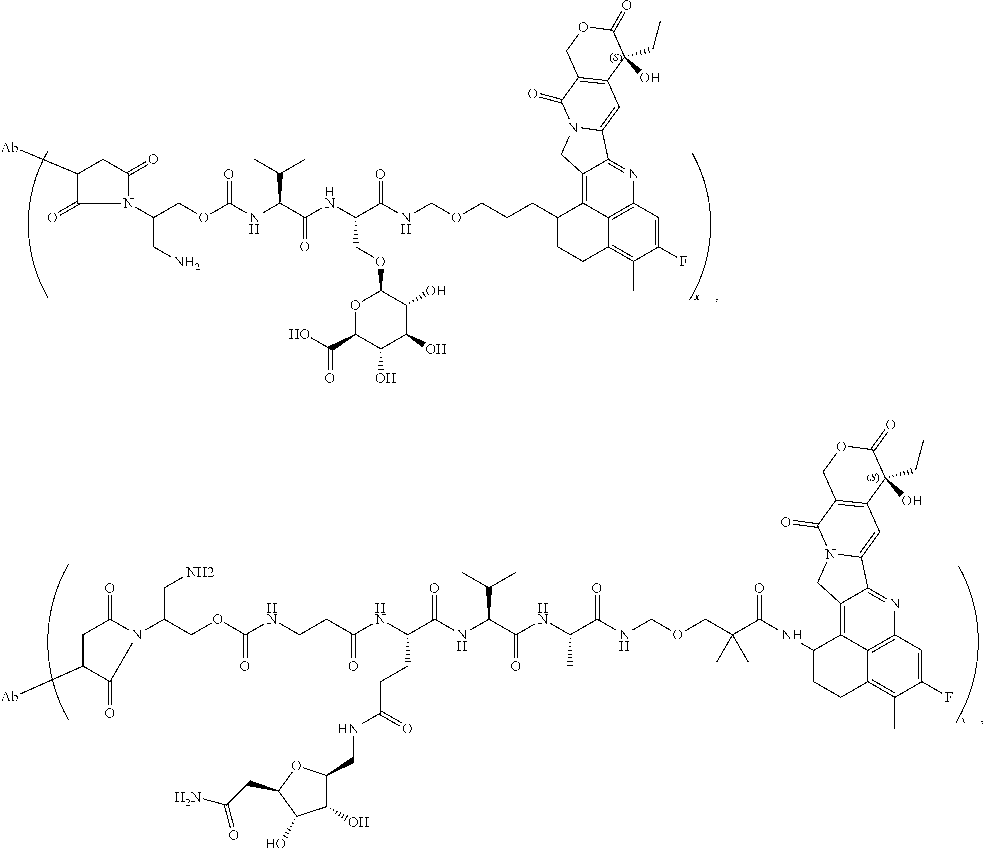

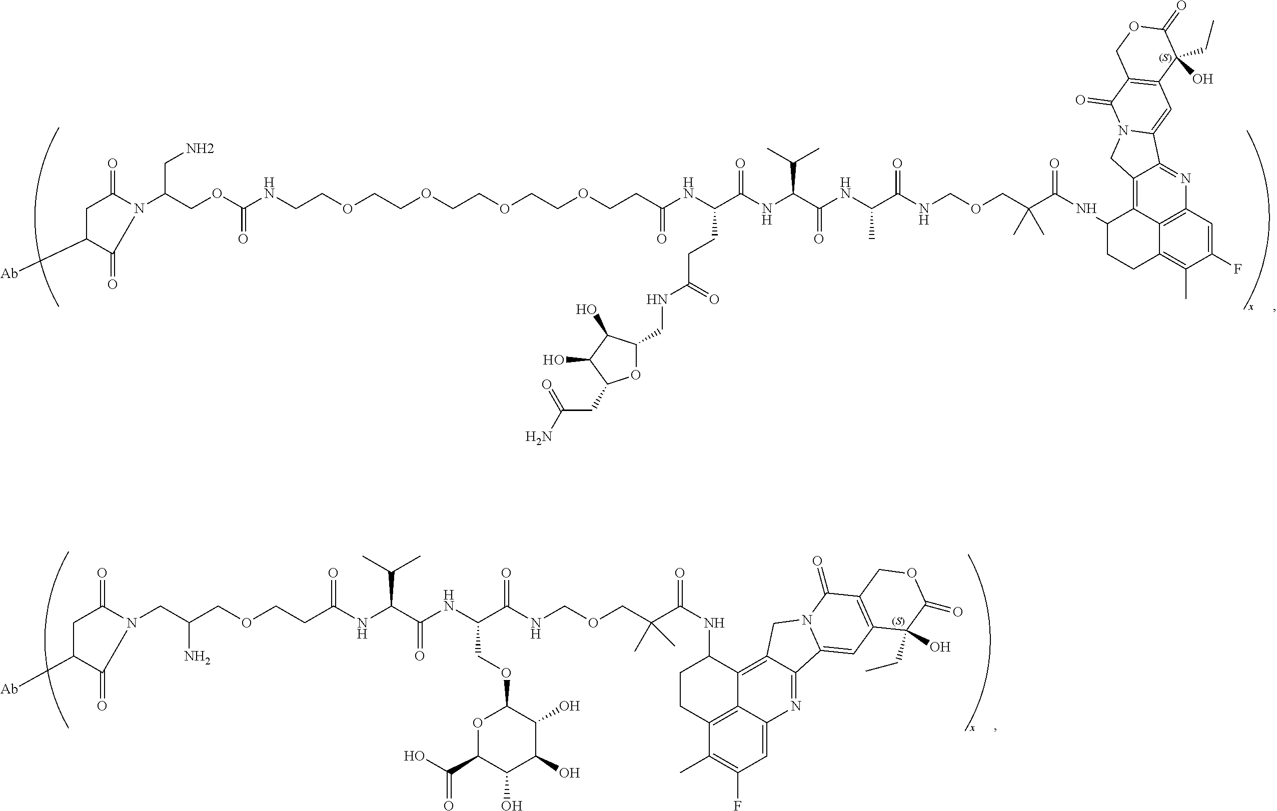

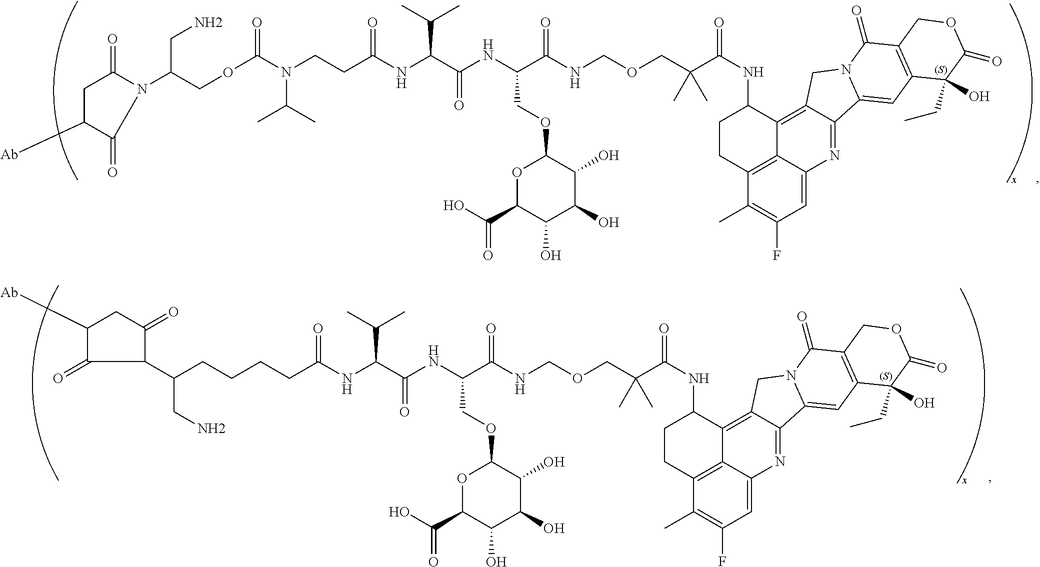

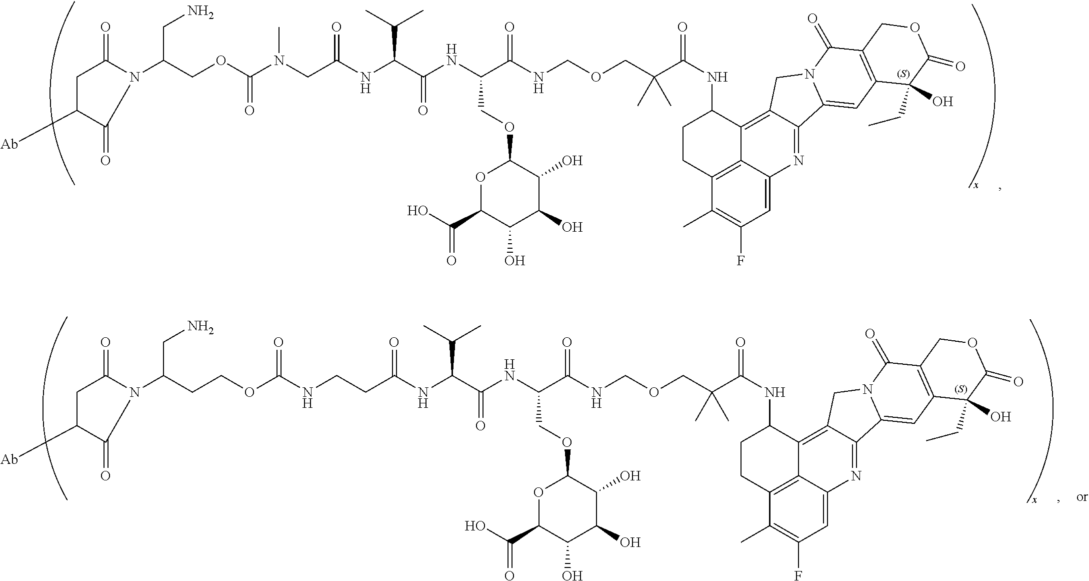

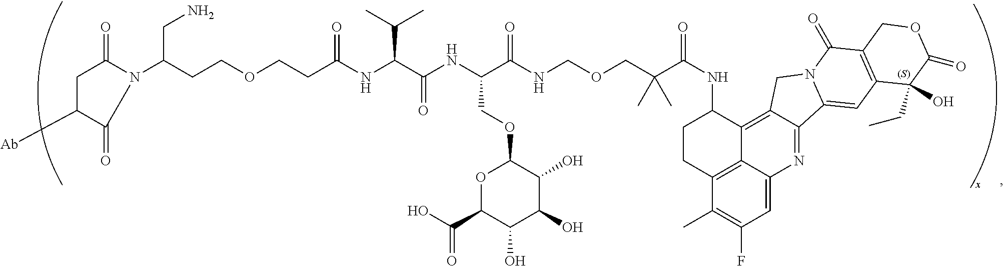

wherein Ab is a humanized, chimeric, or human antibody or an antigen binding fragment thereof, and x is from 1 to 15.

In some embodiments, Ab is ifinatamab.

In some embodiments, the ADC compound is represented by one of the following formulas, or a pharmaceutically acceptable salt, solvate, and/or stereoisomer thereof:

| No. | Structure |
|---|---|
| ADC4-7 | 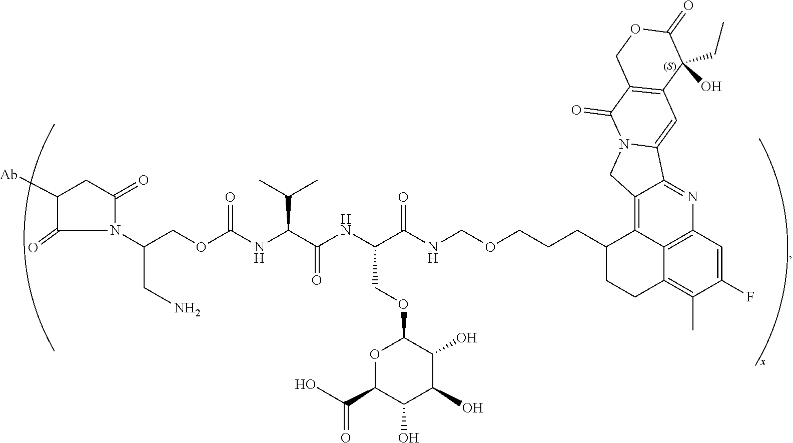 |
| ADC4-8 | 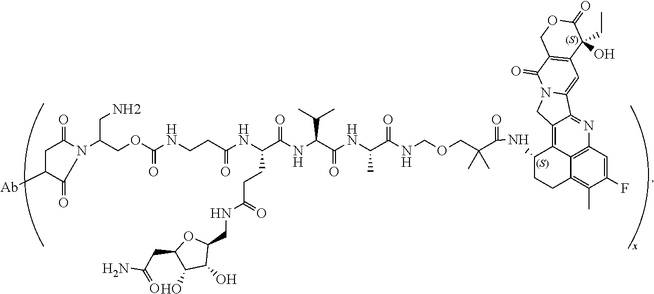 |
| ADC4-9 | 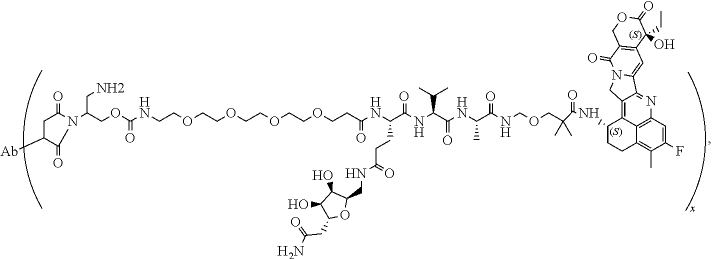 |
| ADC4-10 | 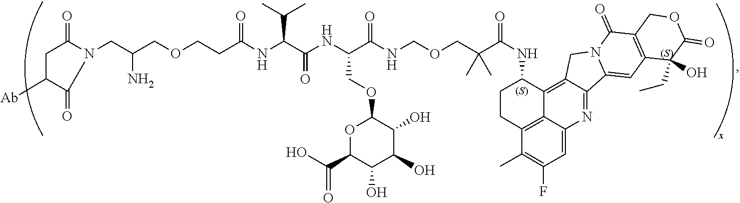 |
| ADC4-11 | 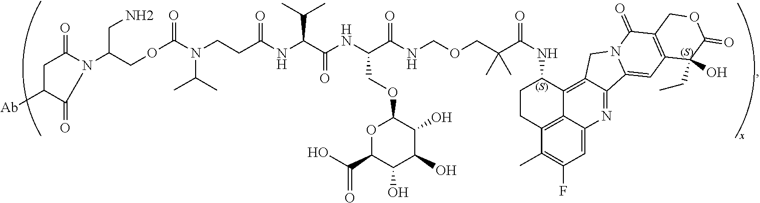 |
| ADC4-12 | 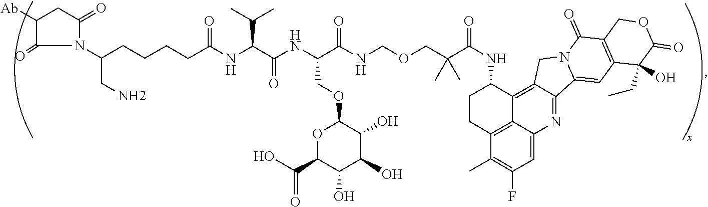 |
| ADC4-13 | 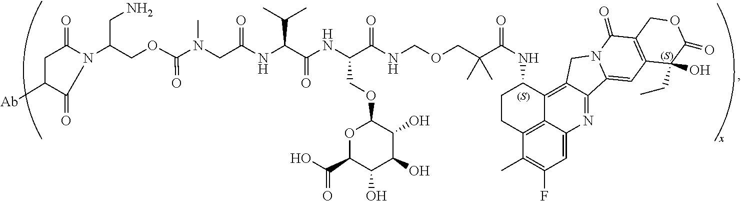 |
| ADC4-14 | 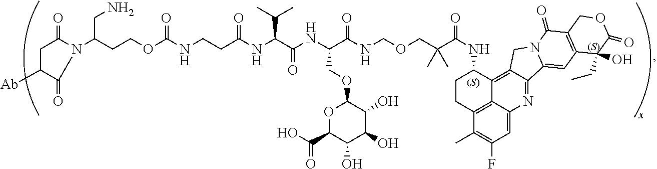 |
| | or |
| ADC4-16 | 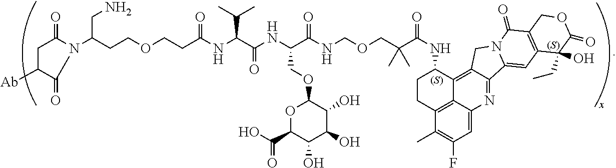 |

wherein Ab is a humanized, chimeric, or human antibody or an antigen binding fragment thereof, and x is from 1 to 15.

In some embodiments, Ab is ifinatamab.

### 7.2.4. Aspect 4

Also provided herein are platforms, e.g., for use in preparing an ADC, such as an ADC described herein.

In some embodiments, the platform is a linker-payload compound of Formula (II):

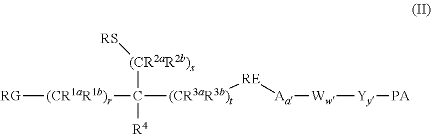

or a pharmaceutically acceptable salt, tautomer, solvate, or stereoisomer thereof,
RG is a reactive group;
RS is an open-ring stabilizing group;
RE is an open-ring enhancer;
each of R^{1a} and R^{1b} is, independently, H; substituted or unsubstituted C₁₋₄ alkyl; substituted or unsubstituted C₃₋₅ cycloalkyl; or R^{1a} and R^{1b} together with the atom to which they are attached form a substituted or unsubstituted C₃₋₅ cycloalkyl;
each of R^{2a} and R^{2b} is, independently, H; substituted or unsubstituted C₁₋₄ alkyl; substituted or unsubstituted C₃₋₅ cycloalkyl; or R^{2a} and R^{2b} together with the atom to which they are attached form a substituted or unsubstituted C₃₋₅ cycloalkyl;
each of R^{3a} and R^{3b} is, independently, H; substituted or unsubstituted C₁₋₄ alkyl; substituted or unsubstituted C₃₋₅ cycloalkyl; or R^{3a} and R^{3b} together with the atom to which they are attached form a substituted or unsubstituted C₃₋₅ cycloalkyl;
R⁴ is H; substituted or unsubstituted C₁₋₄ alkyl; or substituted or unsubstituted C₃₋₅ cycloalkyl;
each of r, s, and tis, independently, 0, 1, or 2;
A is a Stretcher unit residue;
subscript a′ is 0 or 1;
W is a Cleavable unit;
subscript w′ is 0 or 1;
Y is a Spacer unit;
subscript y′ is 0 or 1; and
PA is a payload residue.

In some embodiments, RG is

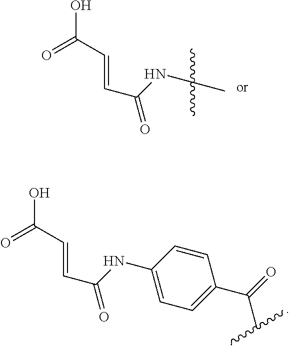 [SMILES: *NC(=O)/C=C/C(=O)O ; *C(=O)c1ccc(NC(=O)/C=C/C(=O)O)cc1]

In some embodiments, RG is

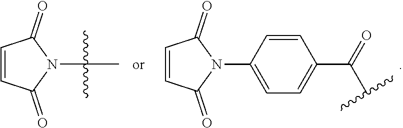 [SMILES: *N1C(=O)C=CC1=O ; *C(=O)c1ccc(N2C(=O)C=CC2=O)cc1]

In some embodiments, each of R^{1a}, R^{1b}, R^{2a}, R^{2b}, R^{3a}, R^{3b}, and R⁴ is, independently, H.

In some embodiments, RS is an amino group, or —NR^{5a}R^{5b}, wherein each of R^{5a} and R^{5b} is, independently, H, or substituted or unsubstituted C₁₋₄ alkyl.

In some embodiments, RS is an amino group, or —N(CH₃)₂.

In some embodiments, RS is an amino group.

In some embodiments, RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—; and R⁶ is H or substituted or unsubstituted C₁₋₄ alkyl.

In some embodiments, R⁶ is H, methyl, ethyl, or isopropyl.

In some embodiments, RE is —OC(═O)NR⁶—.

In some embodiments, RE is —OC(═O)NH—.

In some embodiments, r is 0.

In some embodiments, s is 1.

In some embodiments, t is 1 or 2.

In some embodiments, A is a bond, —(CH₂)ₙ—C(═O)—, —CH₂—C(═O)—NH—(CH₂)ₙ—C(═O)—, —(CH₂CH₂O)ₙ—CH₂CH₂—C(═O)—, —CH [—(CH₂)ₙ—COOH]—C(═O)—, —CH₂—C(═O)—NH—(CH₂)ₙ—C(═O)—NH—(CH₂)—C(═O)—, or —C(═O)—(CH₂)ₙ—C(═O)—, wherein each n independently represents an integer of 1, 2, 3, 4, or 5.

In some embodiments, W_{w′} is one of the following formulas:

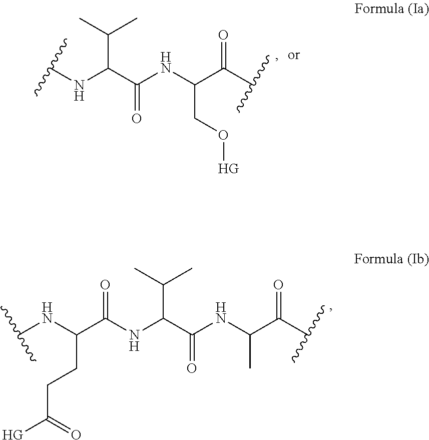

and HG is a hydrophilic moiety or hydrogen.

In some embodiments, HG is a saccharide, phosphate ester, sulfate ester, a phosphodiester, or a phosphonate.

In some embodiments, the saccharide, and the saccharide is β-D-galactose, N-acetyl-P-D-galactosamine, N-acetyl-a-D-galactosamine, N-acetyl-P-D-glucosamine, β-D-glucuronic acid, a-L-iduronic acid, a-D-galactose, a-D-glucose, β-D-glucose, a-D-mannose, β-D-mannose, a-L-fucose, β-D-xylose, a neuraminic acid, sulfate, phosphate, carboxyl, amino, or an O-acetyl modification thereof. In some embodiments, HG is selected from β-D-galactose and β-D-glucuronic acid.

In some embodiments, HG is

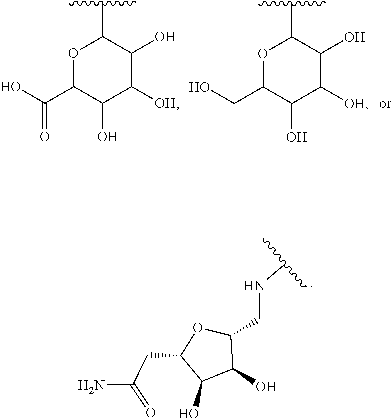 [SMILES: *C1OC(C(=O)O)C(O)C(O)C1O ; *C1OC(CO)C(O)C(O)C1O ; "or" ; *NC[C@H]1O[C@@H](CC(N)=O)[C@H](O)[C@@H]1O]

In some embodiments, Y_{y′} is a PAB unit.

In some embodiments, the compound has the following formula:

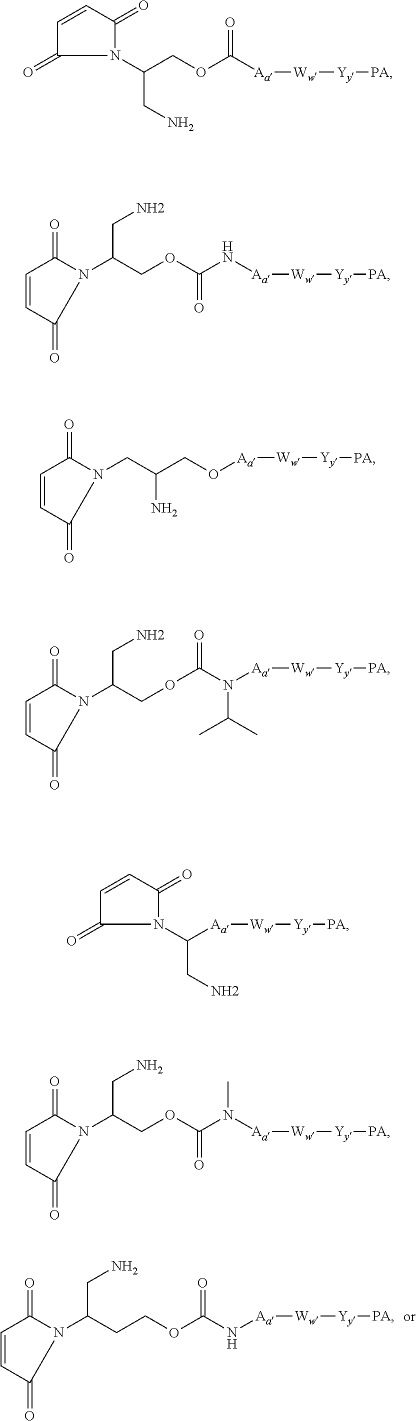

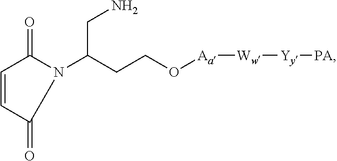

wherein values for the variables (e.g., BA, PA, A, W, Y, a′, w′, y′, x) are as described above.

### 7.2.5. Aspect 5

In some embodiments, the linker-payload compound is represented by one of the following formulas, or a pharmaceutically acceptable salt, solvate, and/or stereoisomer thereof:

| No. | Structure |
|---|---|
| LP 4-7 | 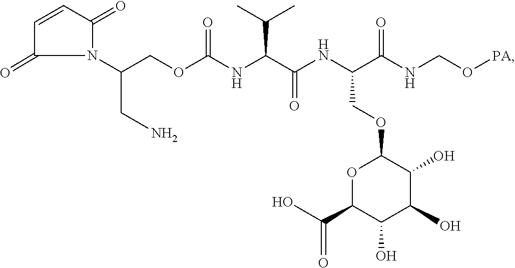 |
| LP 4-8 | 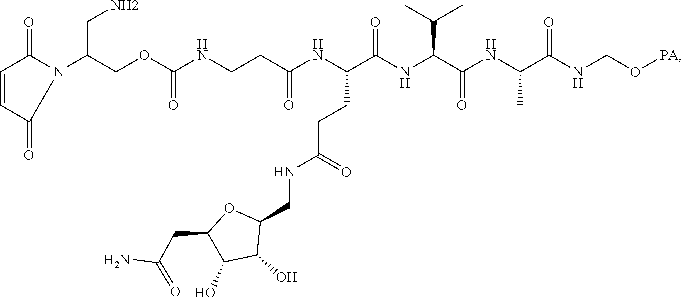 |
| LP 4-9 | 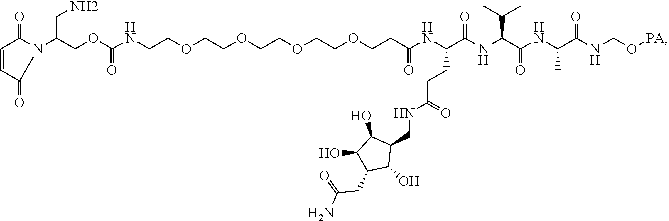 |
| LP 4-10 | 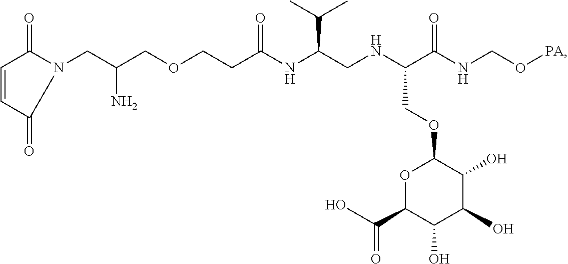 |
| LP 4-11 | 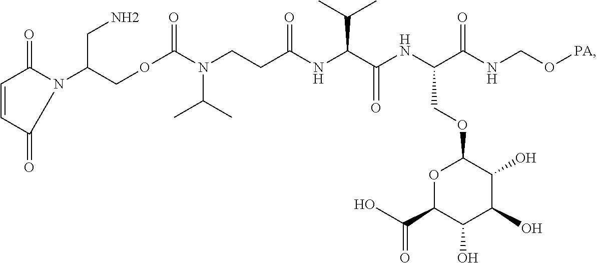 |
| LP 4-12 | 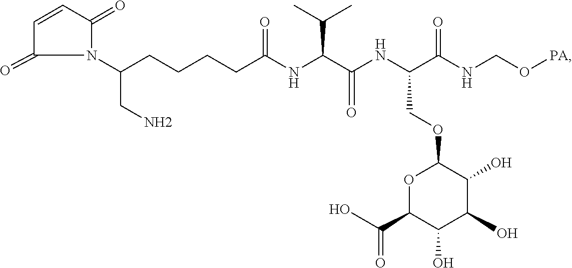 |
| LP 4-13 | 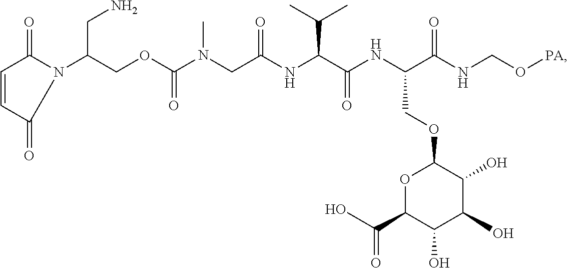 |
| LP 4-14 | 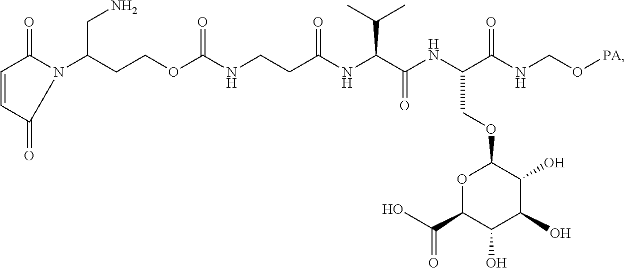 |
| | or |
| LP 4-16 | 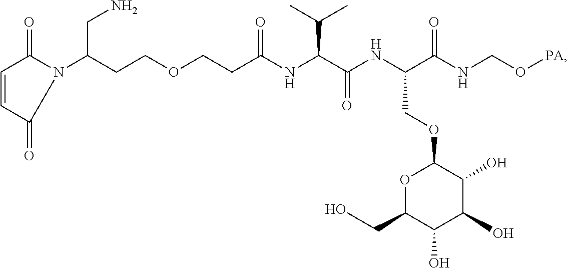 |

wherein PA is as described above for Aspect 1.

In some embodiments, the linker-payload compound is any one of the following, or a pharmaceutically acceptable salt and/or solvate thereof:

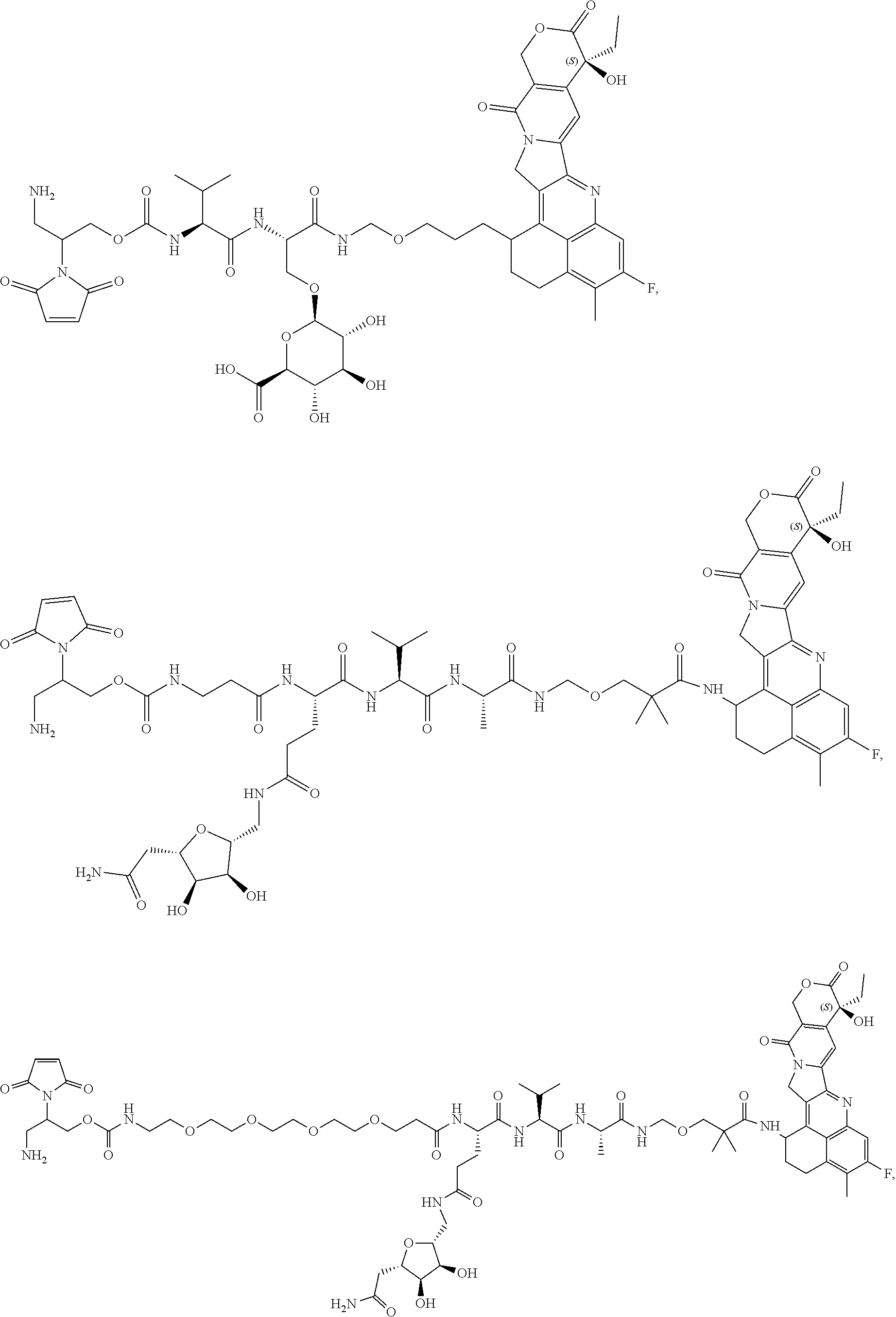

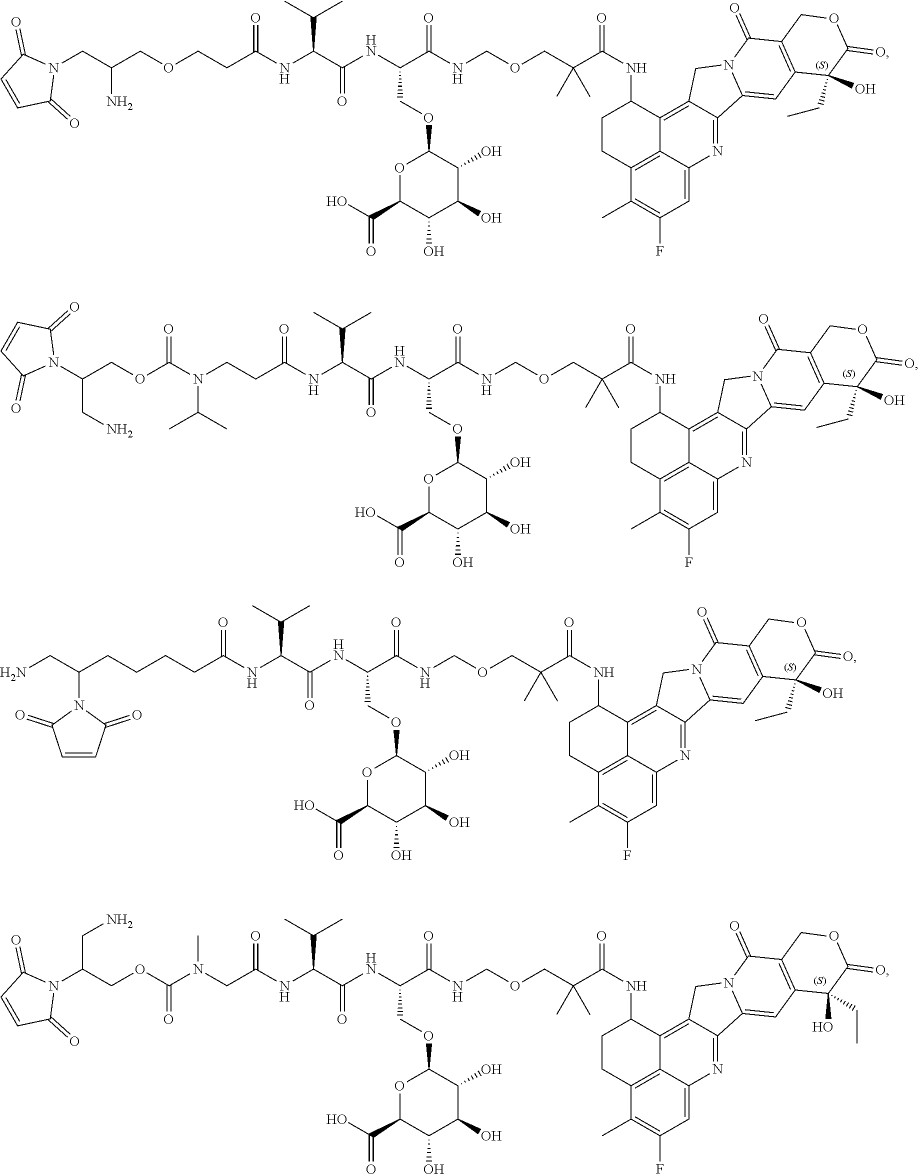

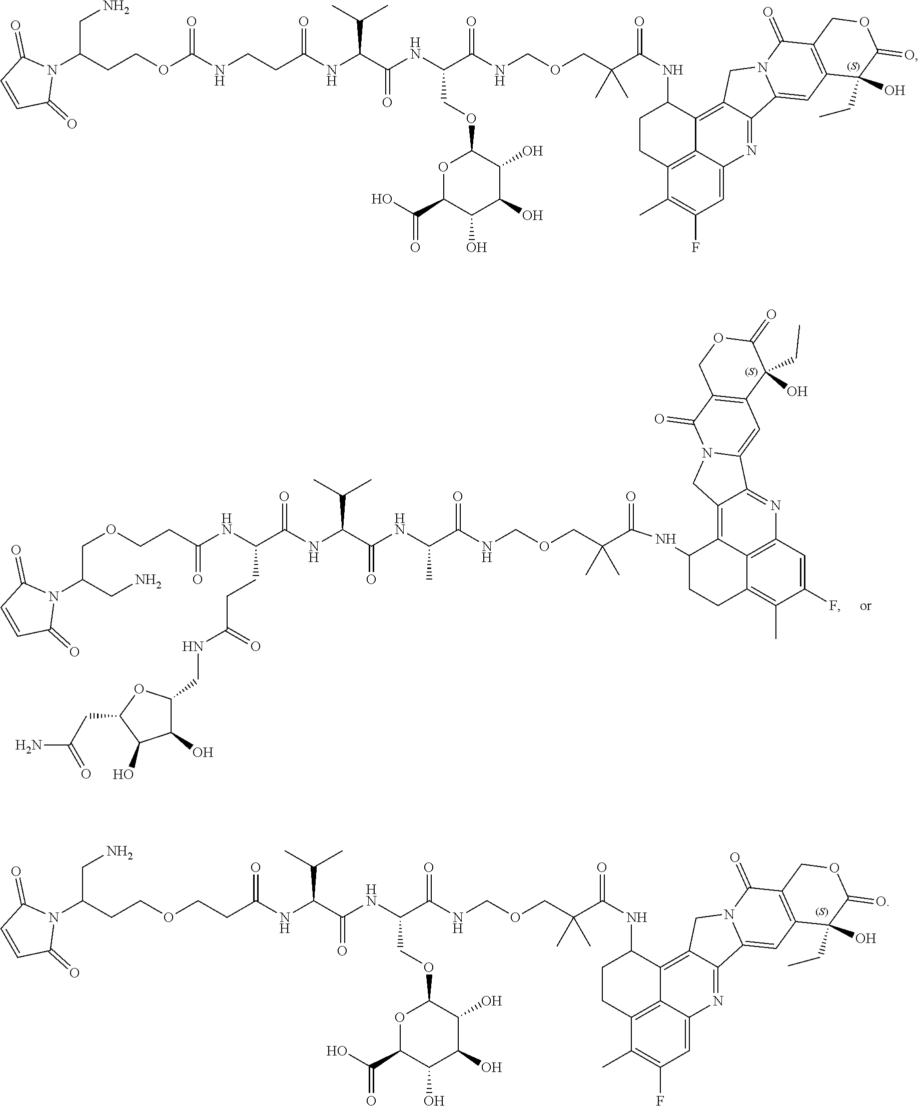

In some embodiments, the linker-payload compound is any one of the following, or a pharmaceutically acceptable salt and/or solvate thereof:

| No. | Structure |
|---|---|
| 2-7 | 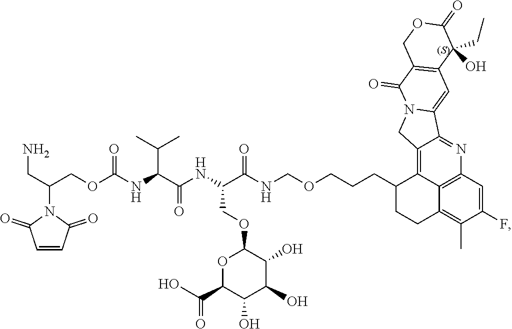 |
| 2-8 | 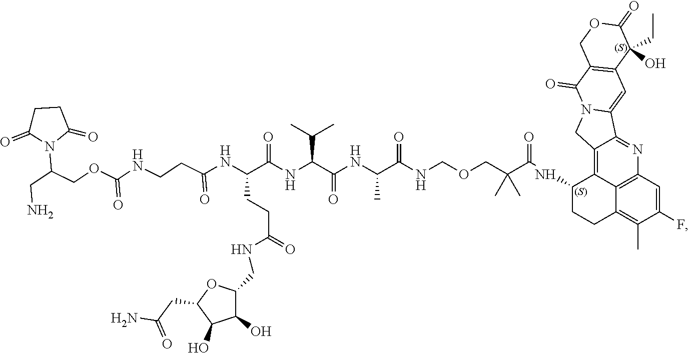 |
| 2-9 | 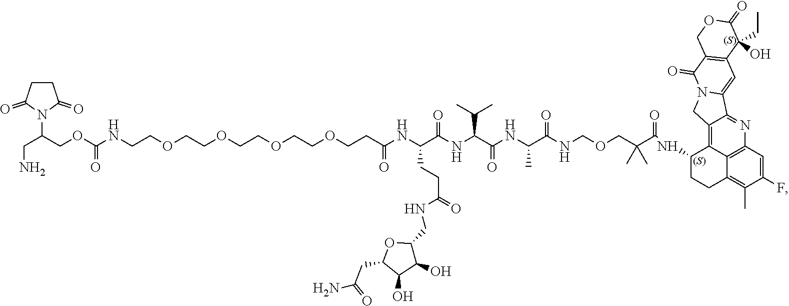 |
| 2-10 | 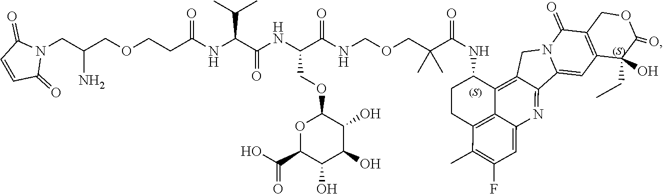 |
| 2-11 | 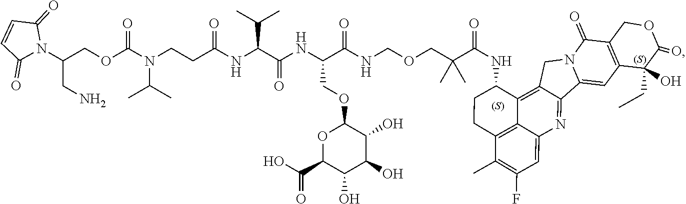 |
| 2-12 | 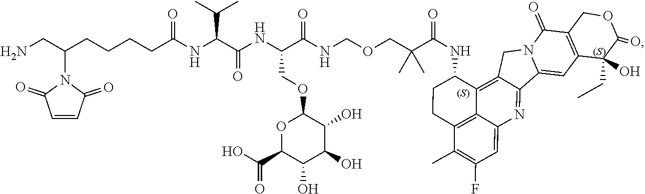 |
| 2-13 | 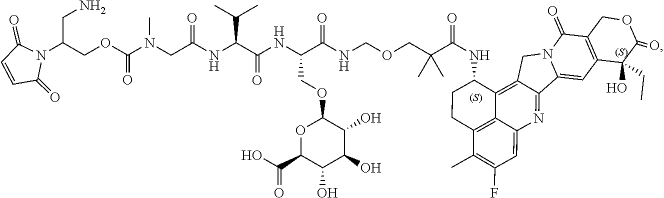 |
| 2-14 | 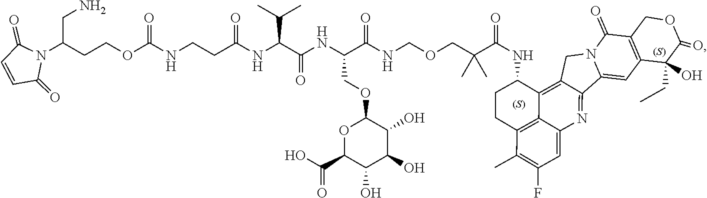 |
| 2-15 | 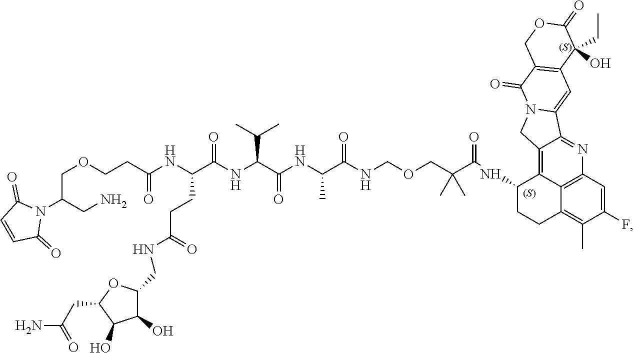 |
| 2-16 | 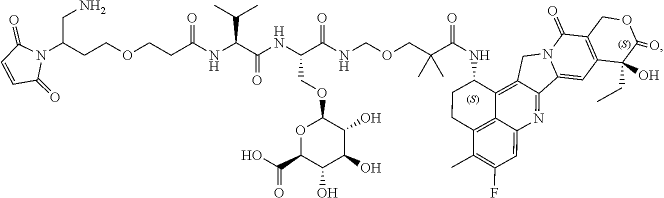 |

### 7.2.6. Aspect 6

Also provided herein are covalent linkers, e.g., for use in a platform and/or ADC described herein.

In some embodiments (e.g., of a linker for use in a platform), the linker is a compound of Formula (II):

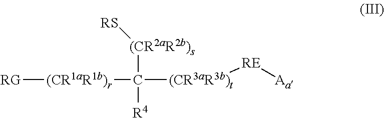

or a pharmaceutically acceptable salt, tautomer, solvate, or stereoisomer thereof,
RG is a reactive group;
RS is an open-ring stabilizing group;
RE is an open-ring enhancer;
each of R^{1a} and R^{1b} is, independently, H; substituted or unsubstituted C₁₋₄ alkyl; substituted or unsubstituted C₃₋₅ cycloalkyl; or R^{1a} and R^{1b} together with the atom to which they are attached form a substituted or unsubstituted C₃₋₅ cycloalkyl;
each of R^{2a} and R^{2b} is, independently, H; substituted or unsubstituted C₁₋₄ alkyl; substituted or unsubstituted C₃₋₅ cycloalkyl; or R^{2a} and R^{2b} together with the atom to which they are attached form a substituted or unsubstituted C₃₋₅ cycloalkyl;
each of R^{3a} and R^{3b} is, independently, H; substituted or unsubstituted C₁₋₄ alkyl; substituted or unsubstituted C₃₋₅ cycloalkyl; or R^{3a} and R^{3b} together with the atom to which they are attached form a substituted or unsubstituted C₃₋₅ cycloalkyl;
R⁴ is H; substituted or unsubstituted C₁₋₄ alkyl; or substituted or unsubstituted C₃₋₅ cycloalkyl;
each of r, s, and tis, independently, 0, 1, or 2;
A is a Stretcher unit; and
subscript a′ is 0 or 1.

In some embodiments, RG is

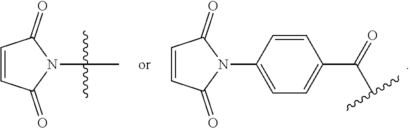 [SMILES: *N1C(=O)C=CC1=O ; *C(=O)c1ccc(N2C(=O)C=CC2=O)cc1]

In some embodiments, each of R^{1a}, R^{1b}, R^{2a}, R^{2b}, R^{3a}, R^{3b}, and R⁴ is, independently, H.

In some embodiments, RS is an amino group or —NR^{5a}R^{5b}, wherein each of R^{5a} and R^{5b} is, independently, H, or substituted or unsubstituted C₁₋₄ alkyl.

In some embodiments, RS is an amino group or —N(CH₃)₂.

In some embodiments, RS is an amino group.

In some embodiments, RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—; and R⁶ is H or substituted or unsubstituted C₁₋₄ alkyl.

In some embodiments, R⁶ is H, methyl, ethyl, or isopropyl.

In some embodiments, RE is —OC(═O)NR⁶—.

In some embodiments, RE is —OC(═O)NH—.

In some embodiments, r is 0.

In some embodiments, s is 1.

In some embodiments, t is 1 or 2.

In some embodiments, A is a bond, —(CH₂)ₙ—C(═O)R⁷, —CH₂—C(═O)—NH—(CH₂)ₙ—C(═O)R⁷, —(CH₂CH₂O)ₙ—CH₂CH₂—C(═O)R⁷, —CH [—(CH₂)ₙ—COOH]—C(═O)R⁷, —CH₂—C(═O)—NH—(CH₂)ₙ—C(═O)—NH—(CH₂)ₙ—C(═O)R⁷, or —C(═O)—(CH₂)ₙ—C(═O)R⁷, wherein each n independently represents an integer of 1, 2, 3, 4, or 5; R⁷ is OH or NR^{8a}R^{8b}, and each of R^{8a} and R^{8b} is, independently, H; substituted or unsubstituted C₁₋₄ alkyl; substituted or unsubstituted C₃₋₅ cycloalkyl; or R^{8a} and R^{8b} together with the atom to which they are attached form a substituted or unsubstituted C₃₋₅ cycloalkyl.

In some embodiments, R⁷ is OH, NH₂, NHCH₃, or N(CH₃)₂.

### 7.2.7. Aspect 7

In some embodiments, the linker compound is any one of the following, or a pharmaceutically acceptable salt and/or solvate thereof:

| No. | Structure |
|---|---|
| 1-2 | 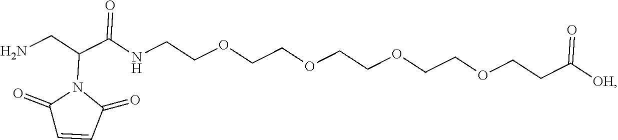 [SMILES: NCC(C(=O)NCCOCCOCCOCCOCCC(=O)O)N1C(=O)C=CC1=O] |
| 1-4 | 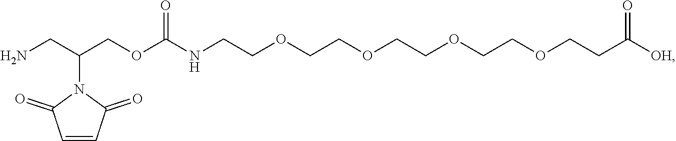 [SMILES: NCC(COC(=O)NCCOCCOCCOCCOCCC(=O)O)N1C(=O)C=CC1=O] |
| 1-5 | 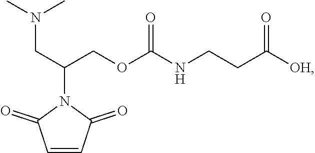 [SMILES: CN(C)CC(COC(=O)NCCC(=O)O)N1C(=O)C=CC1=O] |
| 1-6 | 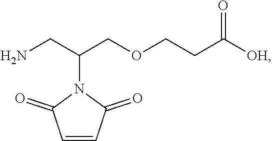 [SMILES: NCC(COCCC(=O)O)N1C(=O)C=CC1=O] |
| 1-7 | 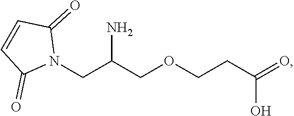 [SMILES: NC(COCCC(=O)O)CN1C(=O)C=CC1=O] |
| 1-8 | 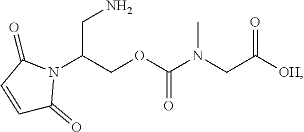 [SMILES: CN(CC(=O)O)C(=O)OCC(CN)N1C(=O)C=CC1=O] |
| 1-9 | 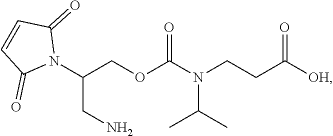 [SMILES: CC(C)N(CCC(=O)O)C(=O)OCC(CN)N1C(=O)C=CC1=O] |
| 1-10 | 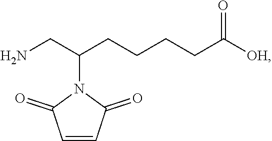 [SMILES: NCC(CCCCC(=O)O)N1C(=O)C=CC1=O] |
| 1-11 | 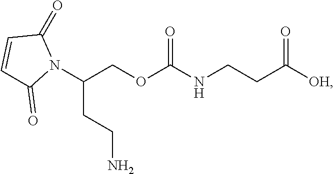 [SMILES: NCCC(COC(=O)NCCC(=O)O)N1C(=O)C=CC1=O] |
| 1-12 | 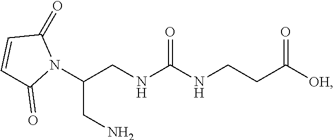 [SMILES: NCC(CNC(=O)NCCC(=O)O)N1C(=O)C=CC1=O] |
| 1-13 | 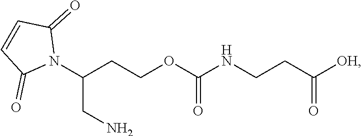 [SMILES: NCC(CCOC(=O)NCCC(=O)O)N1C(=O)C=CC1=O] |
| | or |
| 1-14 | 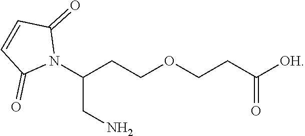 [SMILES: NCC(CCOCCC(=O)O)N1C(=O)C=CC1=O] |

### 7.3. Methods or Processes of Making the Conjugates

Provided herein are methods of preparing a conjugate by contacting a binding agent (BA) with a linker-payload compound under conditions suitable for forming a bond between the binding agent and the linker-payload compound. The reaction conditions may be any suitable reaction conditions known in the art. The binding agent may be an antibody and the bond may form an antibody-drug conjugate.

Examples of such reactions are provided in the Examples below.

In some embodiments, methods of making a conjugate including treating or contacting a compound with a binding agent under coupling conditions. The compound may include a reactive linker bonded to at least one payload. The compound may be any of the linker or platform compounds disclosed herein.

### 7.4. Pharmaceutical Compositions

Also provided herein are compositions, including pharmaceutical compositions, comprising an ADC set forth herein. In some embodiments, the compositions (e.g., pharmaceutical compositions) further comprise a pharmaceutically acceptable excipient.

Pharmaceutical compositions in accordance with the present disclosure can be prepared by mixing an antibody drug conjugate having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to, buffers such as phosphate, citrate, and other organic acids; antioxidants including ascorbic acid and methionine; preservatives (such as octadecyl dimethyl benzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody drug conjugate, which matrices are in the form of shaped articles, e.g. films, or microcapsules.

The formulations to be used for in vivo administration are generally sterile. Sterility can be readily accomplished, e.g., by filtration through sterile filtration membranes.

### 7.5. Methods of Using

In some embodiments, set forth herein is a method of treating a disease or disorder (e.g., a cancer) in a subject (e.g., patient) in need thereof, comprising administering to the patient an effective amount of an ADC disclosed herein.

The antibody drug conjugates disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injection, such as intravenous or subcutaneous injection, depending in part on whether the administration is brief or chronic. Various dosing schedules, including but not limited to, single or multiple administrations over various time-points, bolus administration, and pulse infusion are contemplated herein.

Antibody drug conjugates of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

### 8. EXAMPLES

The examples below are intended to be exemplary and should not be considered limiting in any way. Unless otherwise specified, the experimental methods in the Examples described below are conventional methods. Unless otherwise specified, the reagents and materials are commercially available. All solvents and chemicals employed were of analytical grade or chemical purity. Solvents were redistilled before use. Anhydrous solvents were prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v) and visualized by iodine or the solution of molybdphosphoric acid in ethanol unless otherwise specified. All extraction solvents, unless otherwise specified, were dried over anhydrous Na₂SO₄. ¹H NMR spectra were recorded on Bruck-400, Varian 400MR nuclear magnetic resonance spectrometer with TMS (tetramethylsilane) as the internal standard. Coupling constants were given in hertz. Peaks were reported as singlet(s), doublet (d), triplet (t), quartet (q), quintet (p), sextet (h), septet (hept), multiplet (m), or a combination thereof; br stands for broad. LC/MS data was recorded by using Agilent1100, 1200 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw® 18.0.

For the sake of conciseness, certain abbreviations are used herein. One example is the single letter abbreviation to represent an amino acid. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

In the following examples, the following abbreviations are used:

| | |
|---|---|
| TEA | Triethyl amine |
| THF | Tetrahydrofuran |
| MeOH | Methanol |
| HOBt | 1-hydroxybenzotriazole |
| DIPEA | N,N-diisopropylethylamine |
| DMF | N,N-dimethylformamide |
| NH₄Cl | Ammonium chloride |
| LiOH | Lithium hydroxide |
| HCl | Hydrochloric acid |
| TFA | Trifluoroacetic acid |
| K₂CO₃ | Potassium carbonate |
| Na₂SO₄ | Sodium sulfate |
| EtOAc | Ethyl acetate |
| prep-HPLC | Preparative high performance liquid chromatography |
| Cu(OAc)₂ | Anhydrous cupric acetate |
| Pb(OAc)₄ | Lead tetraacetate |
| TSTU | 2-succinimido-1,1,3,3-tetra-methyluronium |
| | tetrafluor |
| Et₂N | Diethylamine |
| r.t. | Room temperature |
| MS | Mass spectrometry |
| ESI | Electron spray ionization |
| FA | Formic acid |
| MTBE | Methyl tert-butyl ether |
| HPLC | High performance liquid chromatography |
| FCC | Flash column chromatography |
| DMSO | Dimethylsulfoxide |
| DSC | N,N′-Disuccinimidyl carbonate |
| AcOH | Acetic acid |
| Tol | Toluene |
| DMA | Dimethylacetamide |
| DCM | Dichloromethane |
| TBAB | Tetrabutylammonium bromide |
| HATU | Hexafluorophosphate azabenzotriazole |
| | tetramethyl uronium |

### UPLC Analysis Methods

Method A: Mobile phase A: 0.1% FA in water, B: MeCN; Gradient: 10% B maintain 0.2 min, 10%-95% B, 5.8 min, 95% B maintain 0.5 min; Flow rate: 0.6 mL/min; Column: ACQUITY UPLC® BEH C18 1.7 μm.

Method B: Mobile phase A: 0.1% FA in water, B: MeCN; Gradient: 10% B maintain 0.5 min, 10%-90% B, 2.5 min, 90% B maintain 0.2 min; Flow rate: 0.6 mL/min; Column: ACQUITY UPLC® BEH C18 1.7 μm.

Method C: Mobile phase A: 0.1% FA in water, B: MeCN; Gradient: 10% B maintain 0.2 min, 10%-90% B, 1.3 min, 90% B maintain 0.3 min; Flow rate: 0.6 mL/min; Column: ACQUITY UPLC® BEH C18 1.7 μm.

Commercially available compounds 1-1 and 1-3 were purchased from MedChemExpress.

### Example 1-2

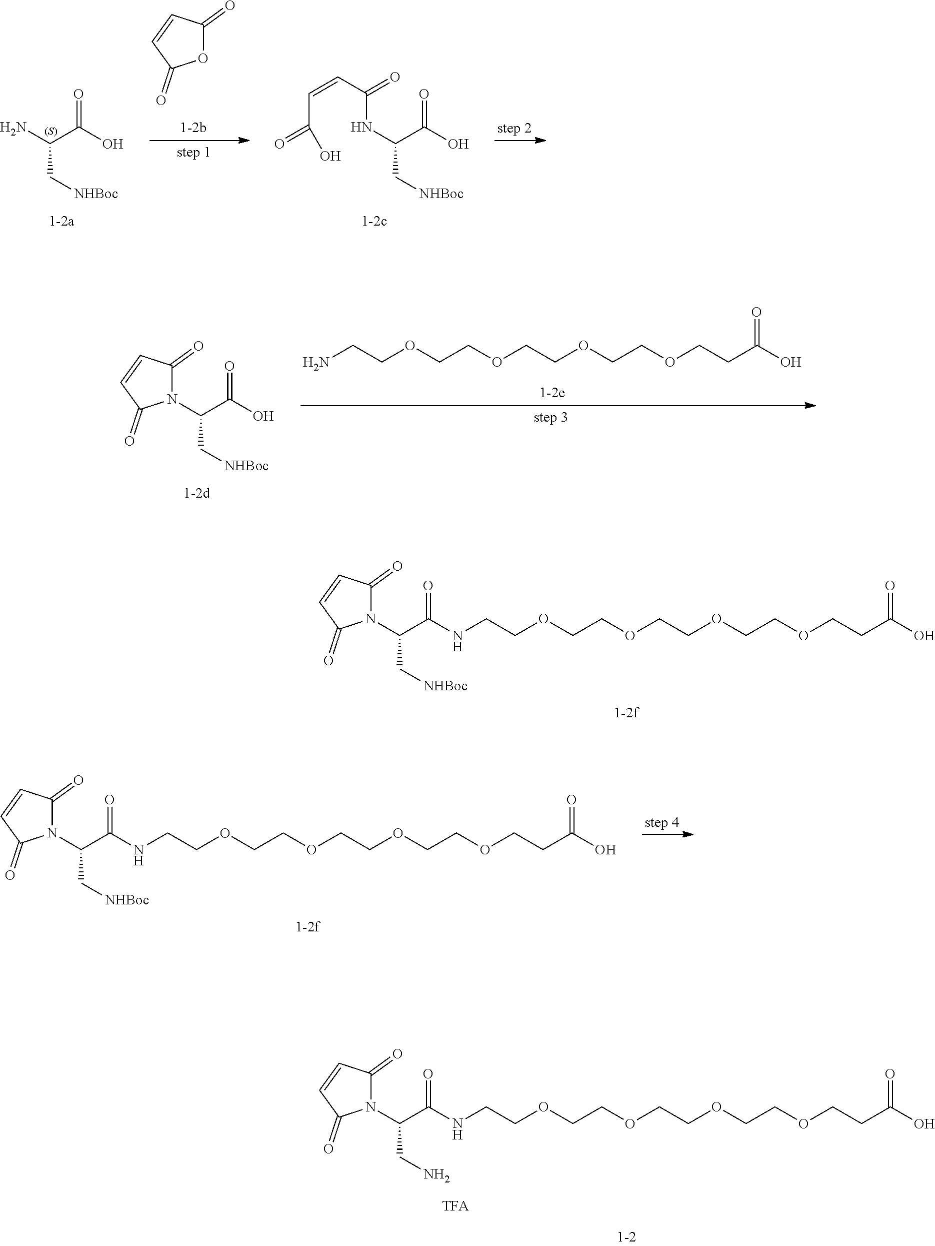

### Step 1: (S,Z)-4-((2-((tert-butoxycarbonyl)amino)-1-carboxyethyl)amino)-4-oxobut-2-enoic acid (1-2c)

A mixture of 1-2a (445.0 mg, 2.18 mmol) and 1-2b (213.7 mg, 2.18 mmol) in glacial acetic acid (10 mL) was prepared. The mixture was stirred at r.t. for 2.5 hr. DCM/hexane (1:1, 30 mL) was added to the reaction mixture, then a white solid was precipitated, filtered, and the solid was co-evaporated with Tol two times to remove the AcOH to give 1-2c (530 mg, ~80.4% yield) as a white solid.

MS (ESI) m/z: 301.2 [M−H].

### Step 2: (S)-3-((tert-butoxycarbonyl)amino)-2-(2,5-dioxo-2,5-dihydro-1H-pyrrol-1-yl)propanoic acid (1-2d)

To a suspension of 1-2c (530 mg, 1.75 mmol) in Tol (18 mL) and DMA (1 mL) were added TEA (532.3 mg, 5.26 mmol) and 4 Å molecular sieve, and then the mixture was heated to 120° C. and stirred at this temperature for 4 hr. The mixture was concentrated under vacuum to remove Tol, then purified by prep-HPLC (FA). The fraction was lyophilized to give 1-2d (201 mg, 40.3% yield) as a white powder.

MS (ESI) m/z: 304.3 [M+Na]⁺.

1H NMR (400 MHz, DMSO) δ 7.09 (s, 2H), 6.99 (t, J=6.4 Hz, 1H), 4.60 (dd, J=10.8, 4.0 Hz, 1H), 3.59-3.53 (m, 1H), 3.46-3.38 (m, 1H), 1.32 (s, 9H).

### Step 3: (S)-7-(2,5-dioxo-2,5-dihydro-1H-pyrrol-1-yl)-2,2-dimethyl-4,8-dioxo-3,12,15,18,21-pentaoxa-5,9-diazatetracosan-24-oic acid (1-2f)

To a solution of 1-2d (25 mg, 0.09 mmol) in DMF (1 mL) were added TSTU (26.5 mg, 0.09 mmol) and DIPEA (45.5 mg, 0.35 mmol). The mixture was stirred at r.t. for 10 min. 1-2d was converted to active ester. 1-2e was added. The mixture was stirred at r.t. for 1 hr. The reaction mixture was purified by prep-HPLC (FA 0.1%), and the fraction was lyophilized to give 1-2f (22 mg, 46.5% yield) as a white powder.

MS (ESI) m/z: 554.5 [M+Na]⁺.

### Step 4: (S)-1-amino-2-(2,5-dioxo-2,5-dihydro-1H-pyrrol-1-yl)-3-oxo-7,10,13,16-tetraoxa-4-azanonadecan-19-oic acid TFA salt (1-2)

A mixture of 1-2f (22.0 mg, 0.04 mmol) in DCM/TFA (1:1, 2 mL) was stirred at r.t. for 1 hr. The mixture was concentrated under vacuum to give crude product and then dissolved in water for lyophilization. 1-2 (17.5 mg, TFA salt) was obtained as a yellow foamy solid.

MS (ESI) m/z: 432.4 [M+H]⁺.

### Example 1-4

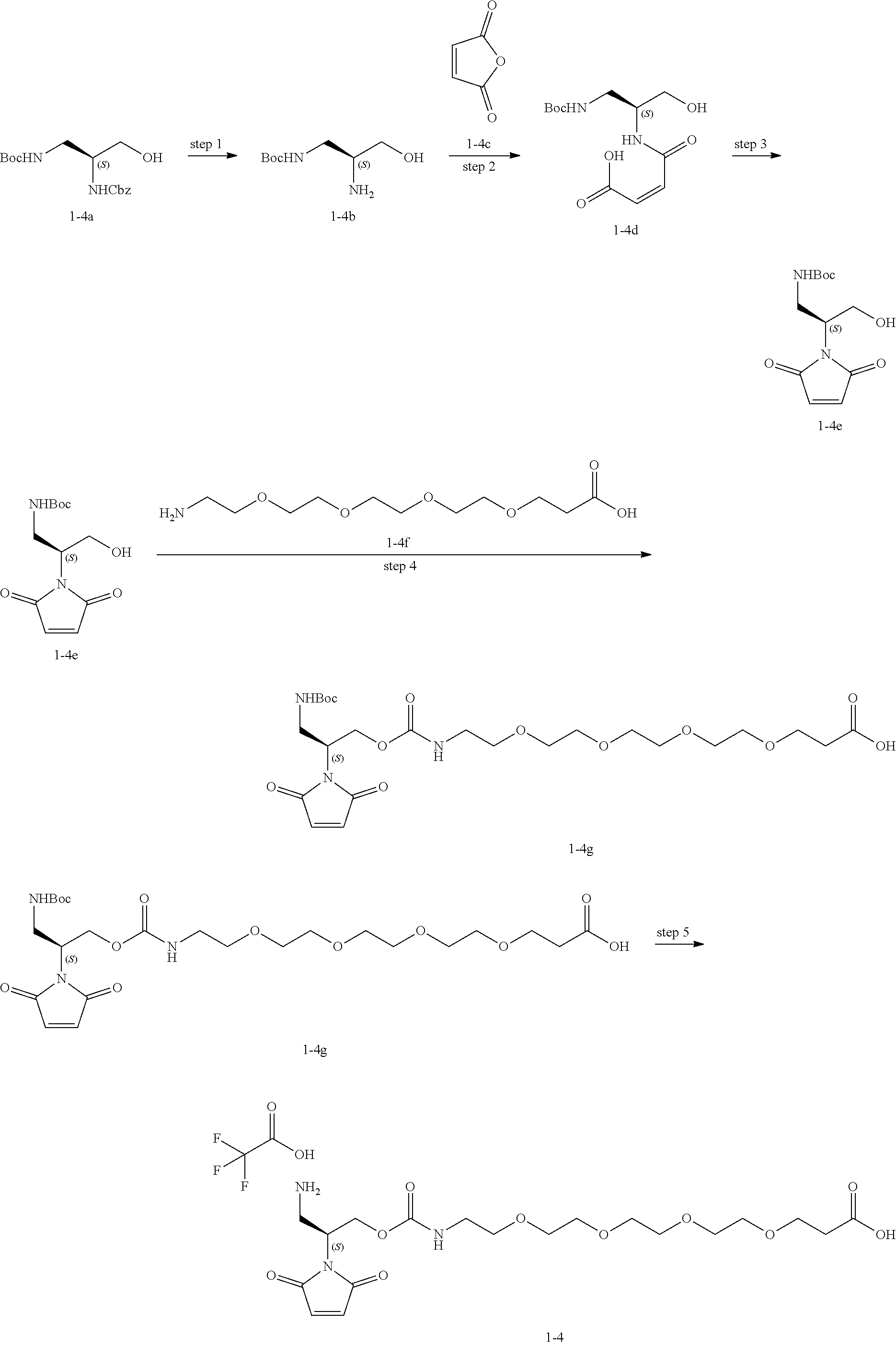

### Step 1: tert-butyl(S)-(2-amino-3-hydroxypropyl)carbamate (1-4b)

To a mixture of 1-4a (2.0 g, 6.17 mmol) (Synthetic reference: Journal of Medicinal Chemistry, 1998, vol. 41, #15, p. 2786-2805) in MeOH (10 mL) was added wet Pd/C (106 mg). The black suspension was purged with an H₂ balloon for 3 times then stirred at r.t. for 1 hr under an H₂ balloon. The mixture was filtered with a syringe head and washed with MeOH/H₂O (5:1, 10 mL). Combined organic layers were concentrated under vacuum to give 1-4b (1.3 g, crude) as a colorless oil.

MS (ESI) m/z: 191.2 [M+H]⁺.

### Step 2: (S,Z)-4-((1-((tert-butoxycarbonyl)amino)-3-hydroxypropan-2-yl)amino)-4-oxobut-2-enoic acid (1-4d)

To a solution of 1-4b (934 mg, 4.91 mmol) in DCM (15 mL) were added dropwise 1-4c (481.43 mg, 4.91 mmol) in DCM (20 mL) at r.t. The mixture was concentrated under vacuum to give a residue. It was dissolved in ACN and purified by prep-HPLC (FA 0.1%). The fraction was concentrated under vacuum to give 1-4d (1010 mg, 71.4% yield) as a colorless oil.

MS (ESI) m/z: 311.3 [M+Na]⁺.

¹H NMR (400 MHZ, CDCl₃) δ 7.98 (d, J=7.4 Hz, 1H), 6.36 (s, 2H), 5.22 (t, J=6.3 Hz, 1H), 3.99 (m, 1H), 3.81 (dd, J=12.2, 2.8 Hz, 1H), 3.64 (dd, J=12.2, 3.2 Hz, 1H), 3.45-3.24 (m, 3H), 1.45 (s, 9H).

### Step 3: tert-butyl(S)-(2-(2,5-dioxo-2,5-dihydro-1H-pyrrol-1-yl)-3-hydroxypropyl)carbamate (1-4e)

To a solution of 1-4d (1010.0 mg, 3.50 mmol) in Tol (40 mL) were added 4 Å molecular sieve (300 mg) and TEA (1063.5 mg, 10.51 mmol). The suspension was purged with an N₂ balloon for three times and heated to 120° C. for overnight. The mixture was filtered and washed with EtOAc (50 mL), washed with 1N KHSO₄ (30 mL*2), dried over Na₂SO₄, filtered and purified by FCC using (petroleum ether/EtOAc=0%~65%) as a mobile phase and the fraction was concentrated under vacuum to give 1-4e (470 mg, 49% yield) as an off-white solid.

MS (ESI) m/z: 293.2 [M+Na]⁺.

¹H NMR (400 MHZ, cdcl₃) δ 6.71 (s, 2H), 4.93 (m, 1H), 4.28 (td, J=9.6, 4.8 Hz, 1H), 3.90 (d, J=5.2 Hz, 2H), 3.66 (dt, J=14.8, 7.6 Hz, 1H), 3.46-3.39 (m, 1H), 1.40 (s, 9H).

### Step 4: (S)-7-(2,5-dioxo-2,5-dihydro-1H-pyrrol-1-yl)-2,2-dimethyl-4,10-dioxo-3,9,14,17,20,23-hexaoxa-5,11-diazahexacosan-26-oic acid (1-4g)

To a solution of 1-4c (110 mg, 0.41 mmol) in DMF (3 mL) were added DSC (114.7 mg, 0.45 mmol) and DIPEA (57.9 mg, 0.45 mmol). The mixture was stirred at r.t. for 2 hr and the mixture turned to brown mixture. 1-4f (140.4 mg, 0.53 mmol) was added and stirred at r.t. for 30 min. The mixture was purified by prep-HPLC (FA), and the fraction was lyophilized to give 1-4g (64 mg, 28% yield) as a white solid.

MS (ESI) m/z: 584.5 [M+Na]⁺.

### Step 5: (S)-1-amino-2-(2,5-dioxo-2,5-dihydro-1H-pyrrol-1-yl)-5-oxo-4,9,12,15,18-pentaoxa-6-azahenicosan-21-oic (TFA salt) (1-4)

A solution of 1-4g (32 mg, 0.057 mmol) in TFA/DCM (1:1, 1 mL) was stirred at r.t. for 10 min. The mixture was concentrated under vacuum and dissolved in water/ACN (2 mL), and lyophilized to give 1-4 (25.2 mg) as a pale-yellow oil.

MS (ESI) m/z: 462.4 [M+H]⁺.

### Example 1-5

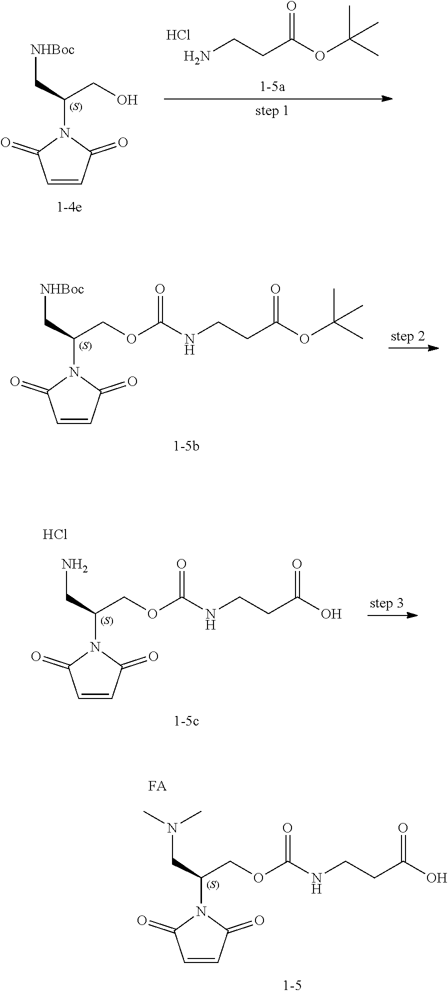

### Step 1: tert-butyl(S)-7-(2,5-dioxo-2,5-dihydro-1H-pyrrol-1-yl)-2,2-dimethyl-4,10-dioxo-3,9-dioxa-5,11-diazatetradecan-14-oate (1-5b)

To a solution of 1-4e (107 mg, 0.40 mmol) in DMF (3 mL) were added DSC (121.7 mg, 0.48 mmol) and DIPEA (76.8 mg, 0.59 mmol). The mixture was stirred at RT for 2 hr and turned to brown mixture. 1-5a (93.5 mg, 0.52 mmol) was added and stirred at r.t. for 30 min. It was purified by prep-HPLC (FA 0.1%), and the fraction was lyophilized to give 1-5b (69 mg, 40% yield) as a white solid.

MS (ESI) m/z: 464.4 [M+Na]⁺.

### Step 2: (S)-3-(((3-amino-2-(2,5-dioxo-2,5-dihydro-1H-pyrrol-1-yl)propoxy)carbonyl)amino)propanoic acid HCl salt (1-5c)

A solution of 1-5b (69 mg, 0.16 mmol) in 4M HCl/EtOAc (3 mL) was stirred in an ice-bath for 1 hr. The mixture was concentrated under vacuum to give 1-5c (55 mg, crude) as an off-white solid.

MS (ESI) m/z: 286.2 [M+H]⁺.

### Step 3: (S)-3-(((3-(dimethylamino)-2-(2,5-dioxo-2,5-dihydro-1H-pyrrol-1-yl)propoxy)carbonyl)amino)propanoic acid (1-5)

To a suspension of 1-5c (55 mg, 0.19 mmol) in DCM was added 30% formaldehyde in water (5.8 g, 200 eq.). The mixture was stirred at r.t. for 10 min, then NaBH(OAc)₃ (102.1 mg, 0.48 mmol) was added and stirred at r.t. for 1 hr. N₂ bubble was used to remove DCM and the remainder was purified by prep-HPLC (FA) to give 1-5 (13.4 mg, 22% yield) as a pale-yellow oil.

MS (ESI) m/z: 314.3 [M+H]⁺.

### Example 1-6

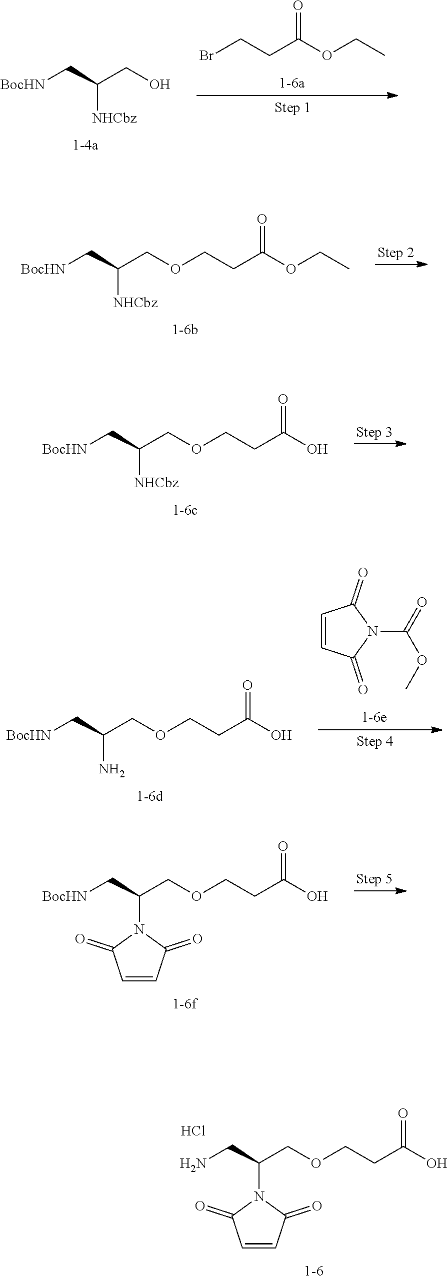

### Step 1: ethyl(S)-3-(2-(((benzyloxy)carbonyl)amino)-3-((tert-butoxycarbonyl)amino)propoxy)propanoate (1-6b)

To a mixture of 1-4a (9.3 g, 28.7), TBAB (924.2 mg, 2.87 mmol), and 1-6a (26.0 g, 143.4 mmol) in DCM (100 mL) was added dropwise 50% wt NaOH aqueous (40 mL) in an ice-bath. The mixture was allowed to warm to r.t. and stirred at r.t. overnight. The mixture was acidified with 1N KHSO₄ in an ice-bath to pH=3, and extracted with DCM (100 mL*3). Combined organic layers were washed with brine (50 mL*2), dried over Na₂SO₄, filtered, and concentrated under vacuum to give 1-6b (12 g, crude) as a yellow oil.

MS (ESI) m/z: 447.4 [M+Na]⁺.

### Step 2: (S)-3-(2-(((benzyloxy)carbonyl)amino)-3-((tert-butoxycarbonyl)amino)propoxy)propanoic acid (1-6c)

To a solution of 1-6b (12 g, crude) in MeOH (50 mL) was added 2N LiOH (10 mL). The mixture was stirred at r.t. for 4 hr. The mixture was acidified with 1N KHSO₄ in an ice-bath to pH=3, and extracted with EA (100 mL*3). Combined organic layers were washed with brine (100 mL), dried over Na₂SO₄, and filtered, and the filtrate was concentrated under vacuum to give a residue. It was purified by FCC (EA/PE=40%~100%) as a mobile phase to give 1-6c (2.7 g crude) as a colorless transparent oil.

MS (ESI) m/z: 419.3 [M+Na]⁺.

### Step 3: (S)-3-(3-((tert-butoxycarbonyl)amino)-2-(2,5-dioxo-2,5-dihydro-1H-pyrrol-1-yl)propoxy)propanoic acid (1-6d)

To a solution of 1-6c (2 g, crude) in MeOH (30 mL) was added wet Pd/C (200 mg), then the mixture was stirred at r.t. for 30 min. The black suspension was filtered through a syringe head, washed with H₂O, then concentrated under vacuum to give a residue, then dissolved in water (30 mL), and extracted with EA (50 mL*3). The aqueous phase was concentrated under vacuum to give 1-6d (930 mg) as colorless transparent oil.

MS (ESI) m/z: 263.4 [M+H]+.

### Step 4: (S)-3-(3-((tert-butoxycarbonyl)amino)-2-(2,5-dioxo-2,5-dihydro-1H-pyrrol-1-yl)propoxy)propanoic acid (1-6f)

To a mixture of 1-6d (930 mg, crude) in sat. NaHCO₃ (10 mL) was added 1-6e (827.3 mg, 5.9 mmol) in an ice-bath and stirred for 20 min, then allowed to warm to r.t. for 1 hr. The mixture was acidified with 1N KHSO₄ to pH=3 in an ice-bath, and extracted with EA (20 mL*3), and combined organic layers were dried over Na₂SO₄, filtered, and the filtrate was concentrated under vacuum to give a residue. It was purified by prep-HPLC (FA) to give 1-6f (189 mg) as a white solid.

MS (ESI) m/z: 365.3 [M+H]⁺.

### Step 5: (S)-3-(3-((tert-butoxycarbonyl)amino)-2-(2,5-dioxo-2,5-dihydro-1H-pyrrol-1-yl)propoxy)propanoic acid HCl salt (1-6)

A solution of 1-6f (26 mg, 0.08 mmol) in 4N HCl/EtOAc (1 mL) was stirred at r.t. for 1 hr. The suspension was filtered and washed with EtOAc to give 1-6 (12.1 mg) as a white solid.

MS (ESI) m/z: 243.2 [M+H]⁺.

¹H NMR (400 MHZ, dmso) δ 12.25 (s, 1H), 8.16 (s, 3H), 7.12 (s, 1H), 4.47-4.37 (m, 1H), 3.69 (d, J=10.8 Hz, 2H), 3.66-3.57 (m, 2H), 3.34 (m, 1H), 3.15 (d, J=11.6 Hz, 1H), 2.45 (t, J=6.4 Hz, 2H).

### Example 1-7

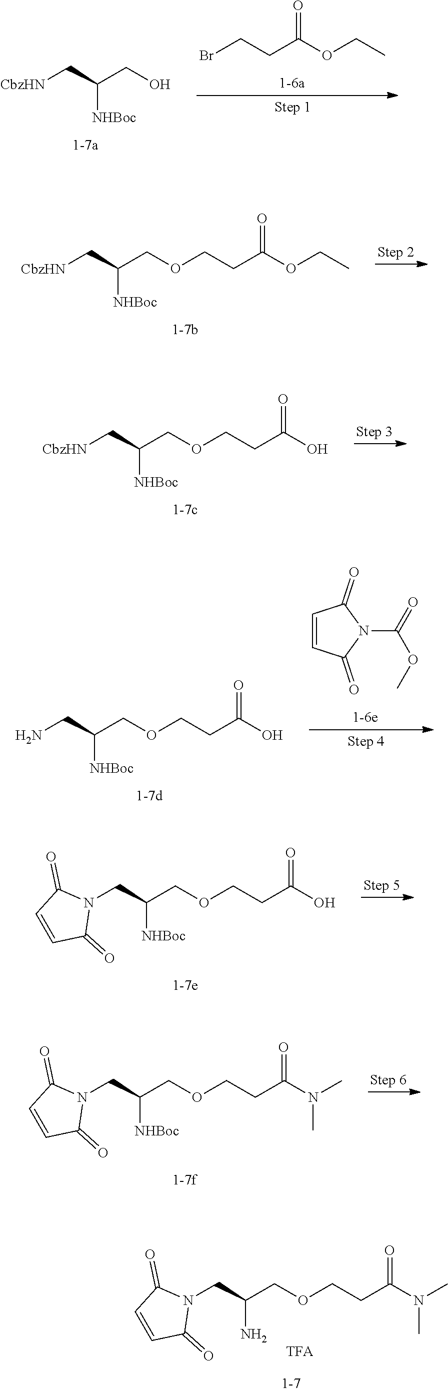

### Step 1: ethyl(S)-3-(3-(((benzyloxy)carbonyl)amino)-2-((tert-butoxycarbonyl)amino)propoxy)propanoate (1-7b)

To a mixture of 1-7a (5.0 g, 15.41 mmol) (purchased from WUXI), TBAB (496.9 mg, 1.54 mmol), and 1-6a (14.0 g, 77.07 mmol) in DCM (50 mL) was added dropwise 50% wt NaOH aqueous (7.2 g, 77.07 mmol). The mixture was stirred at r.t. overnight. The mixture was diluted with DCM (100 mL), and extracted with DCM (50 mL*2). Combined organic layers were washed with brine (50 mL*2), dried over Na₂SO₄, filtered, and concentrated under vacuum to give a residue. It was purified by silica gel column chromatography (PE/EA=0%~30%), and the fraction was concentrated under vacuum to give 1-7b (3.06 g, ~46.8% purity) as a colorless transparent oil.

MS (ESI) m/z: 447.4 [M+Na]⁺.

¹H NMR (400 MHZ, CDCl₃) ¿ 7.32 (m, 5H), 5.35 (s, 1H), 5.18-5.04 (m, 2H), 4.19-4.09 (dq, J=6.8, 1.6 Hz 2H), 3.82 (m, 1H), 3.69 (m, 2H), 3.56 (dd, J=9.2, 3.6 Hz, 1H), 3.47 (dd, J=9.6, 5.2 Hz, 1H), 3.45-3.37 (m, 1H), 3.36-3.25 (m, 1H), 2.55 (t, J=6.0 Hz, 2H), 1.43 (s, 9H), 1.24 (t, J=7.2 Hz, 3H).

### Step 2: (S)-3-(3-(((benzyloxy)carbonyl)amino)-2-((tert-butoxycarbonyl)amino)propoxy)propanoic acid (1-7c)

To a solution of 1-7b (3.06 g, 7.21 mmol) in MeOH (30 mL) was added 4N NaOH (2.7 mL, 10.8 mmol) and then stirred at r.t. for 12 hr. The mixture was diluted with water (30 mL) and acidified with 1N KHSO₄ to pH=3 in an ice-bath, and extracted with EtOAc (50 mL*4). Combined organic layers were washed with brine (50 mL*3), dried over Na₂SO₄, filtered, and the filtrate was concentrated under vacuum to give 1-7c (2.8 g, 98% yield) as a colorless transparent oil.

MS (ESI) m/z: 419.3 [M+Na]⁺.

### Step 3: (S)-3-(3-amino-2-((tert-butoxycarbonyl)amino)propoxy)propanoic acid (1-7d)

To a solution of 1-7c (2.8 g, 7.1 mmol) in MeOH (30 mL) was added wet Pd/C (250 mg) and then stirred at r.t. for 30 min. The black suspension was filtered through a syringe head washed with H₂O and then concentrated under vacuum to give 1-7d (1.85 g) as a white solid.

MS (ESI) m/z: 263.2 [M+H]⁺.

¹H NMR (400 MHZ, d₂o) 8 4.1~3.9 (m, 1H), 3.73 (t, J=6.0 Hz, 2H), 3.64 (dd, J=10.4, 4.4 Hz, 1H), 3.57 (dd, J=10.4, 5.2 Hz, 1H), 3.22 (dd, J=13.2, 4.4 Hz, 1H), 3.08 (dd, J=13.2, 8.0 Hz, 1H), 2.45 (t, J=6.0 Hz, 2H), 1.44 (s, 9H).

### Step 4: (S)-3-(2-((tert-butoxycarbonyl)amino)-3-(2,5-dioxo-2,5-dihydro-1H-pyrrol-1-yl)propoxy)propanoic acid (1-7e)

To a solution of 1-7d (500 mg, 1.91 mmol) in 1N NaHCO₃ (10 mL) was added 1-6e (397.7 mg, 2.86 mmol). The mixture was stirred at 0° C. for 30 min then slowly warmed to r.t. for 2 hr. The mixture was acidified with 1N KHSO₄ to pH=3 in an ice-bath, and extracted with EtOAc (20 mL*2). Combined organic layers were washed with brine (20 mL), dried over Na₂SO₄, filtered, and the filtrate was concentrated under vacuum to give 1-7e (560 g, 85.8% yield) as a white solid.

MS (ESI) m/z: 365.3 [M+Na]⁺.

¹H NMR (400 MHZ, cdcl₃) δ 6.69 (s, 2H), 5.06 (d, J=9.2 Hz, 1H), 4.08~3.97 (m, 1H), 3.82~3.68 (m, 3H), 3.59 (dd, J=14.0, 3.6 Hz, 2H), 3.51 (dd, J=9.6, 4.4 Hz, 1H), 2.64 (t, J=6.2 Hz, 1H), 1.38 (s, 9H).

### Step 5: tert-butyl(S)-(1-(3-(dimethylamino)-3-oxopropoxy)-3-(2,5-dioxo-2,5-dihydro-1H-pyrrol-1-yl) propan-2-yl) carbamate (1-7f)

To a solution of 1-7e (50 mg, 0.15 mmol) in DMF (2 mL) were added HATU (55.5 mg, 0.15 mmol), DIPEA (56.6 mg, 0.44 mmol), and dimethylamine hydrochloride (11.9 mg, 0.15 mmol). The mixture was stirred at r.t. for 30 min. The mixture was diluted with EtOAc (20 mL), washed with brine (15 mL*3), dried over Na₂SO₄, filtered, and the filtrate was concentrated under vacuum to give a residue. It was purified by FCC (MeOH/DCM=0~5%) as a mobile phase to give 1-7f (35 mg, 64.9% yield) as a yellow oil.

MS (ESI) m/z: 392.4 [M+Na].

### Step 6: (S)-3-(2-amino-3-(2,5-dioxo-2,5-dihydro-1H-pyrrol-1-yl) propoxy)-N,N-dimethylpropanamide TFA salt (1-7)

A solution of 1-7f (35 mg, 0.095 mmol) in DCM/TFA (1:1, 2 mL) was stirred at r.t. for 10 min. The mixture was concentrated under vacuum to give a residue, then dissolved in water (20 mL), and extracted with EtOAc (20 mL*3). The aqueous phase was lyophilized to give 1-7 (38.5 mg) as a brown oil.

MS (ESI) m/z: 392.4 [M+Na]⁺.

1H NMR (400 MHZ, dmso) δ 8.05 (s, 3H), 7.09 (s, 2H), 3.73-3.55 (m, 6H), 3.53-3.42 (m, 1H), 2.97 (s, 3H), 2.83 (s, 3H), 2.58 (t, J=6.4 Hz, 2H).

### Example 1-8

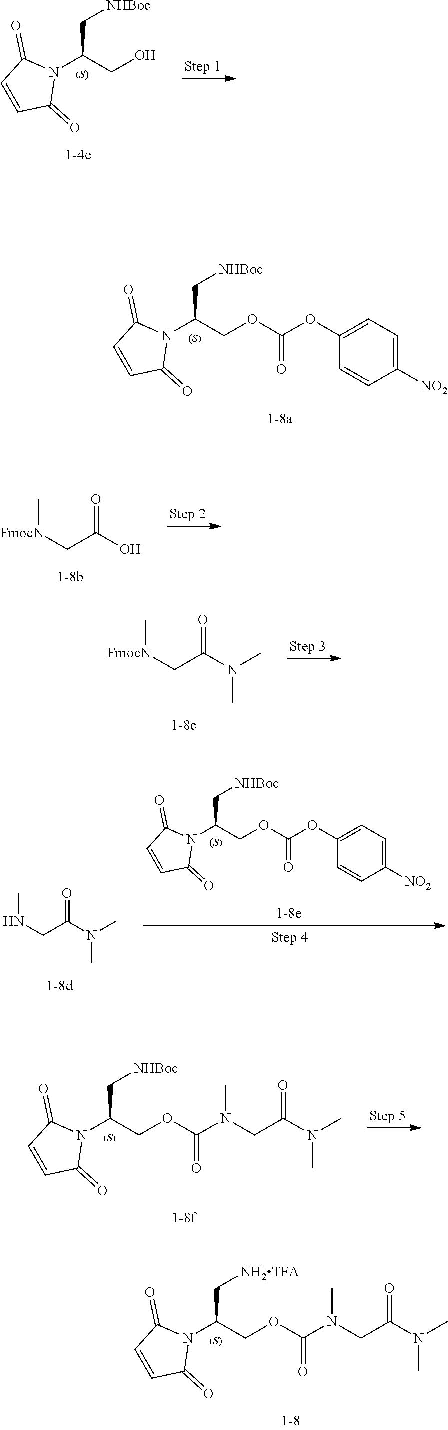

### Step 1: tert-Butyl(S)-(2-(2,5-dioxo-2,5-dihydro-1H-pyrrol-1-yl)-3-(((4-nitrophenoxy)carbonyl)oxy)propyl)carbamate (1-8a)

To a solution of 1-4e (81 mg, 0.3 mmol) and bis(4-nitrophenyl) carbonate (100.2 mg, 0.33 mmol) in DMF (2.3 mL) was added DIPEA (81.6 ml, 0.45 mmol) at r.t. After addition, the mixture was stirred at r.t. overnight. The mixture was diluted with EtOAc and the organic layer was washed with sat. NH₄Cl aq., H₂O, and brine, dried over anhydrous Na₂SO₄, filtered and the filtrate was concentrated under vacuum to give a residue. The crude product was triturated with MTBE and filtered to give the target product 1-8a (91 mg, 70% yield) as a white solid.

MS (ESI) m/z: 458.4 [M+Na]⁺.

### Step 2: (9H-Fluoren-9-yl)methyl (2-(dimethylamino)-2-oxoethyl)(methyl)carbamate (1-8c)

To a solution of 1-8b (1.0 g, 3.2 mmol) in DMF (15 mL) were added HATU (1.34 g, 3.53 mmol), DIPEA (1.24 g, 9.63 mmol), and dimethylamine hydrochloride (314 mg, 3.85 mmol). The reaction was stirred at r.t. for 30 min. The mixture was diluted with EtOAc (100 mL), washed with brine (15 mL×3), dried over Na₂SO₄, filtered and the filtrate was concentrated under vacuum to give a residue. It was purified by FCC (MeOH/DCM=0~5%) as a mobile phase to give 1-8c (1.75 g, >100% yield) as a yellow oil (containing 1-hydroxy-7-azabenzotriazole (HOAt)).

MS (ESI) m/z: 339.3 [M+H]⁺.

### Step 3: N, N-Dimethyl-2-(methylamino) acetamide (1-8d)

To a solution of 1-8c (100.0 mg, 0.30 mmol) in DMF (3 mL) was added Et₂NH (252 mg, 3.0 mmol). The mixture was stirred at r.t. for 20 min. The reaction mixture was concentrated under vacuum and co-evaporated with toluene two times to give 1-8d as a brown solid, which was used in the next step directly.

### Step 4: (S)-3-((tert-Butoxycarbonyl)amino)-2-(2,5-dioxo-2,5-dihydro-1H-pyrrol-1-yl)propyl (2-(dimethylamino)-2-oxoethyl)(methyl)carbamate (1-8f)

To a solution of 1-8e (50.0 mg, 0.115 mmol) in DMF (1 mL) was added crude solid 1-8d and DIPEA (44.5 mg 0.345 mmol) at r.t. The mixture was stirred at r.t. for 30 min. The reaction mixture was diluted with DMF and acidified with 0.1 N HCl. The mixture was purified by prep-HPLC (FA), and the fraction was lyophilized to give 1-8f (17.2 mg, 36.3% yield) as a pale-yellow solid.

MS (ESI) m/z: 435.4 [M+Na]⁺.

### Step 5: (S)-3-amino-2-(2,5-dioxo-2,5-dihydro-1H-pyrrol-1-yl)propyl (2-(dimethylamino)-2-oxoethyl)(methyl)carbamate (1-8)

A solution of 1-8f (7.7 mg, 0.021 mmol) in TFA/DCM (1:1, 0.6 mL) was stirred at r.t. for 60 min. The mixture was concentrated under vacuum and dissolved in water/ACN (1:1, 2 mL), and lyophilized to give 1-8 (7.8 mg, ~100% yield) as a white solid.

MS (ESI) m/z: 313.3 [M+H]⁺.

### Example 1-9

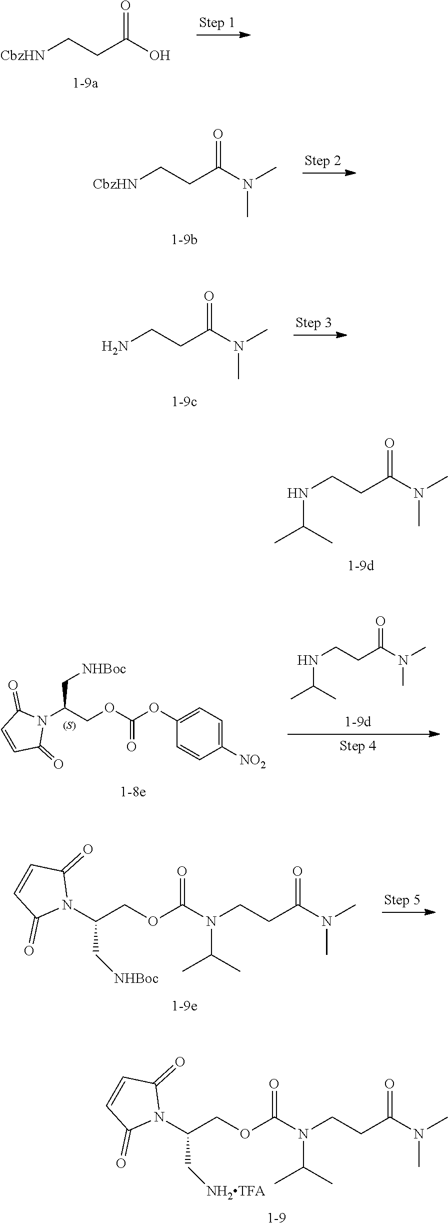

### Step 1: Benzyl (3-(dimethylamino)-3-oxopropyl)carbamate (1-9b)

To a solution of 1-9a (1.116 g, 5 mmol) in DMF (25 mL) were added HATU (2.09 g, 5.5 mmol), DIPEA (1.94 g, 15 mmol), and dimethylamine hydrochloride (489 mg, 6 mmol). The reaction was stirred at r.t. for 30 min. The mixture was diluted with EtOAc (100 mL), washed with brine (15 mL×3), dried over Na₂SO₄, filtered and the filtrate was concentrated under vacuum to give a residue. It was purified by FCC (MeOH/DCM=0~5%) as a mobile phase to give 1-9b (0.987 g, 79% yield) as a yellow oil.

MS (ESI) m/z: 251.2 [M+H]⁺.

### Step 2:3-Amino-N, N-dimethylpropanamide (1-9c)

To a suspension of 1-9b (500 mg, 2.0 mmol) in MeOH (5 mL) was added Pd/C (50 mg, 10% wt/wt) at r.t. The atmosphere was replaced with H₂ and the mixture was stirred at r.t. for 2 h. The mixture was filtered through a pad of celite, and the filtrate was concentrated under vacuum to give crude product 1-9c (233 mg, ~100% yield) as a colorless oil, which was used for the next step directly.

MS (ESI) m/z: 117.0 [M+H]⁺.

### Step 3:3-(Isopropylamino)-N, N-dimethylpropanamide (1-9d)

To a solution of 1-9c (233 mg, 2.0 mmol) and acetone (232.3 mg, 4.0 mmol) in MeOH (9 mL) was added NaBH₃CN (510.8 mg, 8.0 mmol) at r.t. The reaction was stirred at r.t. for 18 h. The mixture was concentrated in vacuo and the residue was re-dissolved in DCM (20 mL) and washed with sat. NaHCO₃ (10 mL), H₂O (10 mL), brine (10 mL), dried over Na₂SO₄, filtered and the filtrate was concentrated under vacuum to give crude target product 1-9d (164 mg, 52% yield) as a pale-green oil.

MS (ESI) m/z: 159.1 [M+H]⁺.

### Step 4: (S)-3-((tert-Butoxycarbonyl)amino)-2-(2,5-dioxo-2,5-dihydro-1H-pyrrol-1-yl)propyl (3-(dimethylamino)-3-oxopropyl)(isopropyl)carbamate (1-9e)

To a solution of 1-8e (30.0 mg, 0.115 mmol) in DMF (1 mL) was added crude oil 1-9d (33 mg, 0.21 mmol) and DIPEA (27 mg, 0.21 mmol) at r.t. The reaction was stirred at r.t. for 30 min. The reaction mixture was diluted with DMF and acidified with 0.1 N HCl. The mixture was purified by prep-HPLC (FA), and the fraction was lyophilized to give 1-9e (22.5 mg, 58.8% yield) as a white solid.

MS (ESI) m/z: 477.4 [M+Na]⁺.

### Step 5: (S)-3-amino-2-(2,5-dioxo-2,5-dihydro-1H-pyrrol-1-yl)propyl (3-(dimethylamino)-3-oxopropyl)(isopropyl)carbamate trifluoroacetate (1-9)

A solution of 1-9e (22.5 mg, 0.0495 mmol) in TFA/DCM (1:1, 1.2 mL) was stirred at r.t. for 60 min. The mixture was concentrated under vacuum and dissolved in water/ACN (1:1, 2 mL), and lyophilized to give 1-9 (23.2 mg, ~100% yield) as a white solid.

MS (ESI) m/z: 355.3 [M+H]⁺.

### Example 1-10

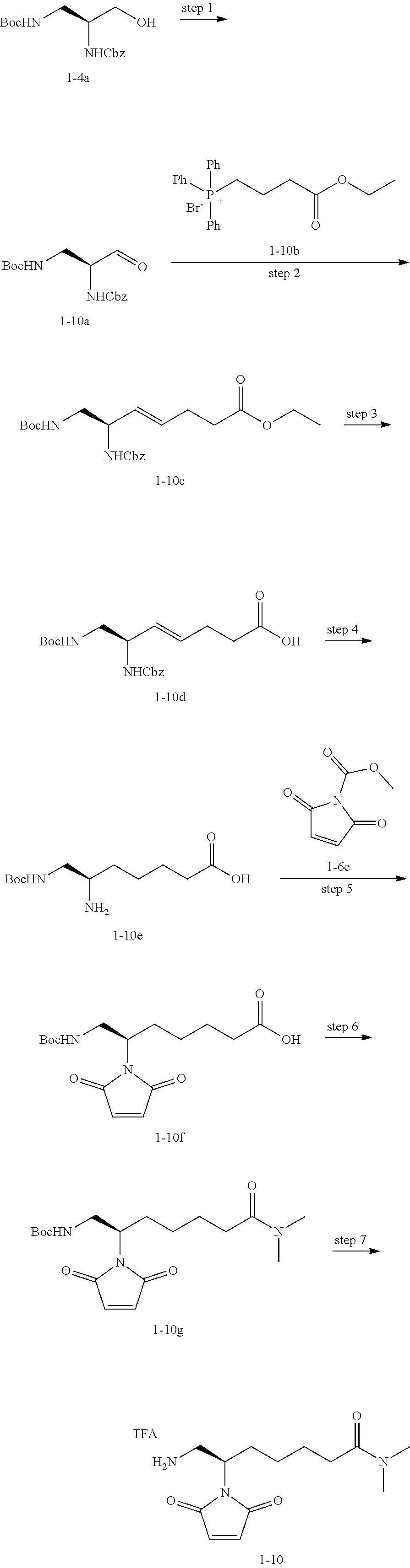

### Step 1: benzyl tert-butyl (3-oxopropane-1,2-diyl) (S)-dicarbamate (1-10a)

A solution of (COCl)₂ (3.9 g, 30.83 mmol) in DCM (50 mL) was cooled to −70° C., DMSO (3.6 g, 46.24 mmol) was added dropwise, then stirred at −70° C. for 30 min. 1-4a (5.0 g, 15.41 mmol) was dissolved in DMSO (10 mL) and was added through syringe then stirred at −70° C. for 30 min. TEA (7.8 g, 77.1 mmol) was added dropwise and then slowly warmed to r.t. The mixture was poured into water (100 mL) and extracted with EtOAc (50 mL*2). Combined organic layers were washed with brine (100 mL*2), dried over Na₂SO₄, filtered and the filtrate was concentrated under vacuum to give a residue. It was purified by FCC (EA/PE=1%~50%) as mobile phase, and the fraction was concentrated under vacuum to give 1-10a (4.86 g, 97.8% yield) as a yellow oil.

1H NMR (400 MHZ, cdcl₃) δ 9.64 (s, 1H), 7.39-7.30 (m, 5H), 5.93 (s, 1H), 4.86 (s, 1H), 4.35-4.22 (m, 1H), 3.77-3.64 (m, 1H), 3.56 (dt, J=14.8, 4.8 Hz, 1H), 1.40 (s, 9H).

### Step 2: ethyl (R)-6-(((benzyloxy)carbonyl)amino)-7-((tert-butoxycarbonyl)amino)hept-4-enoate (1-10c)

To a suspension of 1-10b (6.5 g, 14.3 mmol) in Tol (30 mL) was added dropwise t-BuOK (1M solution in THF, 14.5 mL, 14.5 mmol) at 0° C. The mixture turned a deep color over 30 min. 1-10a (2.0 g, 6.2 mmol) in Tol (20 mL) was added dropwise at −70° C., then slowly warmed to r.t. The mixture was quenched by addition of sat. NH₄Cl (50 mL). After separation, the Tol phase was washed with brine (30 mL*2), dried over Na₂SO₄, filtered and the filtrate was concentrated under vacuum to give a residue. Purification by FCC (EA/PE=0%~30%~50%) gave 1-10c (808 mg, 30.8% yield) as a white solid.

MS (ESI) m/z: 443.4 [M+Na]⁺.

¹H NMR (400 MHZ, cdcl₃) δ 7.40-7.27 (m, 5H), 5.72~5.60 (m, 0.2H), 5.60-5.46 (m, 0.8H), 5.40 (dd, J=16.0 Hz, 6.4 Hz, 0.2H), 5.34~5.20 (m, 1.8H), 4.95~4.70 (m, 1H), 5.16~5.01 (m, 2H), 4.60~4.40 (m, 1H), 4.12 (q, J=7.2 Hz, 2H), 3.39-3.08 (m, 2H), 2.6~2.2 (m, 4H), 1.42 (s, 9H), 1.24 (t, J=7.2 Hz, 1H).

### Step 3: (R)-6-(((benzyloxy)carbonyl)amino)-7-((tert-butoxycarbonyl)amino)hept-4-enoic acid (1-10d)

To a solution of 1-10c (808.0 mg, 1.92 mmol) in MeOH (10 mL) was added 4N NaOH (1.92 mL, 7.69 mmol). The mixture was stirred at r.t. for 3.5 hr. The mixture was diluted with water (20 mL), acidified with sat. KHSO₄ to pH=3, and extracted with EtOAc (30 mL*3). Combined organic layers were washed with brine (50 mL), dried over Na₂SO₄, filtered and the filtrate was concentrated under vacuum to give 1-10d (781 mg) as a white solid.

MS (ESI) m/z: 415.4 [M+Na]⁺.

### Step 4: (R)-6-amino-7-((tert-butoxycarbonyl)amino)heptanoic acid (1-10e)

To a solution of 1-10d (781.0 mg, 1.99 mmol) in MeOH (8 mL) was added wet Pd/C (67 mg) then stirred at r.t. for 24 hr. The black suspension was filtered through a syringe head washed with MeOH/H₂O (5:1, 30 mL) then concentrated under vacuum to give 1-10e (510 mg, 98.5% yield) as a white solid.

MS (ESI) m/z: 261.3 [M+H]⁺.

### Step 5: (R)-7-((tert-butoxycarbonyl)amino)-6-(2,5-dioxo-2,5-dihydro-1H-pyrrol-1-yl) heptanoic acid (1-10f)

To a mixture of 1-10e (510 mg, 1.96 mmol) in 1N NaHCO₃ (10 mL) was added 1-6e (455.8 mg, 2.94 mmol) at 0° C., then stirred at 0° C. for 30 min and allowed to warm to r.t. for 3 hr. The reaction was acidified with sat. KHSO₄ to pH=3, and extracted with EtOAc (30 mL*3). Combined organic layers were washed with brine (50 mL), dried over Na₂SO₄, filtered and the filtrate was concentrated to give a residue. It was purified by FCC MeOH/DCM=0%~5%). The fraction was concentrated under vacuum to give 1-10f (590 mg, 85% yield) as a pale-yellow oil.

MS (ESI) m/z: 363.3 [M+Na]⁺.

### Step 6: tert-butyl (R)-(7-(dimethylamino)-2-(2,5-dioxo-2,5-dihydro-1H-pyrrol-1-yl)-7-oxoheptyl) carbamate (1-10g)

To a solution of 1-10f (53 mg, 0.16 mmol) in DMF (2 mL) were added HATU (71.6 mg, 0.19 mmol), DIPEA (44.3 mg, 0.34 mmol), and dimethylamine hydrochloride (14.0 mg, 0.17 mmol). The mixture was stirred at r.t. for 30 min. The mixture was diluted with EtOAc (20 mL), washed with brine (15 mL*3), dried over Na₂SO₄, filtered and the filtrate was concentrated under vacuum to give a residue. It was purified by FCC (MeOH/DCM=0~5%) as a mobile phase to give 1-10g (53 mg, 92.1% yield) as a yellow oil.

MS (ESI) m/z: 390.4 [M+Na]⁺.

### Step 7: (R)-7-amino-6-(2,5-dioxo-2,5-dihydro-1H-pyrrol-1-yl)-N,N-dimethylheptanamide TFA salt (1-10)

A solution of 1-10g (30 mg, 0.08 mmol) in DCM/TFA (1:1, 2 mL) was stirred at r.t. for 10 min. The mixture was concentrated under vacuum to give a residue then dissolved in water (20 mL) and extracted with EtOAc (20 mL*3). The aqueous phase was lyophilized to give 1-10 (33 mg) as a brown oil.

MS (ESI) m/z: 268.3 [M+H]⁺.

### Example 1-11

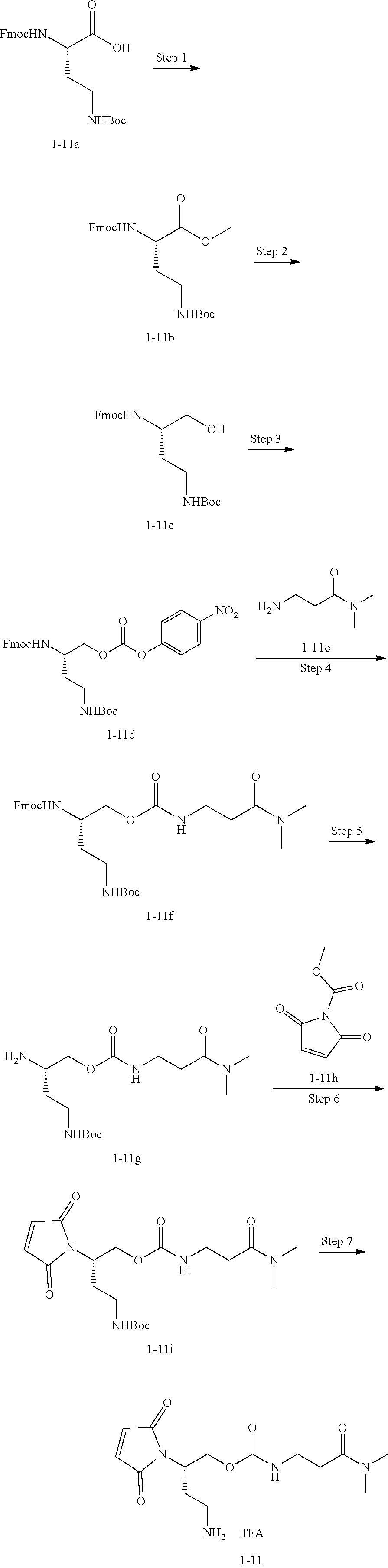

### Step 1: Methyl(S)-2-((((9H-fluoren-9-yl)methoxy)carbonyl)amino)-4-((tert-butoxycarbonyl)amino)butanoate (1-11b)

1-11a (4.00 g, 9.08 mmol) and K₂CO₃ (1.38 g, 9.99 mmol) were added to DMF (10 mL) followed by the dropwise addition of CH₃I (2.58 g, 18.16 mmol) at 0° C. The resulting mixture was stirred at 0° C. for 20 min and allowed to warm to 25° C. and further stirred at 25° C. for 30 min. After a complete reaction, the reaction mixture was diluted with EA (50 mL) and washed with brine (25 mL*3) and H₂O (25 mL*2). The organic layer was dried over anhydrous Na₂SO₄, filtered, and concentrated under reduced pressure to afford 1-11b (4.08 g, quant.) as a light-yellow solid.

MS (ESI) m/z: 477.4 [M+Na]⁺.

### Step 2: (9H-fluoren-9-yl)methyl tert-butyl (4-hydroxybutane-1,3-diyl) (S)-dicarbamate (1-11c)

1-11b (1.00 g, 2.20 mmol) was dissolved in THF (55 mL) and EtOH (55 mL) followed by the addition of NaBH₄ (499 mg, 13.20 mmol) and LiCl (616 mg, 14.52 mmol) successively at 0° C. The resulting mixture was stirred at 25° C. for 1.5 h. After a complete reaction, saturated aqueous NH₄Cl (10 mL) was added to quench the reaction. The reaction mixture was diluted with H₂O (50 mL) and extracted with EA (35 mL*3). The combined organic layers were washed with brine (30 mL*2) and water (30 mL*2), dried over anhydrous Na₂SO₄, filtered, and concentrated under reduced pressure to afford 1-11c as a white solid (938 mg, quant.). The product was used directly in the next step without purification.

MS (ESI) m/z: 449.4 [M+Na]⁺.

¹H NMR (400 MHZ, d₆-DMSO) δ 7.86 (d, J=7.2 Hz, 2H), 7.70-7.67 (m, 2H), 7.39 (t, J=7.2 Hz, 2H), 7.32-7.29 (m, 2H), 7.07-7.05 (m, 1H), 6.67 (t, J=5.2 Hz, 1H), 6.51 (s, 1H), 4.62 (br s, 1H), 4.29-4.17 (m, 3H), 3.41-3.38 (m, 1H), 3.25-3.24 (m, 1H), 2.98-2.95 (m, 1H), 2.86-2.83 (m, 1H), 1.33 (s, 9H).

### Step 3: (9H-fluoren-9-yl)methyl tert-butyl (4-(((4-nitrophenoxy) carbonyl)oxy) butane-1,3-diyl) (S)-dicarbamate (1-11d)

1-11c (850 mg, 1.87 mmol) and bis(4-nitrophenyl) carbonate (1.14 g, 3.74 mmol) were dissolved in DMF (5 mL) followed by the addition of DIPEA (363 mg, 2.81 mmol). The resulting mixture was stirred at 25° C. for 1.5 h. After a complete reaction, the reaction mixture was diluted with EA (100 mL) and washed with brine (35 mL*2) and water (35 mL*2). The organic layer was dried over anhydrous Na₂SO₄, filtered, and concentrated under reduced pressure to afford 1-11d as a white solid (890 mg, 80.4% yield).

MS (ESI) m/z: 614.4 [M+Na]⁺.

### Step 4: (9H-fluoren-9-yl)methyl tert-butyl (4-(((3-(dimethylamino)-3-oxopropyl) carbamoyl)oxy) butane-1,3-diyl) (S)-dicarbamate (1-11f)

1-11d (360 mg, 0.61 mmol) and 1-11e (141 mg, 1.22 mmol) was dissolved in DMF (5 mL) followed by the addition of DIPEA (157 mg, 1.22 mmol). The resulting mixture was stirred at 25° C. for 2.5 h. After complete reaction, the reaction mixture was concentrated and purified by flash column chromatography (DCM/MeOH) to afford 1-11f as a pale-yellow solid (260 mg, 75.1% yield).

MS (ESI) m/z: 592.5 [M+Na]⁺.

### Step 5: tert-butyl(S)-(3-amino-4-(((3-(dimethylamino)-3-oxopropyl)carbamoyl)oxy)butyl)carbamate (1-11g)

1-11f (260 mg, 0.46 mmol) was dissolved in DMF (3 mL) followed by the addition of Et₂NH (334 mg, 4.57 mmol). The resulting mixture was stirred at 25° C. for 15 min. After a complete reaction, the reaction mixture was concentrated under reduced pressure to afford 1-11g as a light-brown syrup (158 mg, quant.) which was used directly in the next step.

MS (ESI) m/z: 347.4 [M+H]⁺.

### Step 6: (S)-4-((tert-butoxycarbonyl)amino)-2-(2,5-dioxo-2,5-dihydro-1H-pyrrol-1-yl)butyl (3-(dimethylamino)-3-oxopropyl)carbamate (1-11i)

1-11g (64 mg, 0.18 mmol) and 1-11h (43 mg, 0.28 mmol) were dissolved in a mixed solution of ACN (2 mL) and aqueous NaHCO₃ (1M, 4 mL). The mixture was firstly stirred at 0° C. for 30 min and further stirred at 25° C. for 40 min. After a complete reaction, the reaction mixture was adjusted to pH~4 with aqueous KHSO₄ (2 M), diluted with EA (60 mL) and washed with brine (30 mL) and H₂O (30 mL*2). The organic layer was dried over anhydrous Na₂SO₄, filtered, and concentrated to afford a yellow oil as the crude product which was purified by flash column chromatography (DCM/MeOH) to afford 1-11i (60 mg, 76.2% yield) as a white solid.

MS (ESI) m/z: 449.5 [M+Na]⁺.

¹H NMR (400 MHZ, CD₃OD) δ 6.79 (s, 2H), 6.56-6.54 (m, 1H), 4.34-4.31 (m, 2H), 4.23-4.16 (m, 1H), 3.31-3.30 (m, 2H), 3.01-2.99 (m, 5H), 2.92 (s, 3H), 2.55-2.52 (m, 2H), 2.17-2.08 (m, 1H), 1.86-1.77 (m, 1H), 1.41 (s, 9H).

### Step 7: (S)-4-amino-2-(2,5-dioxo-2,5-dihydro-1H-pyrrol-1-yl)butyl (3-(dimethylamino)-3-oxopropyl)carbamate (1-11)

1-11i (55 mg, 0.13 mmol) was dissolved in DCM (3 mL) followed by the addition of TFA (1 mL). The resulting mixture was stirred at 25° C. for 20 min. After a complete reaction, the reaction mixture was concentrated under reduced pressure to afford 1-11 as a clear oil (TFA salt, 54 mg, quant.).

MS (ESI) m/z: 327.3 [M+H]⁺.

¹H NMR (400 MHZ, d₆-DMSO) δ 7.78 (s, 3H), 7.05 (s, 2H), 4.28-4.13 (m, 3H), 3.12 (dd, J¹=12.8 Hz, J₁=6.8 Hz, 2H), 2.91 (s, 3H), 2.79-2.73 (m, 5H), 2.39 (t, J=7.2 Hz, 2H), 2.12-2.07 (m, 1H), 1.96-1.90 (m, 1H).

### Example 1-12

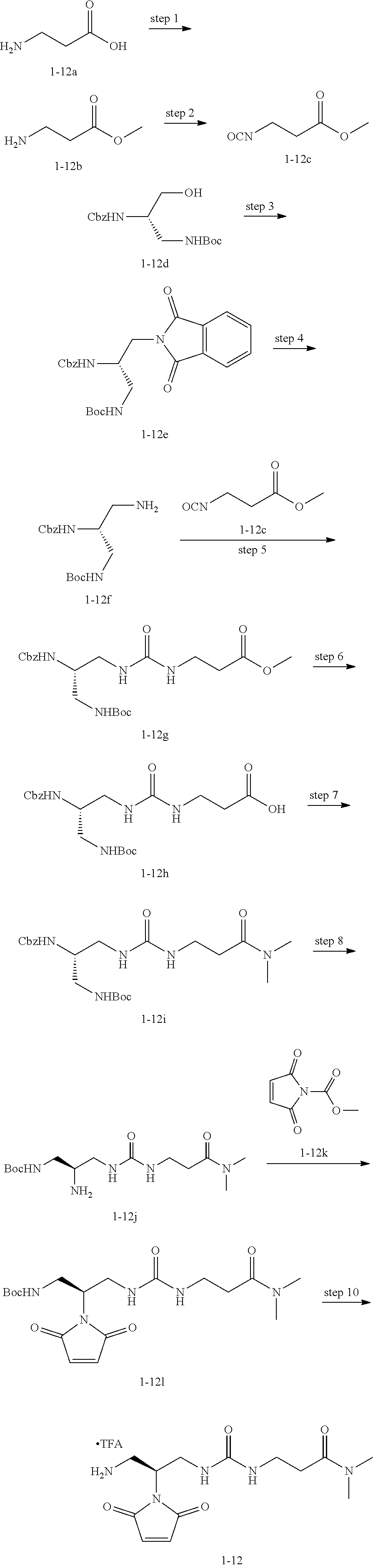

### Step 1: methyl 3-aminopropanoate (1-12b)

SOCl₂ (13.4 g, 112.2 mmol) was added dropwise to a suspension of compound 1-12a (5 g, 56.1 mmol) in MeOH (100 mL). The mixture was stirred at 70° C. for 16 h. The mixture was concentrated to give the crude compound. The crude compound was further purified by flash column chromatography (eluted with DCM/MeOH=0-20%). Compound 1-12b (3.2 g, 55.3%) was obtained as an off-white solid.

MS (ESI) m/z: 130.1 [M+Na]⁺.

### Step 2: methyl 3-isocyanatopropanoate (1-12c)

Compound 1-12b (500 mg, 4.85 mmol) in CH₂Cl₂ (5 mL) and saturated NaHCO₃(aq.) solution (5 mL) was degassed, and the flask was cooled in an ice water bath. Triphosgene (1.44 g, 4.85 mmol) was added in one portion under inert atmosphere at 0° C. The reaction stirred at 0° C. to r.t. over 2.5 h. The reaction was diluted with water (100 mL) and poured into a separatory funnel. The layers separated and the aqueous layer was extracted with CH₂Cl₂. The combined organic extracts were washed with brine, dried over Na₂SO₄, filtered, and concentrated under reduced pressure to obtain crude compound 1-12c (426 mg, 68.1% yield) as a yellow liquid, which was directly used for the next reaction without purification.

### Step 3: benzyl tert-butyl (3-(1,3-dioxoisoindolin-2-yl) propane-1,2-diyl) (S)-dicarbamate (1-12e)

Triphenylphosphine (2.91 g, 11.1 mmol) and phthalimide (1.63 g, 11.1 mmol) were added to a flask containing dry THF (30 mL). Compound 1-12d (3 g, 9.25 mmol) was added and the flask was cooled to 0° C. Diisopropyl azodicarboxylate DIAD (2.24 mg, 11.1 mmol) was added dropwise and the reaction was stirred for 30 minutes at 0° C. and overnight at room temperature. The mixture was concentrated under reduced pressure and the residue was purified by flash column chromatography (eluted with PE/EA=0-70%) to give compound 1-12e (3.6 g, 85.9%) as a white solid.

MS (ESI) m/z: 476.4 [M+Na]⁺.

### Step 4: benzyl tert-butyl (3-aminopropane-1,2-diyl) (R)-dicarbamate (1-12f)

Compound 1-12e (1.3 g, 2.87 mmol) was dissolved in methanol (30 mL) and hydrazine monohydrate (359 mg, 5.73 mmol) was added. The reaction mixture was then refluxed for 2 h and cooled to room temperature. The precipitate that formed was filtered and methanol was used to wash the filtrate. The filtrate was concentrated under reduced pressure and the remaining solid purified by flash column chromatography (eluted with DCM/MeOH=0-20%) to give compound 1-12f (640 mg, 69.0% yield) as a colorless oil.

MS (ESI) m/z: 324.3 [M+H]⁺.

### Step 5: methyl (R)-7-(((benzyloxy)carbonyl)amino)-2,2-dimethyl-4,10-dioxo-3-oxa-5,9,11-triazatetradecan-14-oate (1-12g)

To a solution of compound 1-12f (500 mg, 1.55 mmol) in THF (10 ml) were added 1-12c (399 mg, 3.09 mmol) and Et₃N (312.9 mg, 3.09 mmol) at 0° C. and the mixture was at r.t. for 4 h. The mixture was diluted with EA (100 mL) and washed by brine (50 mL*2). The organic layer was dried over anhydrous Na₂SO₄, filtered and concentrated, and the residue was purified by flash column chromatography (eluted with PE/EA=0%-40%) to give compound 1-12g (335 mg, 47.9% yield) as a white solid.

MS (ESI) m/z: 453.5 [M+H]⁺.

### Step 6: (R)-7-(((benzyloxy)carbonyl)amino)-2,2-dimethyl-4,10-dioxo-3-oxa-5,9,11-triazatetradecan-14-oic acid (1-12h)

To a solution of compound 1-12g (335 mg, 0.74 mmol) in MeOH—H₂O (6 mL, v:v=3:1) was added LiOH (35.5 mg, 1.48 mmol). The mixture was stirred at r.t. for 16 h. The mixture was concentrated to remove MeOH and diluted with H₂O (5 mL). The pH of the mixture was adjusted to 5 by HOAc and extracted by EA (50 mL*3). The organic layers were combined and dried and concentrated to give compound 1-12h (289 mg, 88.9% yield) as a white solid.

MS (ESI) m/z: 439.4 [M+H]⁺.

### Step 7: benzyl tert-butyl (3-(3-(3-(dimethylamino)-3-oxopropyl) ureido) propane-1,2-diyl)(R)-dicarbamate (1-12i)

To a solution of compound 1-12h (180 mg, 0.41 mmol) in DCM (6 mL) were added Me₂NH·HCl (67.0 mg, 0.82 mmol), 1-ethyl-3-(3-dimethylaminopropyl) urea (EDCI) (102.3 mg, 0.534 mmol) and HOBt (72.1 mg, 0.534 mmol) and Et₃N (83.08 mg, 0.82 mmol). The mixture was stirred at r.t. for 16 h. The mixture was diluted with EA (100 mL), washed with 1 N HCl (50 mL*3), sat. NaHCO₃ (50 mL*3) and brine (50 mL*3). The organic layer was dried over anhydrous Na₂SO₄, filtered and concentrated to give the crude product 1-12i (131 mg, 68.6% yield) as a white solid.

MS (ESI) m/z: 466.5 [M+H]⁺.

### Step 8: tert-butyl (R)-(2-amino-3-(3-(3-(dimethylamino)-3-oxopropyl) ureido) propyl) carbamate (1-12j)

To a solution of compound 1-12i (131 mg, 0.28 mmol) in MeOH (4 mL) was added Pd/C (10%, 26 mg). The mixture was stirred at r.t. under H₂ for 4 h. The mixture was filtered and concentrated to give crude product 1-12j (93 mg, crude) as a colorless oil.

MS (ESI) m/z: 332.4 [M+H]⁺.

### Step 9: tert-butyl (R)-(3-(3-(3-(dimethylamino)-3-oxopropyl) ureido)-2-(2,5-dioxo-2,5-dihydro-1H-pyrrol-1-yl) propyl) carbamate (1-12l)

To a solution of compound 1-12j (93 mg, 0.281 mmol) in NaHCO₃ (1N, 5 mL) and THF (5 mL) was added 1-12k (132 mg, 0.842 mmol) at 0° C. The mixture was stirred at 0° C. for 30 min, then warmed up to room temperature and stirred for 1 h. The mixture was purified by prep-HPLC (Method: column: XBridge Prep C18 OBD Sum 19*250 mm; Mobile phase: A-water (0.1% TFA): B-acetonitrile; Flow rate: 20 mL/min). Compound 1-12l (32 mg, 27.7% yield) was obtained as a white solid.

MS (ESI) m/z: 412.4 [M+H]⁺.

### Step 10: (S)-3-(3-(3-amino-2-(2,5-dioxo-2,5-dihydro-1H-pyrrol-1-yl) propyl) ureido)-N,N-dimethylpropanamide acetate (1-12)

To a solution of compound 1-12l (30 mg, 0.073 mmol) in DCM (10 mL) was added TFA (1 mL). The mixture was stirred at room temperature for 1 h. The mixture was concentrated to give the crude compound 1-12 (19 mg, 83.7% yield) as a white solid.

MS (ESI) m/z: 312.4 [M+H]⁺.

¹H NMR (400 MHZ, DMSO): δ 7.90 (br s, 3H), 7.04 (s, 2H), 6.32-6.29 (m, 1H), 5.89 (br s, 1H), 4.20-4.17 (m, 1H), 3.37-3.23 (m, 4H), 3.15-3.12 (m, 3H), 3.09 (s, 3H), 2.73 (s, 3H), 2.36-2.33 (m, 2H).

### Example 1-13

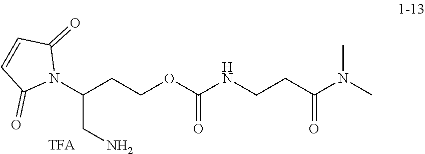 [SMILES: CN(C)C(=O)CCNC(=O)OCCC(CN)N1C(=O)C=CC1=O]

### 4-amino-3-(2,5-dioxo-2,5-dihydro-1H-pyrrol-1-yl)butyl (3-(dimethylamino)-3-oxopropyl) carbamate (1-13)

1-13 (TFA salt, 178 mg, quant.) was synthesized according to the synthetic procedure of Example 1-11.

MS (ESI) m/z: 327.4 [M+H]⁺.

¹H NMR (400 MHZ, d₆-DMSO): δ 8.01 (br s, 3H), 7.04 (s, 2H), 6.83 (t, J=6.0 Hz, 1H), 4.28-4.23 (m, 1H), 3.87 (t, J=6.0 Hz, 2H), 3.34-3.29 (m, 1H), 3.17-3.12 (m, 3H), 2.94 (s, 3H), 2.81 (s, 3H), 2.43 (t, J=7.2 Hz, 2H), 2.13-2.06 (m, 1H), 1.98-1.93 (m, 1H).

### Example 1-14

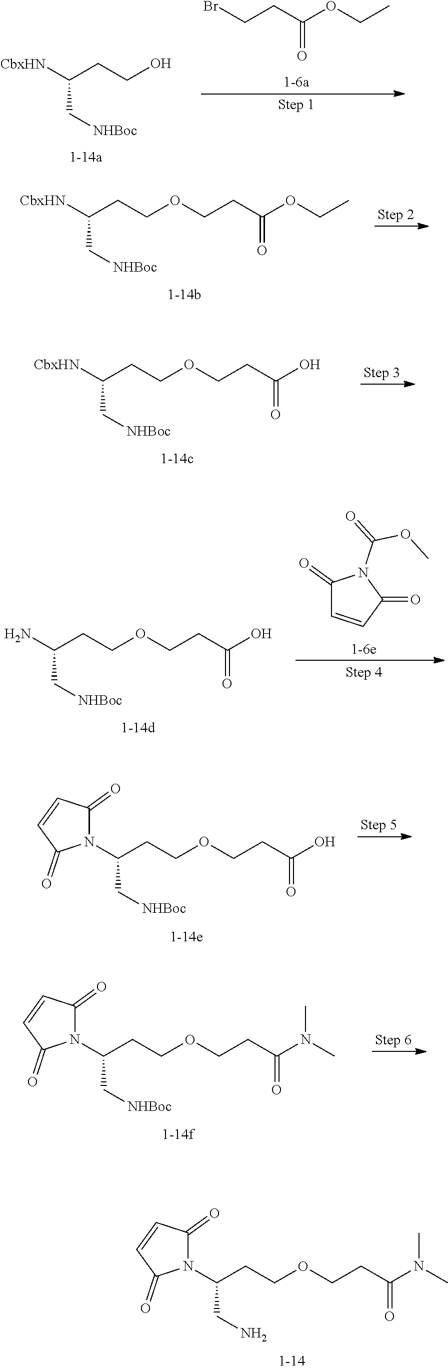

### (R)-3-(4-((tert-butoxycarbonyl)amino)-3-(2,5-dioxo-2,5-dihydro-1H-pyrrol-1-yl)butoxy)propanoic acid 1-14e

1-13e (110 mg) as a white solid was synthesized according to the synthetic procedure of Example 1-7.

MS (ESI) m/z: 379.3 [M+Na]⁺.

1H NMR (400 MHz, cdcl₃) δ 6.68 (s, 2H), 4.87 (s, 1H), 4.42-4.26 (m, 1H), 3.70-3.56 (m, 3H), 3.50 3.52-3.47 (m, 1H), 3.43-3.36 (m, 1H), 3.31 (dt, J=14.0, 4.4 Hz, 1H), 2.56 (t, J=6.0 Hz, 1H), 3.32-2.12 (m, 1H), 1.97-1.92 (m, 1H), 1.40 (s, 9H).

### (R)-3-(4-amino-3-(2,5-dioxo-2,5-dihydro-1H-pyrrol-1-yl) butoxy)-N,N-dimethylpropanamide (TFA salt) (1-14)

1-14 (9.6 mg) as a brown oil was synthesized according to the synthetic procedure of Example 1-7.

MS (ESI) m/z: 384.4 [M+H]⁺.

Reference compounds 2-1 and 2-3 were synthesized according to the methods reported in U.S. Pat. No. 10,973,924B2 and Patent Application Publication No. US 2020/345863A1.

### Example 2-2

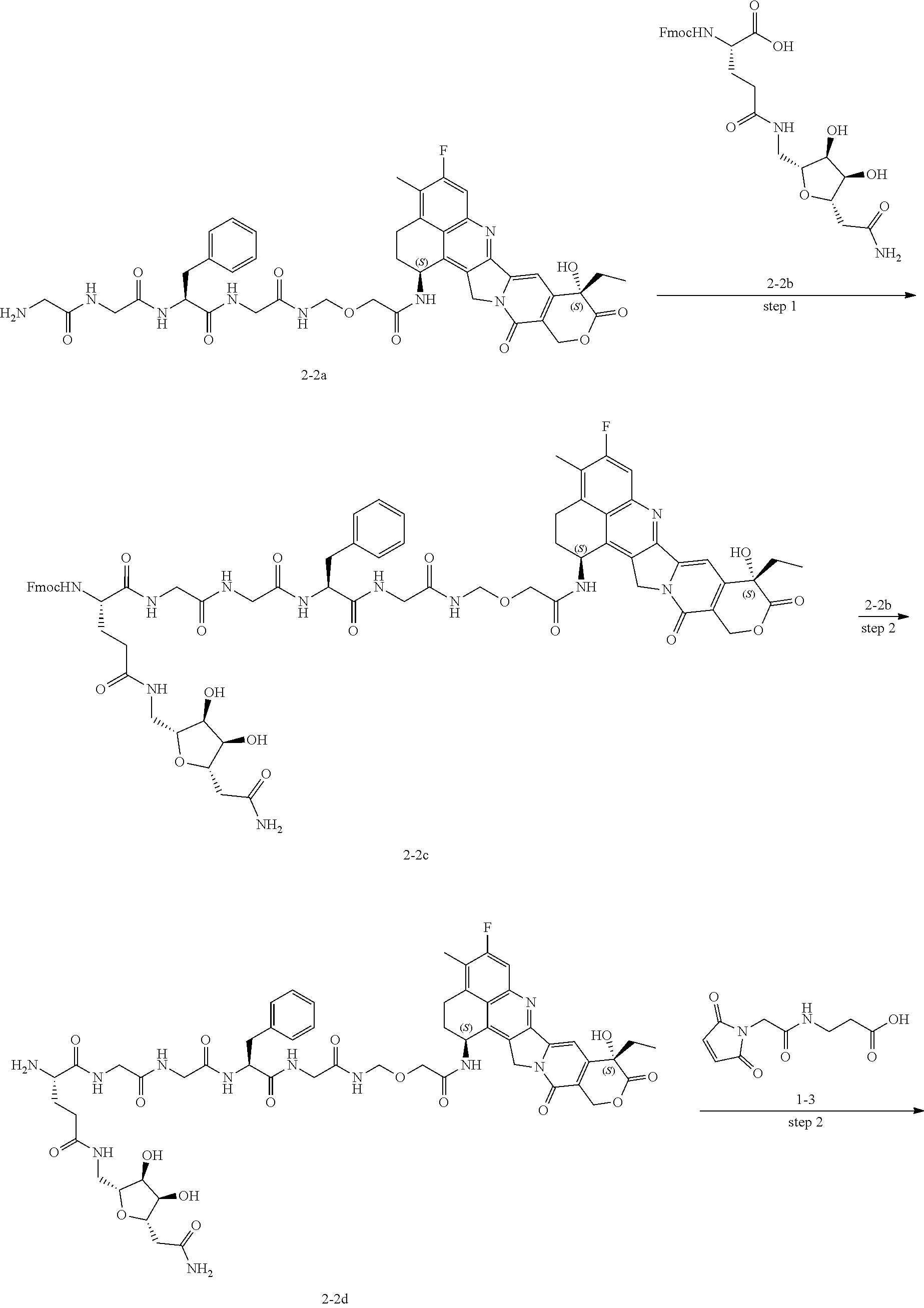

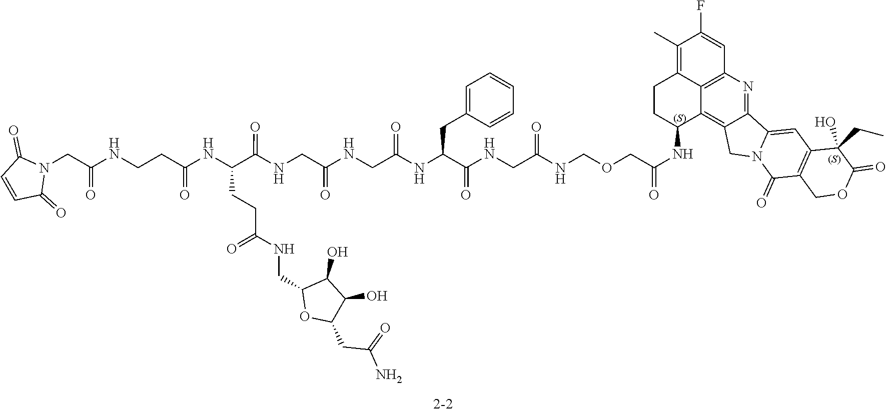

### Step 1: (9H-fluoren-9-yl)methyl ((6S,15S)-1-((2R,3S,4R,5S)-5-(2-amino-2-oxoethyl)-3,4-dihydroxytetrahydrofuran-2-yl)-15-benzyl-24-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-3,7,10,13,16,19,24-heptaoxo-22-oxa-2,8,11,14,17,20-hexaazatetracosan-6-yl) carbamate (2-2c)

Compound 2-2c (20 mg, 36.8% yield) was synthesized according to Step 3 of Example 1-2.

MS (ESI) m/z: 1386.9 [M+Na]⁺.

### Step 2: (S)-2-amino-N5-(((2R,3S,4R,5S)-5-(2-amino-2-oxoethyl)-3,4-dihydroxytetrahydrofuran-2-yl)methyl)-N1-((S)-10-benzyl-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl) pentanediamide (2-2d)

Compound 2-2c (20 mg, 36.8% yield) was synthesized according to Step 5 of Example 1-11. (17 mg, quant.).

MS (ESI) m/z: 1142.8 [M+H]⁺.

### Step 3: (S)-N5-(((2R,3S,4R,5S)-5-(2-amino-2-oxoethyl)-3,4-dihydroxytetrahydrofuran-2-yl)methyl)-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(3-(2-(2,5-dioxo-2,5-dihydro-1H-pyrrol-1-yl)acetamido)propanamido)pentanediamide (2-2)

To a mixture of 1-3 (5 mg, 0.021 mmol) and HATU (8 mg, 0.021 mmol) in DMF (1 mL) was added DIPEA (5 mg, 0.035 mmol). The mixture was reacted at r.t. for 10 min. 2-2d (20 mg, 0.018 mmol) was added and the mixture was further stirred at this temperature for another 15 min. The mixture was filtered, and the filtrate was purified using prep-HPLC (Method: column: XBridge Prep C18 OBD 5 μm 19*250 mm; Mobile phase: A-water (0.1% formic acid): B-acetonitrile; Flow rate: 20 mL/min) to afford 2-2 (4 mg, 16.9% yield).

MS (ESI) m/z: 1372.8 [M+Na]⁺.

### Example 2-4

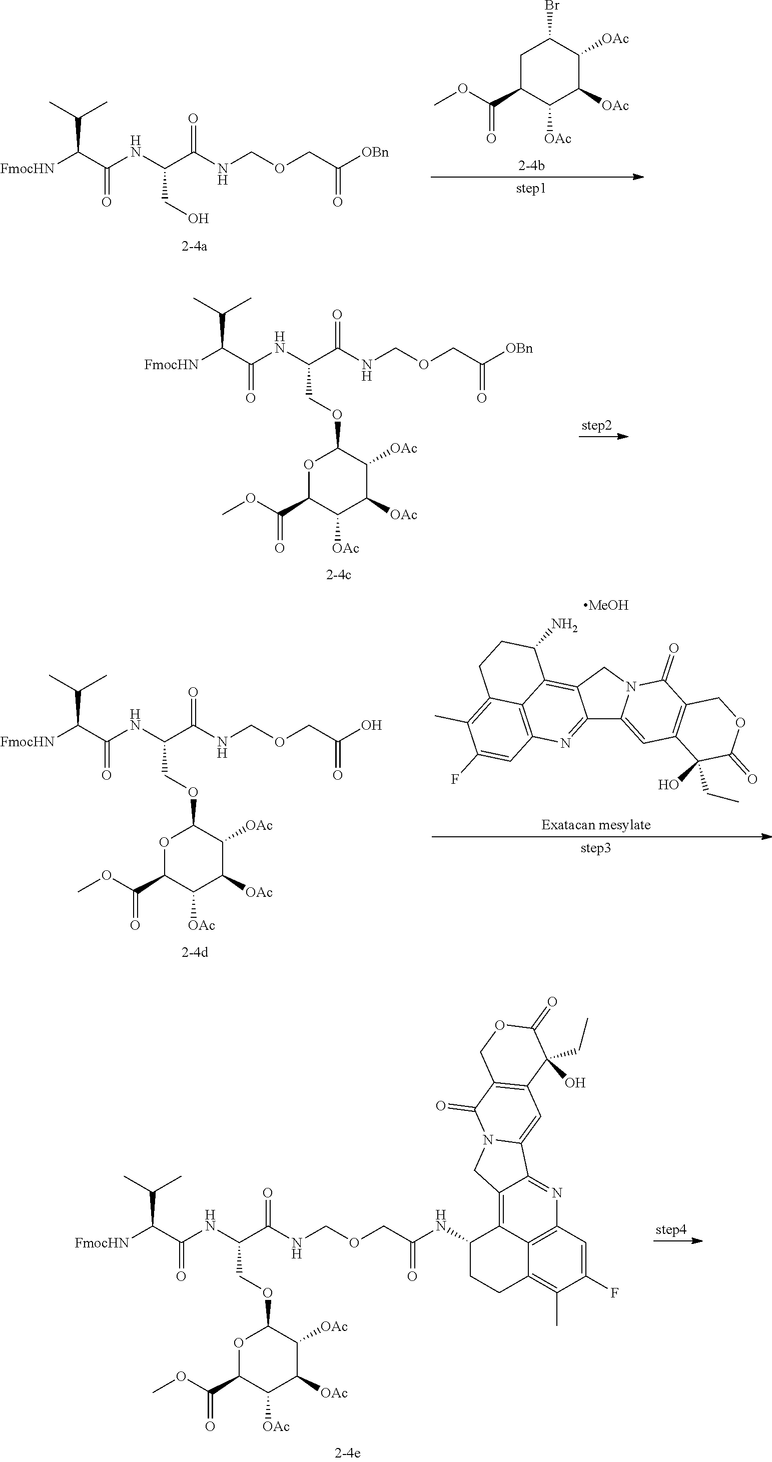

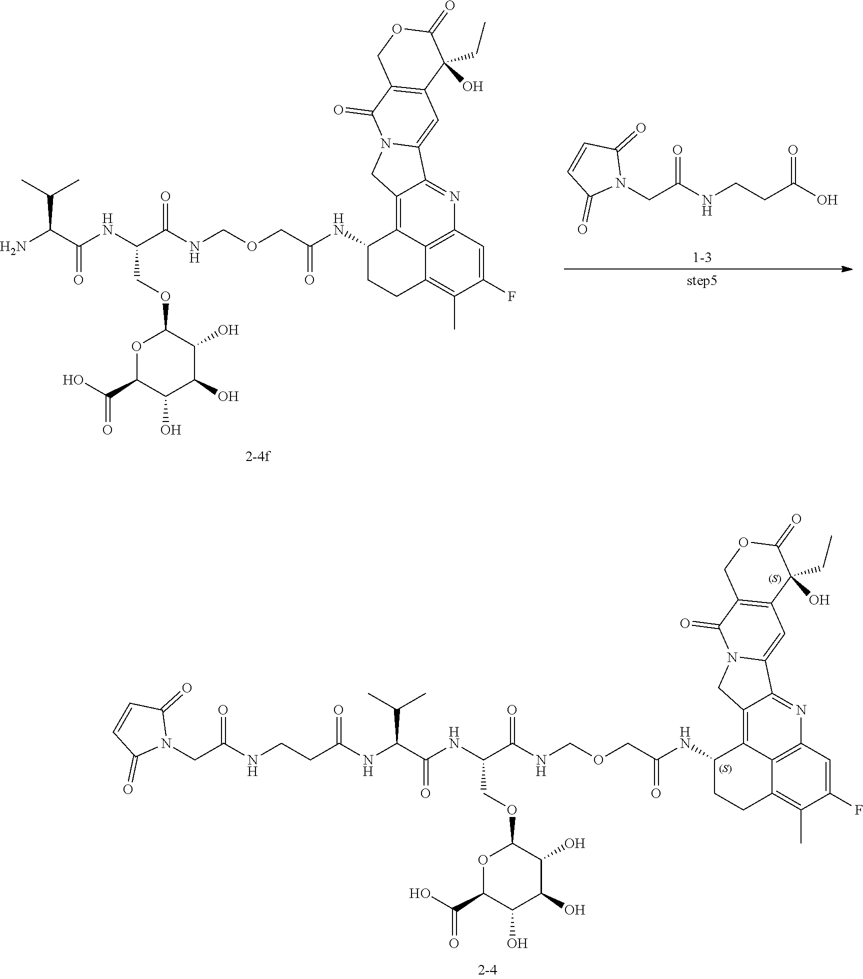

### Step 1: (2R,3R,4S,5S,6S)-2-((S)-2-((S)-2-((((9H-fluoren-9-yl)methoxy)carbonyl)amino)-3-methylbutanamido)-3-(((2-(benzyloxy)-2-oxoethoxy)methyl)amino)-3-oxopropoxy)-6-(methoxycarbonyl)tetrahydro-2H-pyran-3,4,5-triyl triacetate (2-4c)

A mixture of 2-4a (150 mg, 0.25 mmol, purchased from MedChemExpress), 2-4b (148 mg, 0.37 mmol, commercially available) and 4 Å MS (800 mg) in dry DCE (5 mL) was stirred at r.t. for 30 min. AgOTf (83 mg, 0.32 mmol) was added under nitrogen atmosphere and stirred at r.t. for 14 h. The reaction solution was diluted with EtOAc (10 mL), filtered through celite, and washed with EtOAc (5 mL*3). The organic phase was washed with sat. NaHCO₃ (20 mL), concentrated and purified by flash column chromatography (eluent: petroleum ether/EtOAc=70/30 to 0/100 then DCM/MeOH=100/0 to 95/5) to give 2-4c (28 mg, 12.2% yield) as a light-yellow solid.

MS (ESI) m/z: 942.5 [M+Na]⁺.

### Step 2: (5S,8S)-1-(9H-fluoren-9-yl)-5-isopropyl-3,6,9-trioxo-8-((((2R,3R,4S,5S,6S)-3,4,5-triacetoxy-6-(methoxycarbonyl)tetrahydro-2H-pyran-2-yl)oxy)methyl)-2,12-dioxa-4,7,10-triazatetradecan-14-oic acid (2-4d)

To the mixture of 2-4c (114 mg, 0.12 mmol) and 10% Pd/C (26 mg, 0.012 mmol) was added MeOH (3 mL) under nitrogen atmosphere. The reaction solution was stirred at r.t. under H₂ atmosphere for 1 h. The solution was filtered and concentrated under vacuum to afford 2-4d (103 mg, 12.4% yield) as a white solid.

MS (ESI) m/z: 852.5 [M+Na]⁺.

### Step 3: (2R,3R,4S,5S,6S)-2-((S)-2-((S)-2-((((9H-fluoren-9-yl)methoxy)carbonyl)amino)-3-methylbutanamido)-3-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-3-oxopropoxy)-6-(methoxycarbonyl)tetrahydro-2H-pyran-3,4,5-triyl triacetate (2-4e)

To a solution of 2-4d (103 mg, 0.12 mmol), exatecan mesylate (64 mg, 0.12 mmol) and HATU (47 mg, 0.12 mmol) in dry DMF (2 mL) was added DIPEA (61 μL, 0.36 mmol). The solution was stirred at r.t. for 1 h and added into H₂O (20 mL) and extracted with DCM/MeOH (10:1, 11 mL*3). The organic phase was concentrated and purified by flash column chromatography (eluent: DCM/MeOH=100/0 to 95/5) to afford 2-4e (97 mg, 64.0% yield) as a light-brown solid.

MS (ESI) m/z: 1269.5 [M+Na]⁺.

### Step 4: (2S,3S,4S,5R,6R)-6-((S)-2-((S)-2-amino-3-methylbutanamido)-3-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-3-oxopropoxy)-3,4,5-trihydroxytetrahydro-2H-pyran-2-carboxylic acid (2-4f)

To a solution of 2-4e (30 mg, 0.024 mmol) in MeOH/H₂O (0.5/0.5 mL) was added TEA (167 μL, 1.2 mmol). The solution was stirred at r.t. for 9 h. After a complete reaction, the mixture was purified by prep-HPLC (FA) (Method: column: XBridge Prep C18 OBD 5 μm 19*250 mm; Mobile phase: A-water (0.1% formic acid): B-acetonitrile; Flow rate: 20 mL/min). The fraction was lyophilized to give 2-4f (15 mg, 70.6% yield) as a pale-yellow solid.

MS (ESI) m/z: 885.5 [M+H]⁺.

### Step 5: (2S,3S,4S,5R,6R)-6-((S)-2-((S)-2-(3-(2-(2,5-dioxo-2,5-dihydro-1H-pyrrol-1-yl)acetamido)propanamido)-3-methylbutanamido)-3-(((2-(((1S,9S)-9-ethyl-5-fluoro-9-hydroxy-4-methyl-10,13-dioxo-2,3,9,10,13,15-hexahydro-1,12H-benzo[de]pyrano[3′,4′:6,7]indolizino[1,2-b]quinolin-1-yl)amino)-2-oxoethoxy)methyl)amino)-3-oxopropoxy)-3,4,5-trihydroxytetrahydro-2H-pyran-2-carboxylic acid (2-4)

To a solution of 1-3 (8.3 mg, 0.038 mmol) and HATU (11 mg, 0.029 mmol) in dry DMF (0.3 mL) was added DIPEA (5 μL, 0.029 mmol). The resulting mixture was stirred at r.t. for 15 min and added dropwise into the solution of 2-4f (17 mg, 0.019 mmol) in dry DMF (0.7 mL). The resulting mixture was further stirred at r.t. for 15 min. HOAc was added to adjust the pH to 5. The solution was purified by prep-HPLC (FA) (Method: column: XBridge Prep C18 OBD 5 μm 19*250 mm; Mobile phase: A-water (0.1% formic acid): B-acetonitrile; Flow rate: 20 mL/min). The fraction was lyophilized to give 2-4 (13 mg, 63.5% yield) as a beige solid.

MS (ESI) m/z: 1093.5 [M+H]⁺.

### Example 2-5

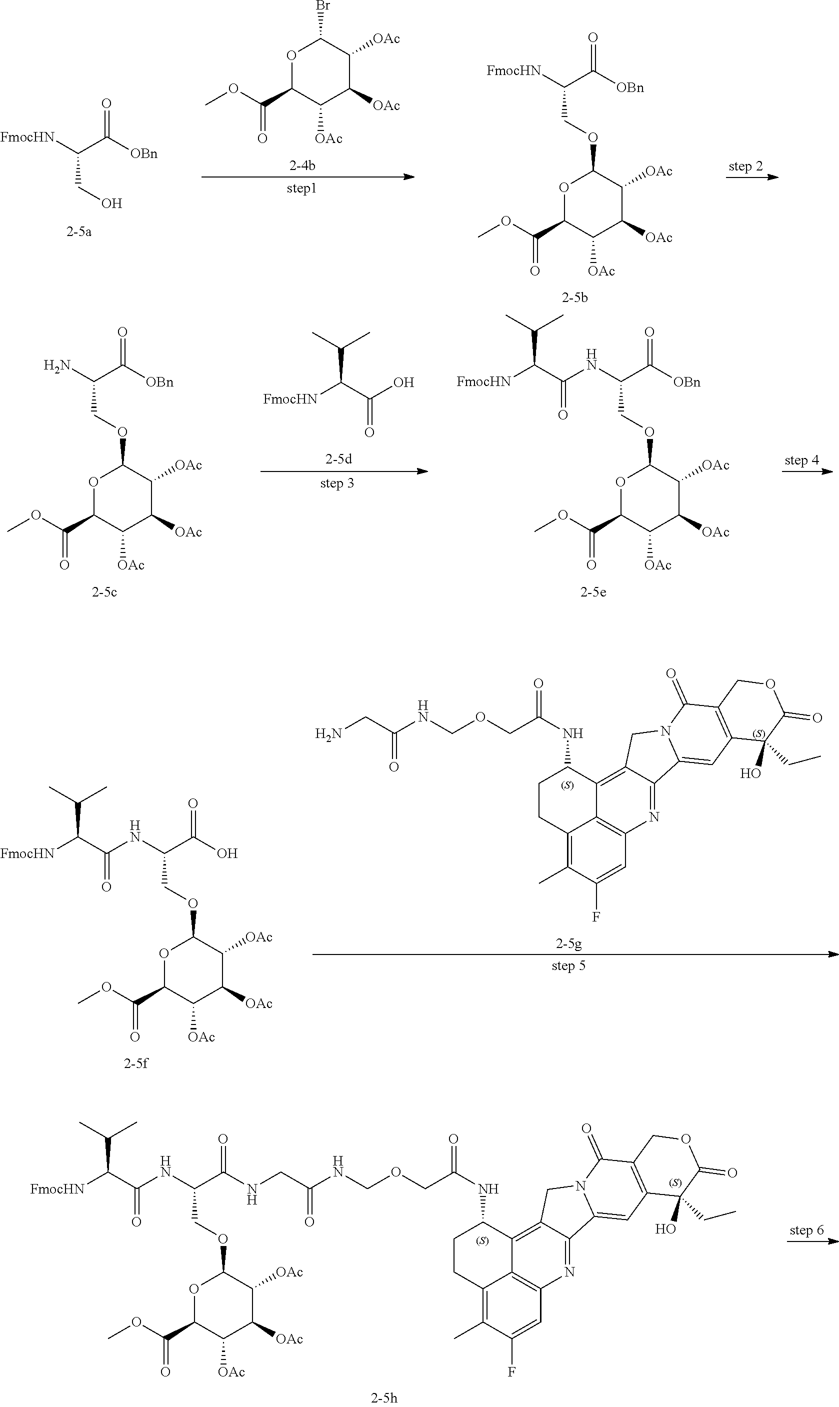

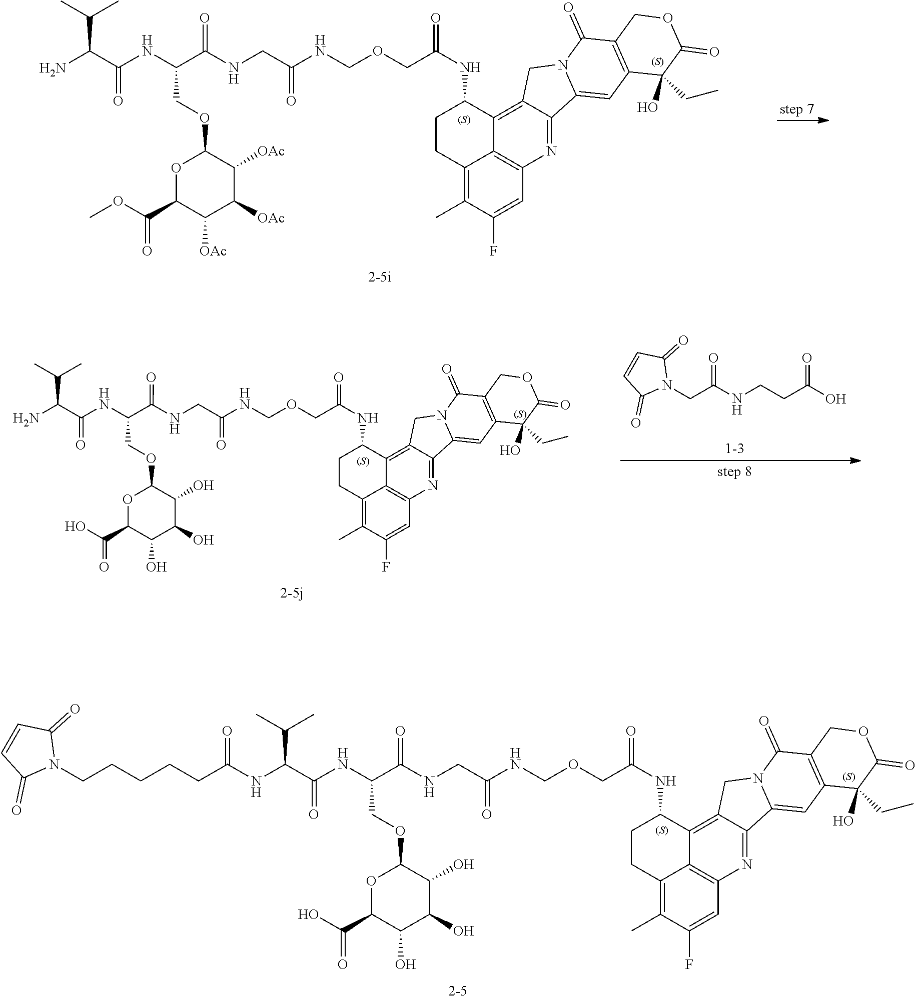

### Step 1: (2R,3R,4S,5S,6S)-2-((S)-2-((((9H-fluoren-9-yl)methoxy)carbonyl)amino)-3-(benzyloxy)-3-oxopropoxy)-6-(methoxycarbonyl)tetrahydro-2H-pyran-3,4,5-triyl triacetate (2-5b)

To a mixture of 2-5a (834 mg, 2 mmol), 2-4b (950 mg, 2.4 mmol) and 4 Å MS (3000 mg) was added dry DCM (30 mL), stirred at r.t. for 30 min. AgOTf (617 mg, 2.4 mmol) was added under nitrogen atmosphere, stirred at r.t. for 16 h. The reaction solution was diluted with EtOAc (30 mL), filtered through Celite, washed with EtOAc (20 mL*3). The organic phase was washed with sat. NaHCO₃ (20 mL), concentrated and purified by flash column chromatography (eluent: petroleum ether/EtOAc=70/30 to 0/100) to give 2-5b (470 mg, 32.1% yield) as a light-yellow solid.

MS (ESI) m/z: 756.3 [M+Na]⁺.

### Step 2: (2R,3R,4S,5S,6S)-2-((S)-2-amino-3-(benzyloxy)-3-oxopropoxy)-6-(methoxycarbonyl)tetrahydro-2H-pyran-3,4,5-triyl triacetate (2-5c)

To a solution of 2-5b (470 mg, 0.64 mmol) in DMF (5 mL) was added Et₂N (1420 μL, 1011 mg, 12.8 mmol). The mixture was stirred at r.t. for 0.5 h. On completion of the reaction, the mixture was concentrated under vacuum to give 2-5c (327 mg, crude) as a yellow solid.

MS (ESI) m/z: 512.4 [M+H]⁺.

### Step 3: (2R,3R,4S,5S,6S)-2-((S)-2-((S)-2-((((9H-fluoren-9-yl)methoxy)carbonyl)amino)-3-methylbutanamido)-3-(benzyloxy)-3-oxopropoxy)-6-(methoxycarbonyl)tetrahydro-2H-pyran-3,4,5-triyl triacetate (2-5e)

To a solution of 2-5d (327 mg) in DMF (5 mL) were added HATU (291 mg, 0.76 mmol) and DIPEA (223 μL, 165 mg, 1.27 mmol). The resulting yellow solution was stirred at r.t. for 5 min then 2-5c (239 mg, 0.70 mmol) was added. The mixture was further stirred at r.t. for 60 min. On completion of the reaction, solvent was evaporated and the residue was purified by flash column chromatography (eluent: petroleum ether/EtOAc=70/30 to 0/100) and prep-HPLC (FA) (Method: column: XBridge Prep C18 OBD Sum 19*250 mm; Mobile phase: A-water (0.1% formic acid): B-acetonitrile; Flow rate: 20 mL/min). The fraction was lyophilized to give 2-5e (300 mg, 56.2% yield) as a white solid.

MS (ESI) m/z: 833.4 [M+H]⁺.

### Step 4: N-((((9H-fluoren-9-yl)methoxy)carbonyl)-L-valyl)-O-((2R,3R,4S,5S,6S)-3,4,5-triacetoxy-6-(methoxycarbonyl)tetrahydro-2H-pyran-2-yl)-L-serine (2-5f)

To a solution of 2-5e (300 mg, 0.36 mmol) in MeOH (10 mL) and THF (8 mL) was added wet Pd/C (30 mg) then purged with H₂ balloon 3 times and stirred at r.t. for 2 h. On completion of the reaction, the mixture was filtered through a syringe filter head and the filtrate was concentrated under vacuum to give 2-5f (265 mg, crude) as a white solid.

MS (ESI) m/z: 765.3 [M+Na]⁺.

### Step 5: (2R,3R,4S,5S,6S)-2-((S)-2-((S)-2-((((9H-fluoren-9-yl)methoxy)carbonyl)amino)-3-methylbutanamido)-3-((2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-3-oxopropoxy)-6-(methoxycarbonyl)tetrahydro-2H-pyran-3,4,5-triyl triacetate (2-5h)

To a solution of 2-5f (265 mg) in DMF (5 mL) were added HATU (120 mg, 0.42 mmol) and DIPEA (124 μL, 92 mg, 0.71 mmol). The resulting yellow solution was stirred at r.t. for 5 min then 2-5g (227 mg, 0.39 mmol) was added. The mixture was stirred at r.t. for 60 min. On completion of the reaction, solvent was evaporated and the residue was purified by flash column chromatography (eluent: DCM/MeOH=95/5 to 90/10) to give 2-5h (280 mg, 59.6% yield) as a brown solid.

MS (ESI) m/z: 1326.4 [M+Na]⁺.

### Step 6: (2R,3R,4S,5S,6S)-2-((S)-2-((S)-2-amino-3-methylbutanamido)-3-((2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-3-oxopropoxy)-6-(methoxycarbonyl)tetrahydro-2H-pyran-3,4,5-triyl triacetate (2-5i)

To a solution of 2-5h (280 mg, 0.21 mmol) in DMF (5 mL) was added Et₂N (478 μL, 340 mg, 4.29 mmol). The mixture was stirred at r.t. for 0.5 h. On completion of the reaction, the mixture was concentrated under vacuum to give 2-5i (220 mg, crude) as a white solid.

MS (ESI) m/z: 1082.4 [M+H].

### Step 7: (2S,3S,4S,5R,6R)-6-((S)-2-((S)-2-amino-3-methylbutanamido)-3-((2-(((2-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-2-oxoethoxy)methyl)amino)-2-oxoethyl)amino)-3-oxopropoxy)-3,4,5-trihydroxytetrahydro-2H-pyran-2-carboxylic acid (2-5j)

To the solution of 2-5i (220 mg) in MeOH/H₂O (5/5 mL) was added Na₂CO₃ (151 mg, 1.42 mmol), and the mixture stirred at r.t. for 9 h. The solution was purified by prep-HPLC (FA) (Method: column: XBridge Prep C18 OBD 5 μm 19*250 mm; Mobile phase: A-water (0.1% formic acid): B-acetonitrile; Flow rate: 20 mL/min). The fraction was lyophilized to give 2-5j (45 mg, 23.8% yield) as a white solid.

MS (ESI) m/z: 942.3 [M+H]⁺.

### Step 8: (2S,3S,4S,5R,6R)-6-((S)-2-((S)-2-(3-(2-(2,5-dioxo-2,5-dihydro-1H-pyrrol-1-yl)acetamido)propanamido)-3-methylbutanamido)-3-((2-(((2-(((1R,9R)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)amino)-3-oxopropoxy)-3,4,5-trihydroxytetrahydro-2H-pyran-2-carboxylic acid (2-5)

To the solution of 1-3 (5.76 mg, 0.026 mmol) and HATU (9.576 mg, 0.025 mmol) in dry DMF (1 mL) was added DIPEA (7.3 μL, 5.4 mg, 0.042 mmol), and the mixture was stirred at r.t. for 15 min. The solution was added dropwise into the solution of 2-5j (20 mg, 0.021 mmol) in dry DMF (1 mL), and stirred at r.t. for 15 min. The solution was purified by prep-HPLC (FA) (Method: column: XBridge Prep C18 OBD Sum 19*250 mm; Mobile phase: A-water (0.1% formic acid): B-acetonitrile; Flow rate: 20 mL/min). The fraction was lyophilized to give 2-5 (7.4 mg, 31.0% yield) as a white solid.

MS (ESI) m/z: 1172.4 [M+Na]⁺.

### Example 2-6

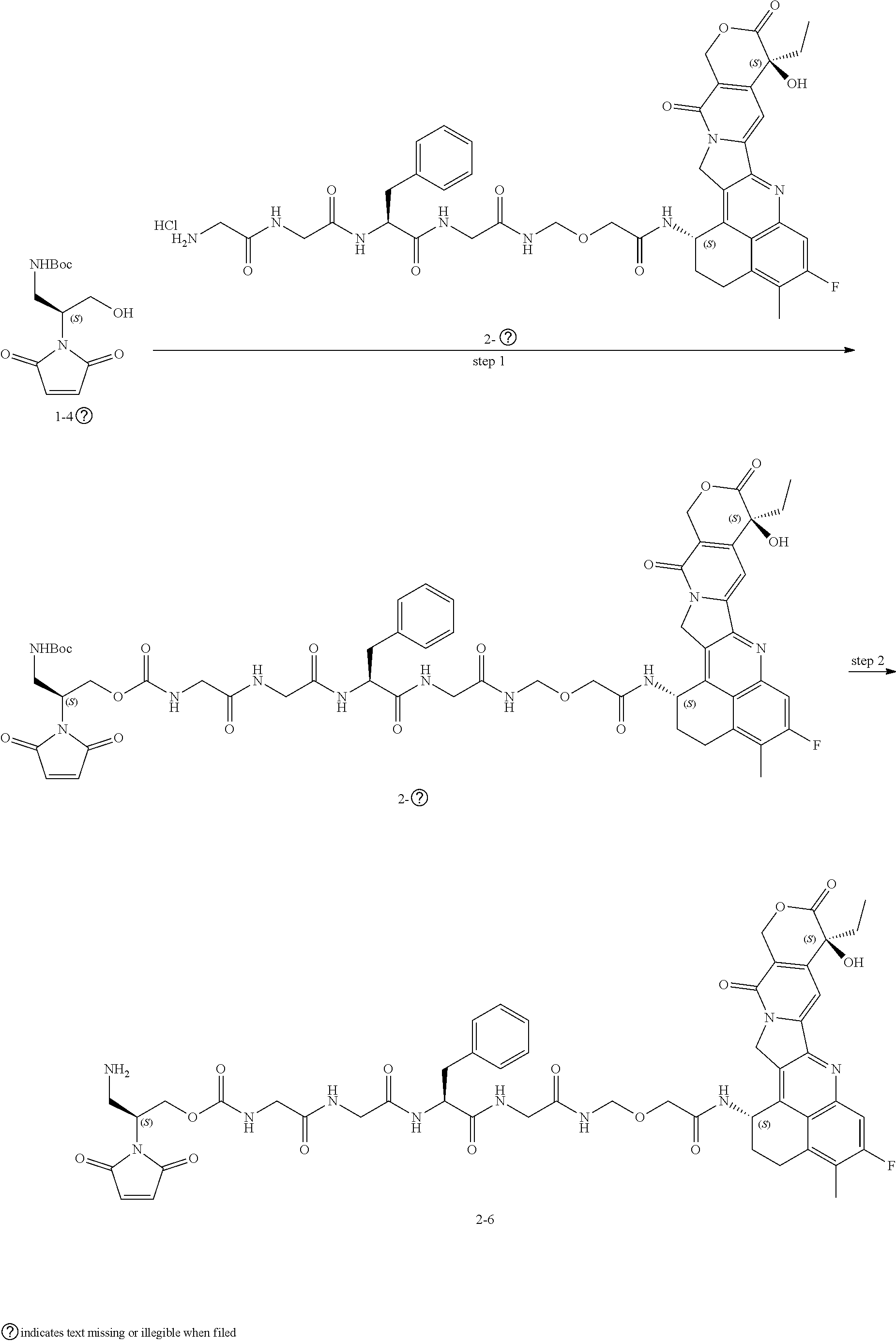

### Step 1: tert-butyl ((10S,21S)-10-benzyl-21-(2,5-dioxo-2,5-dihydro-1H-pyrrol-1-yl)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-1,6,9,12,15,18-hexaoxo-3,19-dioxa-5,8,11,14,17-pentaoxadocosan-22-yl) carbamate (2-6b)

To a mixture of 1-4e (205.7 mg, 0.76 mmol) in DMF (3 mL) were added DSC (195.0 mg, 0.76 mmol) and DIPEA (118.1 mg, 0.91 mmol). The mixture was stirred at r.t. for 2 hr. 2-6a (500.7 mg, 0.57 mmol, purchased from MedChemExpress) was added and then stirred at r.t. for 10 min. The mixture was filtered, and the filtrate was purified by prep-HPLC (FA) to give 2-6 (230 mg) as a dark-yellow solid.

MS (ESI) m/z: 1160.8 [M+Na]⁺.

### Step 2: (S)-3-amino-2-(2,5-dioxo-2,5-dihydro-1H-pyrrol-1-yl)propyl ((S)-10-benzyl-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl) carbamate (2-6)

To a mixture of 2-6b (30.0 mg, 0.026 mmol) in DCM (2 mL) was added ZnBrz (237.7 mg, 1.06 mmol), and the mixture was heated to 45° C. for 16 hr. The mixture was concentrated under vacuum to remove DCM then dissolved in DMSO/H₂O and purified by prep-HPLC (FA). The fraction was lyophilized to give 2-6 (12.6 mg, 45% yield) as a white solid.

MS (ESI) m/z: 1037.8 [M+H]⁺.

### Example 2-7

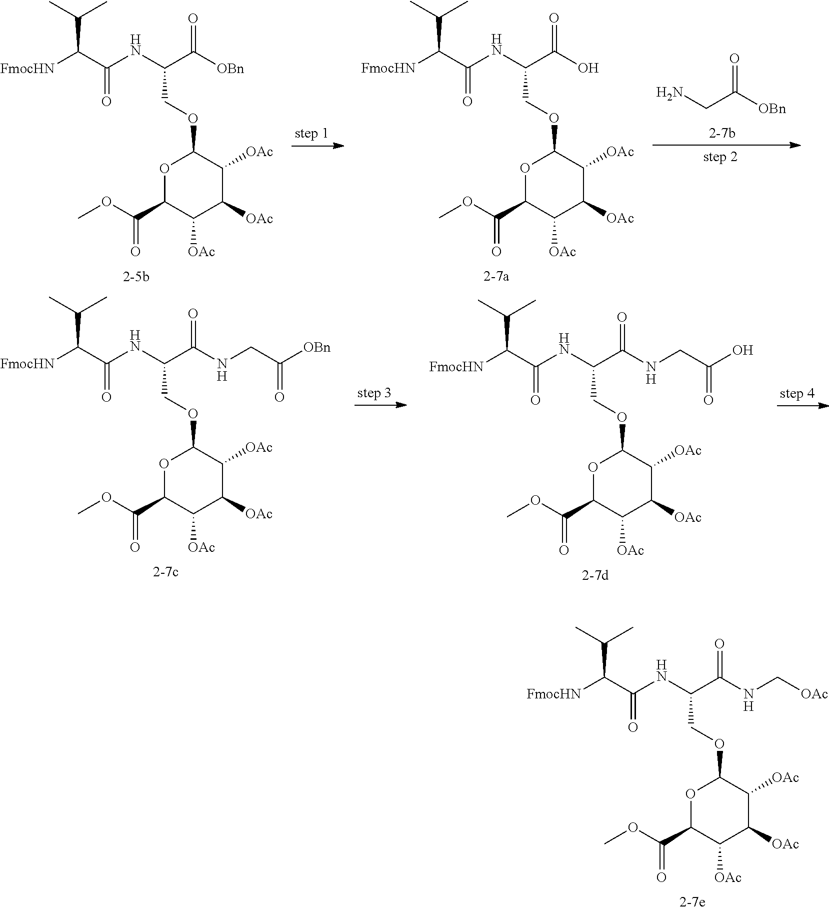

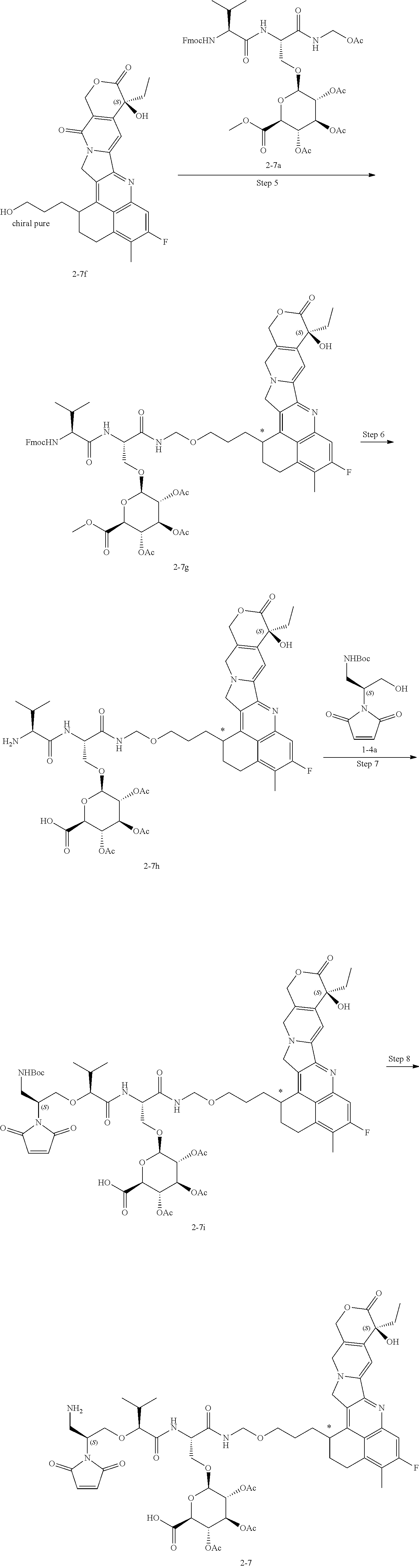

### Step 1: N-((((9H-fluoren-9-yl)methoxy)carbonyl)-L-valyl)-O-((2R,3R,4S,5S,6S)-3,4,5-triacetoxy-6-(methoxycarbonyl)tetrahydro-2H-pyran-2-yl)-L-serine (2-7a)

To a mixture of 2-5b (4.1 g, 4.92 mmol) in MeOH (50 mL), THF (100 mL) and DCM (20 mL) was added wet Pd/C (400 mg, 10% purity). The black suspension was purged with H₂ balloon three times and then stirred at r.t. for 1 hr. The black suspension was filtered through a pad of celite and washed with MeOH (200 mL). Combined organic layers were concentrated under vacuum to give 2-7a (3650 mg, 99.8% yield) as an off-white solid.

MS (ESI) m/z: 743.6 [M+H]⁺.

### Step 2: (2R,3R,4S,5S,6S)-2-((S)-2-((S)-2-((((9H-fluoren-9-yl)methoxy)carbonyl)amino)-3-methylbutanamido)-3-((2-(benzyloxy)-2-oxoethyl)amino)-3-oxopropoxy)-6-(methoxycarbonyl)tetrahydro-2H-pyran-3,4,5-triyl triacetate (2-7c)

To a solution of 2-7a (3.65 g, 4.92 mmol) and 2-7c (1.66 g, 4.92 mmol) in DMF (50 mL) were added HATU (1.87 g, 4.92 mmol) and DIPEA (1.59 g, 12.29 mmol). The mixture was stirred at r.t. for 30 min. After a complete reaction, the mixture was purified by FCC (MeOH/DCM=0~10%), the fraction was concentrated under vacuum to give 2-7c (3.8 g, 86.9% yield) as an off-white foamed solid.

MS (ESI) m/z: 890.7 [M+H]⁺.

### Step 3: N-((((9H-fluoren-9-yl)methoxy)carbonyl)-L-valyl)-O-((2R,3R,4S,5S,6S)-3,4,5-triacetoxy-6-(methoxycarbonyl)tetrahydro-2H-pyran-2-yl)-L-serylglycine (2-7d)

To a mixture of 2-7c (3.8 g, 4.27 mmol) in MeOH (150 mL) and DCM (50 mL) was added wet Pd/C (400 mg, 10% purity). The black suspension was purged with H₂ balloon three times and then stirred at r.t. for 40 min. The black suspension was filtered through a pad of celite and washed with MeOH (150 mL). Combined organic layers were concentrated under vacuum to give 2-7d (3.3 g, 96.6% yield) as an off-white solid.

MS (ESI) m/z: 800.7 [M+H]⁺.

### Step 4: (2R,3R,4S,5S,6S)-2-((S)-2-((S)-2-((((9H-fluoren-9-yl)methoxy)carbonyl)amino)-3-methylbutanamido)-3-((acetoxymethyl)amino)-3-oxopropoxy)-6-(methoxycarbonyl)tetrahydro-2H-pyran-3,4,5-triyl triacetate (2-7e)

To a solution of 2-7d (3.3 g, 4.13 mmol) in DMF (30 mL) were added Pb(OAc)₄ (2.74 g, 6.19 mmol), Cu(OAc)₂ (74.9 mg, 0.41 mmol) and HOAc (247.8 mg, 4.13 mmol). The resulting dark-colored mixture was purged with N₂ balloon three times and then stirred at 65° C. for 40 min; the mixture turned deep blue. The mixture was diluted with EtOAc (300 mL), washed with brine (100 mL*3), dried over Na₂SO₄, filtered and concentrated under vacuum to give a residue. It was purified by FCC (MeOH/DCM=0~10%), and the fraction was concentrated under vacuum to give 2-7e (2.8 g, 83.4% yield) as a pale-yellow solid.

MS (ESI) m/z: 836.6 [M+Na]⁺.

### Step 5: (2S,3S,4S,5R,6R)-6-((2S)-2-((S)-2-amino-3-methylbutanamido)-3-(((3-((9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)propoxy)methyl)amino)-3-oxopropoxy)-3,4,5-trihydroxytetrahydro-2H-pyran-2-carboxylic acid (2-7g)

A white suspension mixture of 2-7e (301 mg, 0.37 mmol), 2-7f (177 mg, 0.37 mmol) and 4 Å molecular sieve (500 mg) in anhydrous THF (10 mL) was stirred at r.t. for 10 min. Sc(OTf)₃ (218 mg, 0.44 mmol) was added and the resulting yellow suspension was stirred at r.t. for 4 hr. The yellow suspension mixture was filtered through a pad of celite and washed with EA. Combined organic layers were washed with sat. NaHCO₃ (30 mL) and brine (30 mL), dried over Na₂SO₄, filtered and the filtrate was concentrated under vacuum to give a residue. It was purified by silica gel column (MeOH/DCM=0%~5%). The fraction was concentrated under vacuum to give 2-7g (353 mg, 77.5% yield) as a white foam.

MS (ESI) m/z: 1254.8 [M+Na]⁺.

### Step 6: (2S,3S,4S,5R,6R)-6-((2S)-2-((S)-2-amino-3-methylbutanamido)-3-(((3-((9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)propoxy)methyl)amino)-3-oxopropoxy)-3,4,5-trihydroxytetrahydro-2H-pyran-2-carboxylic acid (2-7h)

2-7h (65 mg, 54.3% yield) as a white solid was synthesized according to Step 6 of Example 2-5.

MS (ESI) m/z: 870.7 [M+H]⁺.

### Step 7: (2S,3S,4S,5R,6R)-6-(((7S,12S,15S)-7-(2,5-dioxo-2,5-dihydro-1H-pyrrol-1-yl)-15-(((3-((9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)propoxy)methyl) carbamoyl)-12-isopropyl-2,2-dimethyl-4,10,13-trioxo-3,9-dioxa-5,11,14-triazahexadecan-16-yl)oxy)-3,4,5-trihydroxytetrahydro-2H-pyran-2-carboxylic acid (2-7i)

2-7i (18 mg, 40.1% yield) as a white solid was synthesized according to Step 1 of Example 2-6.

MS (ESI) m/z: 1166.9 [M+H]⁺.

### Step 8: (2S,3S,4S,5R,6R)-6-((2S)-2-((S)-2-((((S)-3-amino-2-(2,5-dioxo-2,5-dihydro-1H-pyrrol-1-yl) propoxy) carbonyl)amino)-3-methylbutanamido)-3-(((3-((9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)propoxy)methyl)amino)-3-oxopropoxy)-3,4,5-trihydroxytetrahydro-2H-pyran-2-carboxylic acid (2-7)

2-7 (4.7 mg, 50.1% yield) as clear syrup was synthesized according to Step 2 of Example 2-6.

MS (ESI) m/z: 1066.8 [M+H]⁺.

### Example 2-8

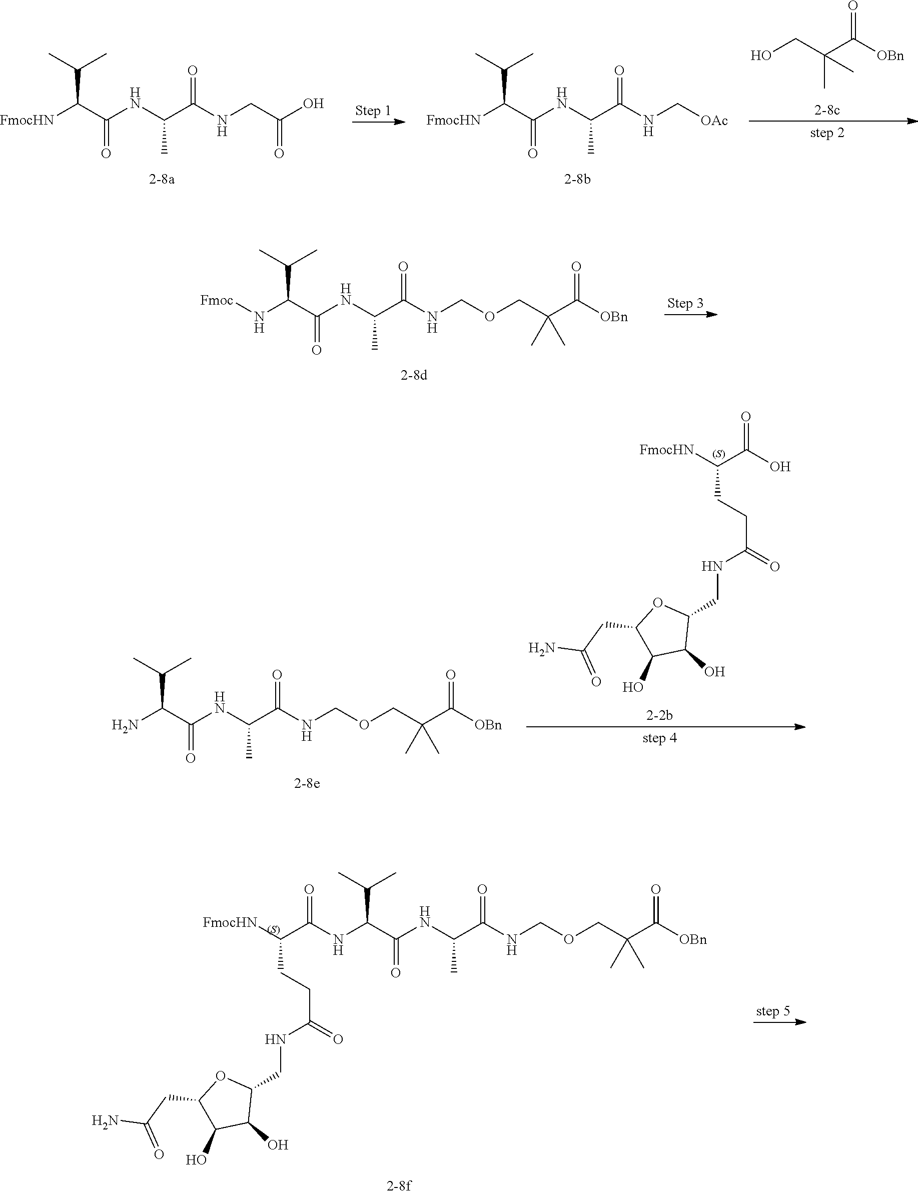

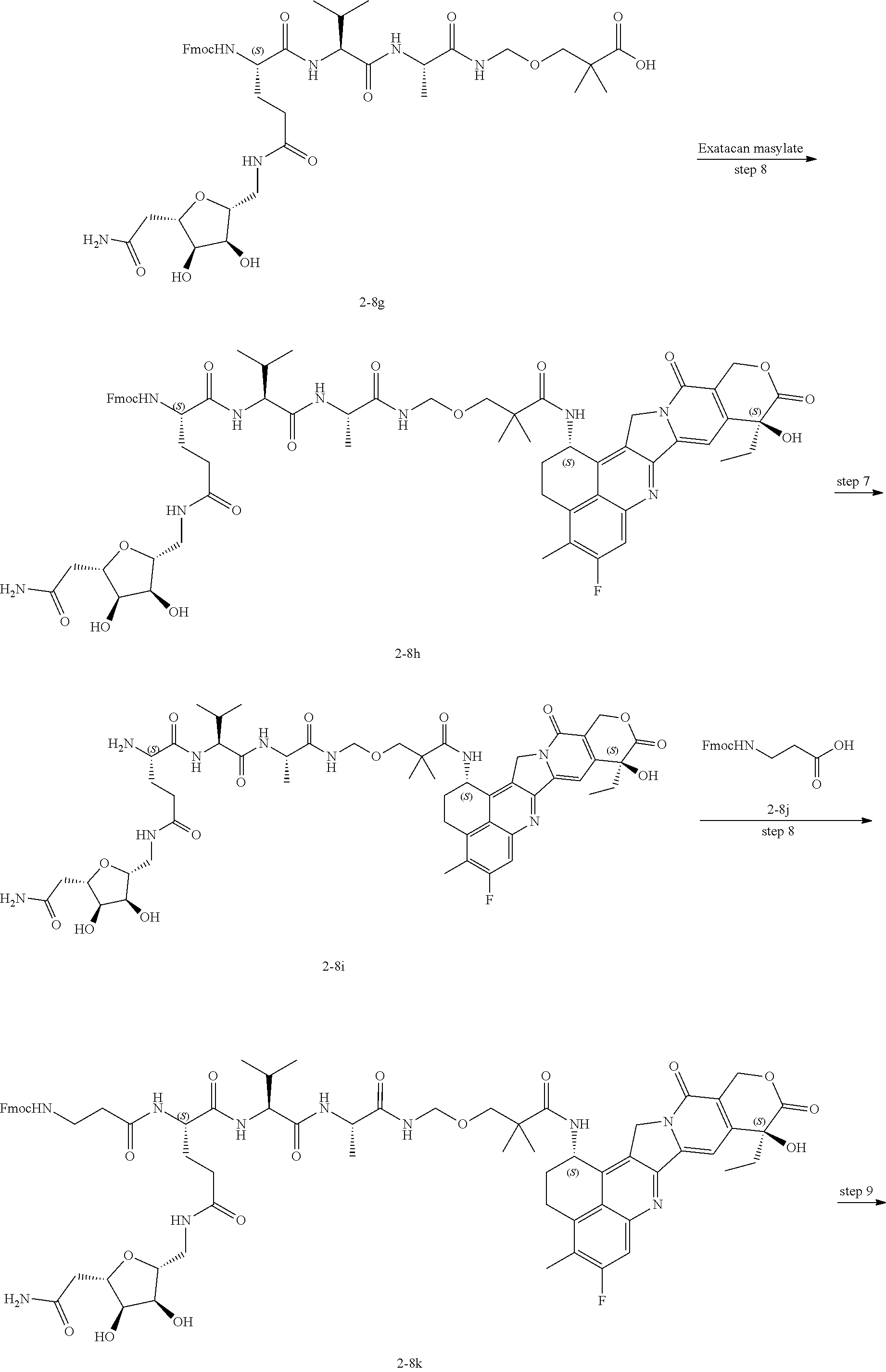

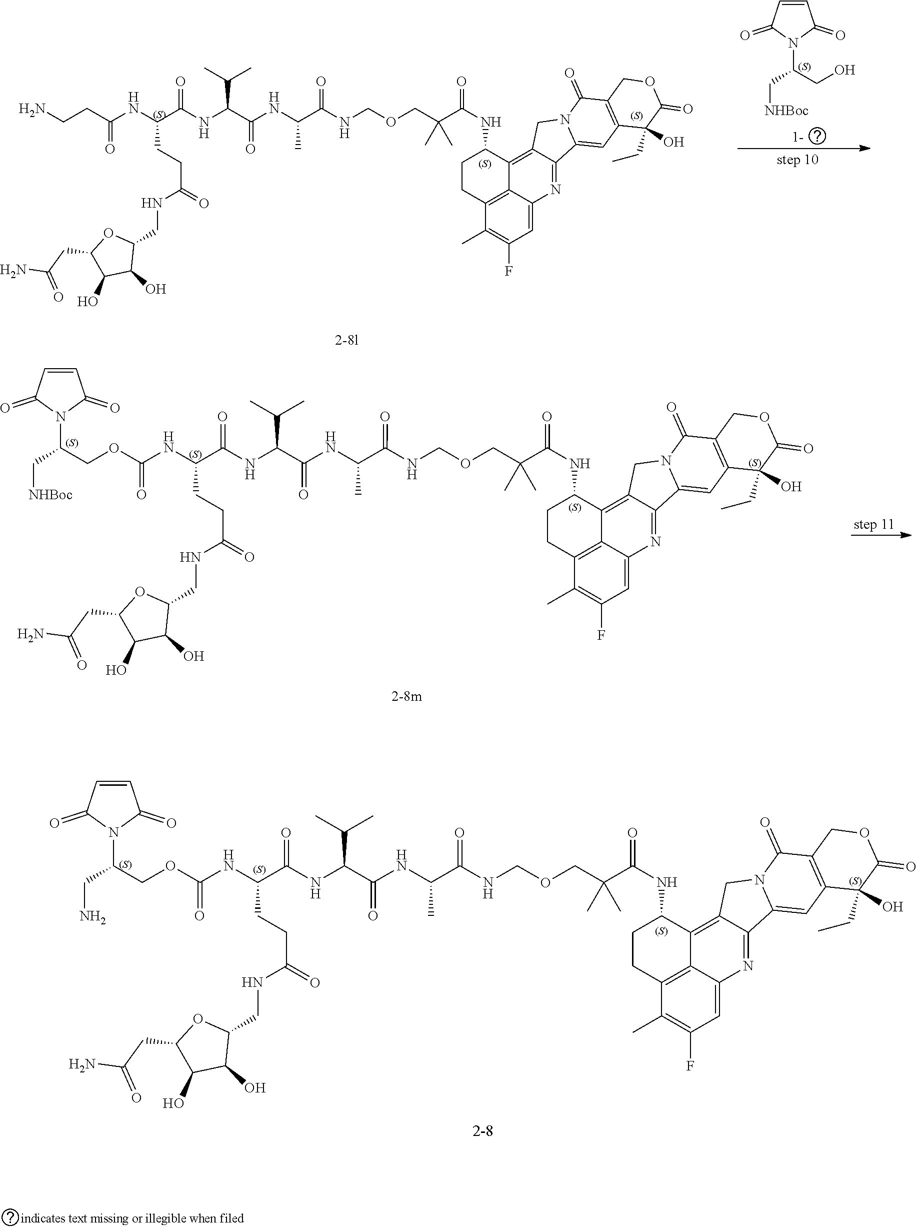

### Step 1: (5S,8S)-1-(9H-fluoren-9-yl)-5-isopropyl-8-methyl-3,6,9-trioxo-2-oxa-4,7,10-triazaundecan-11-yl acetate (2-8b)

To a mixture of 2-8a (5.0 g, 10.70 mmol) in DMF (100 mL) were added Cu(OAc)₂ (738.2 mg, 4.06 mmol), Pb(OAc)₄ (5.41 g, 12.2 mmol) and glacial acetic acid (1.4 mL, 1.5 g, 24.3 mmol). The resulting deep blue mixture was purged with N₂ balloon three times, then heated to 70° C. for 1 hr, and turned to a green mixture. The mixture was diluted with EtOAc (1000 mL), washed with brine (300 mL*3), dried over Na₂SO₄, filtered and the filtrate was concentrated under vacuum to give crude product. It was triturated with MTBE/PE (10/1, 500 mL) and filtered, followed by high vacuum drying of the filter cake to give 2-8b (4.4 g, 85.4% yield) as a pale-yellow solid.

MS (ESI) m/z: 504.5 [M+Na]⁺.

¹H NMR (400 MHZ, dmso) δ 8.89 (t, J=6.8 Hz, 1H), 8.10 (d, J=8.0 Hz, 1H), 7.89 (d, J=7.6 Hz, 2H), 7.75 (t, J=6.4 Hz, 2H), 7.43 (dd, J=14.4, 7.6 Hz, 3H), 7.35-1.30 (m, 2H), 5.13-5.05 (m, 2H), 4.35-4.19 (m, 4H), 3.88 (dd, J=8.8, 7.2 Hz, 1H), 1.98 (s, 2H), 1.26-1.17 (m, 4H), 0.86 (dd, J=9.8, 6.8 Hz, 6H).

### Step 2: benzyl (5S,8S)-1-(9H-fluoren-9-yl)-5-isopropyl-8,14,14-trimethyl-3,6,9-trioxo-2,12-dioxa-4,7,10-triazapentadecan-15-oate (2-8d)

A white suspension mixture of 2-8b (300 mg, 0.623 mmol), 2-8c (259.6 mg, 1.246 mmol) and 4 Å molecular sieves in anhydrous THF (10 mL) was stirred at r.t. for 10 min. Sc(OTf)₃ (368.0 mg, 0.748 mmol) was added and the resulting yellow suspension was stirred at r.t. for 4 hr. The yellow suspension mixture was filtered through a pad of celite and washed with EtOAc (30 mL). Combined organic layers were washed with sat. NaHCO₃ (30 mL) and brine (30 mL), dried over Na₂SO₄, filtered and the filtrate was concentrated under vacuum to give a residue. It was purified by silica gel column (MeOH/DCM=0%~5%). The fraction was concentrated under vacuum to give 2-8d (274 mg, 69.8% yield) as a white solid.

MS (ESI) m/z: 652.6 [M+Na]⁺.

### Step 3: benzyl 3-(((S)-2-((S)-2-amino-3-methylbutanamido) propanamido) methoxy)-2,2-dimethylpropanoate (2-8e)

To a solution of 2-8d (274.0 mg, 0.44 mmol) in DMF (5 mL) was added Et₂N (477.3 mg, 5.53 mmol). The mixture was stirred at r.t. for 20 min. The reaction mixture was concentrated under vacuum and co-evaporated with Tol two times to give 2-8e (275.3 mg, crude) as a brown oil.

MS (ESI) m/z: 430.4 [M+Na]⁺.

### Step 4: benzyl (5S,8S,11S)-5-(3-((((2S,3R,4S,5R)-5-(2-amino-2-oxoethyl)-3,4-dihydroxytetrahydrofuran-2-yl)methyl)amino)-3-oxopropyl)-1-(9H-fluoren-9-yl)-8-isopropyl-11,17,17-trimethyl-3,6,9,12-tetraoxo-2,15-dioxa-4,7,10,13-tetraazaoctadecan-18-oate (2-8f)

To a solution of 2-8e (275.3 mg, crude) and 2-2b (282.3 mg, 0.52 mmol) in DMF (5 mL) were added HATU (198.2 mg, 0.52 mmol) and DIPEA (168.4 mg, 1.30 mmol). The mixture was stirred at r.t. for 10 min. The mixture was purified by reserve phase (C18, 60 g, 30%~70%), and the fraction was freeze-dried to give 2-8f (370 mg, 91.5% yield) as a brown solid.

MS (ESI) m/z: 953.8 [M+Na]⁺.

### Step 5: (5S,8S,11S)-5-(3-((((2S,3R,4S,5R)-5-(2-amino-2-oxoethyl)-3,4-dihydroxytetrahydrofuran-2-yl)methyl)amino)-3-oxopropyl)-1-(9H-fluoren-9-yl)-8-isopropyl-11,17,17-trimethyl-3,6,9,12-tetraoxo-2,15-dioxa-4,7,10,13-tetraazaoctadecan-18-oic acid (2-8g)

To a mixture of 2-8f (370 mg, 0.388 mmol) in DMF/MeOH (1:1, 15 mL) was added Pd/C (35 mg). The black suspension was purged with H₂ balloon three times, then stirred at r.t. for 6 hr under H₂ balloon. The reaction mixture was filtered through a pad of celite and washed with DMF (15 mL) and MeOH (50 mL). Combined organic layers were concentrated under vacuum and co-evaporated with Tol three times to give 2-8g (350 mg, crude) as a light-brown solid.

MS (ESI) m/z: 863.7 [M+Na]⁺.

### Step 6: (9H-fluoren-9-yl)methyl ((6S,9S,12S)-1-((2S,3R,4S,5R)-5-(2-amino-2-oxoethyl)-3,4-dihydroxytetrahydrofuran-2-yl)-19-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-9-isopropyl-12,18,18-trimethyl-3,7,10,13,19-pentaoxo-16-oxa-2,8,11,14-tetraazanonadecan-6-yl) carbamate (2-8h)

2-8h (276.1 mg, 65% yield) as a pale-yellow solid was synthesized according to Step 3 of Example 2-4.

MS (ESI) m/z: 1282.1 [M+Na]⁺.

### Step 7: (S)-2-amino-N₅-(((2S,3R,4S,5R)-5-(2-amino-2-oxoethyl)-3,4-dihydroxytetrahydrofuran-2-yl)methyl)-N1-((S)-1-(((S)-1-(((3-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-2,2-dimethyl-3-oxopropoxy)methyl)amino)-1-oxopropan-2-yl)amino)-3-methyl-1-oxobutan-2-yl) pentanediamide (2-8i)

To a solution of 2-8h (276.1 mg, 0.22 mmol) in DMF (5 mL) was added Et₂N (240.6 mg, 3.29 mmol). The mixture was stirred at r.t. for 20 min. The reaction mixture was concentrated under vacuum and co-evaporated with Tol two times to give 2-8i (278.3 mg, crude) as a brown solid.

MS (ESI) m/z: 1036.9 [M+H]⁺.

### Step 8: (9H-fluoren-9-yl)methyl ((8S,11S,14S)-14-(3-((((2R,3S,4R,5S)-5-(2-amino-2-oxoethyl)-3,4-dihydroxytetrahydrofuran-2-yl)methyl)amino)-3-oxopropyl)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-11-isopropyl-2,2,8-trimethyl-1,7,10,13,16-pentaoxo-4-oxa-6,9,12,15-tetraazaoctadecan-18-yl) carbamate (2-8k)

To a solution of 2-8i (49.9 mg, 0.048 mmol) and 2-8j (15.0 mg, 0.048 mmol) in DMF (3 mL) were added HATU (18.3 mg, 0.048 mmol) and DIPEA (18.7 mg, 0.145 mmol). The mixture was stirred at r.t. for 10 min. The mixture was purified by prep-HPLC (FA 0.1%), and the fraction was freeze-dried to give 2-8k (42 mg, 65.6% purity) as a white solid.

MS (ESI) m/z: 1352.0 [M+Na]⁺.

### Step 9: (S)-N5-(((2R,3S,4R,5S)-5-(2-amino-2-oxoethyl)-3,4-dihydroxytetrahydrofuran-2-yl)methyl)-2-(3-aminopropanamido)-N₁—((S)-1-(((S)-1-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-1-oxopropan-2-yl)amino)-3-methyl-1-oxobutan-2-yl) pentanediamide (2-8l)

To a solution of 2-8k (42 mg, 0.032 mmol) in DMF (5 mL) was added Et₂N (34.7 mg, 0.474 mmol). The mixture was stirred at r.t. for 20 min. The reaction mixture was concentrated under vacuum and co-evaporated with Tol two times to give 2-8l (43 mg, crude) as a brown solid.

MS (ESI) m/z: 1107.9 [M+H]⁺.

### Step 10: tert-butyl ((2S,7S,10S,13S)-7-(3-((((2R,3S,4R,5S)-5-(2-amino-2-oxoethyl)-3,4-dihydroxytetrahydrofuran-2-yl)methyl)amino)-3-oxopropyl)-2-(2,5-dioxo-2,5-dihydro-1H-pyrrol-1-yl)-20-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-10-isopropyl-13,19,19-trimethyl-5,8,11,14,20-pentaoxo-4,17-dioxa-6,9,12,15-tetraazaicosyl) carbamate (2-8m)

To a solution of 1-4e (120 mg, 0.444 mmol) in DMF (3 mL) were added bis(4-nitrophenyl) carbonate (148.7 mg, 0.489 mmol) and DIPEA (94.7 mg, 0.733 mmol). The resulting light-yellow mixture was stirred at r.t. for 2 hr. It was purified by prep-HPLC (FA), and the fraction was concentrated under vacuum to give active carbonate (81.6 mg, 42.2% yield) as a white solid. To active carbonate (13.8 mg, 0.032 mmol) and 2-8l (42.5 mg, crude) in DMF (3 mL) was added DIPEA (8.2 mg, 0.063 mmol). The resulting light-yellow mixture was stirred at r.t. for 1 hr. The reaction mixture was purified by prep-HPLC (FA 0.1%), and the fraction was lyophilized to give 2-8m (28.1 mg, 0.02 mmol) as a pale-yellow solid.

MS (ESI) m/z: 1427.1 [M+Na]⁺.

### Step 11: (S)-3-amino-2-(2,5-dioxo-2,5-dihydro-1H-pyrrol-1-yl)propyl ((6S,9S,12S)-1-((2R,3S,4R,5S)-5-(2-amino-2-oxoethyl)-3,4-dihydroxytetrahydrofuran-2-yl)-19-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-9-isopropyl-12,18,18-trimethyl-3,7,10,13,19-pentaoxo-16-oxa-2,8,11,14-tetraazanonadecan-6-yl) carbamate (2-8)

To a suspension of 2-8m (28.1 mg, 0.02 mmol) in DCM (3 mL) was added ZnBr2 (179.7 mg, 0.798 mmol) then stirred at 45° C. for 4 hr. The mixture was concentrated under vacuum and dissolved in DMSO then purified by prep-HPLC (0.1% FA). The fraction was lyophilized to give 2-8 (15.7 mg, 60.4% yield) as a pale-yellow solid.

MS (ESI) m/z: 1304.1 [M+H]⁺.

### Example 2-9

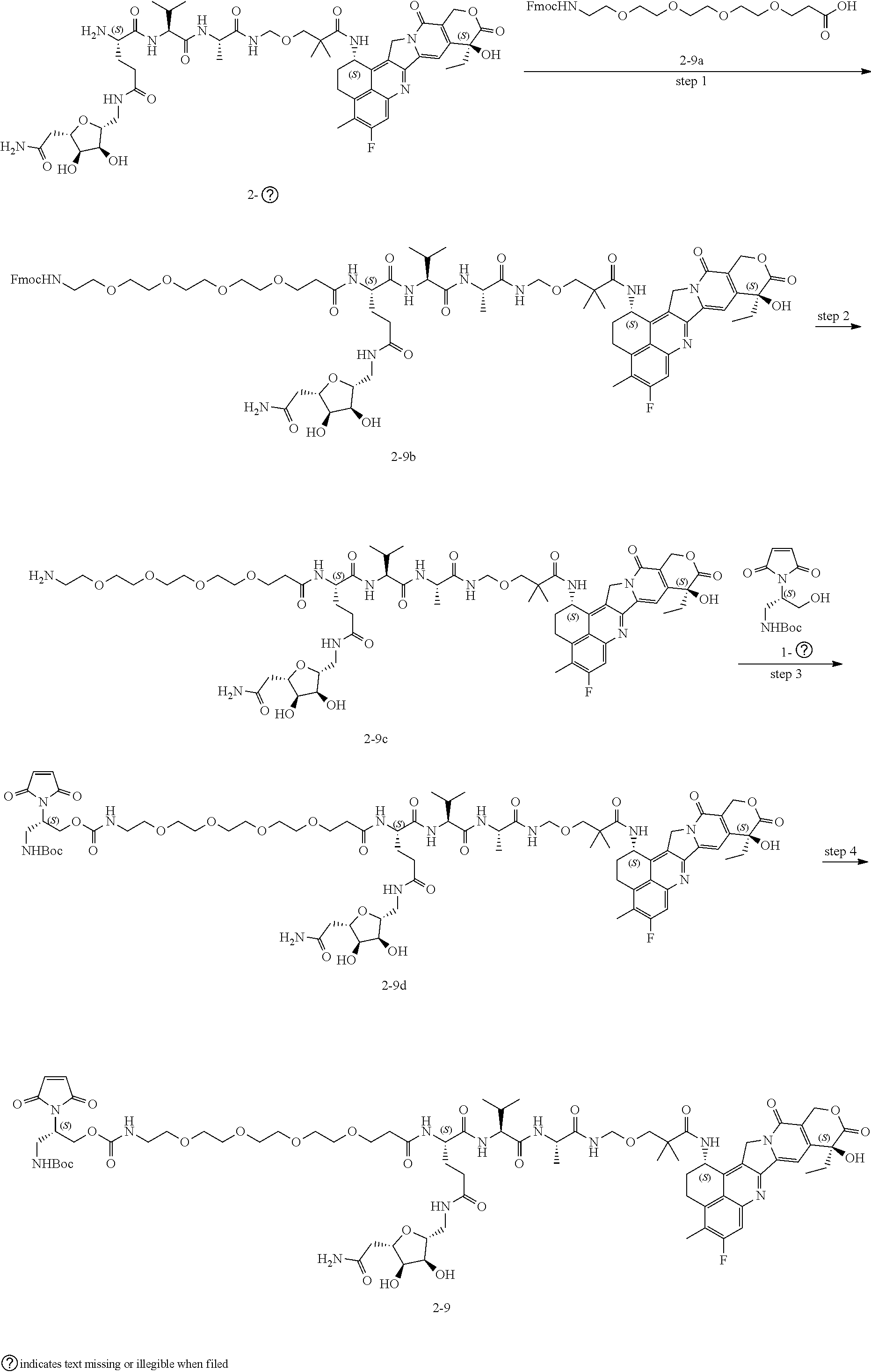

(S)-3-((tert-butoxycarbonyl)amino)-2-(2,5-dioxo-2,5-dihydro-1H-pyrrol-1-yl) propyl ((17S,20S,23S)-17-(3-((((2R,3S,4R,5S)-5-(2-amino-2-oxoethyl)-3,4-dihydroxytetrahydrofuran-2-yl)methyl)amino)-3-oxopropyl)-30-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-20-isopropyl-23,29,29-trimethyl-15,18,21,24,30-pentaoxo-3,6,9,12,27-pentaoxa-16,19,22,25-tetraazatriacontyl) carbamate (2-9)

2-9 (14.4 mg, 61.5% yield) as a pale-yellow solid was synthesized according to the synthetic procedure of Example 2-8.

MS (ESI) m/z: 1480.2 [M+H]⁺.

### Example 2-10

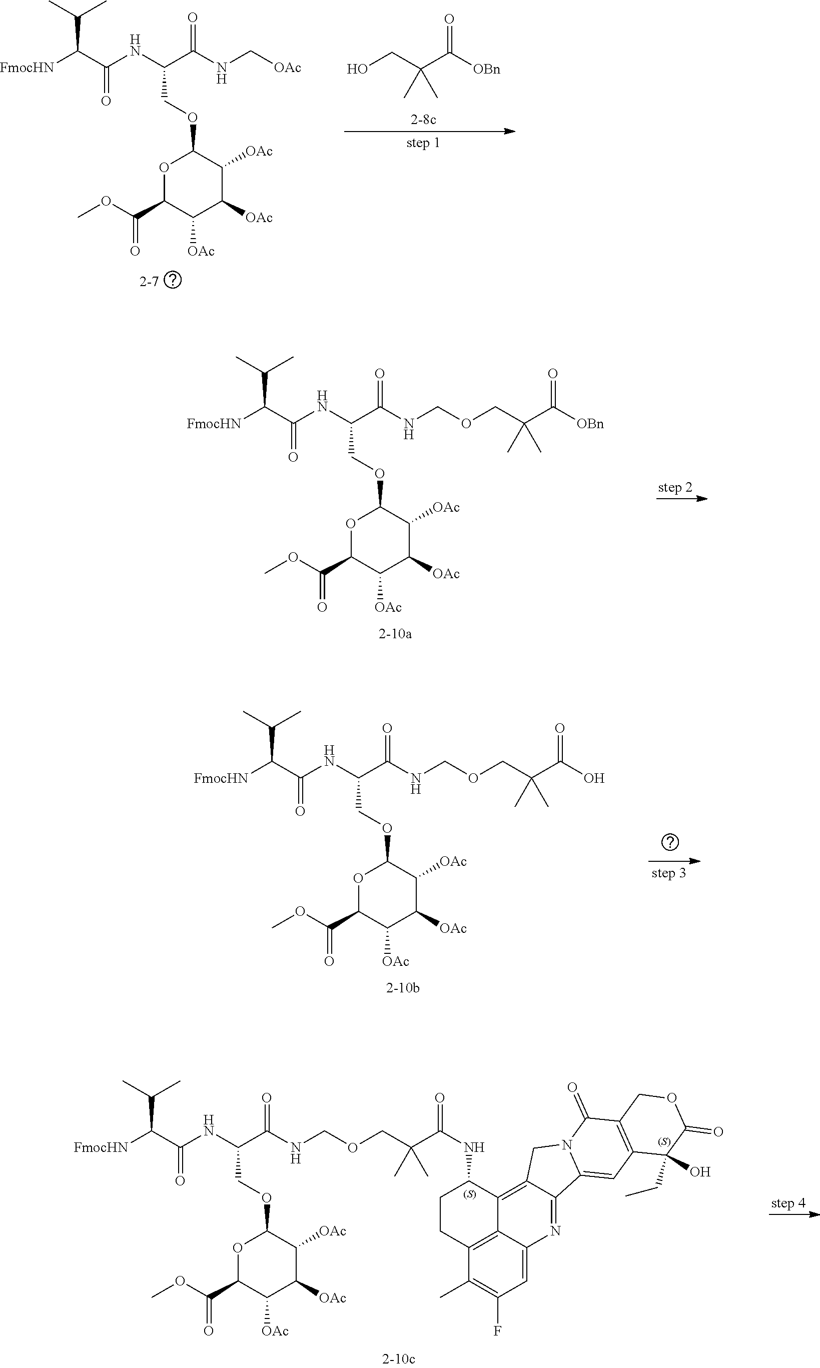

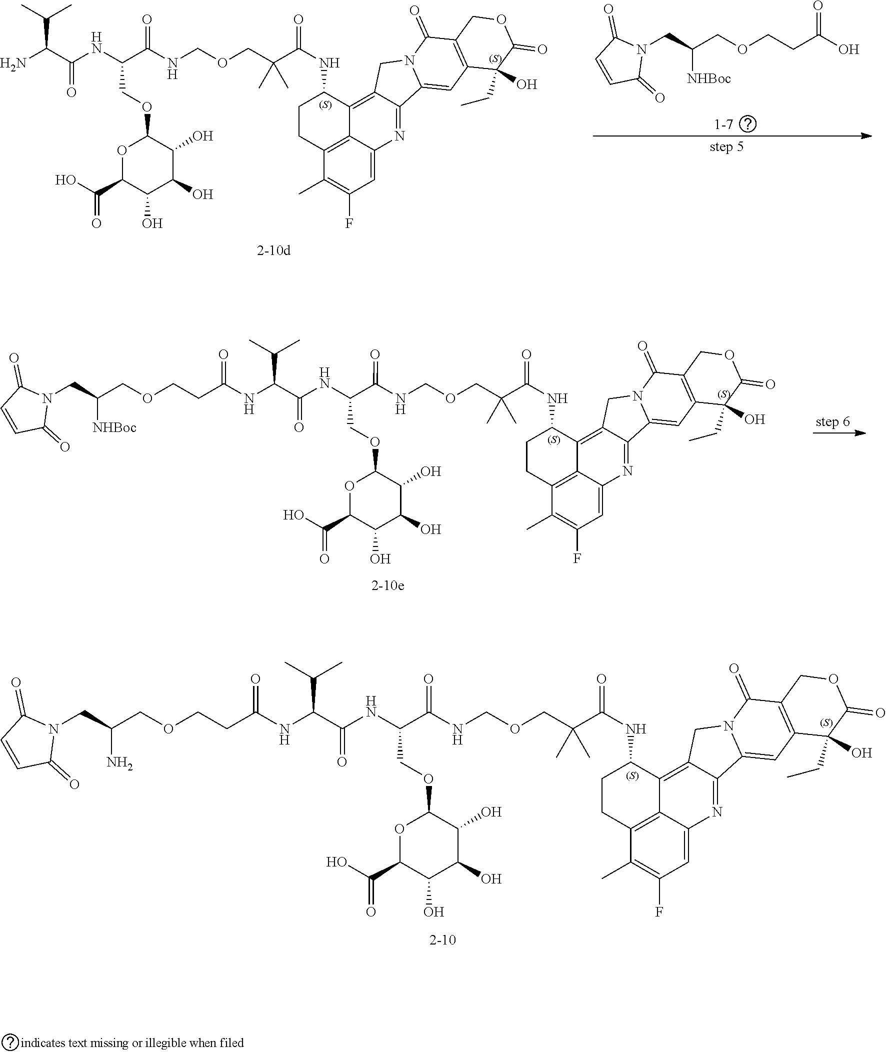

### Step 1: (2R,3R,4S,5S,6S)-2-((S)-2-((S)-2-((((9H-fluoren-9-yl)methoxy)carbonyl)amino)-3-methylbutanamido)-3-(((3-(benzyloxy)-2,2-dimethyl-3-oxopropoxy)methyl)amino)-3-oxopropoxy)-6-(methoxycarbonyl)tetrahydro-2H-pyran-3,4,5-triyl triacetate (2-10a)

A white suspension mixture of 2-7e (300 mg, 0.37 mmol), 2-8c (154 mg, 0.74 mmol) and 4 Å molecular sieves (500 mg) in anhydrous THF (10 mL) was stirred at r.t. for 10 min. Sc(OTf)₃ (217.9 mg, 0.44 mmol) was added and the resulting yellow suspension was stirred at r.t. for 4 hr. The yellow suspension mixture was filtered through a pad of celite and washed with EA. Combined organic layers were washed with sat. NaHCO₃ (30 mL) and brine (30 mL), dried over Na₂SO₄, filtered and the filtrate was concentrated under vacuum to give a residue. It was purified by silica gel column (MeOH/DCM=0%~5%). The fraction was concentrated under vacuum to give 2-10a (275 mg, 77.5% yield) as a white foam solid.

MS (ESI) m/z: 984.8 [M+Na]⁺.

### Step 2: (5S,8S)-1-(9H-fluoren-9-yl)-5-isopropyl-14,14-dimethyl-3,6,9-trioxo-8-((((2R,3R,4S,5S,6S)-3,4,5-triacetoxy-6-(methoxycarbonyl)tetrahydro-2H-pyran-2-yl)oxy)methyl)-2,12-dioxa-4,7,10-triazapentadecan-15-oic acid (2-10b)

To a solution of 2-10a (275 mg, 0.29 mmol) in MeOH (10 mL) was added wet Pd/C (55 mg, 10% purity). The black suspension was purged with H₂ balloon three times then stirred at r.t. for 2 hr. The mixture was filtered through a syringe head and washed with MeOH (15 mL), and concentrated under vacuum to give 2-10b (230 mg, crude) as a white foam solid.

MS (ESI) m/z: 894.6 [M+Na]⁺.

### Step 3: (2R,3R,4S,5S,6S)-2-((S)-2-((S)-2-((((9H-fluoren-9-yl)methoxy)carbonyl)amino)-3-methylbutanamido)-3-(((3-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-2,2-dimethyl-3-oxopropoxy)methyl)amino)-3-oxopropoxy)-6-(methoxycarbonyl)tetrahydro-2H-pyran-3,4,5-triyl triacetate (2-10c)

To a mixture of 2-10b (230 mg, crude), exatecan mesylate (139.9 mg, 0.26 mmol) and HATU (100.3 mg, 0.26 mol) in DMF (5 mL) was added DIPEA (102.3 mg, 0.79 mmol). The resulting brown mixture was stirred at r.t. for 1 hr. The mixture was diluted with EtOAc (20 mL), washed with brine (20 mL*3), dried over Na₂SO₄, filtered and concentrated under vacuum to give a residue. It was purified by FCC (MeOH/DCM=0%~3%), and concentrated under vacuum to give 2-10c (325 mg, 95.6% yield) as an off-white foam solid.

MS (ESI) m/z: 1289.9 [M+H]⁺.

### Step 4: (2S,3S,4S,5R,6R)-6-((S)-2-((S)-2-amino-3-methylbutanamido)-3-(((3-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic acid (2-10d)

To a solution of 2-10c (325 mg, 0.25 mmol) in DMF (5 mL) was added Et₂N (523.2 mg, 5.06 mmol). The mixture was stirred at r.t. for 20 min. After LCMS showed the reaction was completed, the mixture was concentrated under vacuum to give a crude product. It was dissolved in MeOH (6 mL), and K₂CO₃ (174.7 mg, 1.26 mmol) was added and stirred at r.t. for 10 min. Then H₂O (2 mL) was added to the mixture and stirred at r.t. for 30 min. The mixture was acidified with sat. KHSO₄ at 0° C. to pH=3, filtered and purified by prep-HPLC (0.1% FA). The fraction was lyophilized to give 2-10d (140 mg, 59.7% yield) as a pale-yellow solid.

MS (ESI) m/z: 927.4 [M+H]⁺.

¹H NMR (400 MHz, d₆-DMSO) δ 9.56 (s, 1H), 8.39 (s, 1H), 8.07 (d, J=8.4 Hz, 1H), 7.77 (d, J=11.2 Hz, 1H), 7.31 (s, 1H), 6.52 (s, 1H), 5.54 (dd, J=13.2, 7.2 Hz, 1H), 5.43 (s, 2H), 5.18 (dd, J=41.6, 18.8 Hz, 2H), 5.09-5.02 (m, 1H), 4.96 (s, 1H), 4.62 (dd, J=10.0, 6.8 Hz, 1H), 4.56-4.44 (m, 2H), 4.19 (d, J=7.6 Hz, 1H), 3.82 (dd, J=10.8, 6.8 Hz, 1H), 3.61 (dd, J=11.6, 6.4 Hz, 2H), 3.17-3.05 (m, 4H), 2.94 (t, J=8.0 Hz, 1H), 2.39 (s, 3H), 2.11 (dt, J=21.3, 7.6 Hz, 2H), 2.03-1.93 (m, 2H), 1.92-1.78 (m, 3H), 1.12 (d, J=8.0 Hz, 6H), 0.87 (dd, J=13.0, 6.6 Hz, 9H).

### Step 5: (2S,3S,4S,5R,6R)-6-(((6S,13S,16S)-6-((2,5-dioxo-2,5-dihydro-1H-pyrrol-1-yl)methyl)-16-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)carbamoyl)-13-isopropyl-2,2-dimethyl-4,11,14-trioxo-3,8-dioxa-5,12,15-triazaheptadecan-17-yl)oxy)-3,4,5-trihydroxytetrahydro-2H-pyran-2-carboxylic acid (2-10e)

To a solution of 1-7e (6.7 mg, 0.02 mmol) in DMF (2 mL) were added HATU (8.6 mg, 0.02 mmol) and DIPEA (5.2 mg, 0.04 mmol). The mixture was stirred at r.t. for 10 min. 2-10d (15 mg, 0.02 mmol) was added and stirred at r.t. for 30 min. The mixture was purified by prep-HPLC (FA) to give 2-10e (9.1 mg, 44.4% yield) as an off-white solid.

MS (ESI) m/z: 1252.1 [M+H]⁺.

### Step 6: (2S,3S,4S,5R,6R)-6-((S)-2-((S)-2-(3-((S)-2-amino-3-(2,5-dioxo-2,5-dihydro-1H-pyrrol-1-yl) propoxy) propanamido)-3-methylbutanamido)-3-(((3-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic acid (2-10)

To a suspension of 2-10e (9.1 mg, 0.007 mmol) in DCM (1.5 mL) was added ZnBr₂ (32.4 mg, 0.14 mmol), then stirred at 45° C. for 8 hr. The mixture was concentrated under vacuum and dissolved in DMSO, then purified by prep-HPLC (0.1% FA). The fraction was lyophilized to give 2-10 (7.1 mg, 85.7% yield) as a white solid.

MS (ESI) m/z: 1151.9 [M+H]⁺.

### Example 2-11

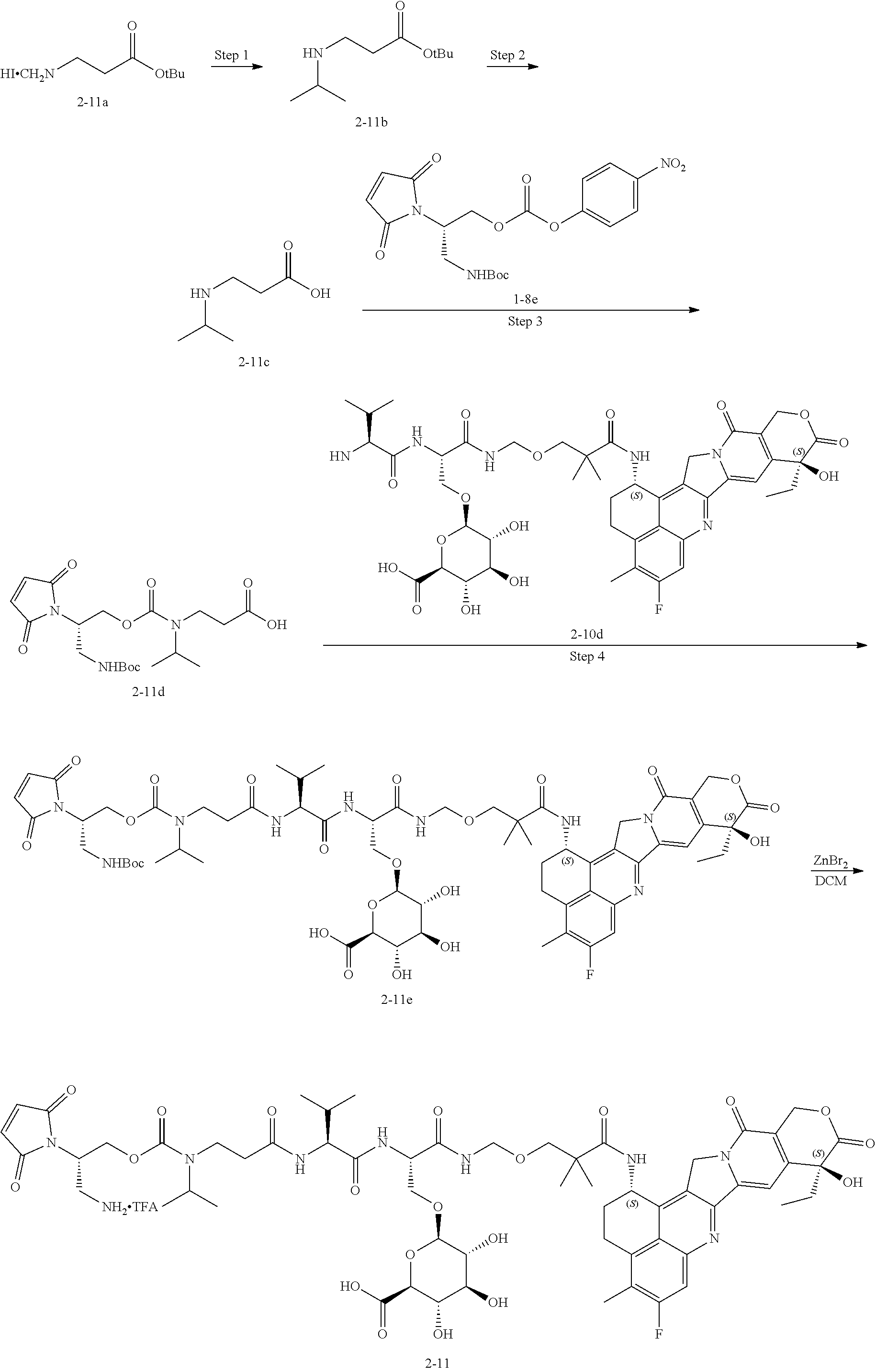

### Step 1: tert-Butyl 3-(isopropylamino)propanoate (2-11b)

To a solution of 2-11a (908 mg, 5.0 mmol) and acetone (580 mg, 10.0 mmol) in MeOH—H₂O (1:1, 20 mL) was added NaBH₃CN (1.28 g, 20.0 mmol) at r.t. The reaction was stirred at r.t. for 18 h. The mixture was concentrated in vacuo and the residue was re-dissolved in DCM (20 mL) and washed with sat. NaHCO₃ (10 mL), H₂O (10 mL), brine (10 mL), dried over Na₂SO₄, filtered and the filtrate was concentrated under vacuum to give a crude target product 1-11b (1.21 g) as a colorless oil, which was used for the next step directly.

MS (ESI) m/z: 188.2 [M+H]⁺.

### Step 2:3-(Isopropylamino) propanoic acid (2-11c)

A solution of 2-11b (600 mg, 2.5 mmol) in TFA/DCM (5:2, 7 mL) was stirred at r.t. overnight. The mixture was concentrated under vacuum, and the residue was re-dissolved in water. The aqueous layer was washed with EtOAc and lyophilized to give 2-11c (444.4 mg, 77.9% yield) as a white solid.

MS (ESI) m/z: 132.1 [M+H]⁺.

### Step 3: (S)-7-(2,5-Dioxo-2,5-dihydro-1H-pyrrol-1-yl)-11-isopropyl-2,2-dimethyl-4,10-dioxo-3,9-dioxa-5,11-diazatetradecan-14-oic acid (2-11d)

To a solution of 2-11c (12.3 mg, 0.094 mmol) and 1-8e (20 mg, 0.046 mmol) in DMF (1 mL) was added DIPEA (30 mg, 0.23 mmol) and HOBt (2 mg, 0.014 mmol) at r.t. The reaction was stirred at r.t. for 16 min. The reaction mixture was diluted with DMF and acidified with 0.1 N HCl. The mixture was purified by prep-HPLC (FA), the fraction was lyophilized to give 2-11d (6.8 mg, 34.6% yield) as a pale-yellow solid.

MS (ESI) m/z: 450.48 [M+Na]⁺.

### Step 4: (2S,3S,4S,5R,6R)-6-(((7S,16S,19S)-7-(2,5-Dioxo-2,5-dihydro-1H-pyrrol-1-yl)-19-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)carbamoyl)-11,16-diisopropyl-2,2-dimethyl-4,10,14,17-tetraoxo-3,9-dioxa-5,11,15,18-tetraazaicosan-20-yl)oxy)-3,4,5-trihydroxytetrahydro-2H-pyran-2-carboxylic acid (2-11e)

To a solution of 2-11d (6.8 mg, 0.0159 mmol) in DMF (0.5 mL) were added HATU (6.0 mg, 0.0159 mmol), DIPEA (4.7 mg, 0.0363 mmol) and 2-10d (13.4 mg, 0.0145 mmol). The reaction was stirred at r.t. for 30 min. The mixture was diluted with DMF (1.5 mL) and acidified with AcOH. It was purified by prep-HPLC (FA). The fraction was lyophilized to give 2-11e (5.1 mg, 26.4% yield) as a white solid.

MS (ESI) m/z: 1358.9 [M+Na]⁺.

### Step 5: (2S,3S,4S,5R,6R)-6-(((2S,5S,14S)-15-Amino-14-(2,5-dioxo-2,5-dihydro-1H-pyrrol-1-yl)-2-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)carbamoyl)-5,10-diisopropyl-4,7,11-trioxo-12-oxa-3,6,10-triazapentadecyl)oxy)-3,4,5-trihydroxytetrahydro-2/H-pyran-2-carboxylic acid (2-11)

To a suspension of 2-11e (5.1 mg, 0.007 mmol) in DCM (1.5 mL) was added ZnBr₂ (32.4 mg, 0.14 mmol), then stirred at 45° C. for 21 h. The mixture was concentrated under vacuum and dissolved in 0.1%/ACN (80:20, 1 mL) then purified by prep-HPLC (0.1% TFA). The fraction was lyophilized to give 2-11 (2.2 mg, 46.6% yield) as a white solid.

MS (ESI) m/z: 1236.8 [M+H]⁺.

### Example 2-12

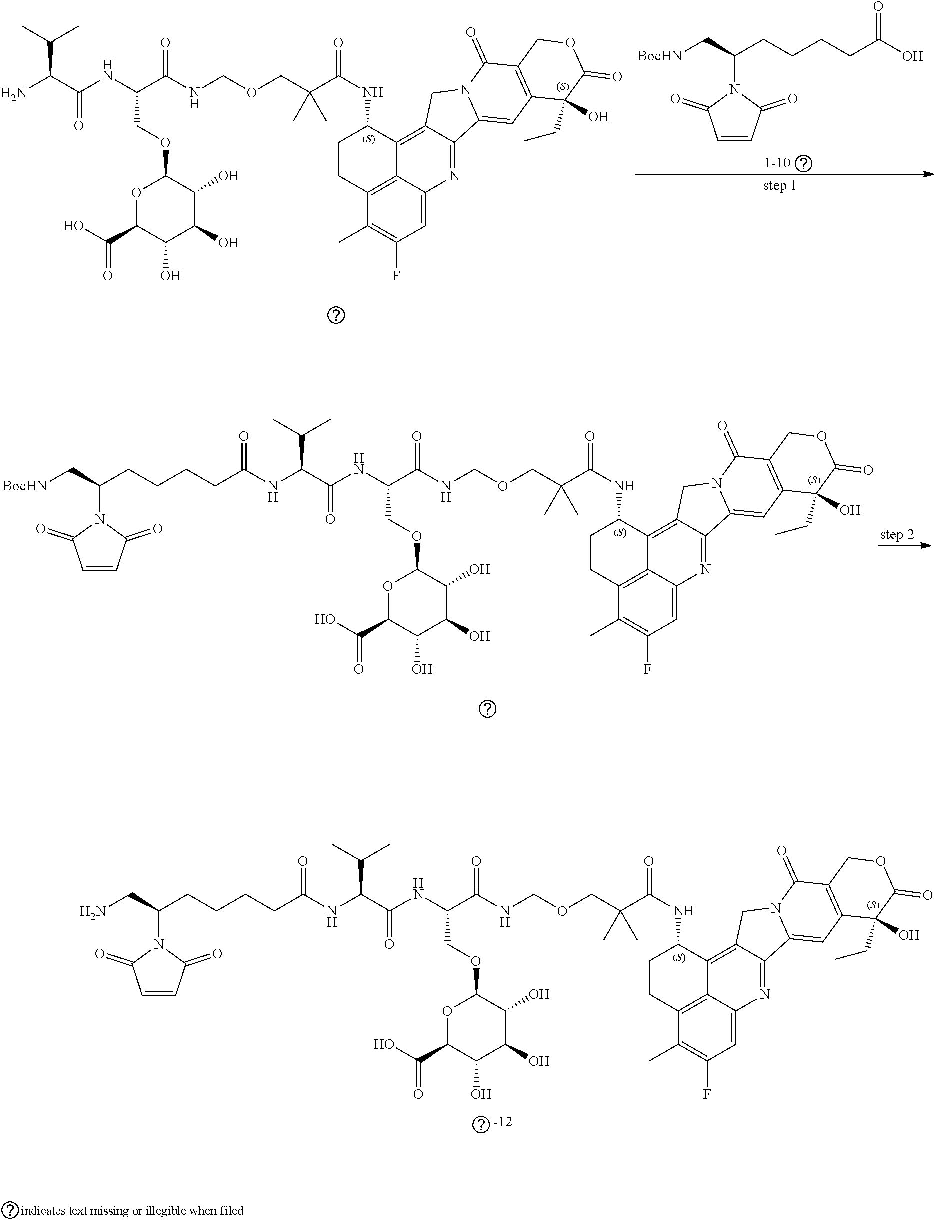

### (2S,3S,4S,5R,6R)-6-((S)-2-((S)-2-((R)-7-amino-6-(2,5-dioxo-2,5-dihydro-1H-pyrrol-1-yl) heptanamido)-3-methylbutanamido)-3-(((3-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic acid (2-12)

2-12 (5.8 mg, 45% yield) as a white solid was synthesized according to the synthetic procedure of Example 2-10.

MS (ESI) m/z: 1149.9 [M+H]⁺.

### Example 2-13

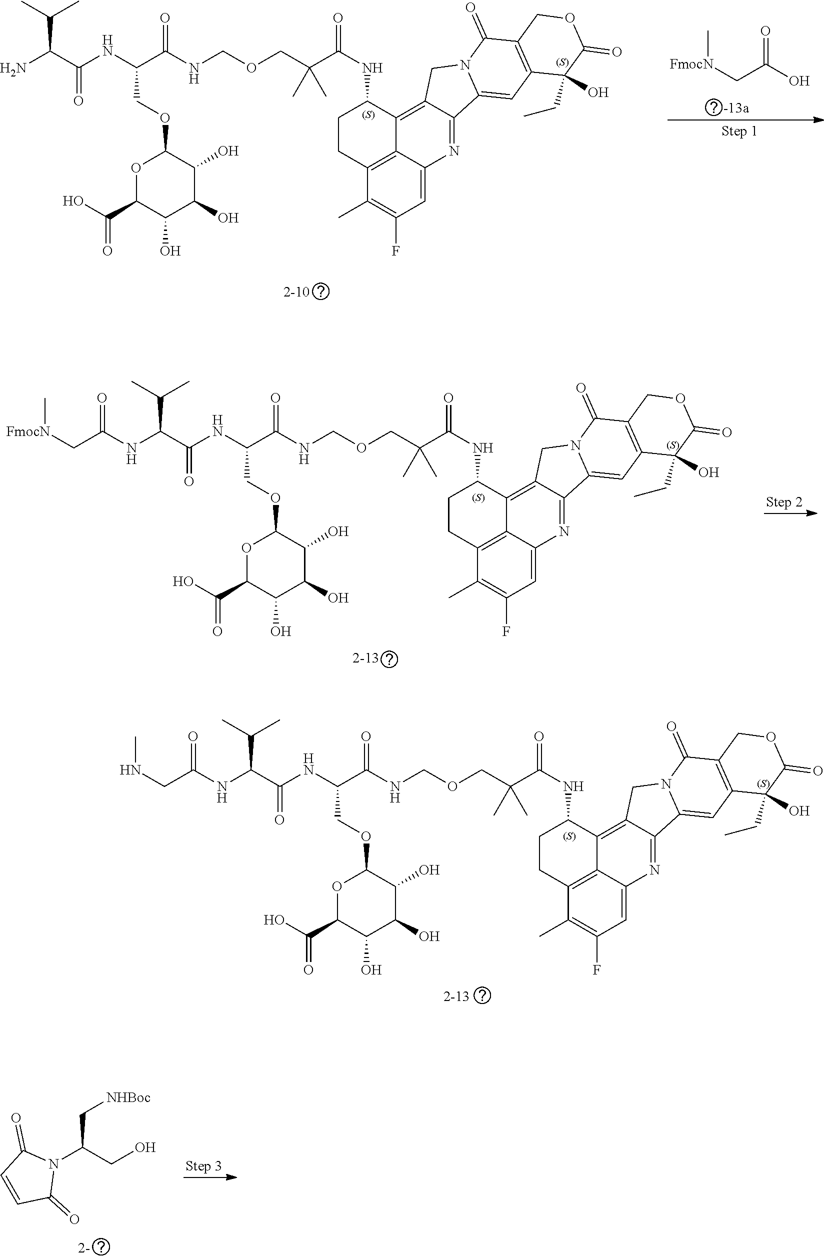

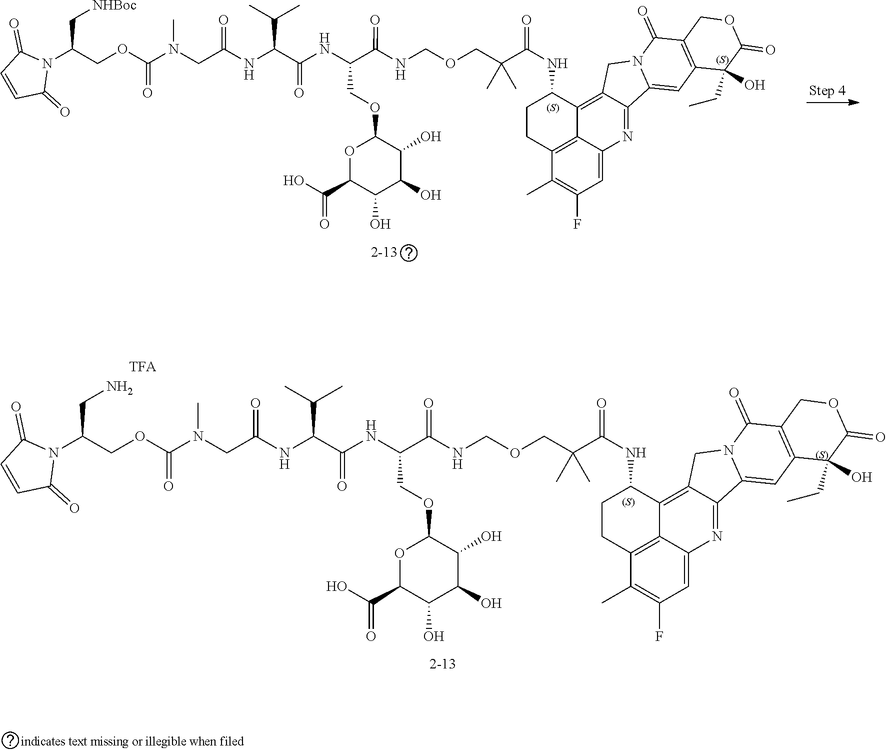

### Step 1: (2S,3S,4S,5R,6R)-6-(((8S,11S)-11-(((3-(((1S,9S)-9-Ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)carbamoyl)-1-(9H-fluoren-9-yl)-8-isopropyl-4-methyl-3,6,9-trioxo-2-oxa-4,7,10-triazadodecan-12-yl)oxy)-3,4,5-trihydroxytetrahydro-2H-pyran-2-carboxylic acid (2-13b)

To a solution of 2-13a (11.1 mg, 0.0356 mmol) in DMF (1 mL) was added HATU (13.5 mg, 0.0356 mmol), DIPEA (10.5 mg, 0.081 mmol) and 2-10d (30 mg, 0.0323 mmol). The reaction was stirred at r.t. for 30 min. The mixture was diluted with DMF (1.5 mL) and acidified with AcOH. It was purified by prep-HPLC (FA). The fraction was lyophilized to give 2-13b (14.6 mg, 36.9% yield) as a pale-yellow solid.

MS (ESI) m/z: 1221.0 [M+H]⁺.

### Step 2: (2S,3S,4S,5R,6R)-6-((S)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-((S)-3-methyl-2-(2-(methylamino) acetamido) butanamido)-3-oxopropoxy)-3,4,5-trihydroxytetrahydro-2H-pyran-2-carboxylic acid (2-13c)

To a solution of 2-13b (14.6 mg, 0.012 mmol) in DMF (0.3 mL) was added EtzNH (8.8 mg, 0.12 mmol). The mixture was stirred at r.t. for 20 min. The reaction mixture was concentrated under vacuum and co-evaporated with toluene two times to give 2-13c as a brown solid, which was used in the next step directly.

### Step 3: (2S,3S,4S,5R,6R)-6-(((7S,15S,18S)-7-(2,5-dioxo-2,5-dihydro-1H-pyrrol-1-yl)-18-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)carbamoyl)-15-isopropyl-2,2,11-trimethyl-4,10,13,16-tetraoxo-3,9-dioxa-5,11,14,17-tetraazanonadecan-19-yl)oxy)-3,4,5-trihydroxytetrahydro-2H-pyran-2-carboxylic acid (2-13d)

To a mixture of 2-4e (3.6 mg, 0.0132 mmol) in DMF (0.2 mL) were added DSC (3.5 mg, 0.0136 mmol) and DIPEA (2.3 mg, 0.018 mmol). The mixture was stirred at r.t. for 3 h. Crude solid 2-13c was then added and stirred at r.t. for another 30 min. The mixture was diluted with DMF (1.5 mL) and acidified with AcOH. It was purified by prep-HPLC (FA) to give 2-13d (5.4 mg, 34.8% yield) as a pale-yellow solid.

MS (ESI) m/z: 1294.9 [M+H]⁺.

### Step 4: (2S,3S,4S,5R,6R)-6-(((2S,5S,13S)-14-amino-13-(2,5-dioxo-2,5-dihydro-1H-pyrrol-1-yl)-2-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)carbamoyl)-5-isopropyl-9-methyl-4,7,10-trioxo-11-oxa-3,6,9-triazatetradecyl)oxy)-3,4,5-trihydroxytetrahydro-2H-pyran-2-carboxylic acid (2-13)

To a suspension of 2-13d (5.4 mg, 0.0042 mmol) in DCM (0.5 mL) was added ZnBr₂ (24 mg, 0.11 mmol), then stirred at 45° C. for 24 h. The mixture was concentrated under vacuum and dissolved in 0.1%/ACN (80:20, 1 mL) then purified by prep-HPLC (0.1% TFA). The fraction was lyophilized to give 2-13 (1.3 mg, 26% yield) as a white solid.

MS (ESI) m/z: 1194.9 [M+H]⁺.

### Example 2-14

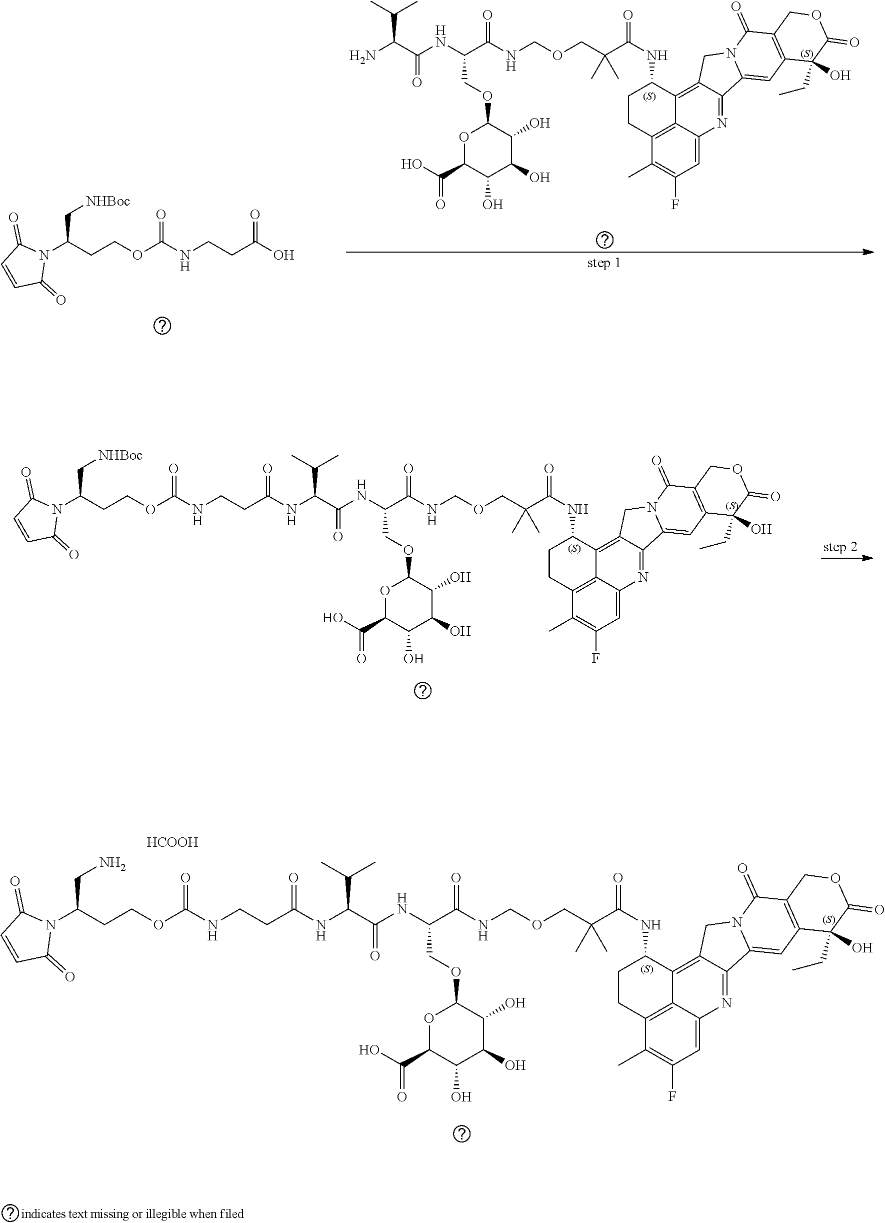

### (R)-3-(((4-amino-3-(2,5-dioxo-2,5-dihydro-1H-pyrrol-1-yl) butoxy) carbonyl)amino) propanoic acid (2-14a)

2-14a (280 mg, 89.6% yield) was synthesized according to the synthetic procedure of Example 1-13.

MS (ESI) m/z: 422.3 [M+Na]⁺.

1H NMR (400 MHZ, d₆-DMSO) δ 12.48 (br s, 1H), 7.03-6.99 (m, 2H), 6.94 (s, 2H), 4.08-4.03 (m, 3H), 3.86-3.83 (m, 2H), 3.17-3.11 (m, 2H), 2.35 (t, J=7.2 Hz, 2H), 2.14-2.09 (m, 1H), 1.91-1.84 (m, 1H), 1.32 (s, 9H).

### (2S,3S,4S,5R,6R)-6-(((2S,5S,15R)-16-amino-15-(2,5-dioxo-2,5-dihydro-1H-pyrrol-1-yl)-2-(((3-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-2,2-dimethyl-3-oxopropoxy)methyl)carbamoyl)-5-isopropyl-4,7,11-trioxo-12-oxa-3,6,10-triazahexadecyl)oxy)-3,4,5-trihydroxytetrahydro-2H-pyran-2-carboxylic acid—formic acid (2-14)

2-14 (13 mg, 64.0% yield) was synthesized according to the synthetic procedure of Example 2-12.

MS (ESI) m/z: 1208.9 [M+H]⁺.

### Example 2-15

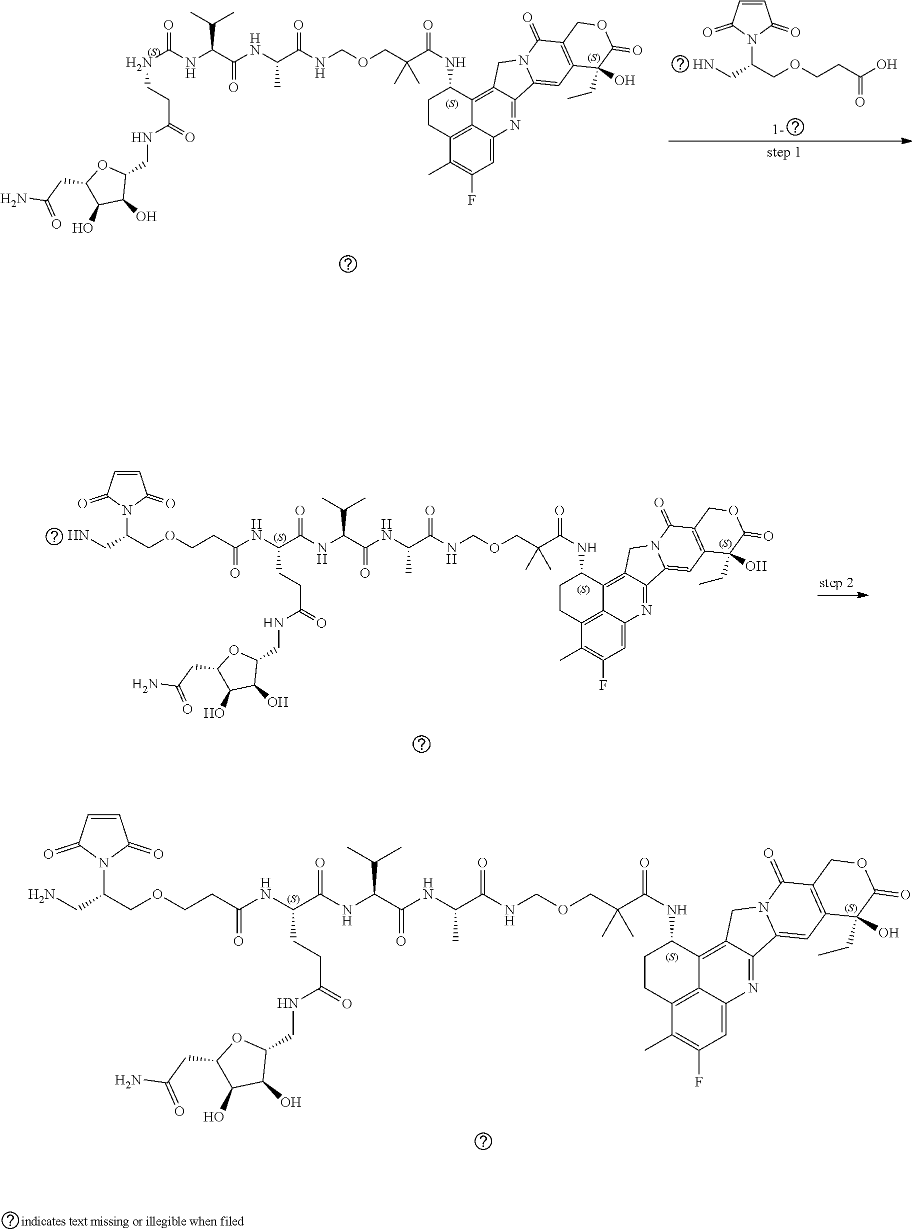

Step 1: tert-butyl ((8S,11S,14S,21S)-14-(3-((((2R,3S,4R,5S)-5-(2-amino-2-oxoethyl)-3,4-dihydroxytetrahydrofuran-2-yl)methyl)amino)-3-oxopropyl)-21-(2,5-dioxo-2,5-dihydro-1H-pyrrol-1-yl)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-11-isopropyl-2,2,8-trimethyl-1,7,10,13,16-pentaoxo-4,19-dioxa-6,9,12,15-tetraoxadocosan-22-yl) carbamate (2-15a)

To a solution of 1-6f (45 mg, 0.13 mmol) in DMF (3 mL) were added HATU (55.0 mg, 0.15 mmol) and DIPEA (34.1 mg, 0.26 mmol). The mixture was stirred at r.t. for 10 min. 2-8i (147 mg, crude) was added to the mixture and stirred at r.t. for 5 min. The mixture was purified by prep-HPLC (FA 0.1%) to give 2-8r (93 mg, 52% yield) as a pale-yellow solid.

MS (ESI) m/z: 1384.1 [M+Na]⁺.

### Step 2: (S)-2-(3-((S)-3-amino-2-(2,5-dioxo-2,5-dihydro-1H-pyrrol-1-yl) propoxy) propanamido)-N₅-(((2R,3S,4R,5S)-5-(2-amino-2-oxoethyl)-3,4-dihydroxytetrahydrofuran-2-yl)methyl)-N₁—((S)-1-(((S)-1-(((3-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-2,2-dimethyl-3-oxopropoxy)methyl)amino)-1-oxopropan-2-yl)amino)-3-methyl-1-oxobutan-2-yl) pentanediamide (2-15)

To a suspension of 2-8r (52 mg, 0.038 mmol) in DCM (4 mL) was added ZnBr₂ (258.2 mg, 1.147 mmol) then stirred at 45° C. for 8 hr. The mixture was concentrated under vacuum and dissolved in DMSO then purified by prep-HPLC (0.1% FA). The fraction was lyophilized to give 2-15 (29.2 mg) as a pale-yellow solid.

MS (ESI) m/z: 1261.0 [M+H]⁺.

### Example 2-16

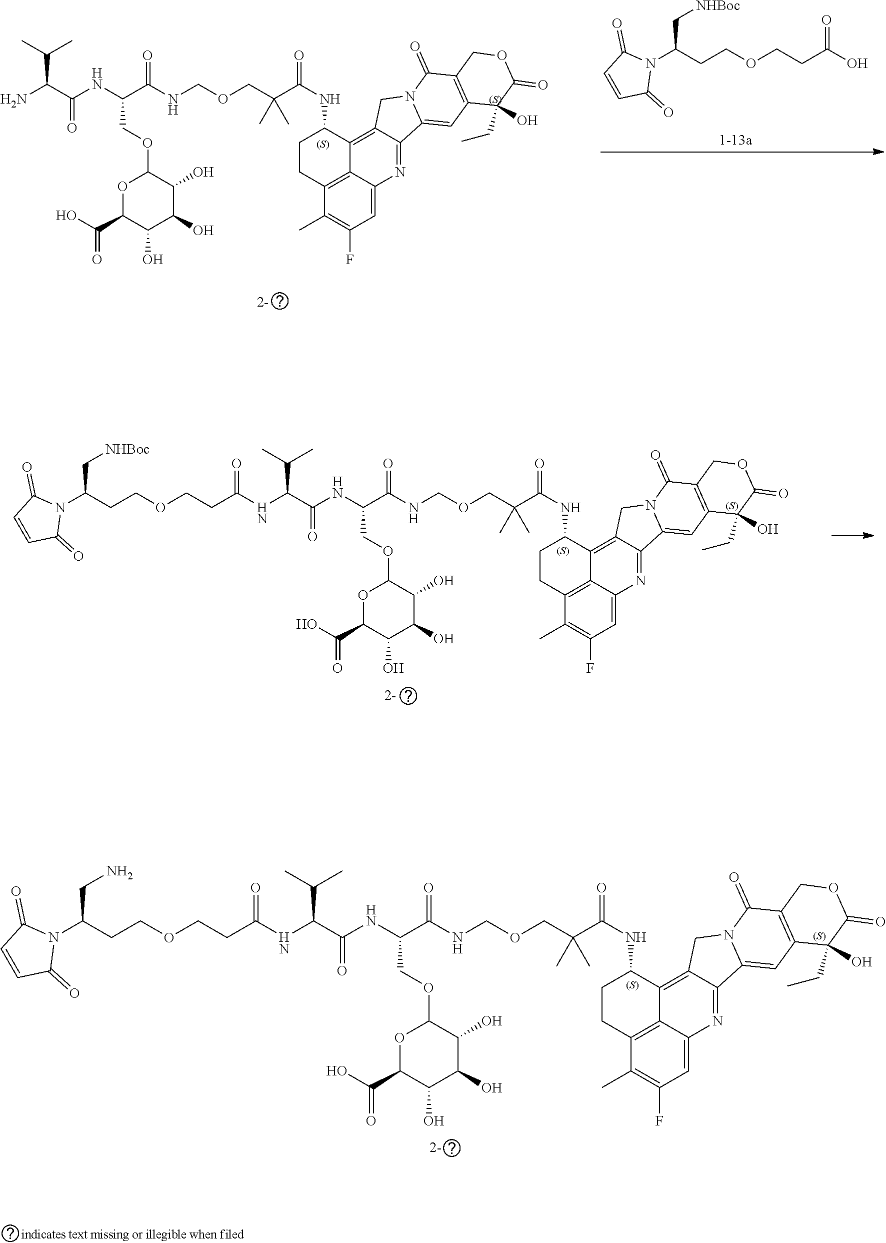

### (2S,3S,4S,5R,6R)-6-((S)-2-((S)-2-(3-((R)-4-amino-3-(2,5-dioxo-2,5-dihydro-1H-pyrrol-1-yl) butoxy) propanamido)-3-methylbutanamido)-3-(((3-(((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic acid (2-16)

2-16 (13.4 mg, 79.1% yield) as a white solid was synthesized according to the synthetic procedure of Example 2-10.

MS (ESI) m/z: 1165.7 [M+H]⁺.

The compounds of Examples 1-2 and 1-4 to 1-14 and reference compounds 1-1 and 1-3 are shown below in Table 1. The compounds of Examples 2-2 and 2-4 to 2-16 and reference compounds 2-1 and 2-3 are shown below in Table 2.

TABLE 1
Conjugators

| Compound No. | Conjugator Structure |
|---|---|
| 1-1 | 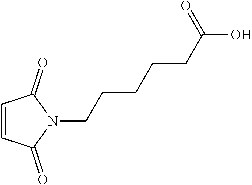 [SMILES: O=C(O)CCCCCN1C(=O)C=CC1=O] |
| 1-2 | 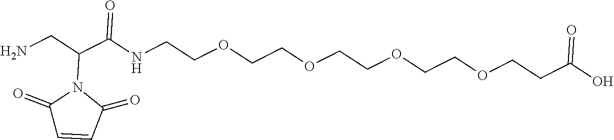 [SMILES: NCC(C(=O)NCCOCCOCCOCCOCCC(=O)O)N1C(=O)C=CC1=O] |
| 1-3 | 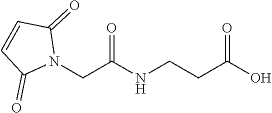 [SMILES: O=C(O)CCNC(=O)CN1C(=O)C=CC1=O] |
| 1-4 | 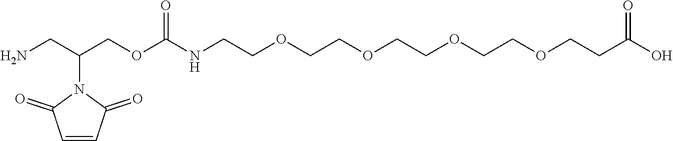 [SMILES: NCC(COC(=O)NCCOCCOCCOCCOCCC(=O)O)N1C(=O)C=CC1=O] |
| 1-5 | 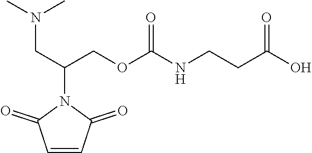 [SMILES: CN(C)CC(COC(=O)NCCC(=O)O)N1C(=O)C=CC1=O] |
| 1-6 | 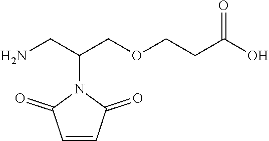 [SMILES: NCC(COCCC(=O)O)N1C(=O)C=CC1=O] |
| 1-7 | 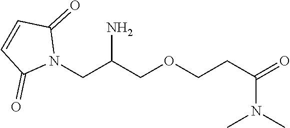 [SMILES: CN(C)C(=O)CCOCC(N)CN1C(=O)C=CC1=O] |
| 1-8 | 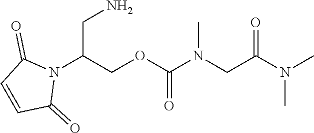 [SMILES: CN(C)C(=O)CN(C)C(=O)OCC(CN)N1C(=O)C=CC1=O] |
| 1-9 | 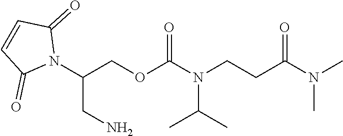 [SMILES: CC(C)N(CCC(=O)N(C)C)C(=O)OCC(CN)N1C(=O)C=CC1=O] |
| 1-10 | 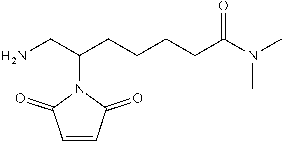 [SMILES: CN(C)C(=O)CCCCC(CN)N1C(=O)C=CC1=O] |
| 1-11 | 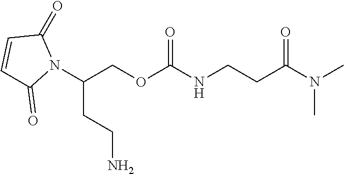 [SMILES: CN(C)C(=O)CCNC(=O)OCC(CCN)N1C(=O)C=CC1=O] |
| 1-12 | 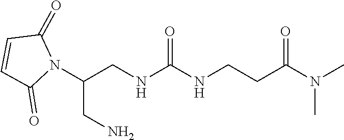 [SMILES: CN(C)C(=O)CCNC(=O)NCC(CN)N1C(=O)C=CC1=O] |
| 1-13 | 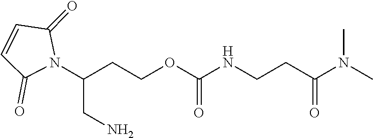 [SMILES: CN(C)C(=O)CCNC(=O)OCCC(CN)N1C(=O)C=CC1=O] |
| 1-14 | 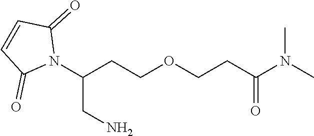 [SMILES: CN(C)C(=O)CCOCCC(CN)N1C(=O)C=CC1=O] |

TABLE 2
Conjugator-linker-payloads

| Compound | |
|---|---|
| No. | Conjugator-Linker-Payload Structure |
| 2-1 | 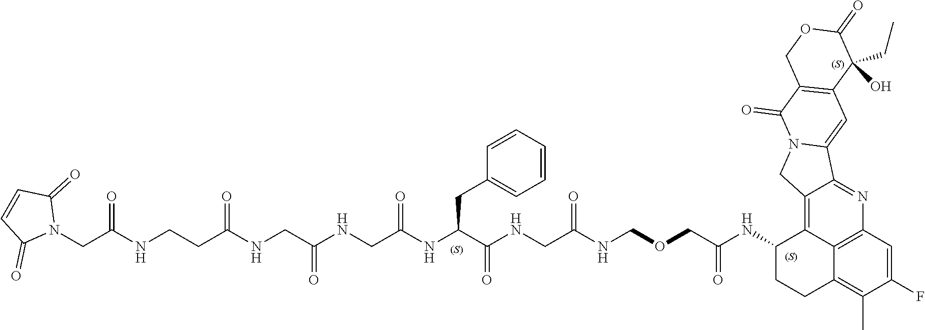 |
| 2-2 | 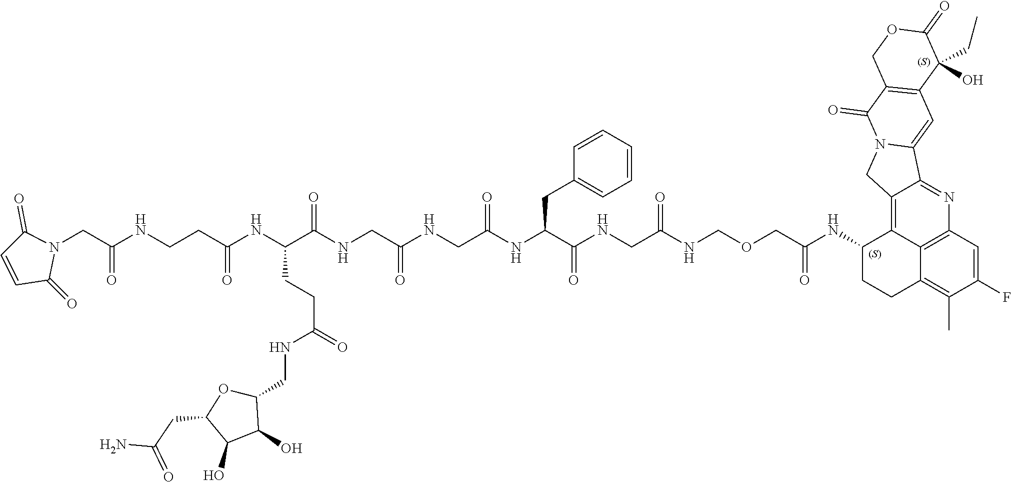 |
| 2-3 | 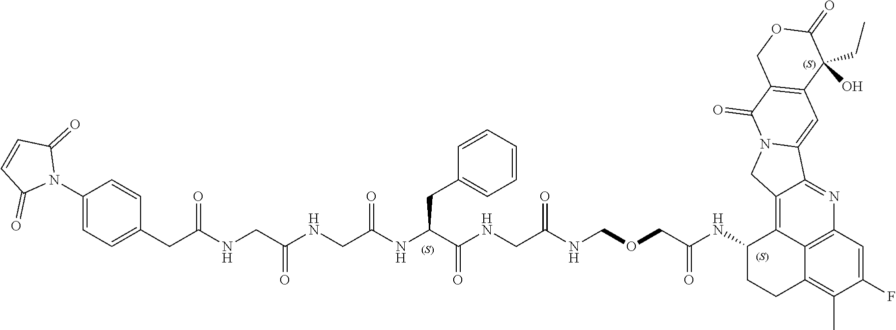 |
| 2-4 | 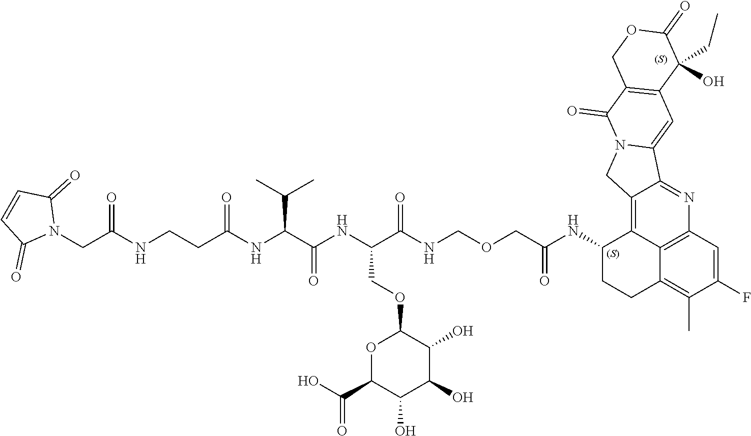 |
| 2-5 | 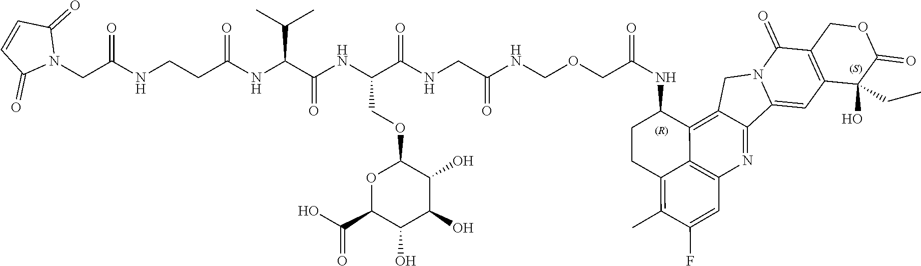 |
| 2-6 | 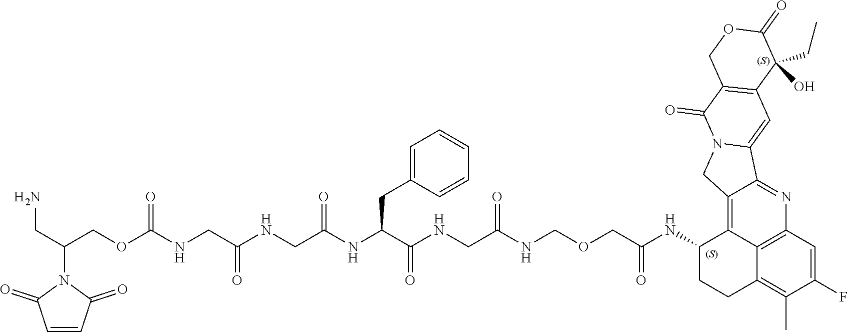 |
| 2-7 | 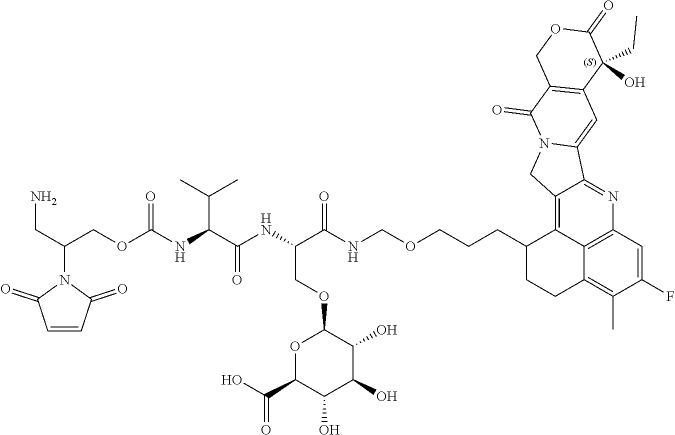 |
| 2-8 | 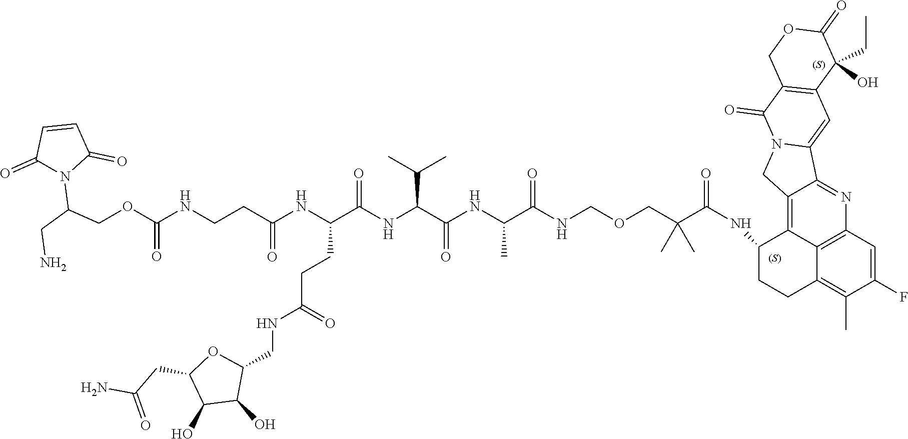 |
| 2-9 | 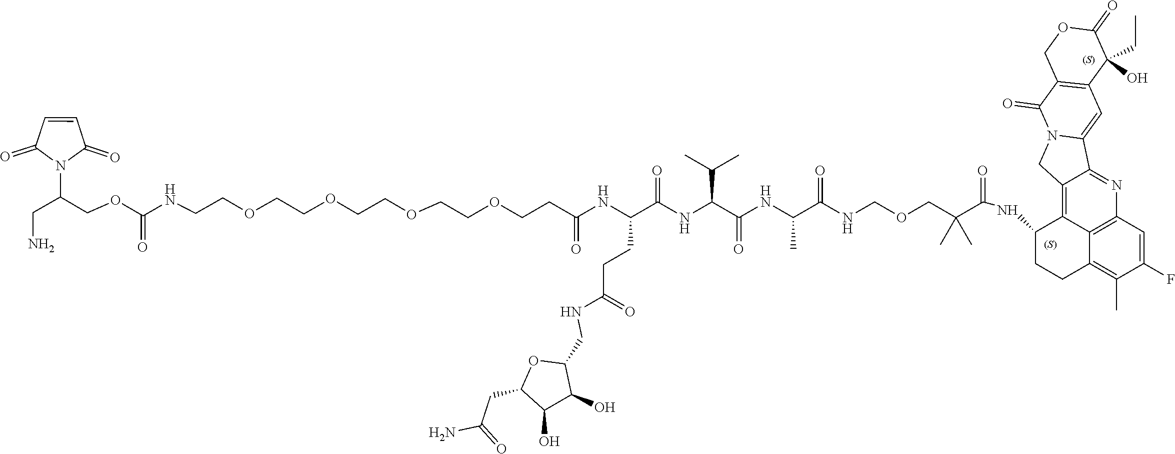 |
| 2-10 | 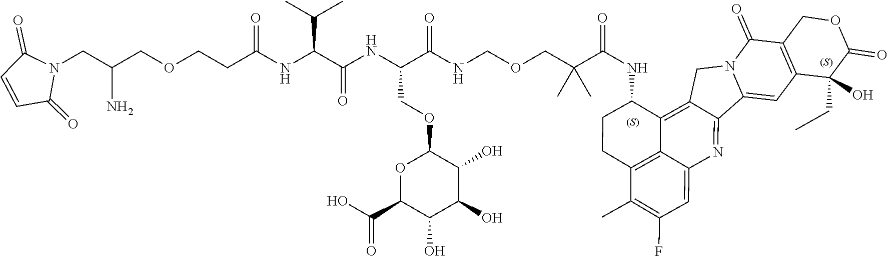 |
| 2-11 | 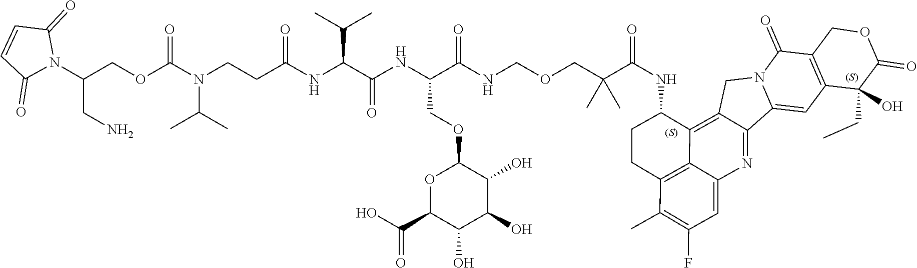 |
| 2-12 | 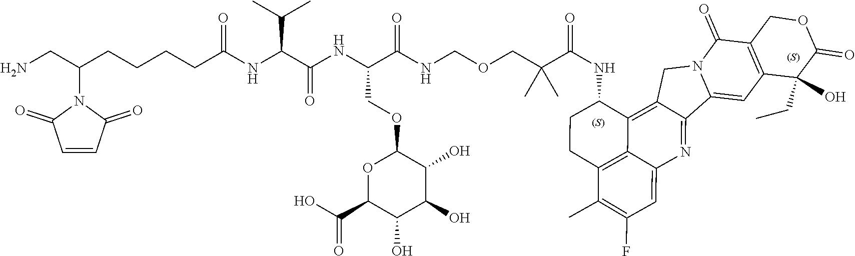 |
| 2-13 | 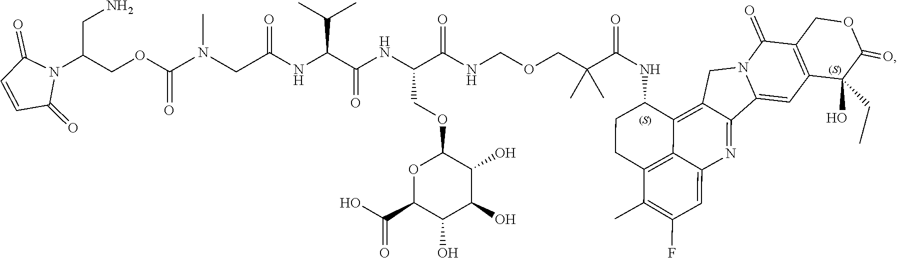 |
| 2-14 | 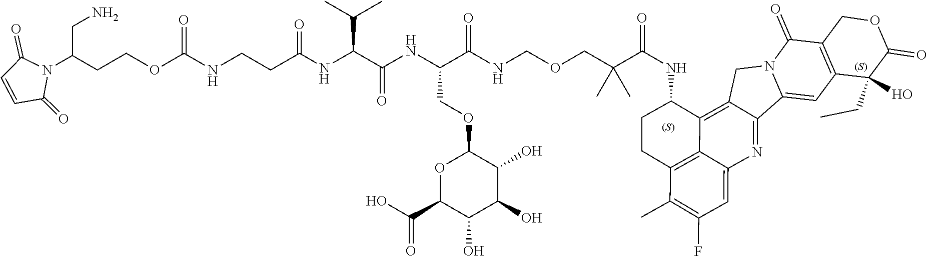 |
| 2-15 | 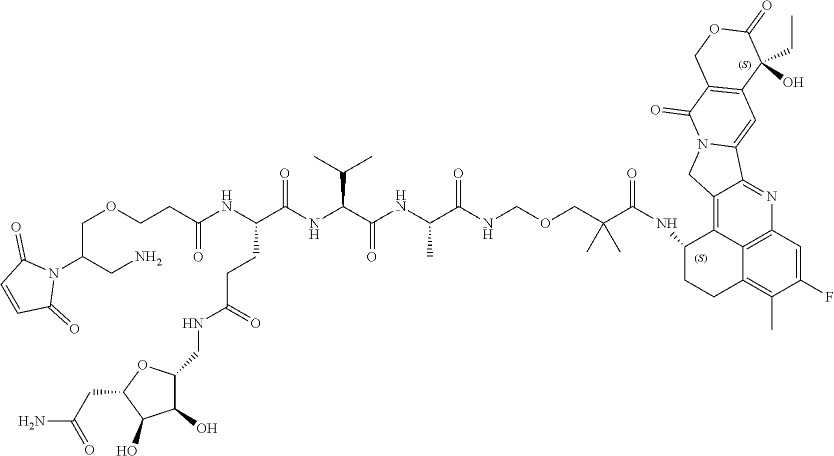 |
| 2-16 | 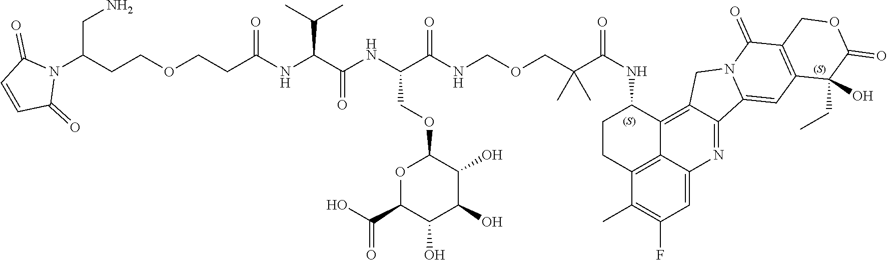 |

### Conjugator-Antibody Conjugate and Antibody Drug Conjugate Preparation and Characterization

DAR8 antibody drug conjugate/conjugator-antibody conjugate preparation. Antibody in conjugation buffer (with concentration 0.5-25 mg/mL, PBS buffer pH 6.0-8.5) was incubated under reduction temperature (0-40° C.) for 10 min. 8-15 eq. TECP solution (5 mM stock in PBS buffer) was added to the reaction mixture and the reduction reaction was left for 1-8 hours at reduction temperature. Organic solvent (e.g., DMSO, DMF, DMA, PG, acetonitrile, 0-25% v/v) and conjugator-linker-payload (see Table 2) or conjugator (see Table 1) stock (10-25 eq, 10 mM stock in organic solvent) were added stepwise after the reduction mixture was cooled to 0-25° C. Conjugation solution was left for 1-3 h at 0-25° C., and the reaction was quenched with N-acetyl cysteine (1 mM stock). The solution was submitted to buffer exchange (spin desalting column, ultrafiltration, and dialysis) into storage buffer (for example, pH 5.5-6.5 histidine acetate buffer, with optional additive such as sucrose, trehalose, tween 20, 60, 80).

After the conjugation step, the ADC (or conjugator-antibody conjugate) was subjected to buffer exchange into ring opening buffer (pH 6.5~9.0, PBS, borate or tris buffer) and the solution was left at 22 or 37° C. for 1~48 h. The maleimide ring opening process was monitored via reduced LCMS. Once the conjugated maleimide hydrolysis was completed, the resulting ADCs were buffer exchanged into basic tris pH 8.0-8.5 buffer or acidic histidine-acetate pH 5.0-6.5 buffer via dialysis.

Conjugator-antibody conjugates and ADCs prepared according to the foregoing method are shown in Tables 3 and 4, respectively. Ring-opening time is shown in Table 3 below.

LCMS method to monitor and determine maleimide hydrolysis. LC-MS analysis was carried out under the following measurement conditions:
LC-MS system: Vanquish Flex UHPLC and Orbitrap Exploris 240 Mass Spectrometer
Column: MAbPac™ RP, 2.1*50 mm, 4 μm, 1,500 Å, Thermo Scientific™
Column temperature: 80° C.
Mobile phase A: 0.1% formic acid (FA) aqueous solution
Mobile phase B: Acetonitrile solution containing 0.1% formic acid (FA)
Gradient program 1: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)
Gradient program 2
Injected sample amount: 2 μg
MS parameters: Intact and denaturing MS data were acquired in HMR mode at setting of R=15k and deconvolved using the ReSpect™ algorithm and Sliding Window integration in Thermo Scientific™ BioPharma Finder™ 4.0 software.

ADC characterization. ADCs were characterized using the following analytical methods. Drug to antibody ratios (DAR) of the ADCs were determined by LCMS method or HIC method. SEC purity of ADCs made were all >95% purity.

LCMS method: LC-MS analysis was carried out under the following measurement conditions:
LC-MS system: Vanquish Flex UHPLC and Orbitrap Exploris 240 Mass Spectrometer
Column: MAbPac™ RP, 2.1*50 mm, 4 μm, 1,500 Å, Thermo Scientific™
Column temperature: 80° C.
Mobile phase A: 0.1% formic acid (FA) aqueous solution
Mobile phase B: Acetonitrile solution containing 0.1% formic acid (FA)
Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)
Injected sample amount: 1 μg
MS parameters: Intact and denaturing MS data were acquired in HMR mode at setting of R=15k and deconvolved using the ReSpect™ algorithm and Sliding Window integration in Thermo Scientific™ BioPharma Finder™ 4.0 software.

HIC method: HPLC analysis was carried out under the following measurement conditions:

### Method 1

HPLC system: Waters ACQUITY ARC HPLC System
Detector: measurement wavelength: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM phosphate buffer, pH 7.0
Mobile phase B: 50 mM phosphate buffer, 25% (V/V) isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 μg

### Method 2

HPLC system: Waters ACQUITY ARC HPLC System
Detector: measurement wavelength: 280 nm
Column: MABPac HIC-10, 5 μm, 4.6×10 mm (Thermo)
Column temperature: 25° C.
Mobile phase A: 1.5 M ammonium sulfate, 50 mM sodium phosphate, pH 7.0
Mobile phase B: 50 mM sodium phosphate, pH 7.0
Gradient program: 20% B-20% B (0 min-1 min), 0% B-0% B (1 min-35 min), 20% B-20% B (35 min-40 min)
Flow rate: 0.5 mL/min
Sample preparation: The sample was diluted with initial mobile phase to 0.5 mg/mL.

SEC method to determine ADC purity: HPLC analysis was carried out under the following measurement conditions:
HPLC system: Waters H-Class UPLC System
Detector: measurement wavelength: 280 nm
Column: ACQUITY UPLC BEH200 SEC 1.7 um 4.6×150 mm, Waters
Column temperature: room temperature
Mobile phase A: 200 mM phosphate buffer, 250 mM potassium chloride, 15% isopropyl alcohol, pH 7.0
Gradient program: under 10 min isocratic elutions with the flow rate of 0.3 mL/min
Injected sample amount: 20 μg

ADC hydrophobicity evaluation: An ADC with a higher hydrophobic property would appear with a later retention time from HIC (hydrophobicity interaction column) chromatography. Results are presented in Table 4 using the DAR8 peak as a reference.

Results for ring opening times measured by the LCMS method described above for monitoring ring hydrolysis are presented in Table 3 as RO T_{1/2} (h) (i.e., the time, in hours, for 50% of the maleimide rings to open).

TABLE 3
Conjugator-antibody conjugates

| Conjugator- | | | | RO |
|---|---|---|---|---|
| Antibody | | | MS | T_{1/2} |
| Conjugate No. | Antibody | Conjugator Structure | DAR | (h) |
| 3-1 | Herceptin | 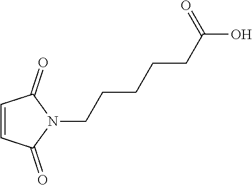 [SMILES: O=C(O)CCCCCN1C(=O)C=CC1=O] | 8.02 | NA |
| 3-3 | Herceptin | 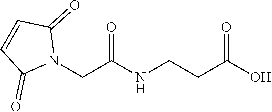 [SMILES: O=C(O)CCNC(=O)CN1C(=O)C=CC1=O] | 8.12 | 36.0 |
| 3-4 | Herceptin | 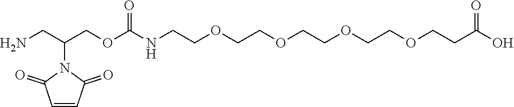 [SMILES: NCC(COC(=O)NCCOCCOCCOCCOCCC(=O)O)N1C(=O)C=CC1=O] | 7.98 | 1.6 |
| 3-5 | Herceptin | 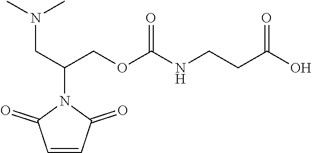 [SMILES: CN(C)CC(COC(=O)NCCC(=O)O)N1C(=O)C=CC1=O] | NA | NA |
| 3-6 | Herceptin | 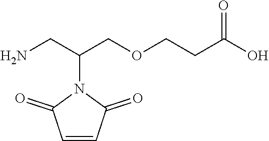 [SMILES: NCC(COCCC(=O)O)N1C(=O)C=CC1=O] | 8.00 | 1.9 |
| 3-7 | Herceptin | 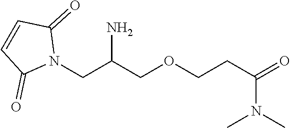 [SMILES: CN(C)C(=O)CCOCC(N)CN1C(=O)C=CC1=O] | 8.00 | 1.7 |
| 3-8 | Herceptin | 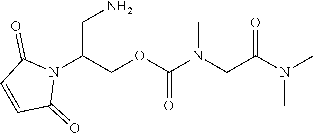 [SMILES: CN(C)C(=O)CN(C)C(=O)OCC(CN)N1C(=O)C=CC1=O] | 7.80 | 1.6 |
| 3-9 | Herceptin | 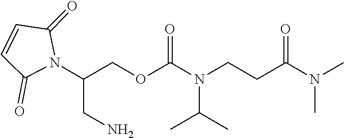 [SMILES: CC(C)N(CCC(=O)N(C)C)C(=O)OCC(CN)N1C(=O)C=CC1=O] | 7.96 | 0.8 |
| 3-10 | Herceptin | 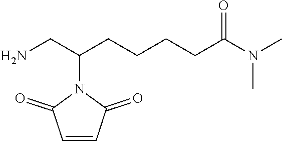 [SMILES: CN(C)C(=O)CCCCC(CN)N1C(=O)C=CC1=O] | 8.00 | 2.0 |
| 3-11 | Herceptin | 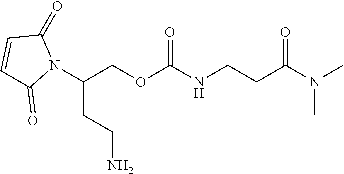 [SMILES: CN(C)C(=O)CCNC(=O)OCC(CCN)N1C(=O)C=CC1=O] | 8.00 | 14.0 |
| 3-12 | Herceptin | 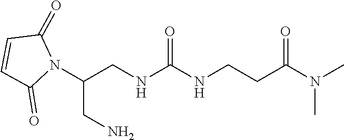 [SMILES: CN(C)C(=O)CCNC(=O)NCC(CN)N1C(=O)C=CC1=O] | 8.00 | 1.6 |
| 3-13 | Herceptin | 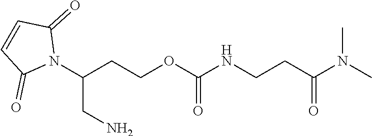 [SMILES: CN(C)C(=O)CCNC(=O)OCCC(CN)N1C(=O)C=CC1=O] | 8.00 | 1.7 |
| 3-14 | Herceptin | 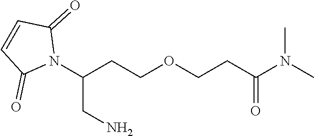 [SMILES: CN(C)C(=O)CCOCCC(CN)N1C(=O)C=CC1=O] | 8.00 | 2.0 |

TABLE 4
ADCs

| | | | DAR8 Species HIC |
|---|---|---|---|
| | | | Retention Time |
| ADC No. | Antibody | ADC Structure | (min) (Method 1) |
| ADC4-B | Ifinatamab | 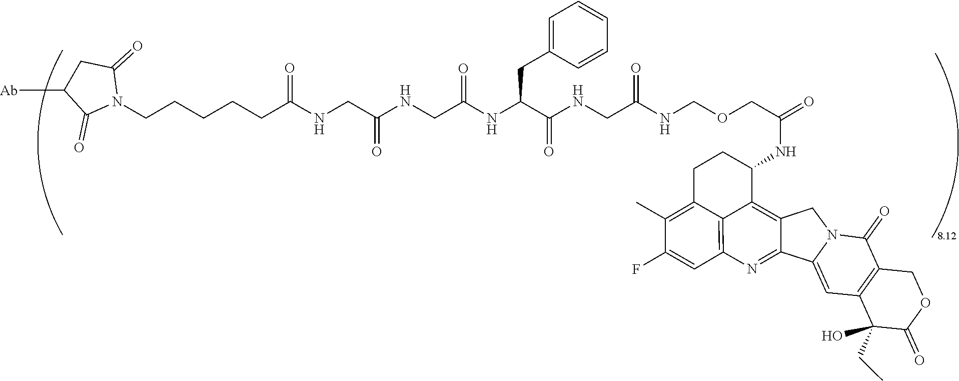 | 10.860 |
| ADC4-4o | Ifinatamab | 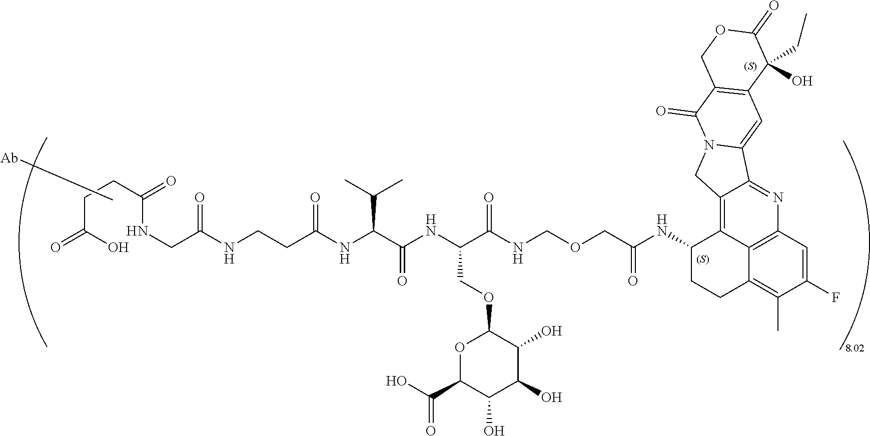 | 9.676 |
| ADC4-5o | Ifinatamab | 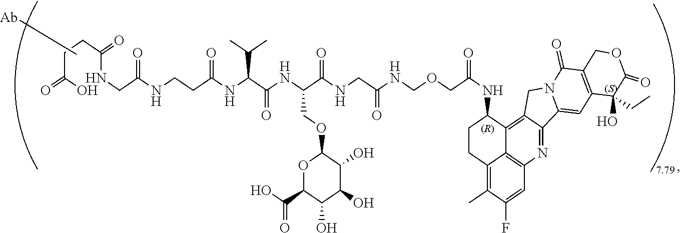 | 9.764 |
| ADC4-6o | Herceptin | 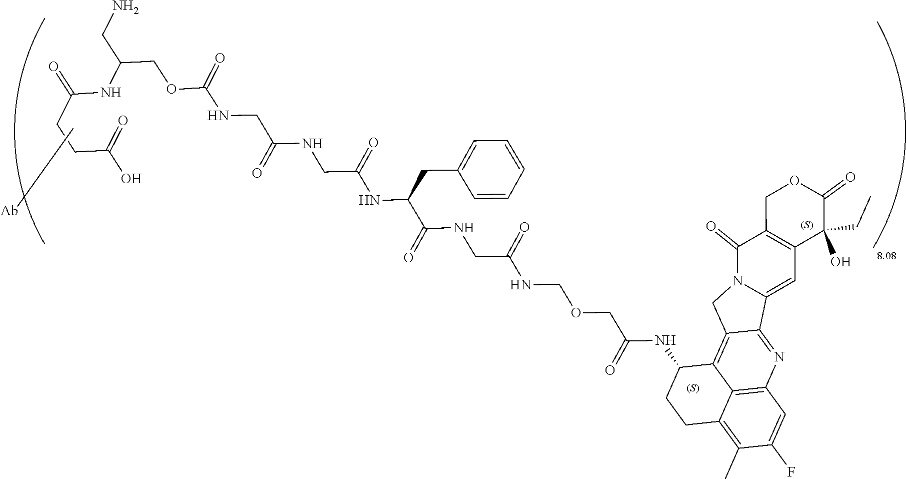 | NA |
| ADC4-7o | Ifinatamab | 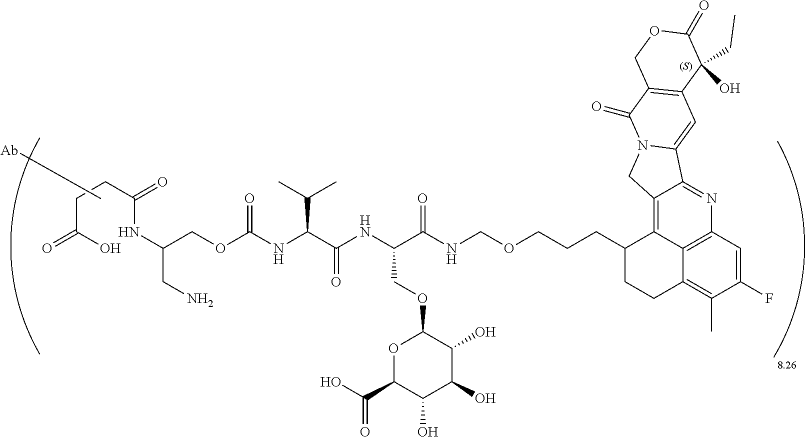 | 9.637 |
| ADC4-8o | Ifinatamab | 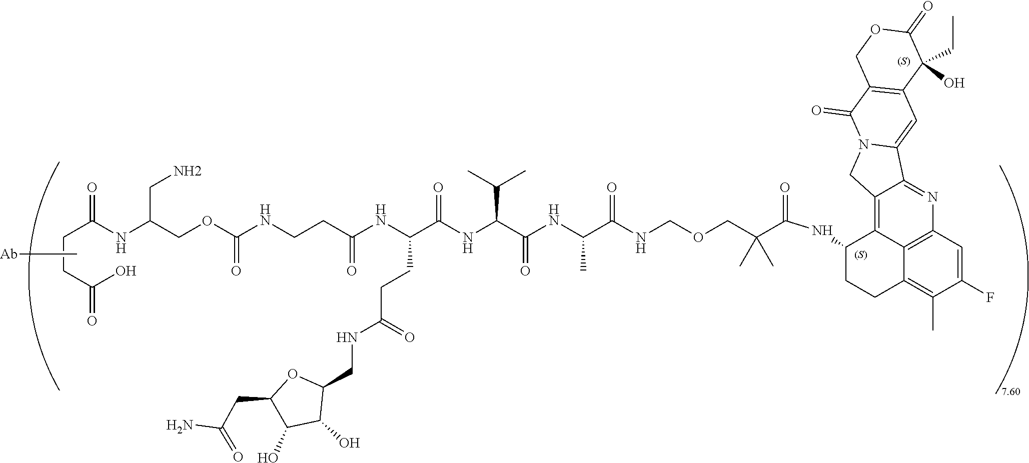 | 10.444 |
| ADC4-9o | Ifinatamab | 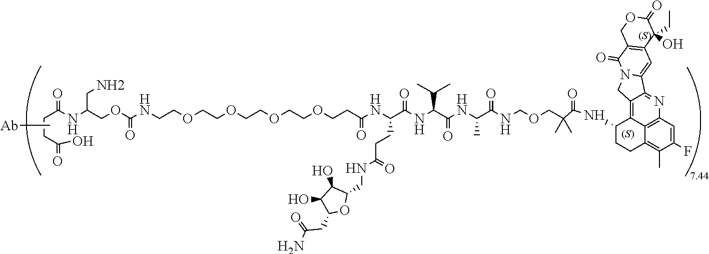 | 10.883 |
| ADC4-10o | Ifinatamab | 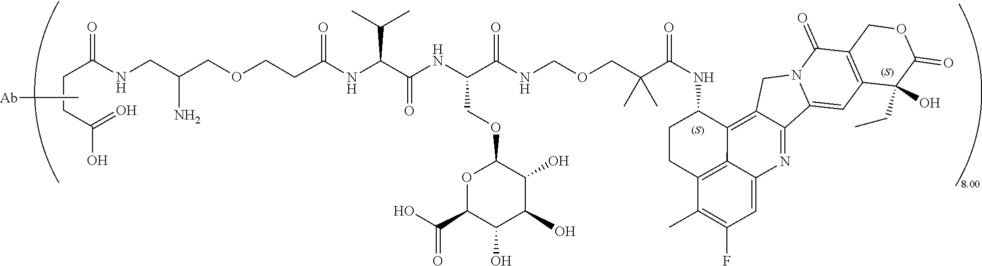 | 9.898 |
| ADC4-11o | Ifinatamab | 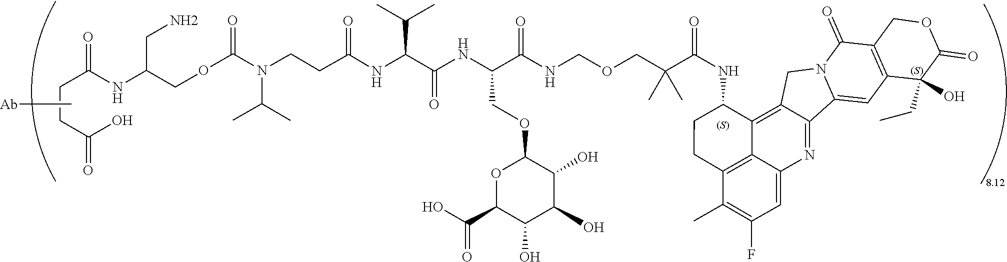 | 9.948 |
| ADC4-12o | Ifinatamab | 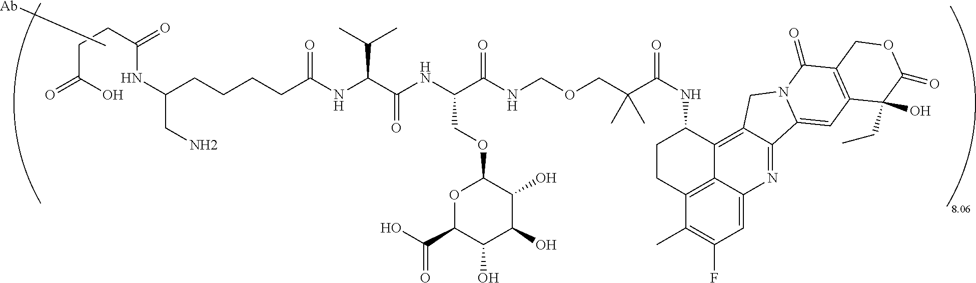 | 9.801 |
| ADC4-13o | Ifinatamab | 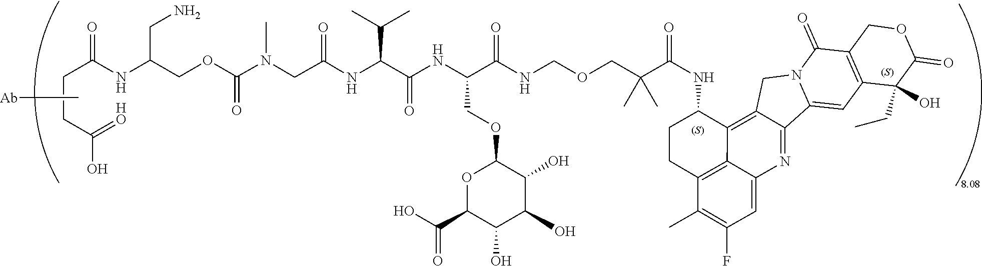 | 9.877 |
| ADC4-14o | Ifinatamab | 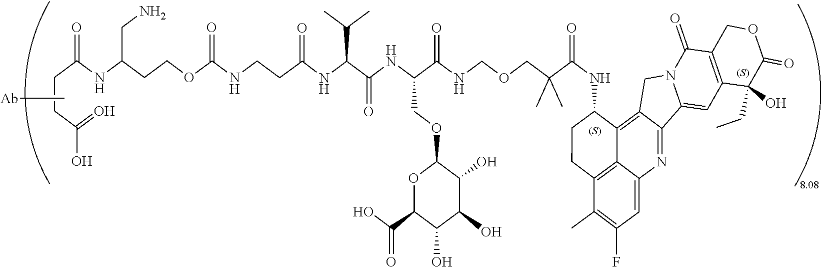 | 9.977 |
| ADC4-16o | Ifinatamab | 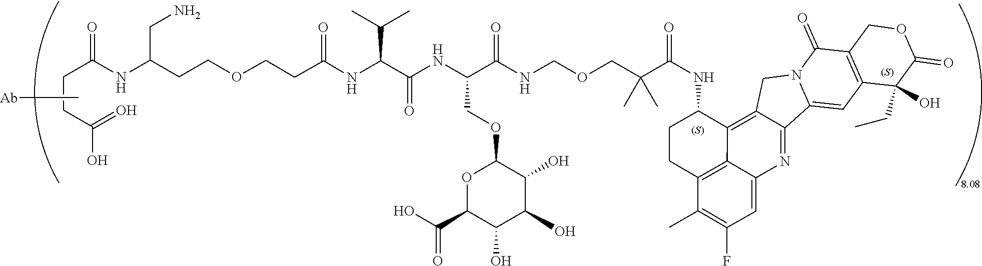 | 9.795 |

### Antibody Information

Ifinatamab MABX-9001a (anti-B7H3 antibody)
Light Chain sequence
(SEQ ID NO: 1)
EIVLTQSPATLSLSPGERATLSCRASSRLIYMHWYQQKPGQAPRPLIYAT
SNLASGIPARFSGSGSGTDFTLTISSLEPEDFAVYYCQQWNSNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
Heavy Chain sequence
(SEQ ID NO: 2)
QVQLVQSGAEVKKPGSSVKVSCKASGYTFTNYVMHWVRQAPGQGLEWMGY
INPYNDDVKYNEKFKGRVTITADESTSTAYMELSSLRSEDTAVYYCARWG
YYGSPLYYFDYWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK

### Conjugator-Antibody Conjugate Hydrolysis Kinetics

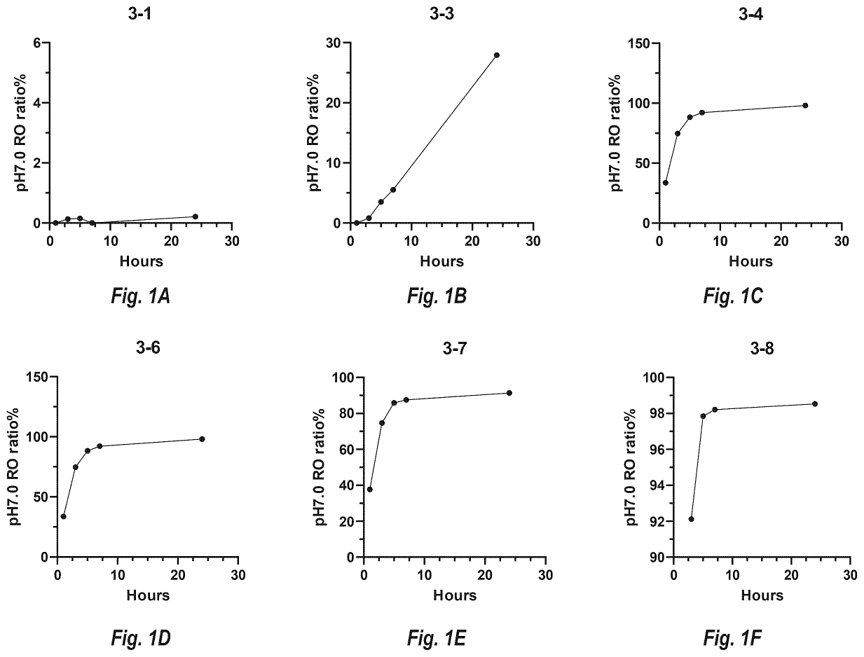

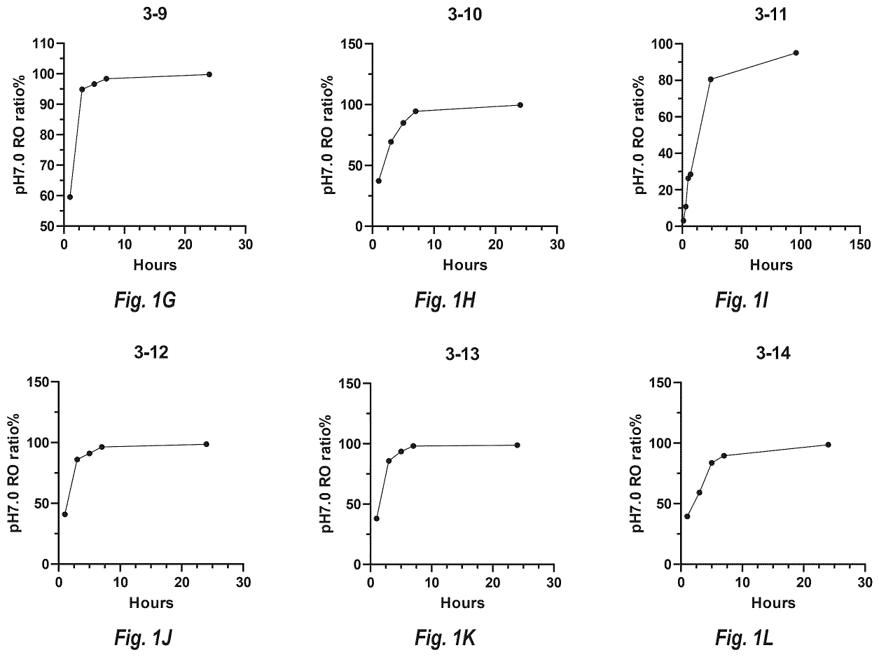

Conjugator-antibody conjugate maleimide hydrolysis (ring opening) kinetics are presented in FIGS. 1A-1L, Table 3 (for pH 7.0 reaction conditions, hydrolysis T_{1/2}), and Table 5. The data in Table 5 was generated by the LCMS method described above and includes the time, in hours, for the maleimide ring to open in more than 95% of the conjugators, after antibody conjugation (“RO Completed Time (>95%) (h)”). The results for conjugator-antibody conjugate 3-1 indicate that after 24 hours, about 0% of maleimide rings were open; the results for conjugator-antibody conjugate 3-3 indicate that after 24 hours, about 28% of maleimide rings were open.

TABLE 5
Conjugator-antibody conjugate time-course kinetics

| Conjugator-Antibody | | Time | MS | RO (%) | | | RO Completed |
|---|---|---|---|---|---|---|---|
| Conjugate No. | pH | point (h) | DAR | LC | HC | Total | Time (>95%) (h) |
| 3-1 | 7 | 1 | 8.02 | 0 | 0 | 0 | 24 h~0% |
| | | 3 | 8.02 | 0 | 0.17 | 0.13 | |
| | | 5 | 8.02 | 0 | 0.2 | 0.15 | |
| | | 7 | 8.02 | 0 | 0 | 0 | |
| | | 24 | 8 | 0 | 0.27 | 0.21 | |
| 3-3 | 7 | 1 | 8.04 | 0 | 0 | 0 | 24 h~28% |
| | | 3 | 8.04 | 0.89 | 0.8 | 0.82 | |
| | | 5 | 8.08 | 2.52 | 3.82 | 3.5 | |
| | | 7 | 8.08 | 3.8 | 6.1 | 5.53 | |
| | | 24 | 8.12 | 13.49 | 32.76 | 27.94 | |
| 3-4 | 7 | 1 | 7.96 | 23.19 | 51.56 | 44.47 | 7 |
| | | 3 | 7.98 | 75.33 | 88.19 | 84.98 | |
| | | 5 | 7.98 | 87.91 | 90.01 | 89.49 | |
| | | 7 | 7.98 | 95.37 | 99.07 | 98.15 | |
| | | 24 | 7.98 | 99.66 | 99.08 | 99.23 | |
| 3-6 | 7 | 1 | 8 | 15.27 | 39.88 | 33.73 | 24 |
| | | 3 | 8 | 53.78 | 81.63 | 74.67 | |
| | | 5 | 8 | 73.23 | 93.34 | 88.31 | |
| | | 7 | 8 | 79.56 | 96.47 | 92.24 | |
| | | 24 | 8 | 93.88 | 99.41 | 98.03 | |
| 3-7 | 7 | 1 | 8.52 | 28.33 | 40.83 | 37.71 | >24 |
| | | 3 | 8 | 60.08 | 79.61 | 74.73 | |
| | | 5 | 8 | 77.85 | 88.54 | 85.87 | |
| | | 7 | 8 | 86.34 | 87.98 | 87.57 | |
| | | 24 | 8 | 92.99 | 90.8 | 91.35 | |
| 3-8 | 7 | 1 | 7.74 | 44.94 | / | / | 5 |
| | | 3 | 7.88 | 80.15 | 96.11 | 92.12 | |
| | | 5 | 7.78 | 92.96 | 99.49 | 97.85 | |
| | | 7 | 7.8 | 94.42 | 99.47 | 98.21 | |
| | | 24 | 7.8 | 95.48 | 99.55 | 98.53 | |
| 3-9 | 7 | 1 | 7.94 | 40.06 | 66.09 | 59.58 | 5 |
| | | 3 | 7.94 | 79.93 | 99.84 | 94.86 | |
| | | 5 | 7.96 | 89.36 | 99.07 | 96.64 | |
| | | 7 | 7.96 | 93.56 | 100 | 98.39 | |
| | | 24 | 7.96 | 99.17 | 100 | 99.79 | |
| 3-10 | 7 | 1 | 8 | 21.63 | 42.51 | 37.29 | 7 |
| | | 3 | 8 | 49.46 | 76.09 | 69.43 | |
| | | 5 | 8 | 64.05 | 91.86 | 84.91 | |
| | | 7 | 8 | 83.47 | 98.26 | 94.56 | |
| | | 24 | 8 | 100 | 99.52 | 99.64 | |
| 3-11 | 7 | 1 | 8 | 1.97 | 3.41 | 3.05 | 96 |
| | | 3 | 8 | 6.37 | 12.32 | 10.83 | |
| | | 5 | 8 | 10.15 | 31.72 | 26.33 | |
| | | 7 | 8 | 14.6 | 33.03 | 28.42 | |
| | | 24 | 8 | 68.64 | 84.46 | 80.51 | |
| | | 96 | 8 | 89.26 | 96.94 | 95.02 | |
| 3-12 | 7 | 1 | 8 | 27.31 | 45.46 | 40.92 | 7 |
| | | 3 | 8 | 74.84 | 89.82 | 86.08 | |
| | | 5 | 8 | 78.30 | 95.33 | 91.07 | |
| | | 7 | 8 | 88.53 | 99.07 | 96.44 | |
| | | 24 | 8 | 95.87 | 99.64 | 98.70 | |
| 3-13 | 7 | 1 | 8 | 21.50 | 43.63 | 38.10 | 7 |
| | | 3 | 8 | 64.20 | 92.96 | 85.77 | |
| | | 5 | 8 | 74.30 | 100.00 | 93.58 | |
| | | 7 | 8 | 93.68 | 99.68 | 98.18 | |
| | | 24 | 8 | 94.99 | 100.00 | 98.75 | |
| 3-14 | 7 | 1 | 8 | 17.39 | 46.79 | 39.44 | 24 |
| | | 3 | 8 | 32.97 | 67.87 | 59.15 | |
| | | 5 | 8 | 77.46 | 85.71 | 83.65 | |
| | | 7 | 8 | 72.00 | 95.45 | 89.59 | |
| | | 24 | 8 | 95.83 | 99.69 | 98.73 | |

The data demonstrate that the presently disclosed conjugator-antibody conjugates (e.g., 3-4 to 3-14) readily undergo maleimide hydrolysis under mild conditions (e.g., pH 7.0). The presently disclosed conjugators are not only conjugated with an antibody under conventional condition (e.g., pH 6.5-7.0) but also maleimide hydrolysis can occur under conjugation conditions. Buffer exchange into a basic buffer for hydrolysis is not required, which may help reduce ADC manufacturing costs. Adding a quenching reagent can be sufficient to stop the conjugation reaction. The presently disclosed conjugates can be subjected to buffer exchange into formulation buffer after maleimide hydrolysis is completed by monitoring via reduced LCMS.

### Stabilities of Self-Hydrolyzing Conjugator-Antibody Conjugates

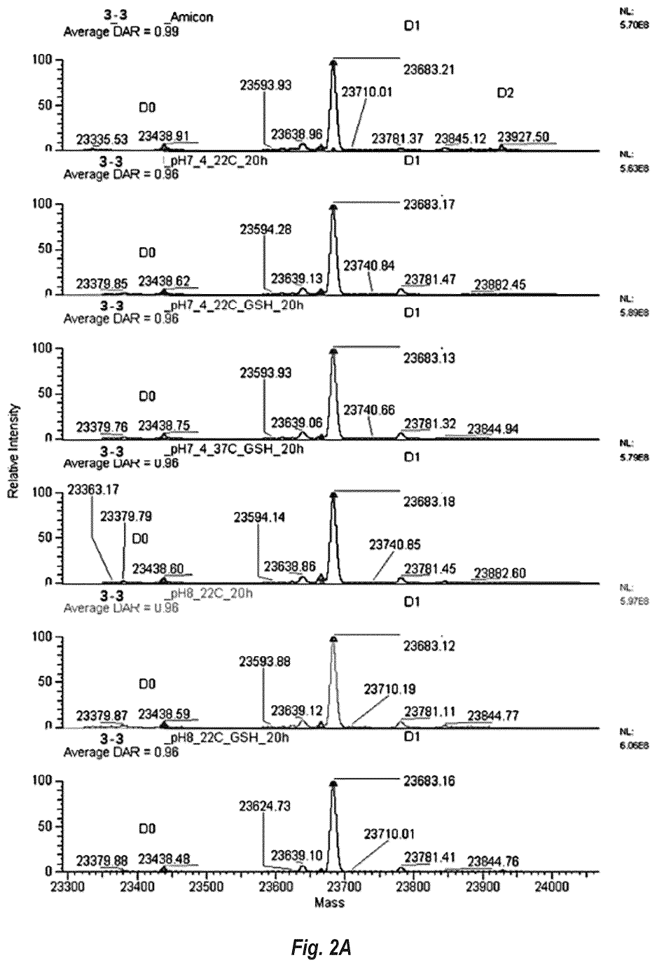

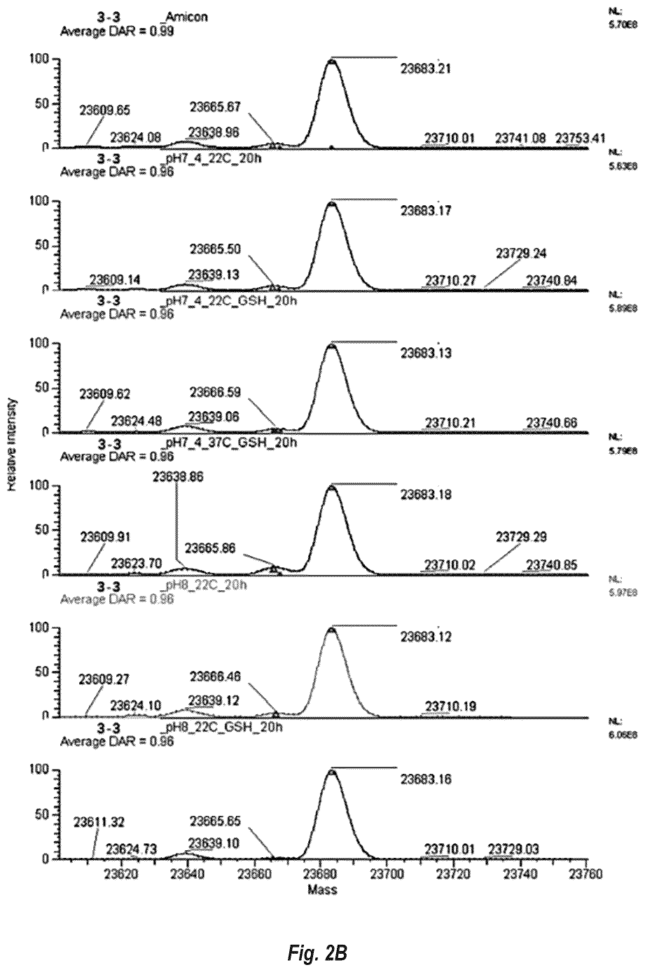

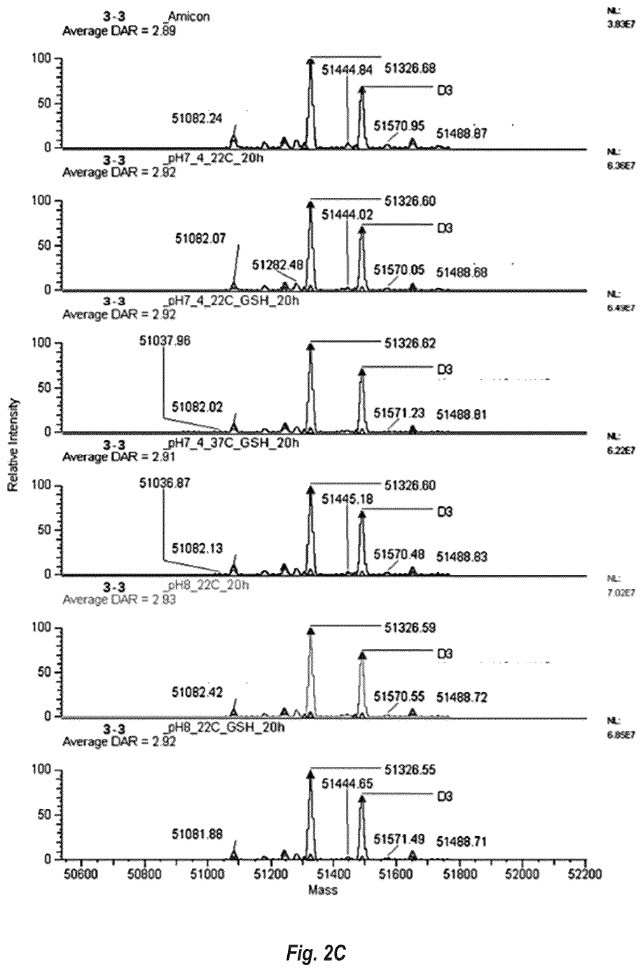

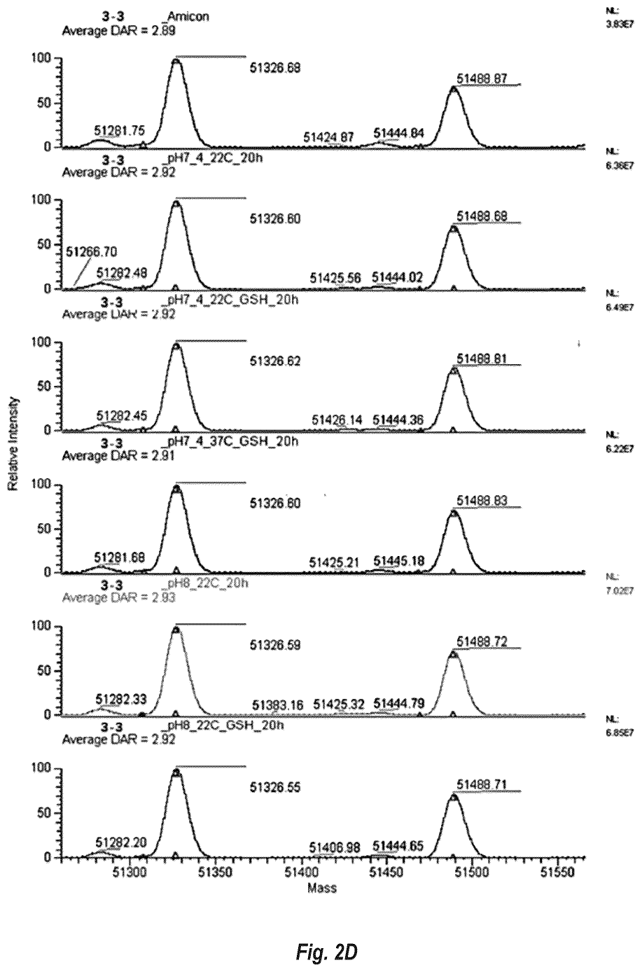

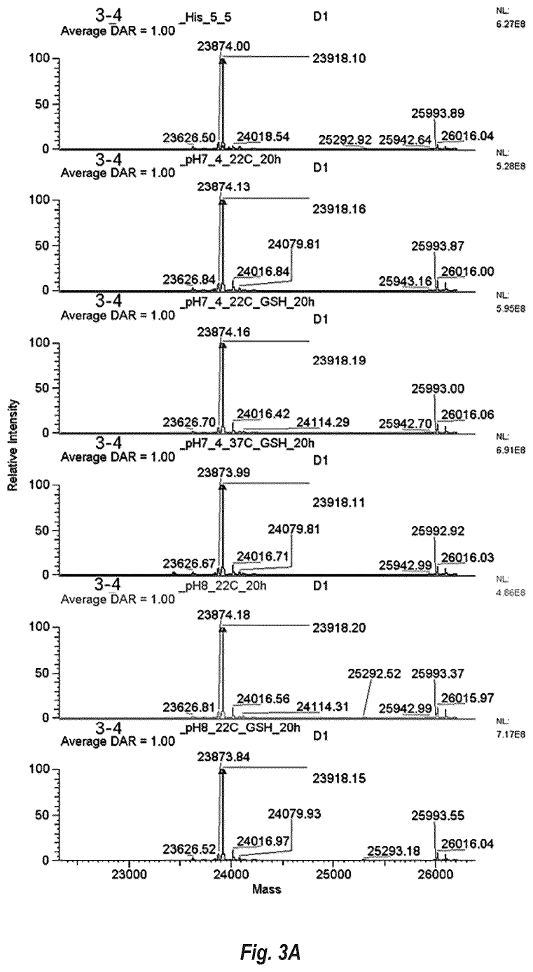

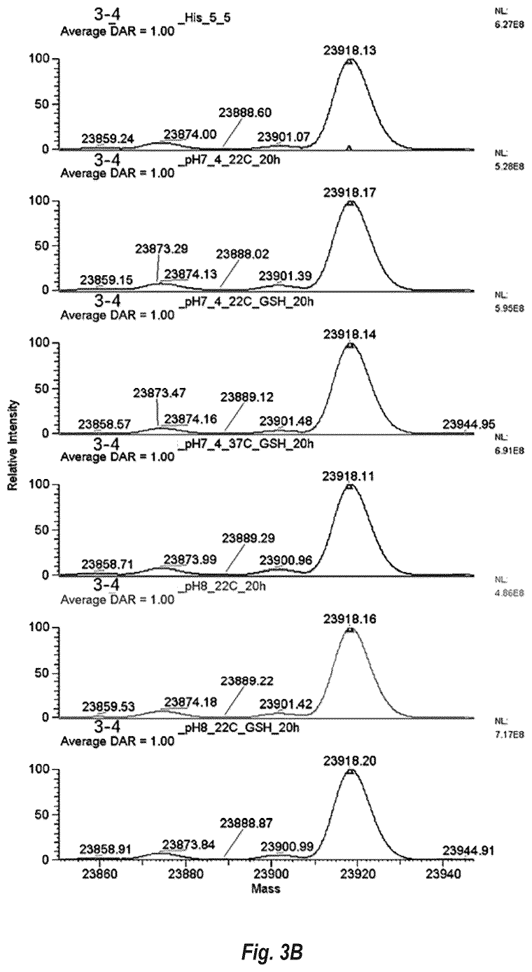

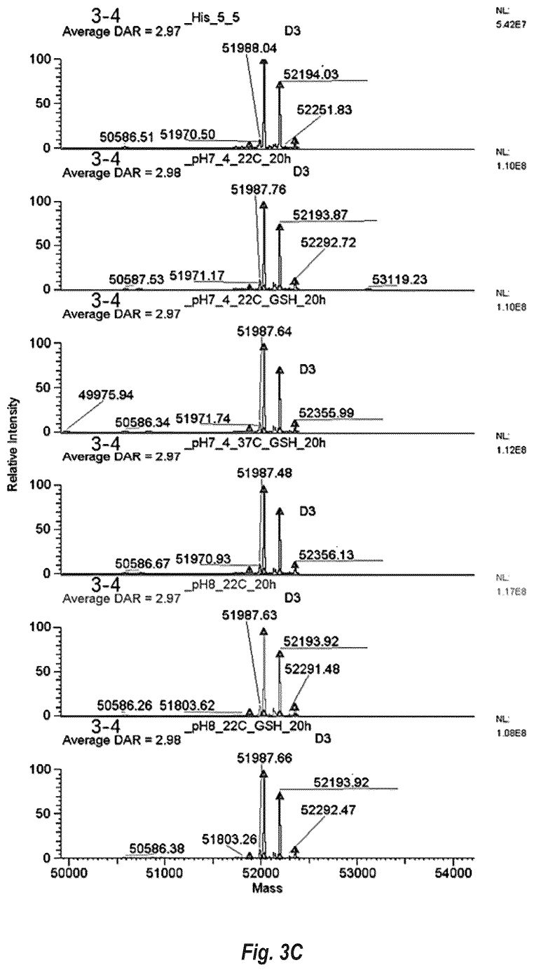

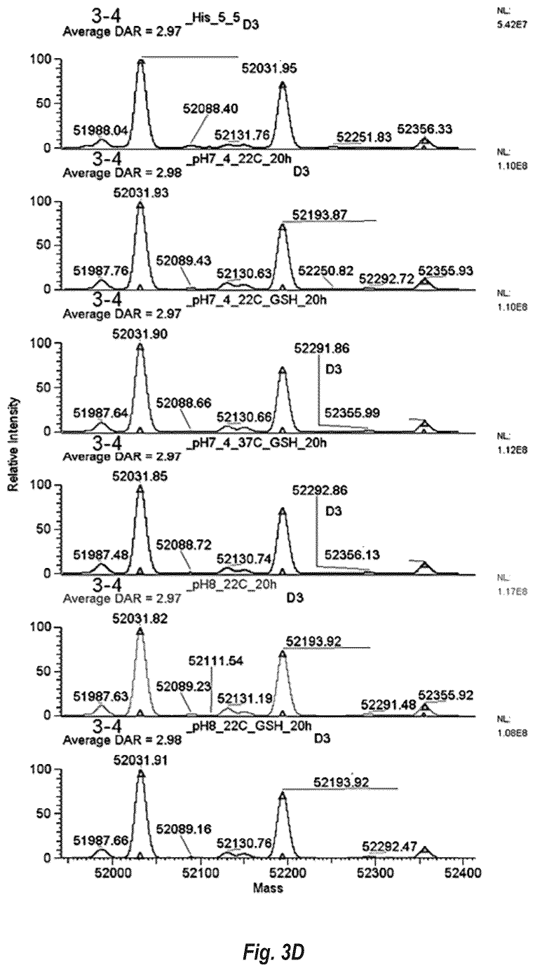

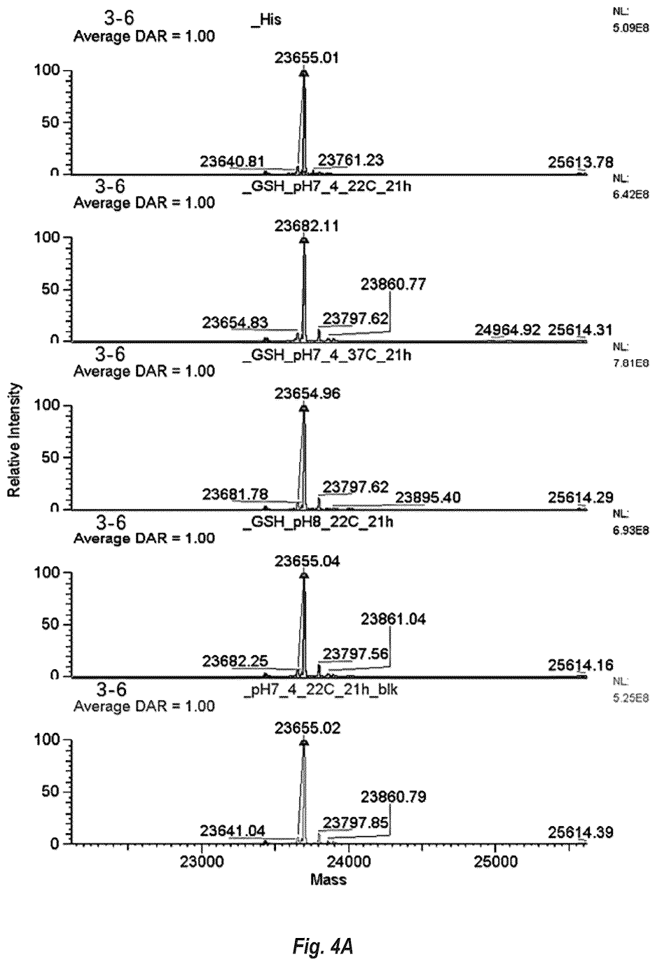

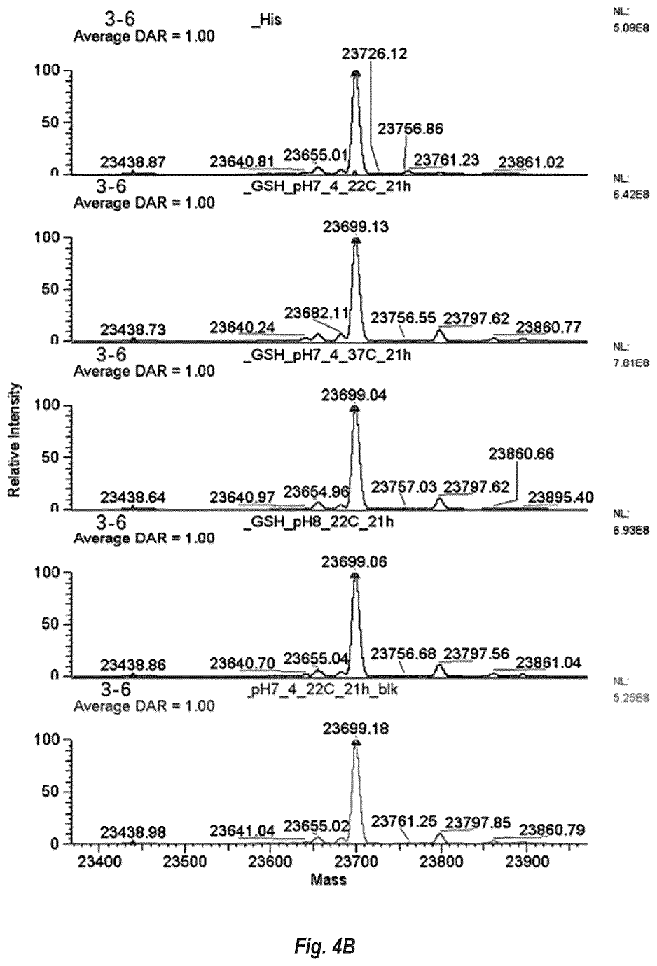

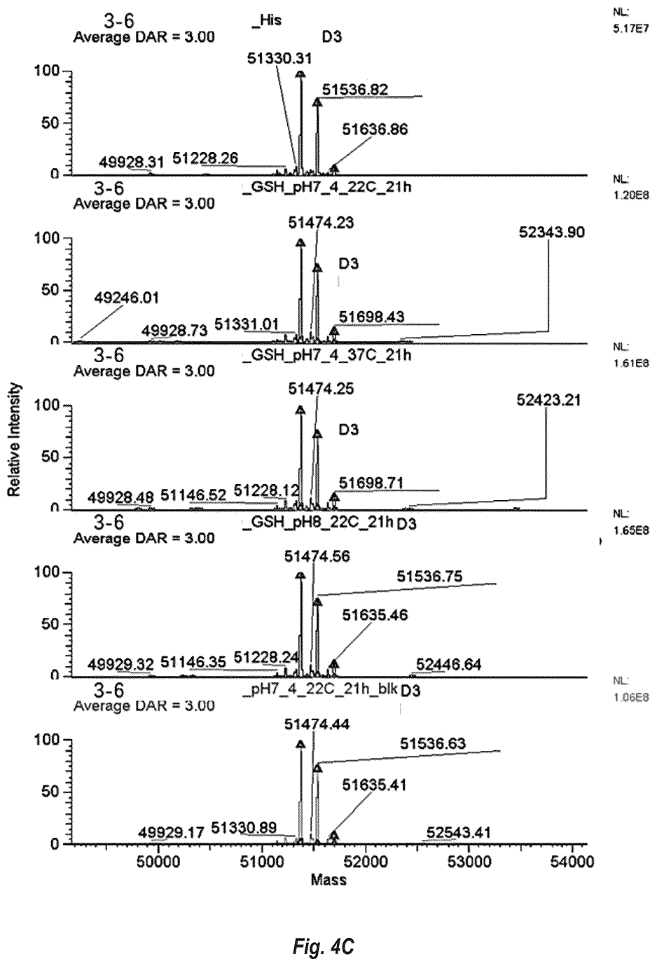

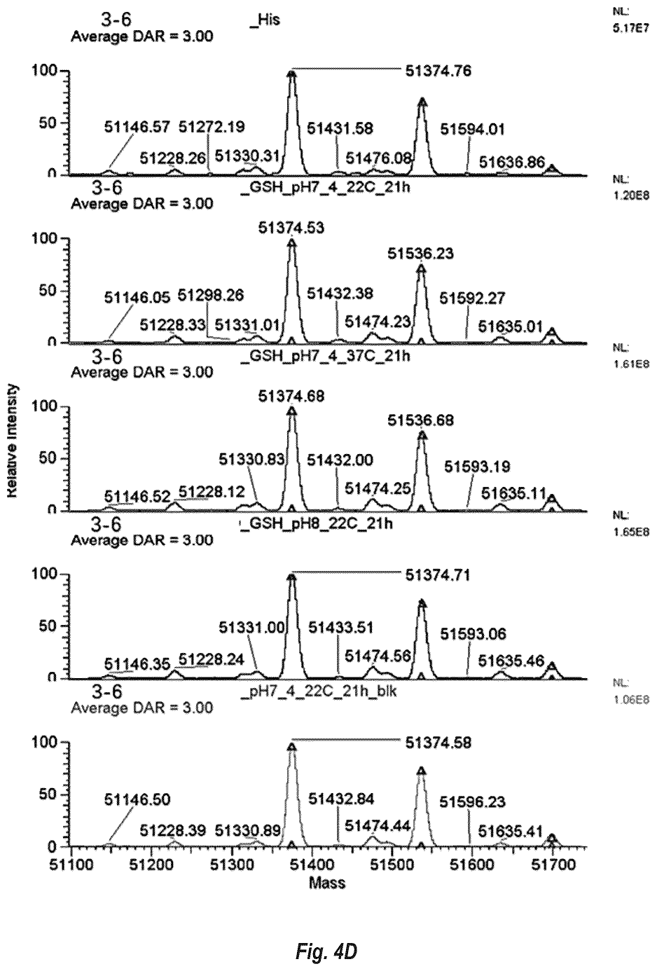

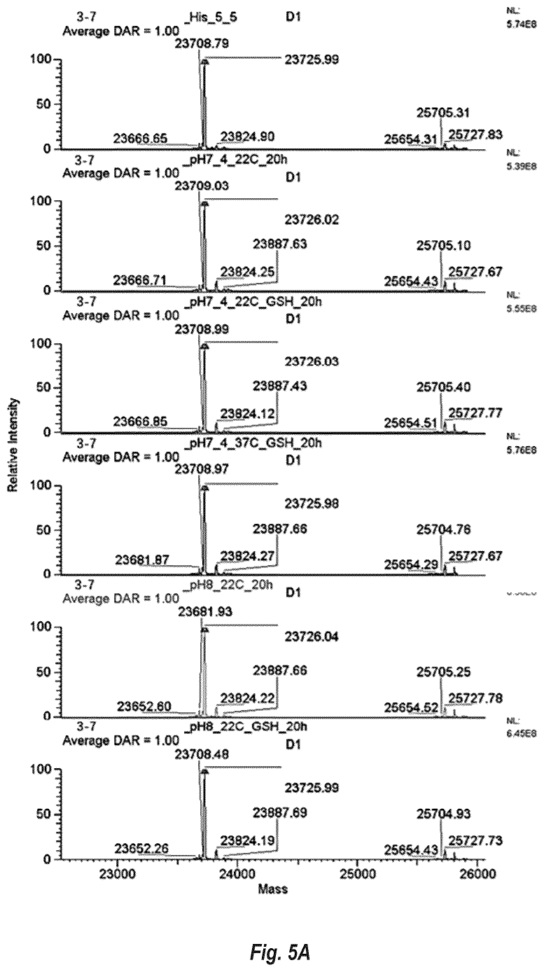

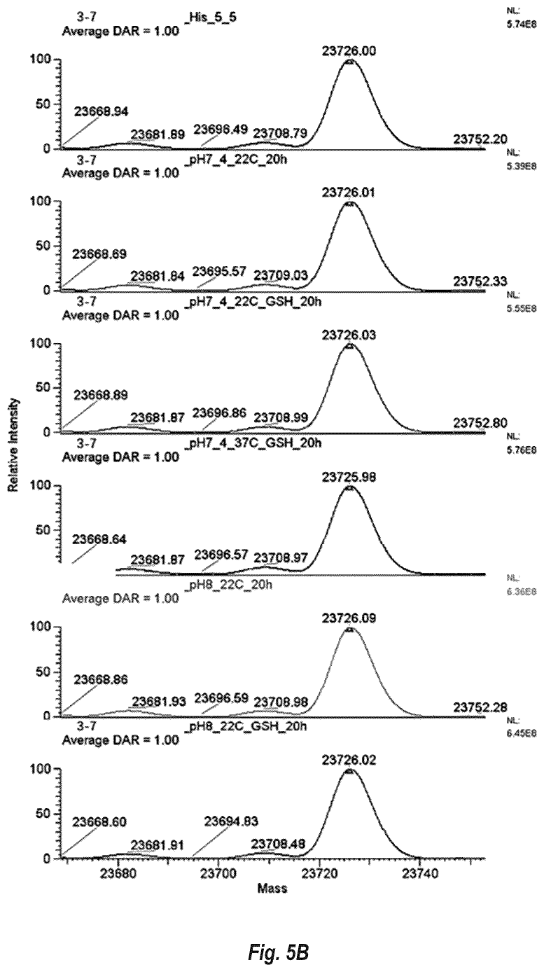

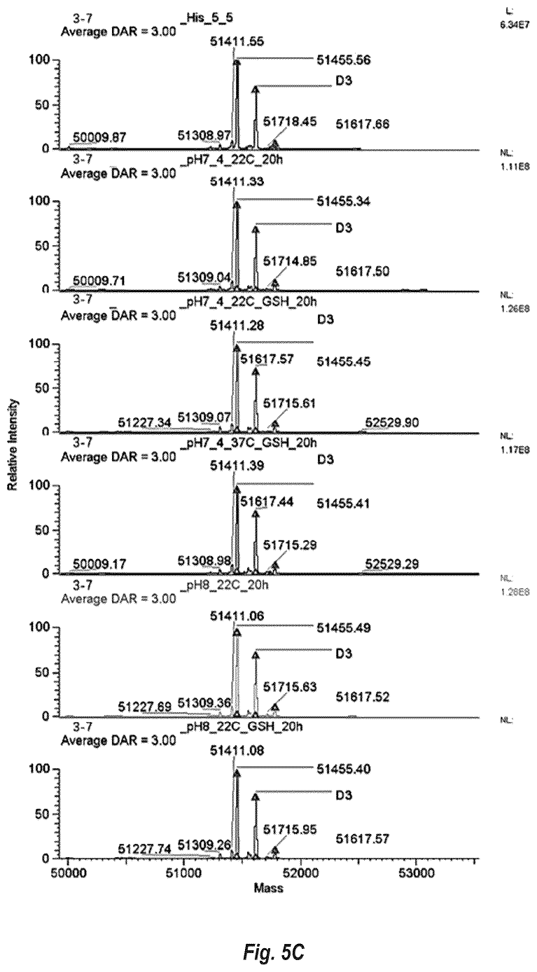

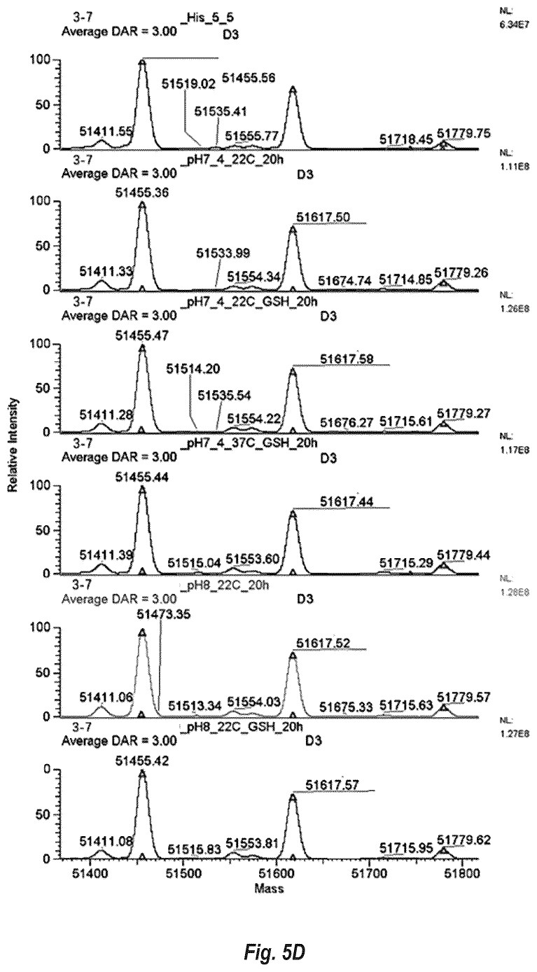

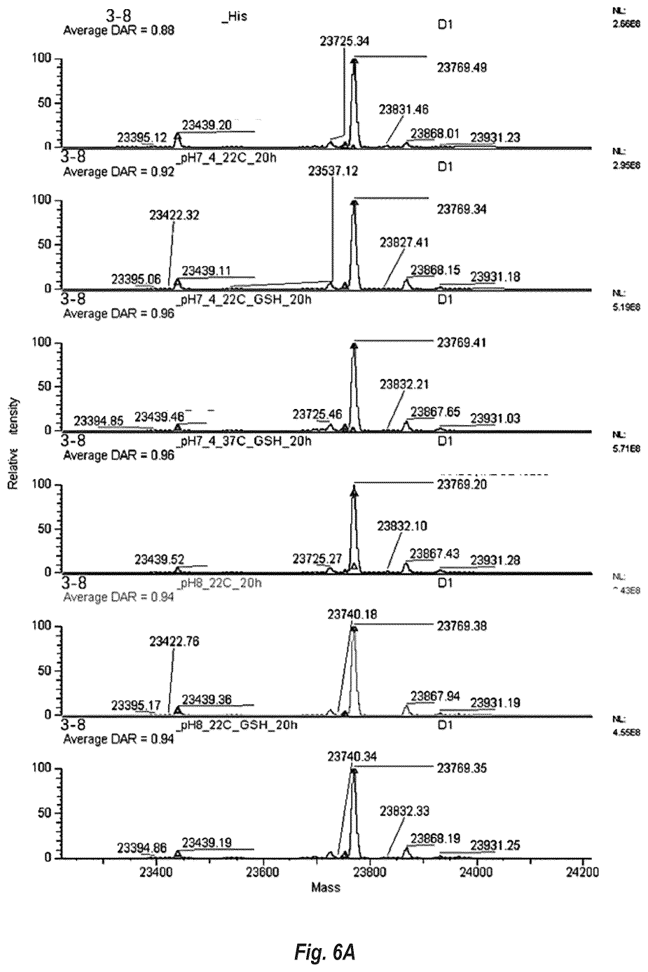

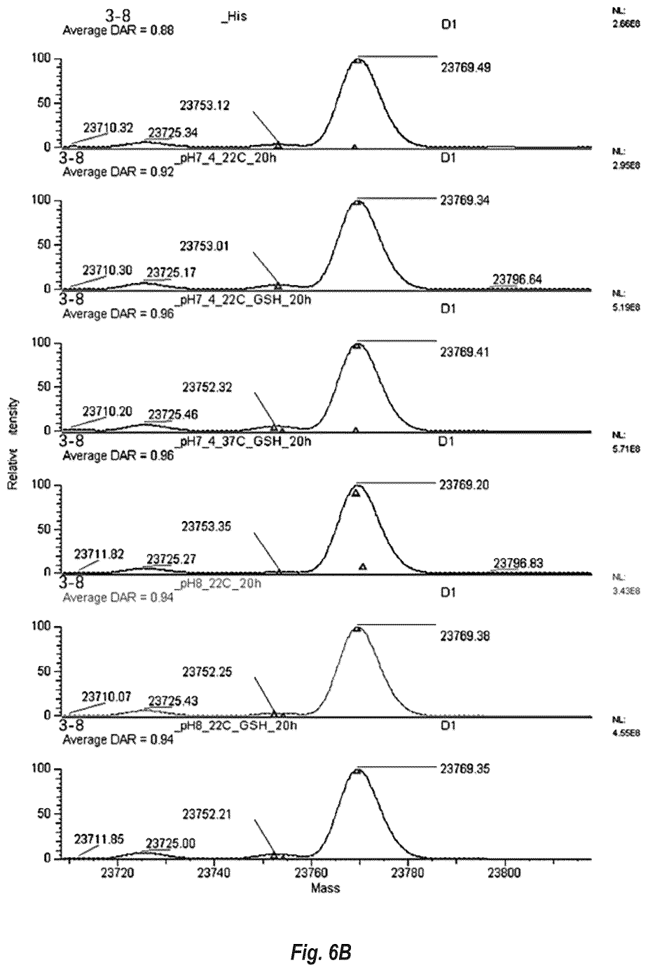

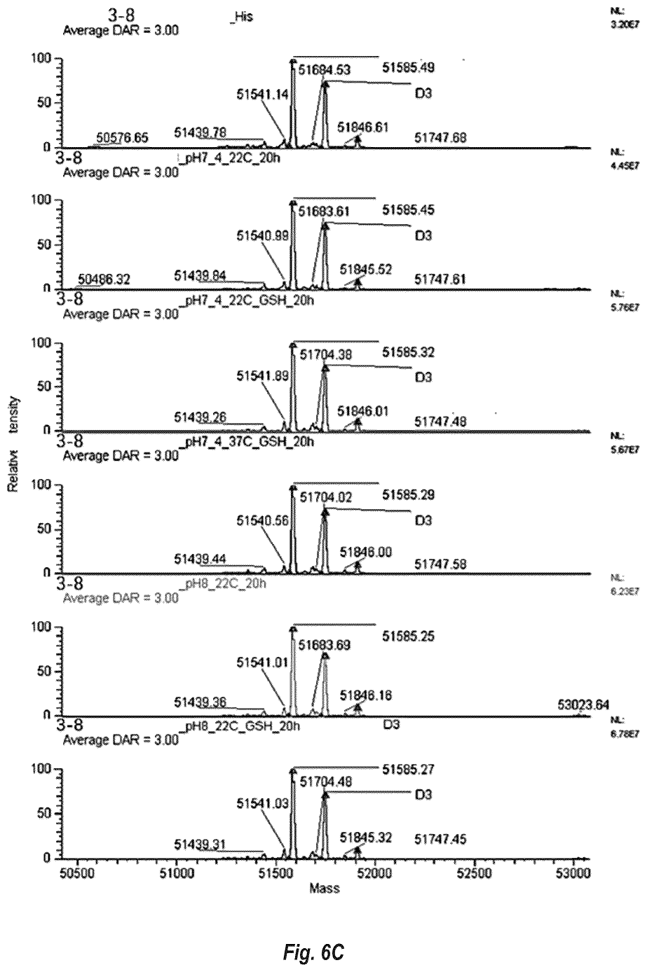

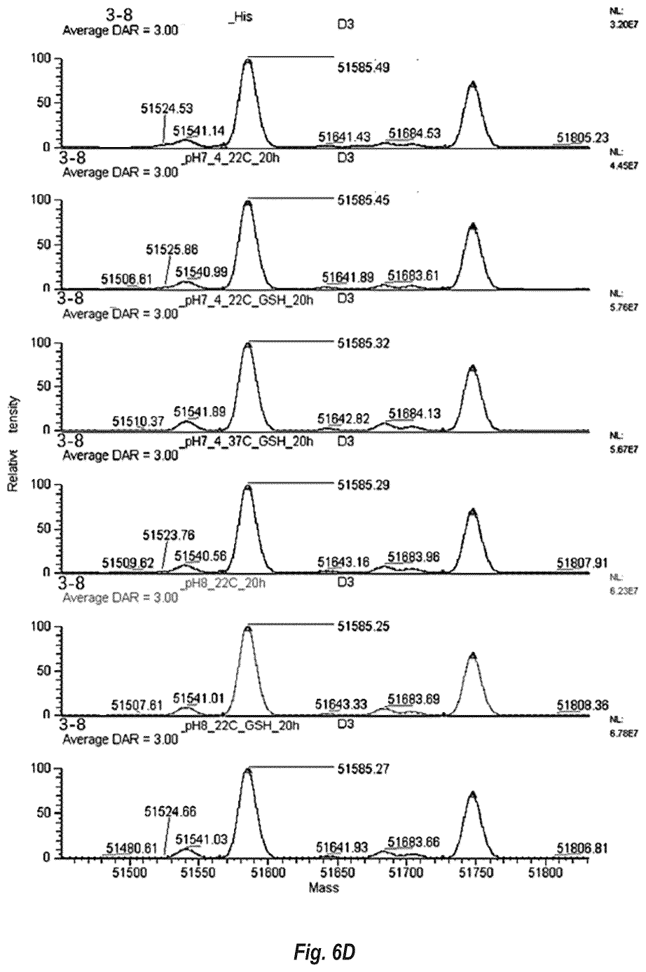

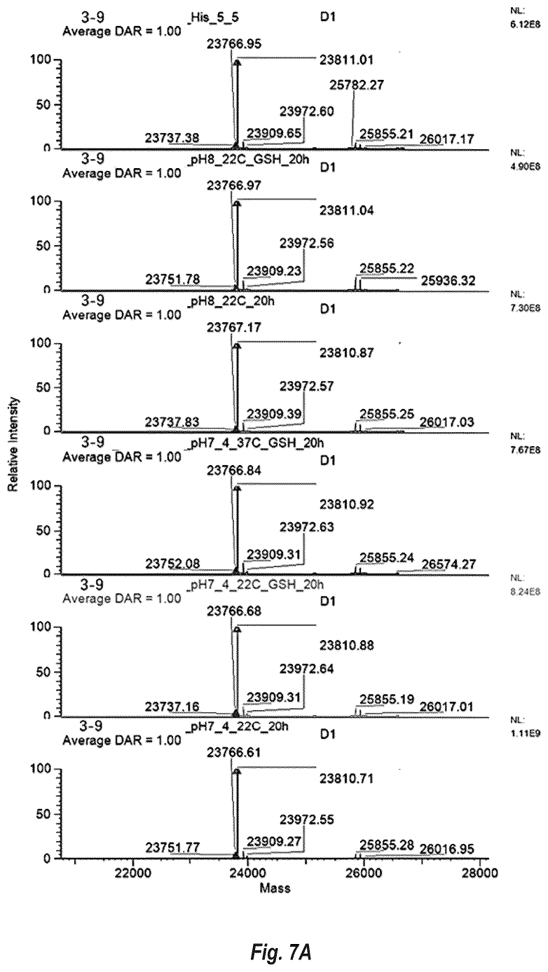

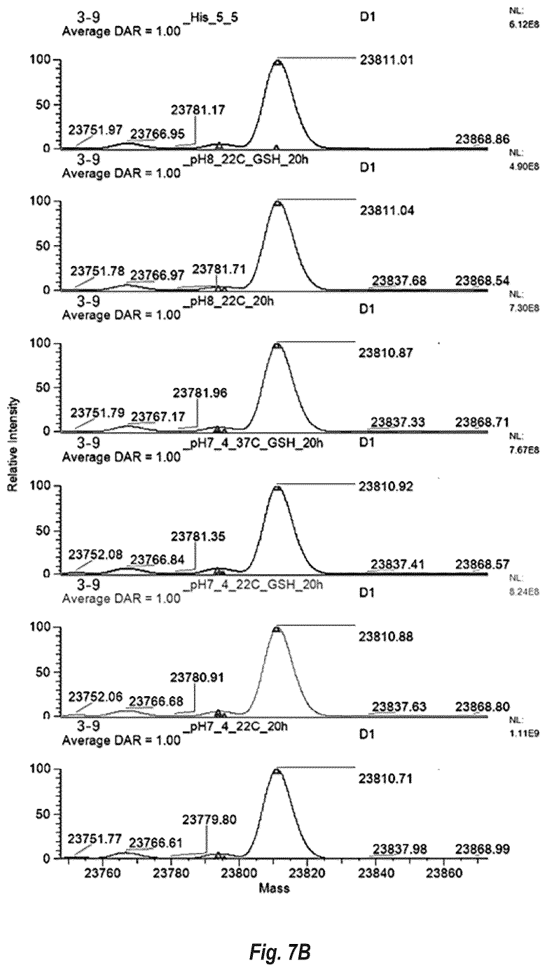

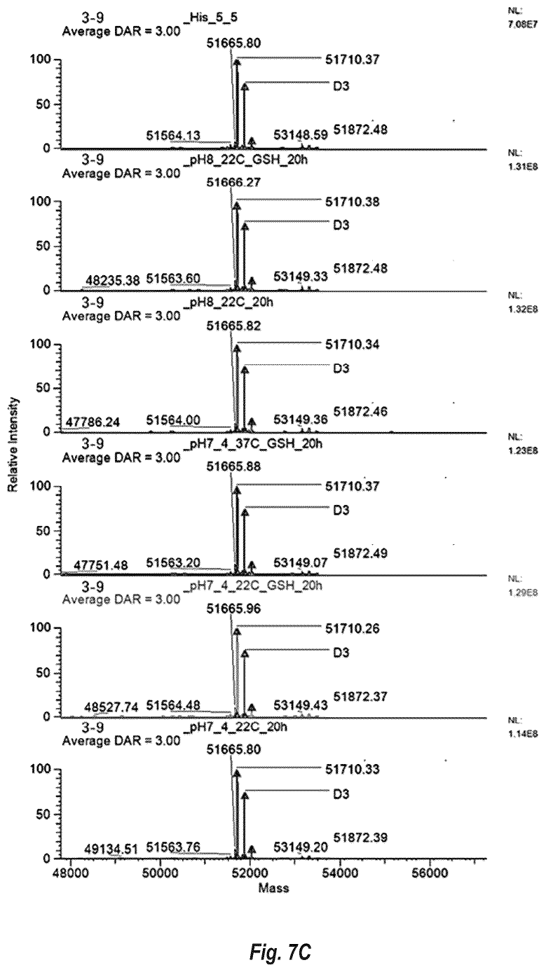

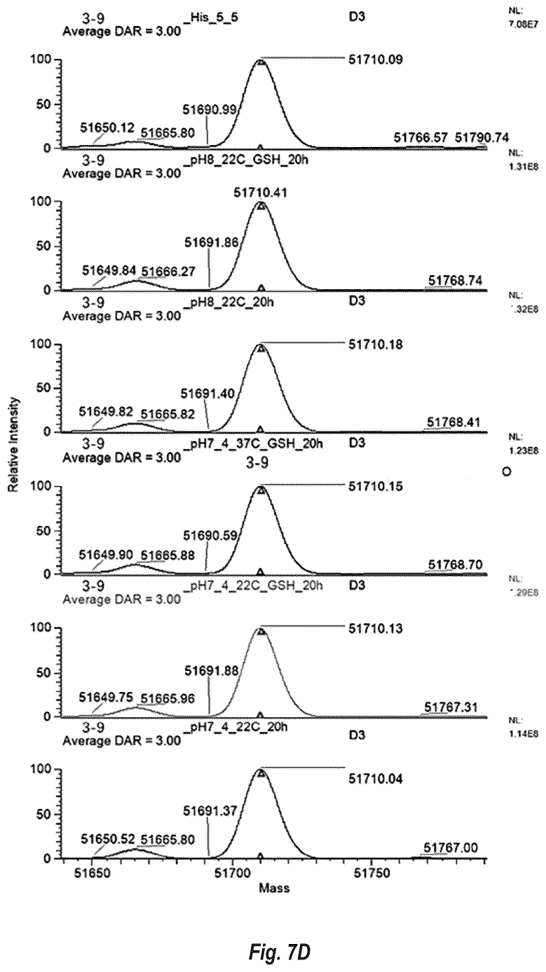

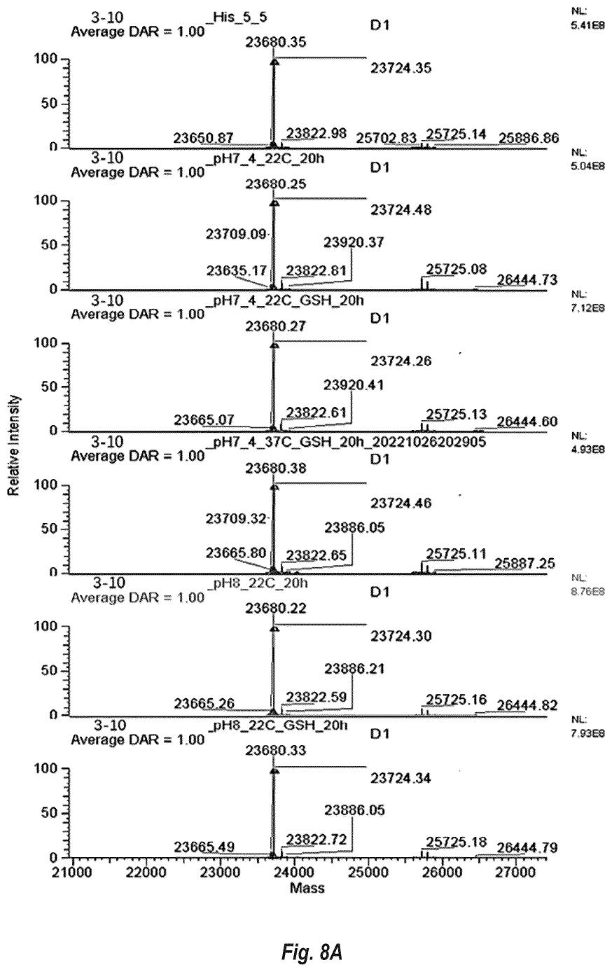

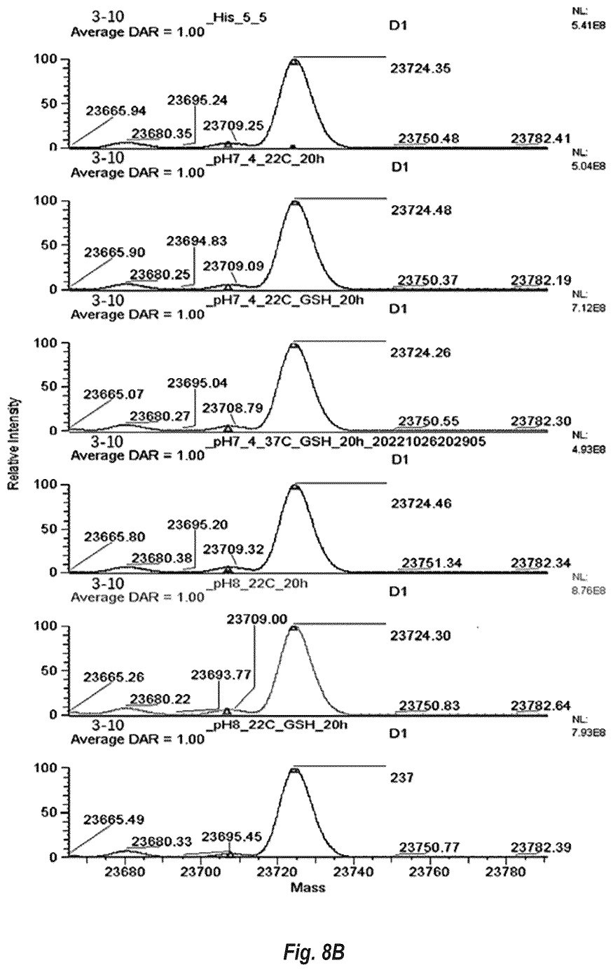

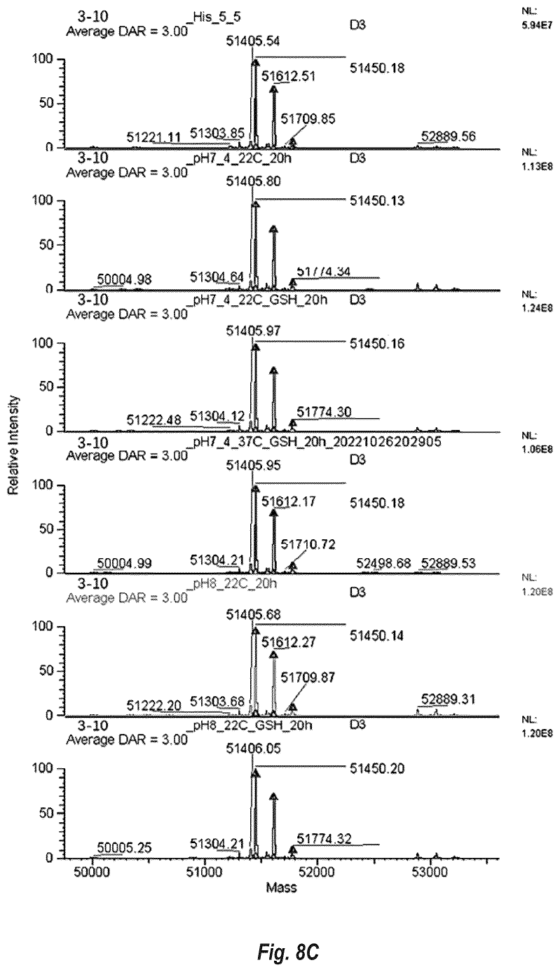

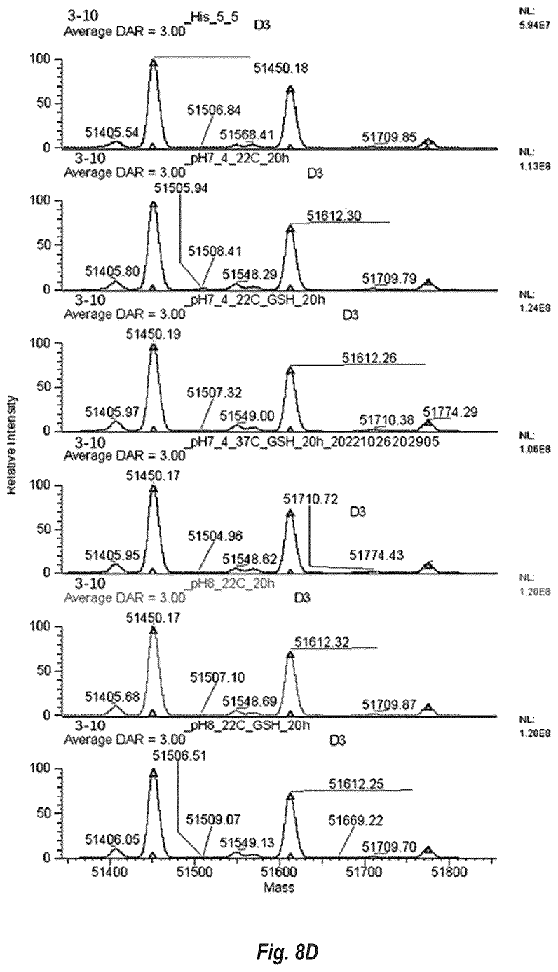

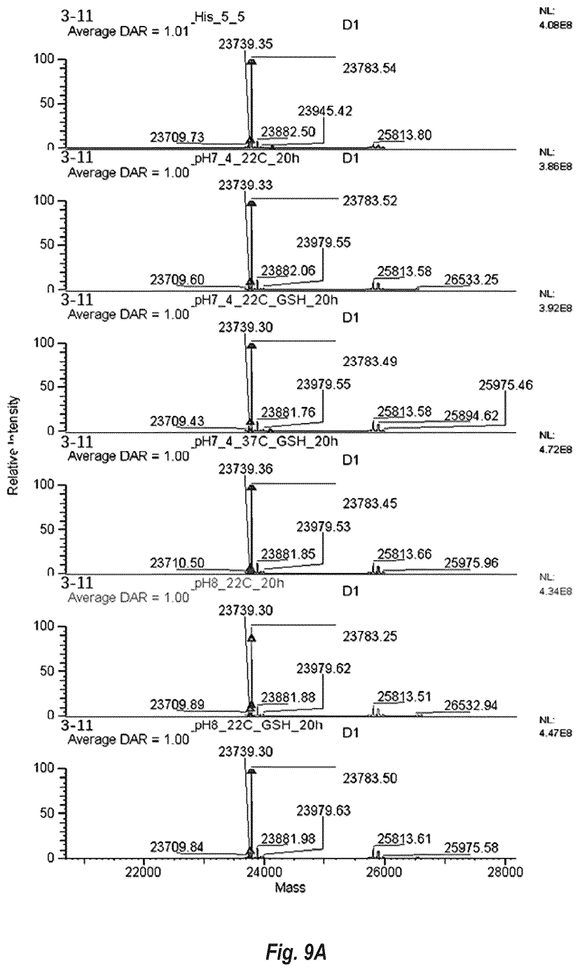

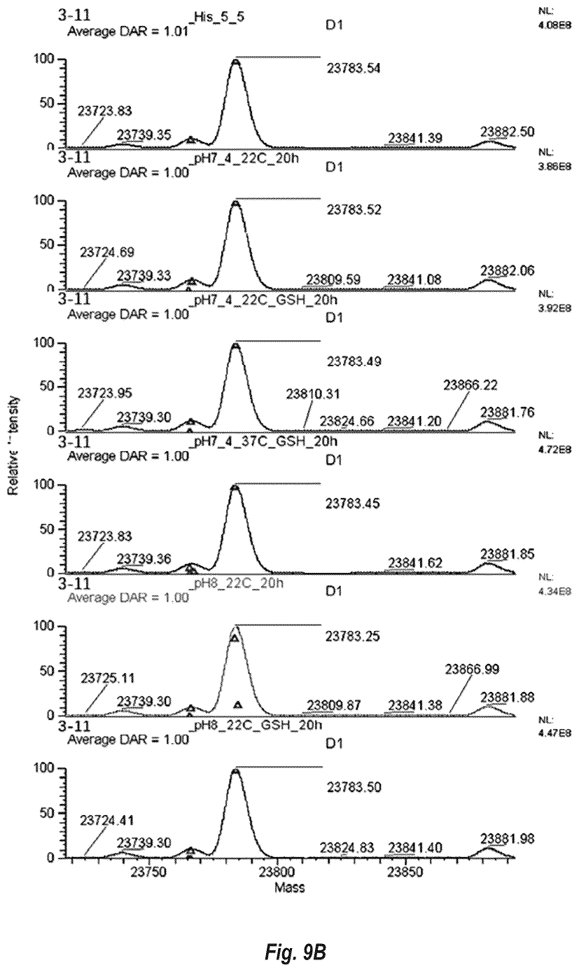

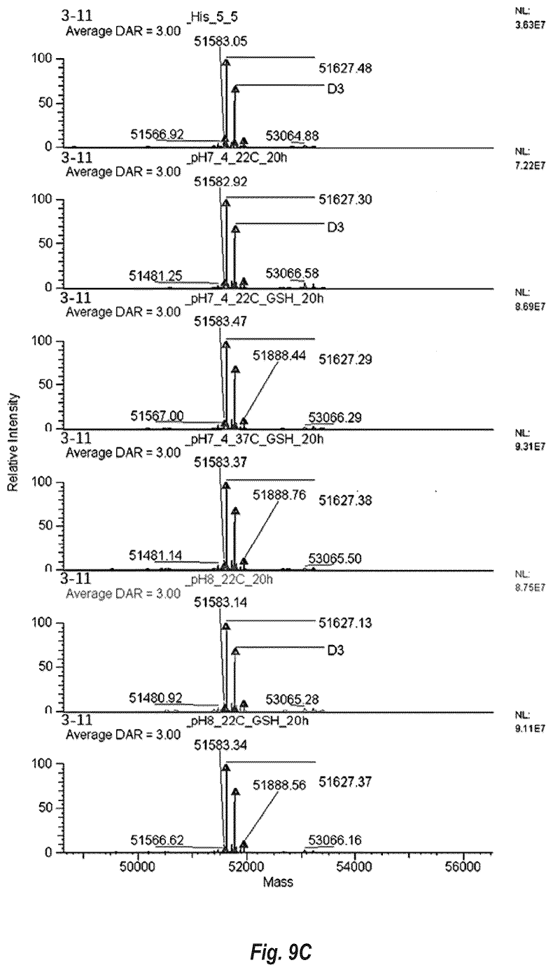

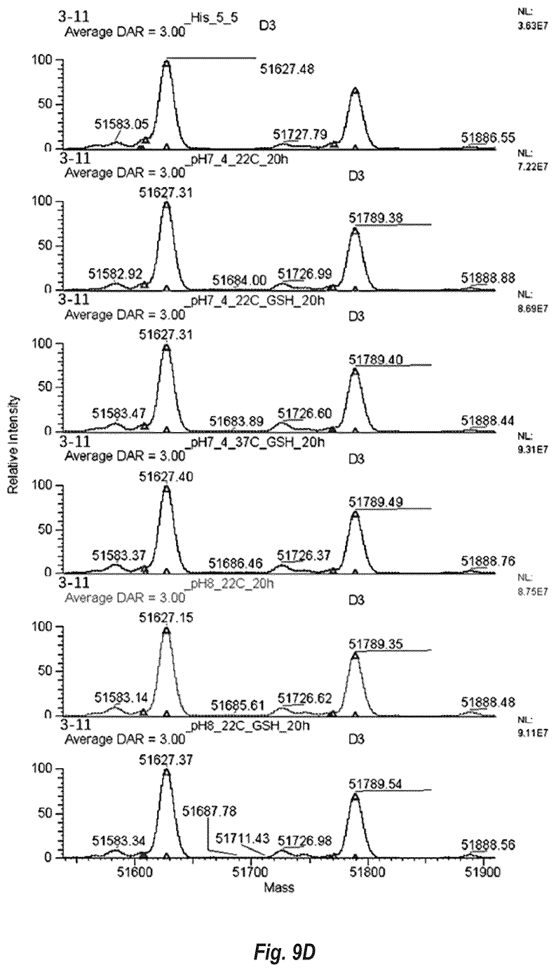

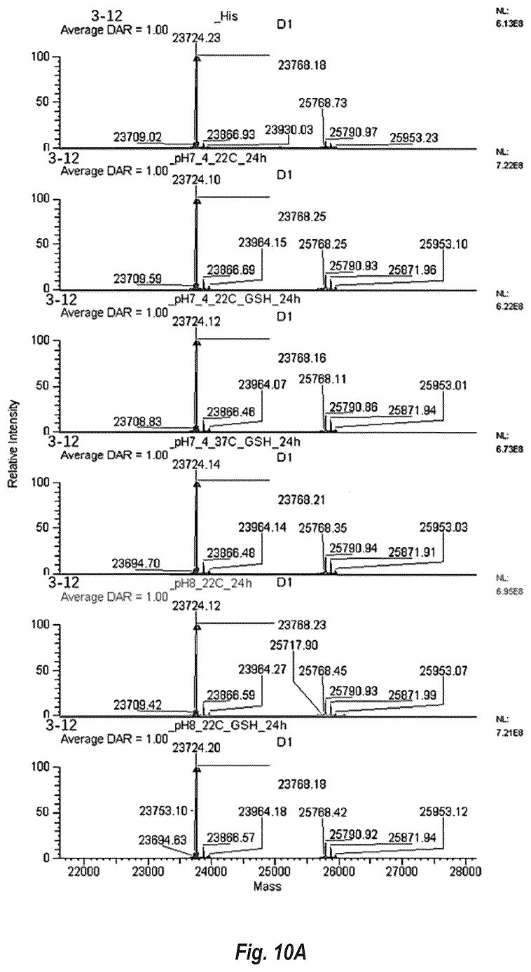

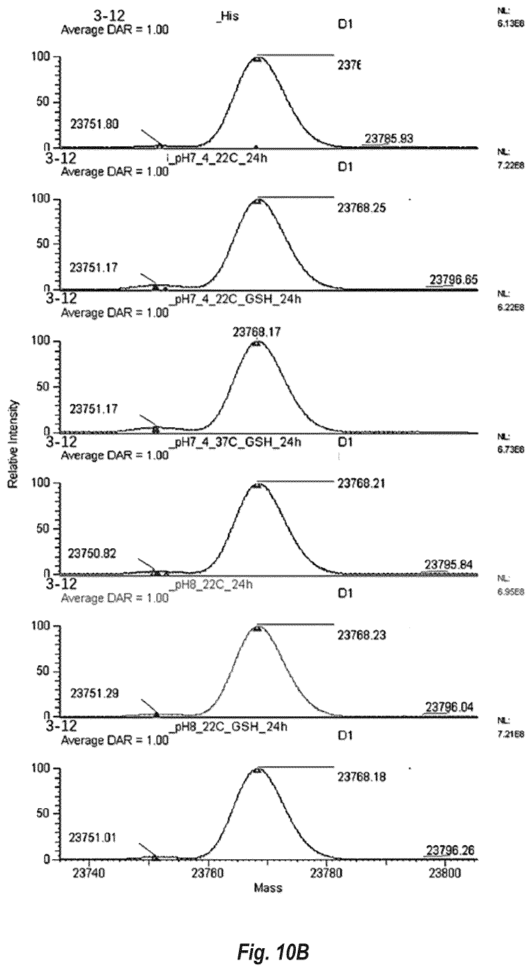

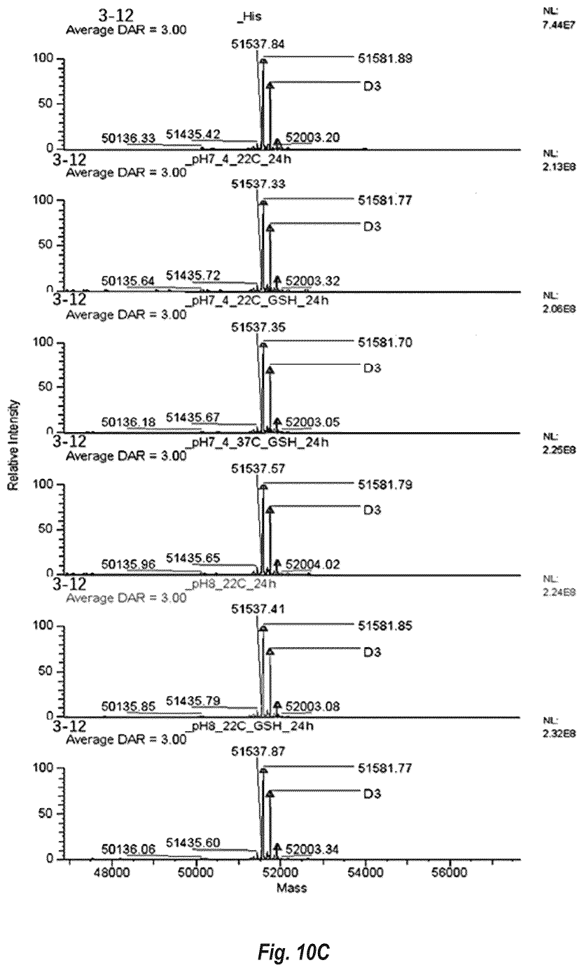

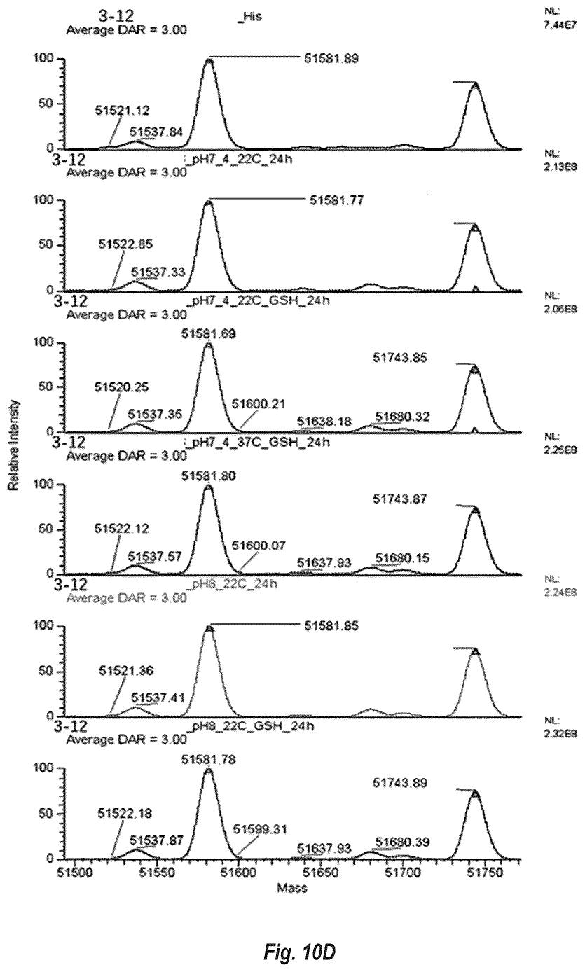

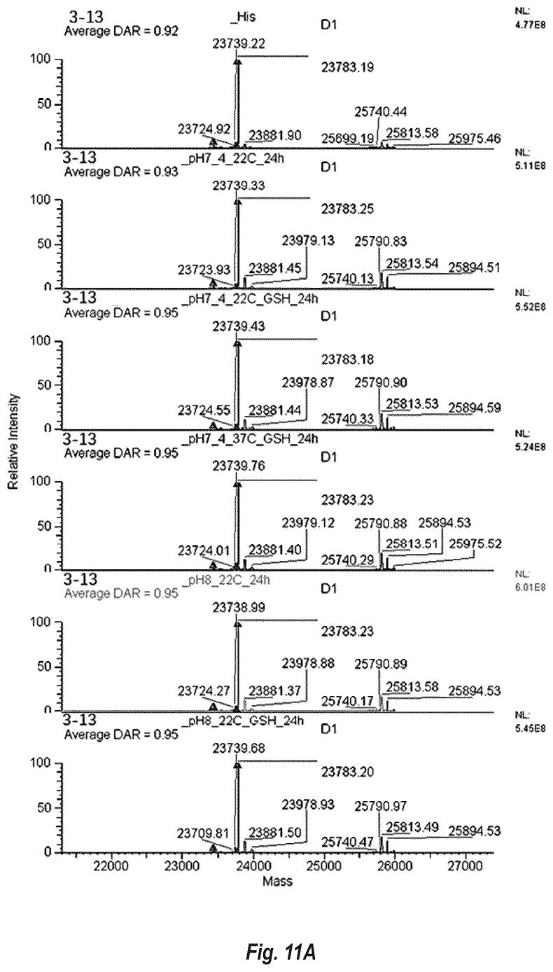

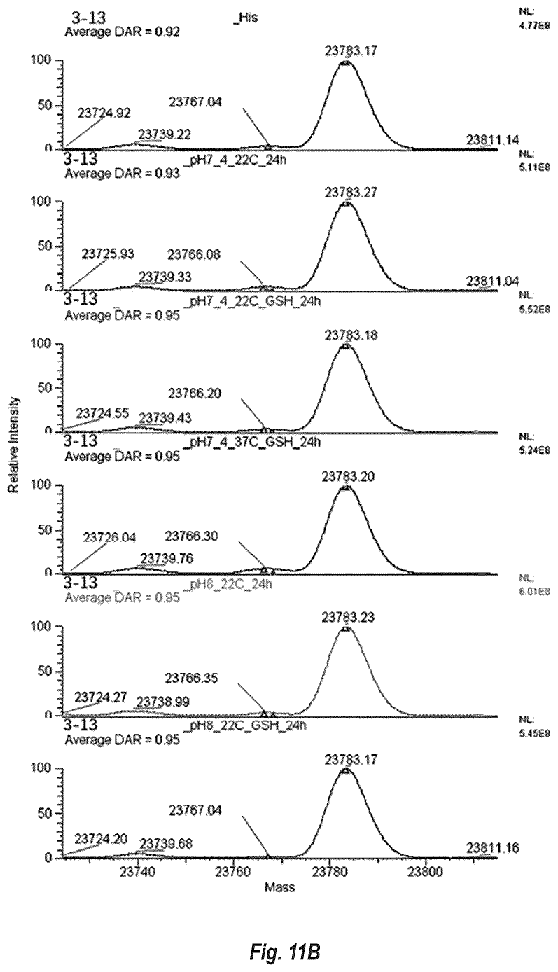

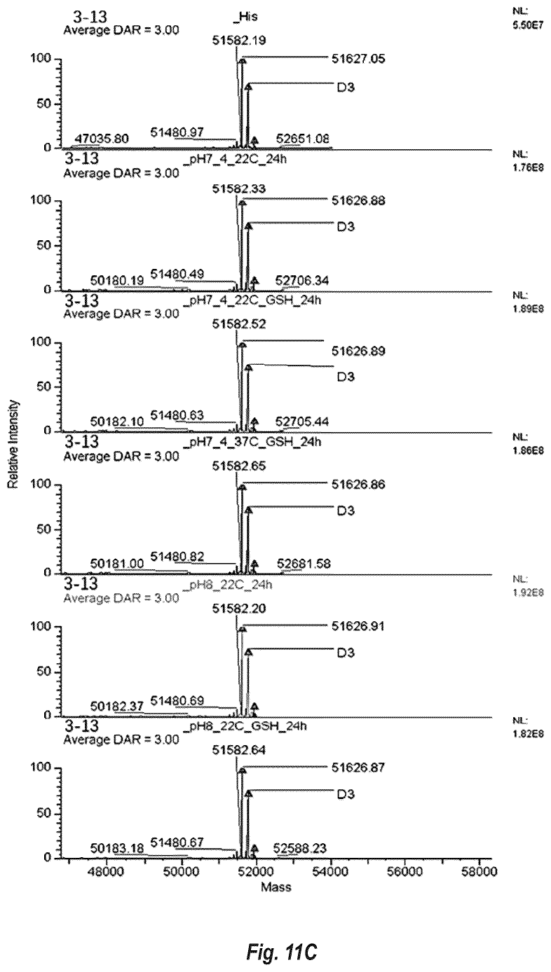

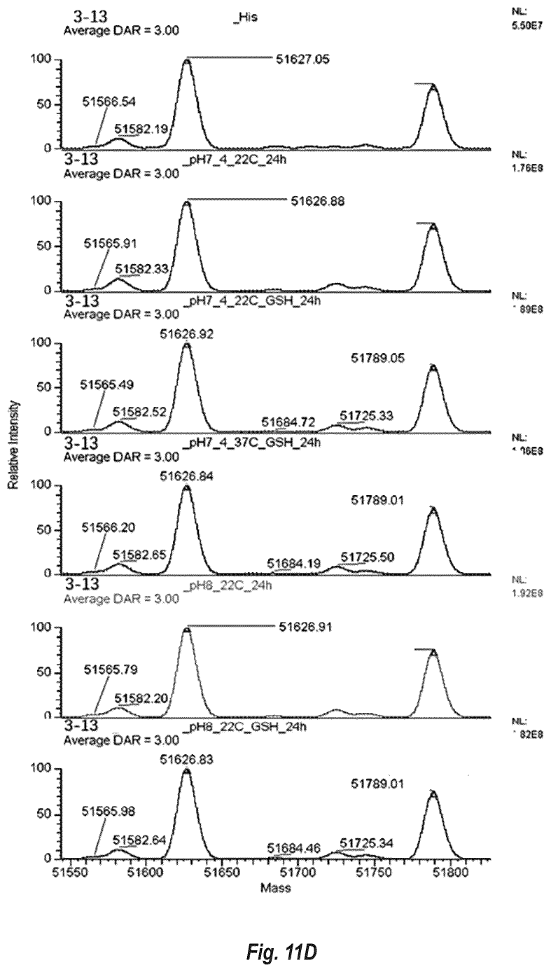

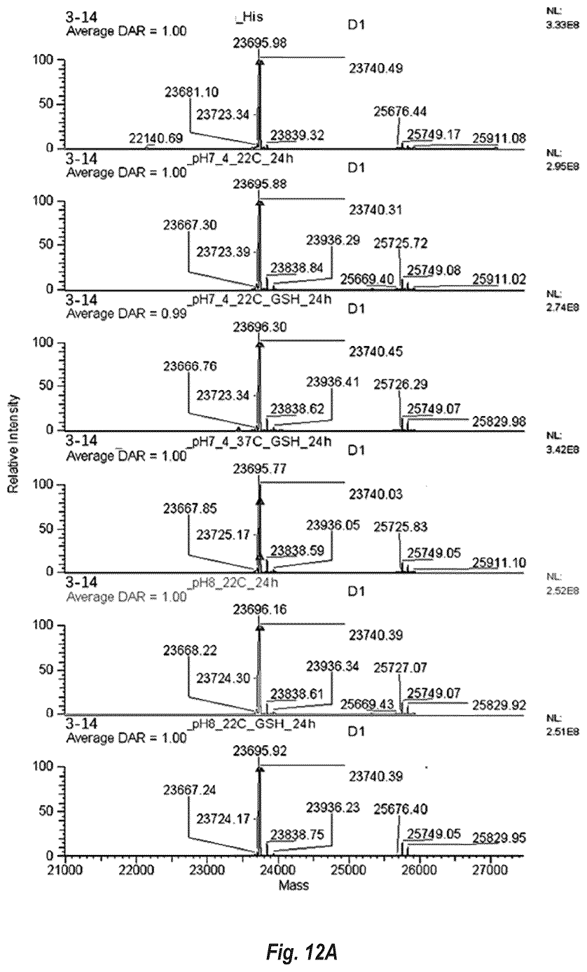

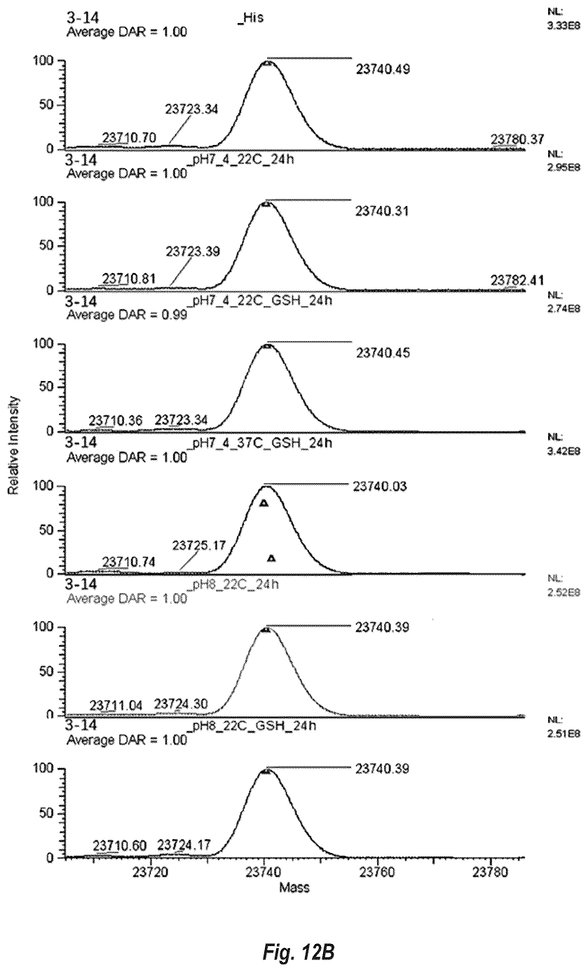

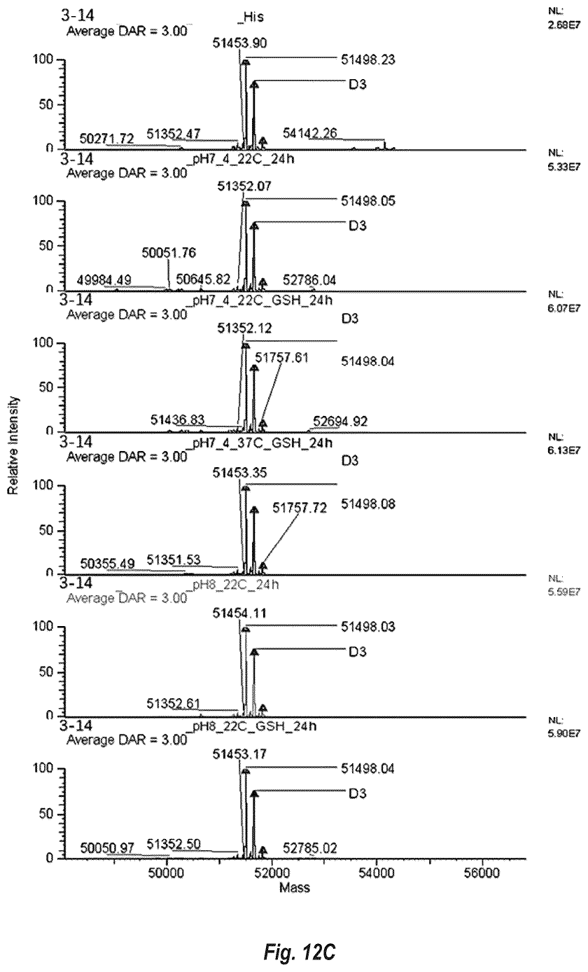

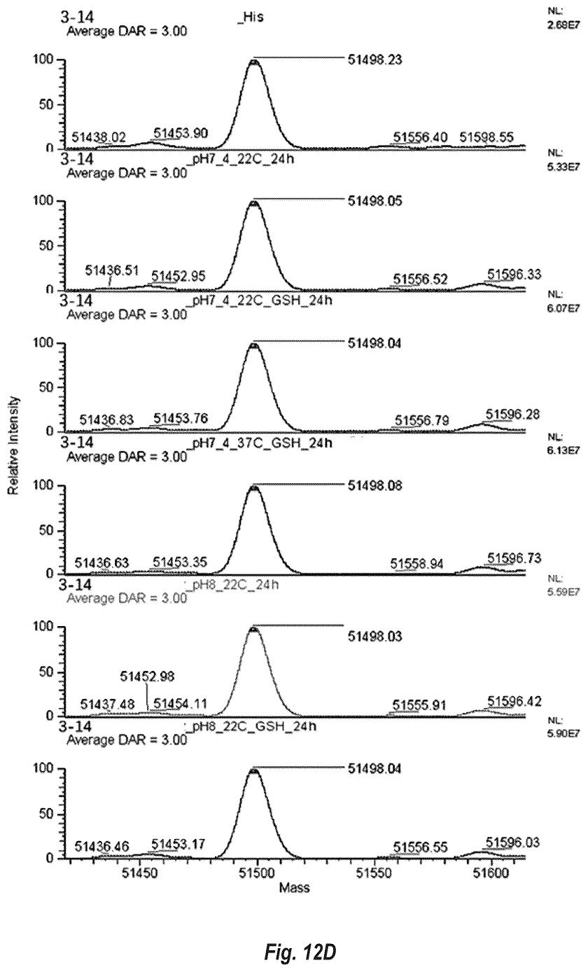

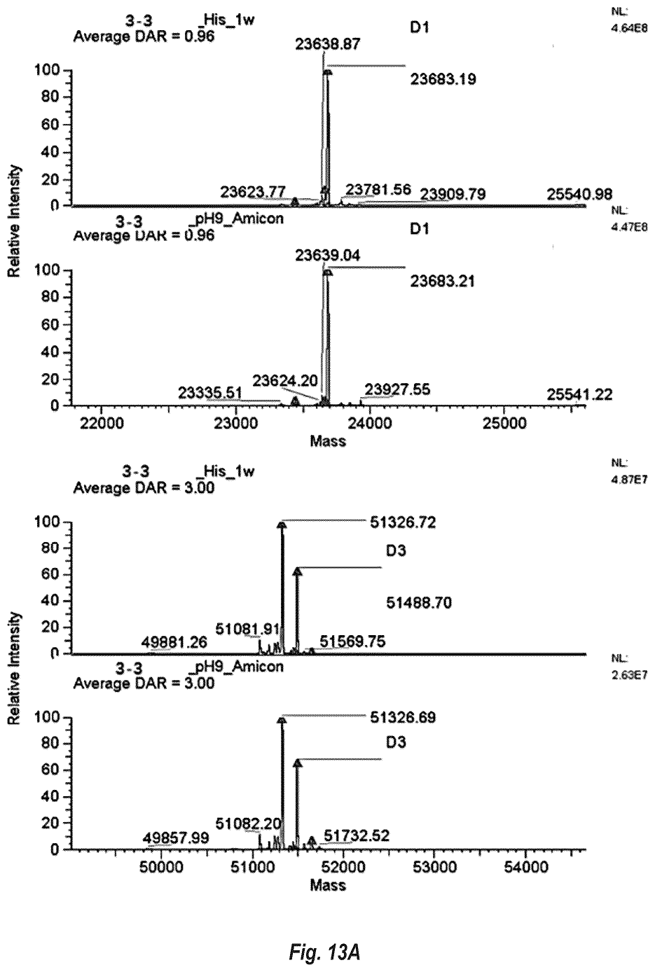

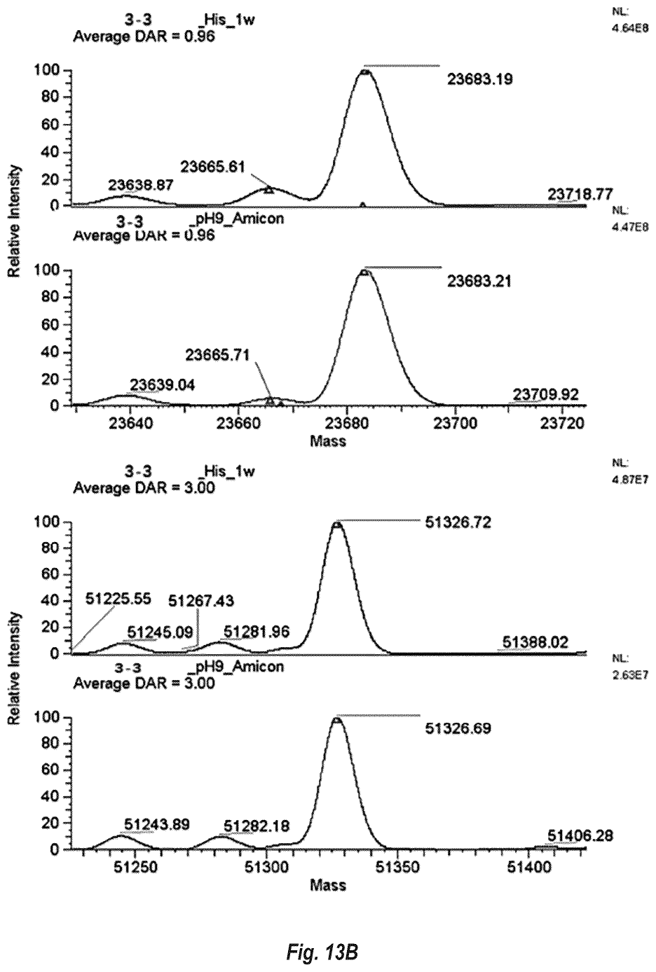

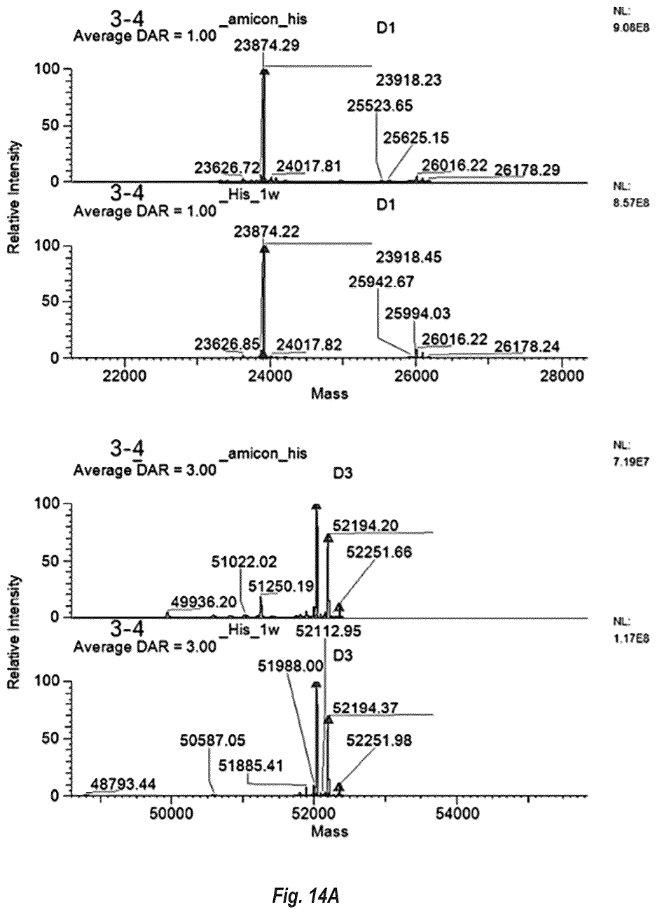

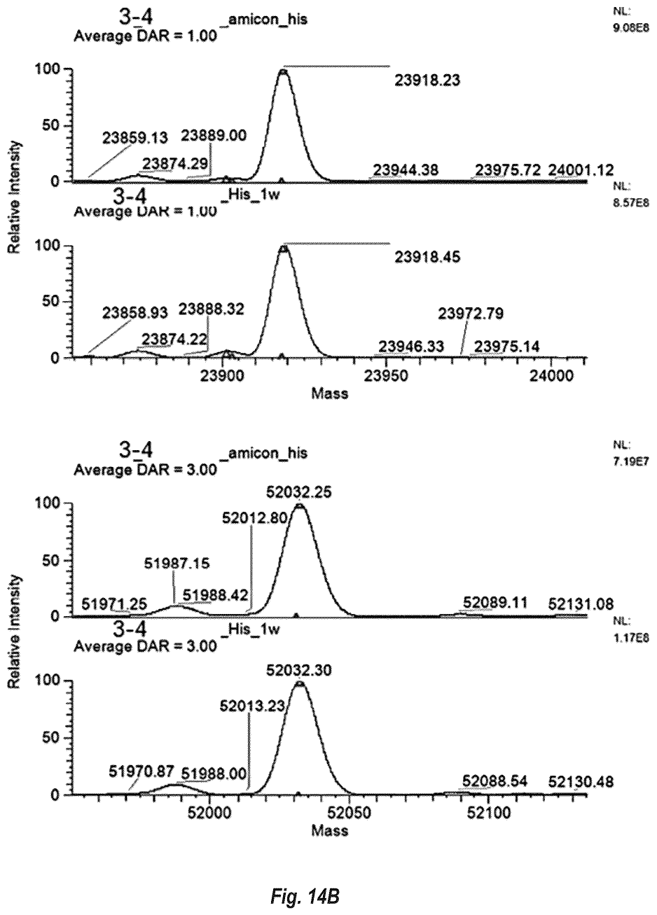

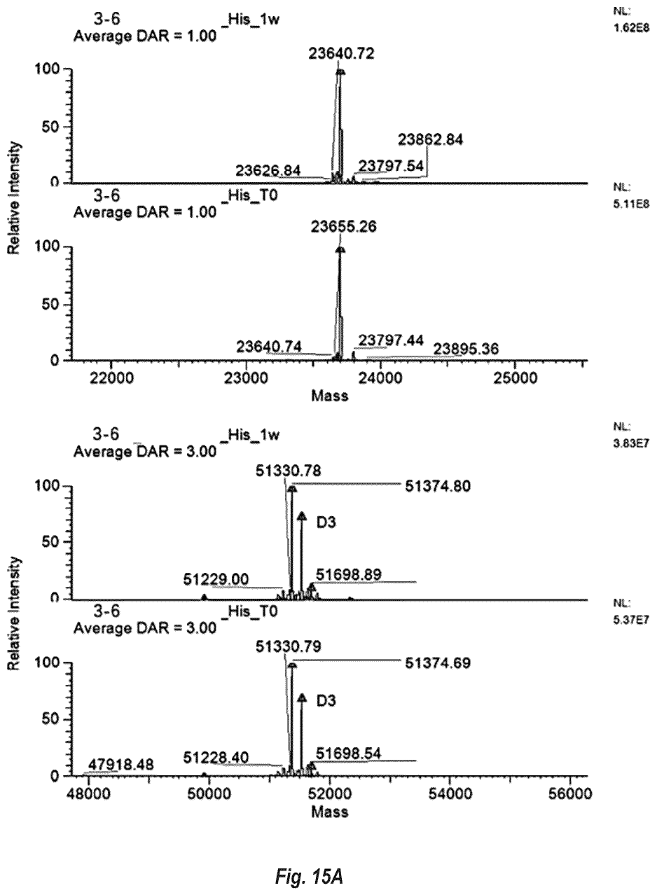

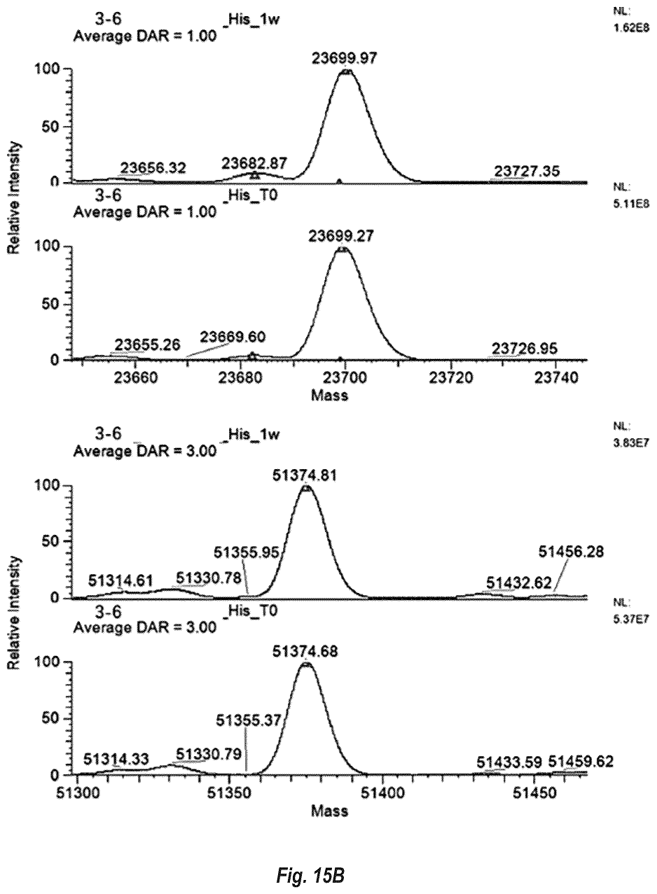

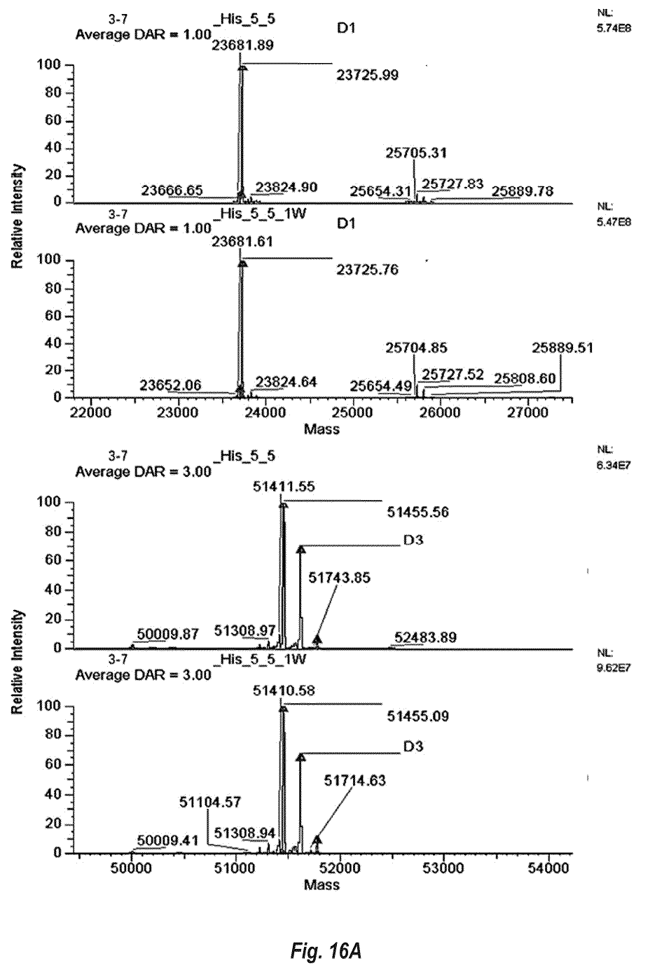

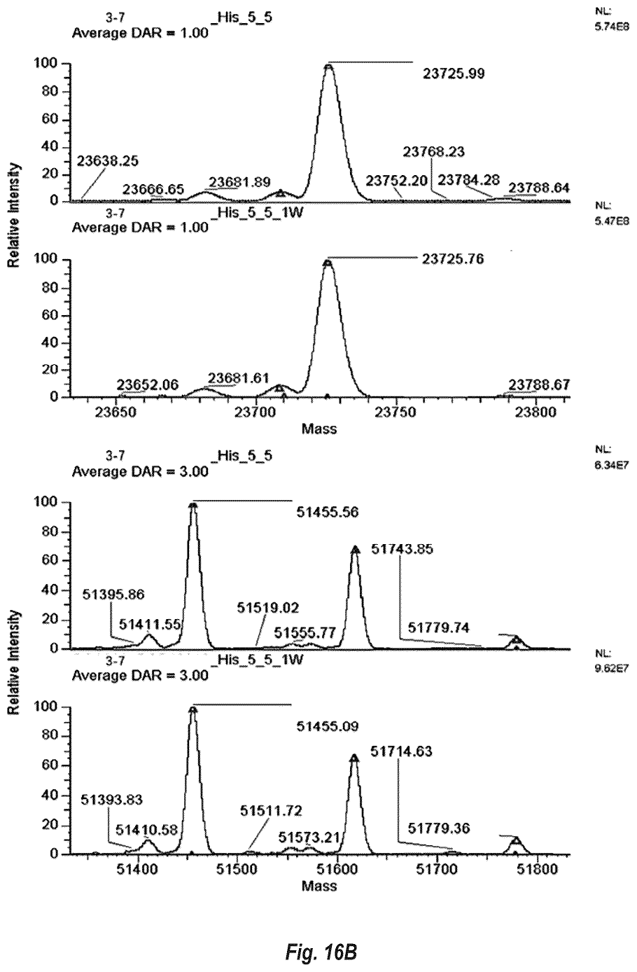

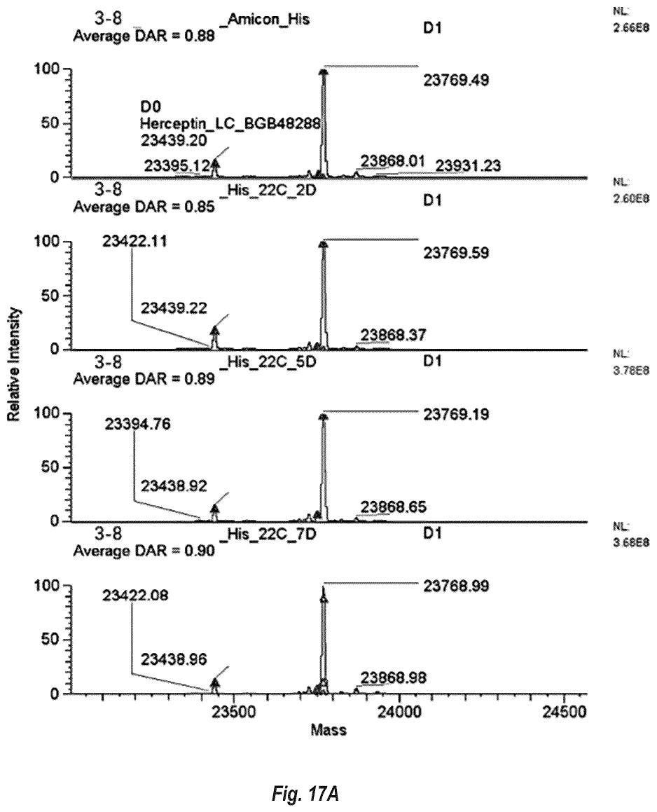

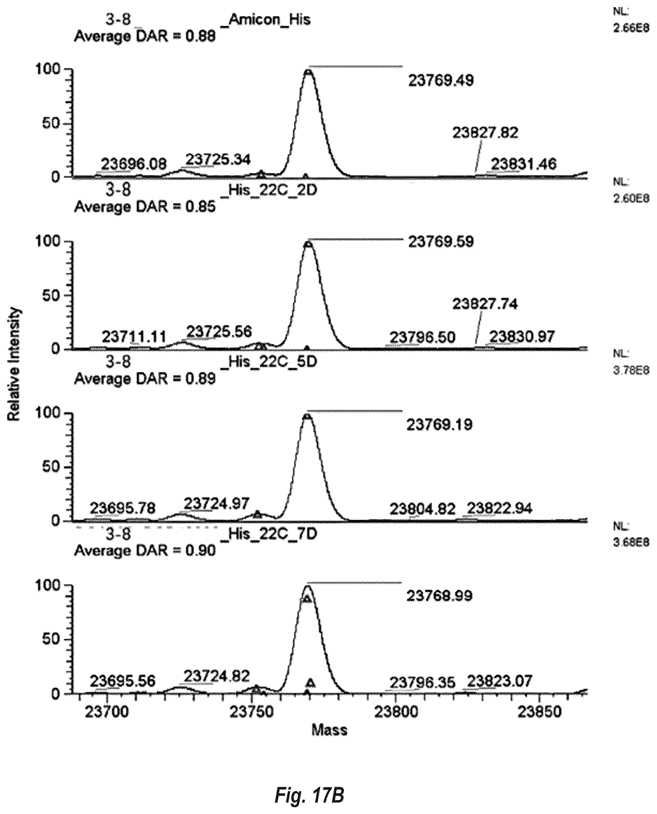

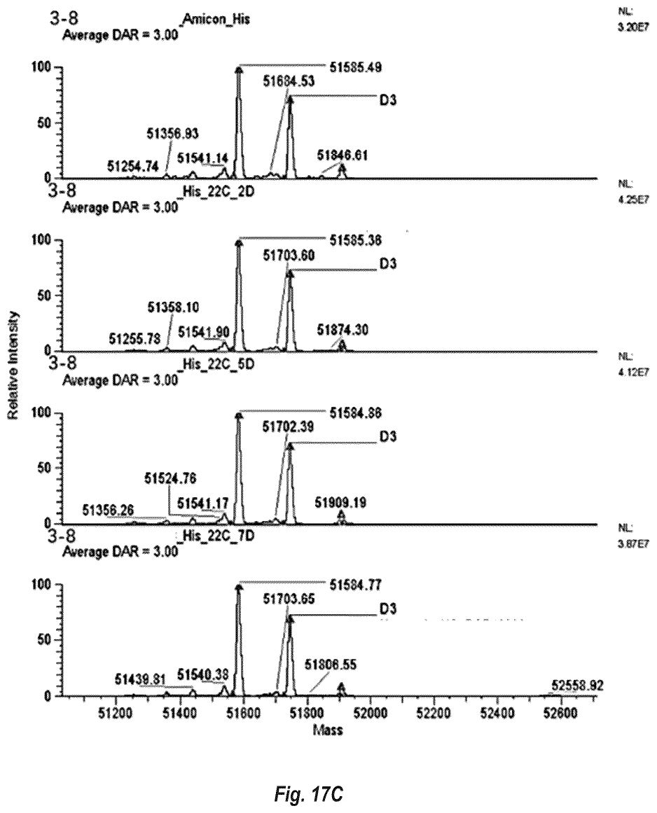

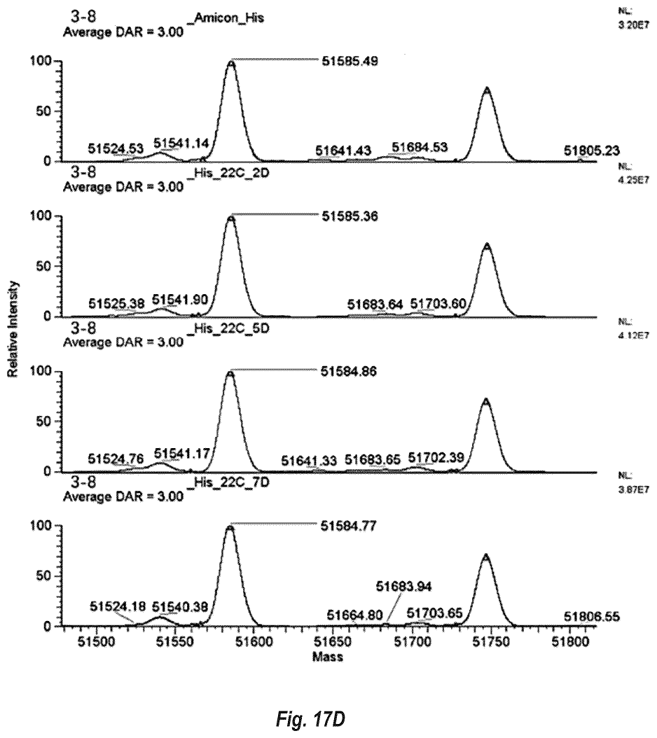

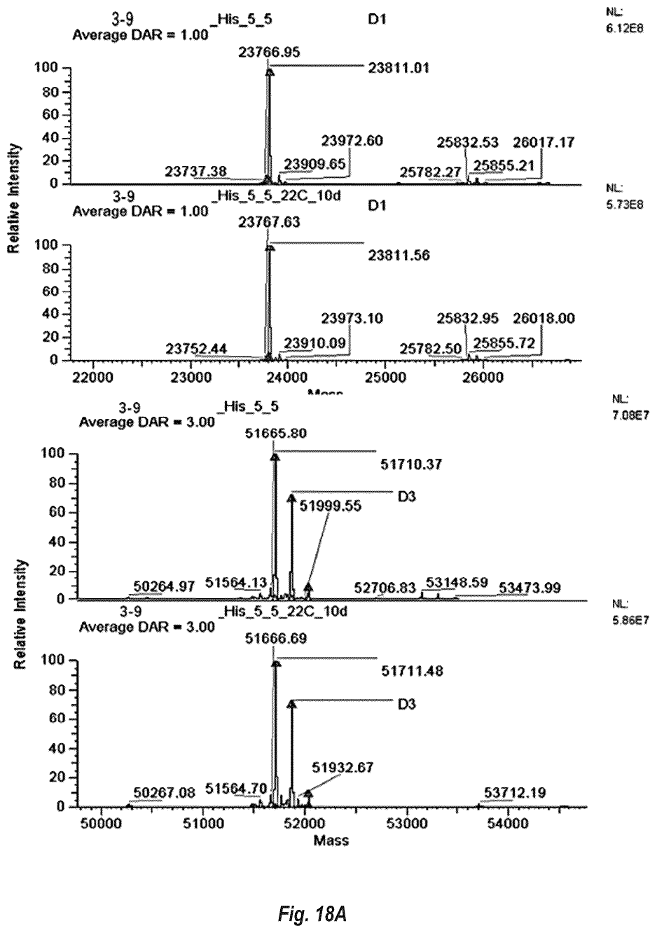

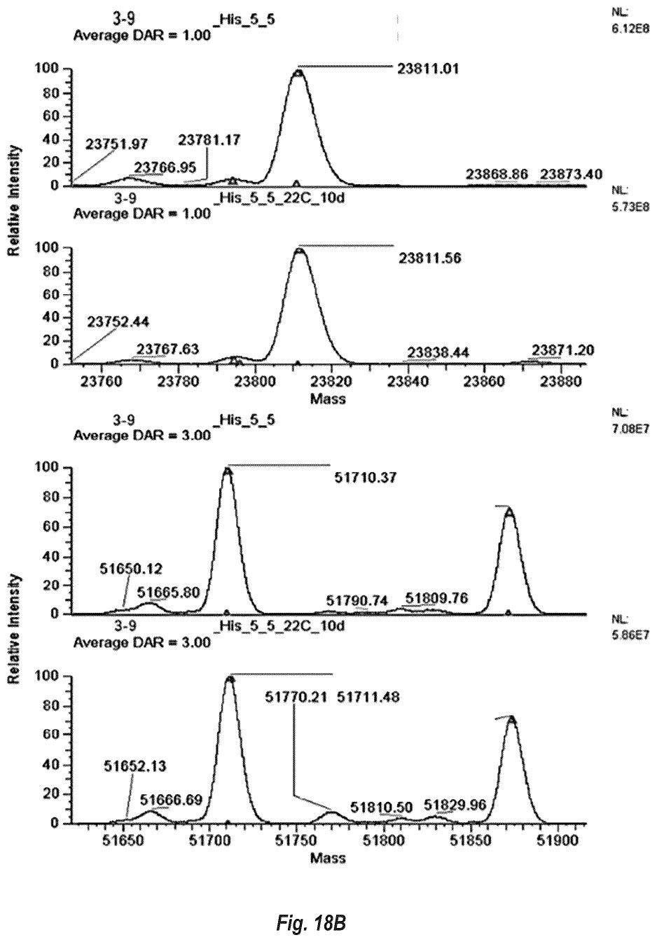

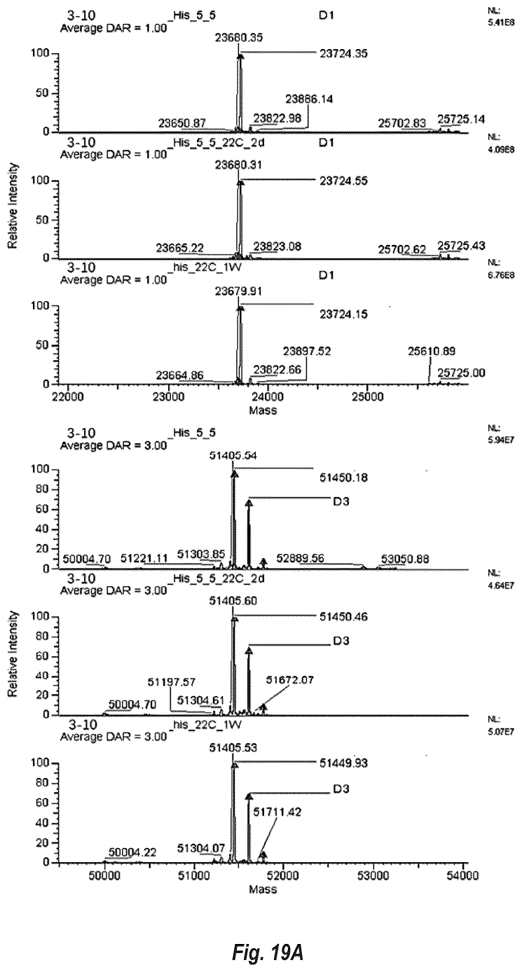

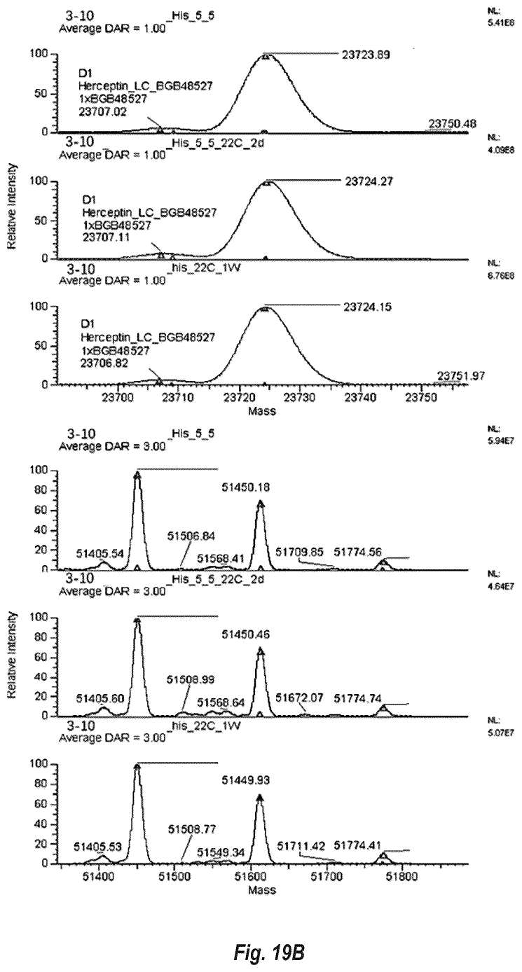

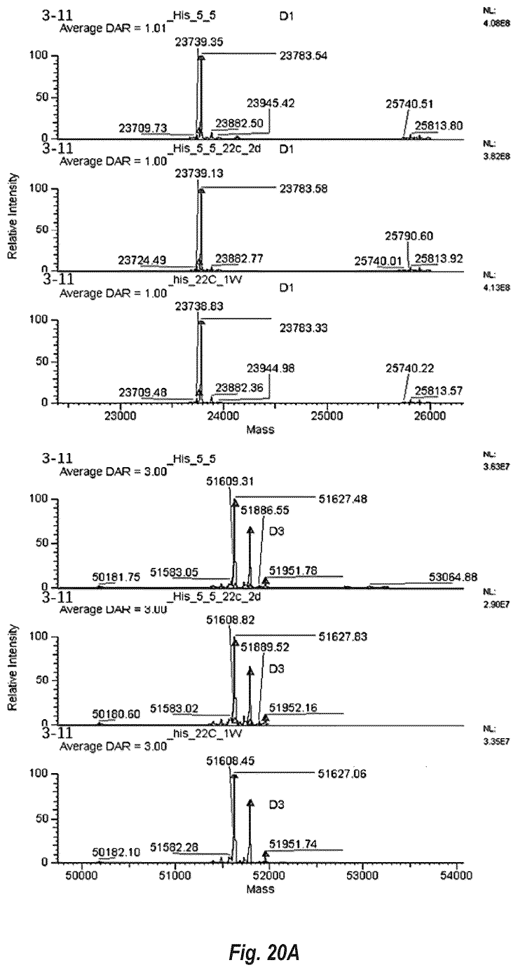

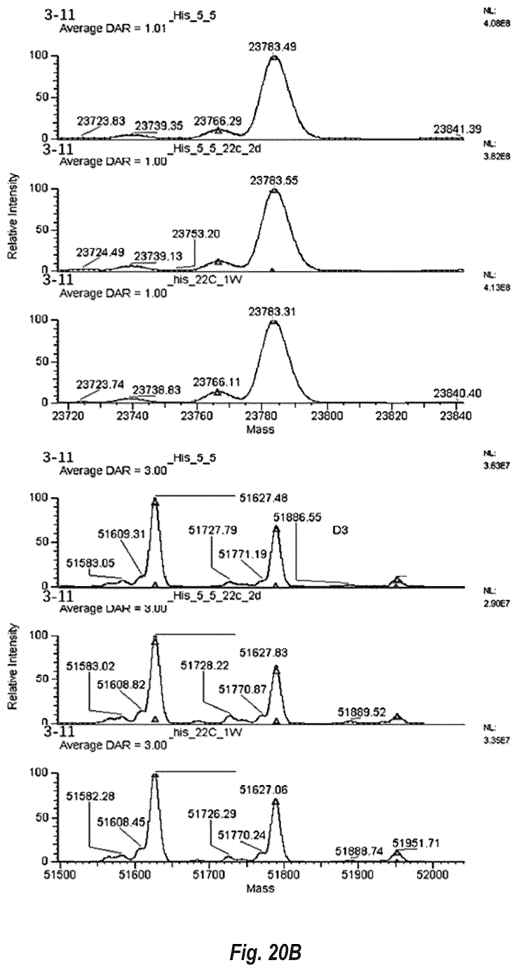

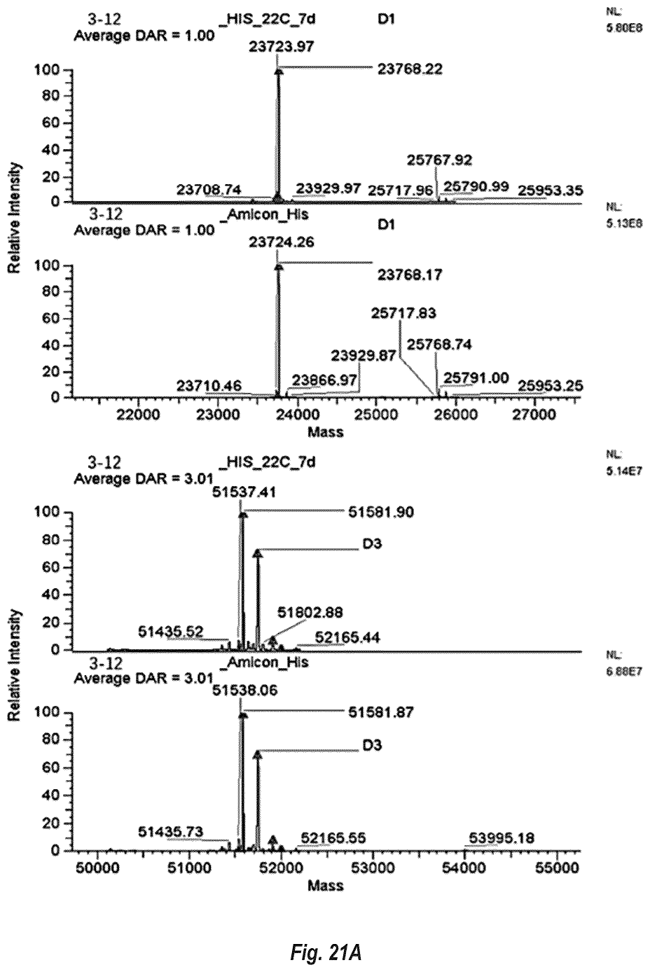

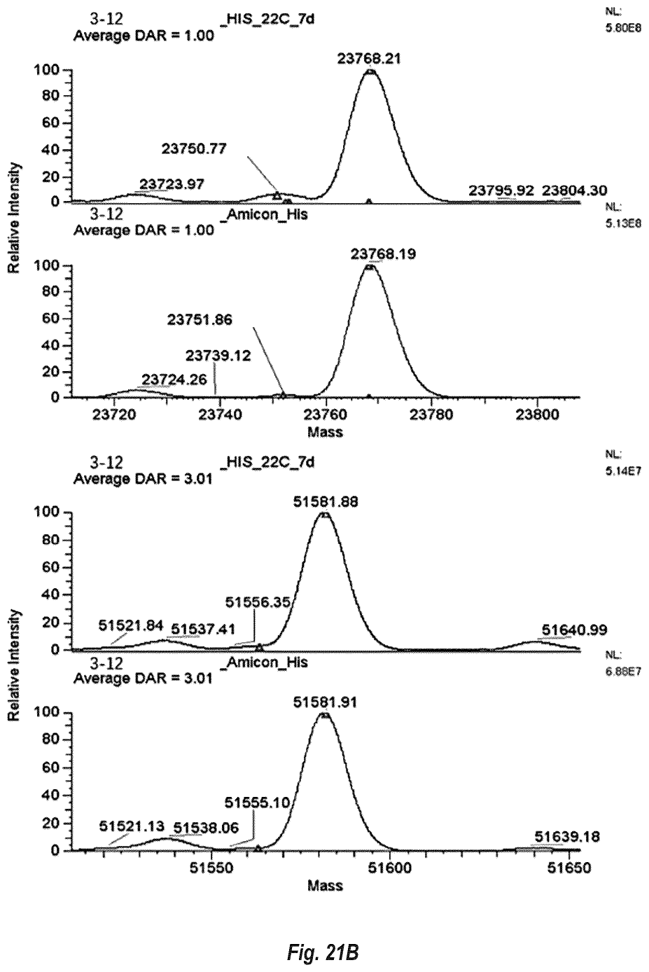

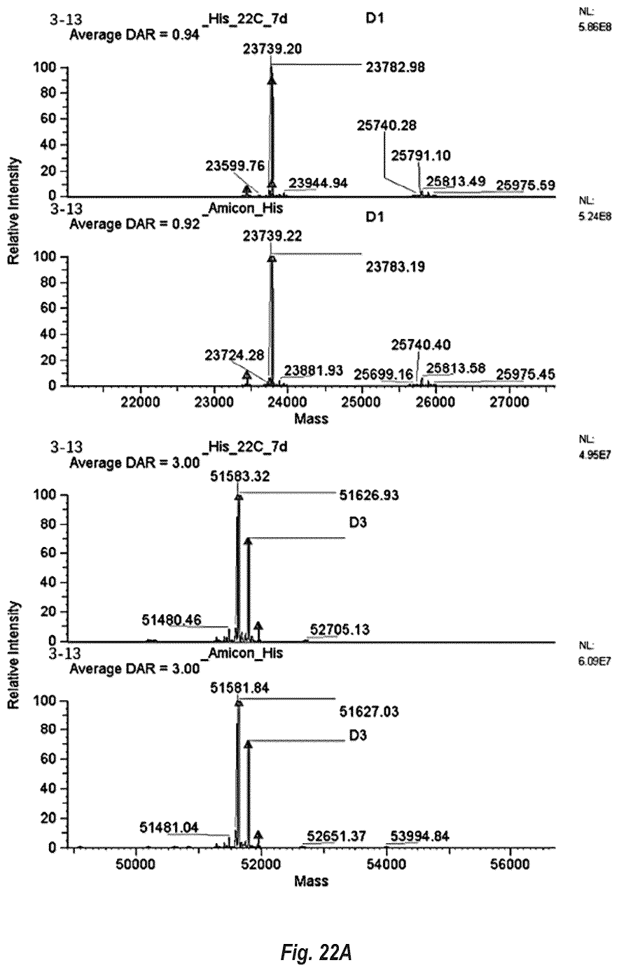

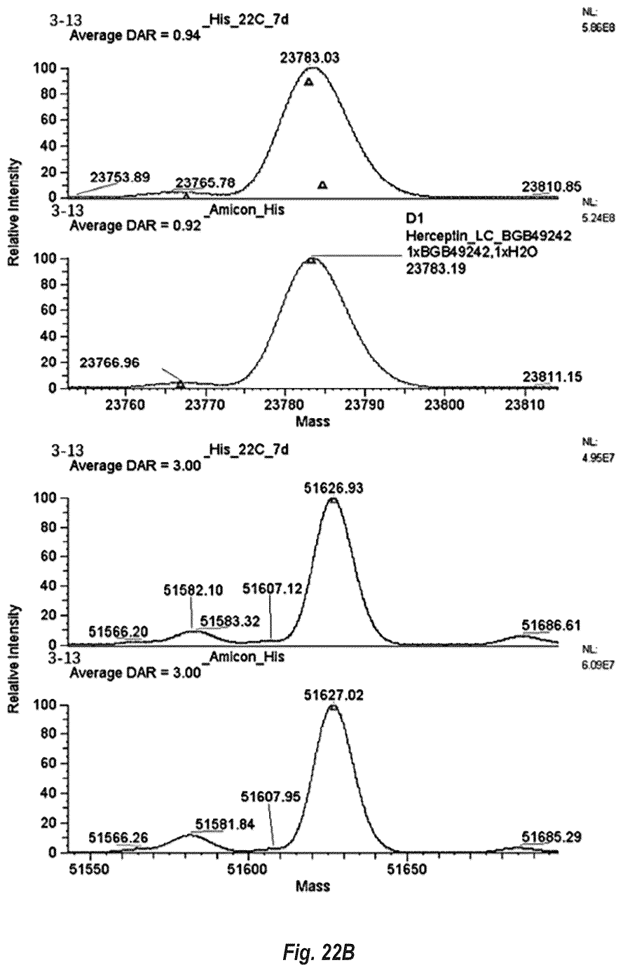

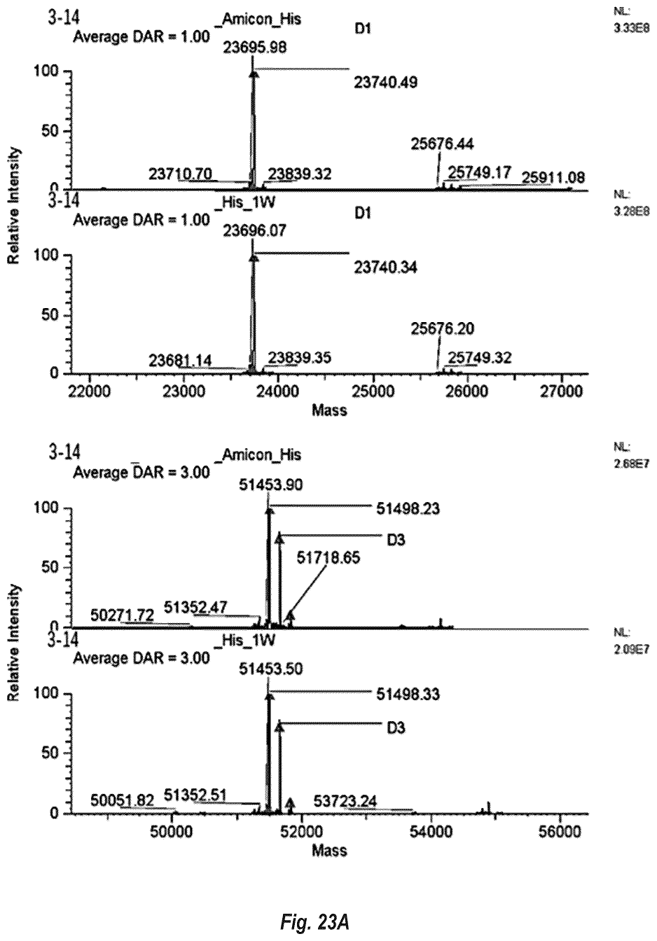

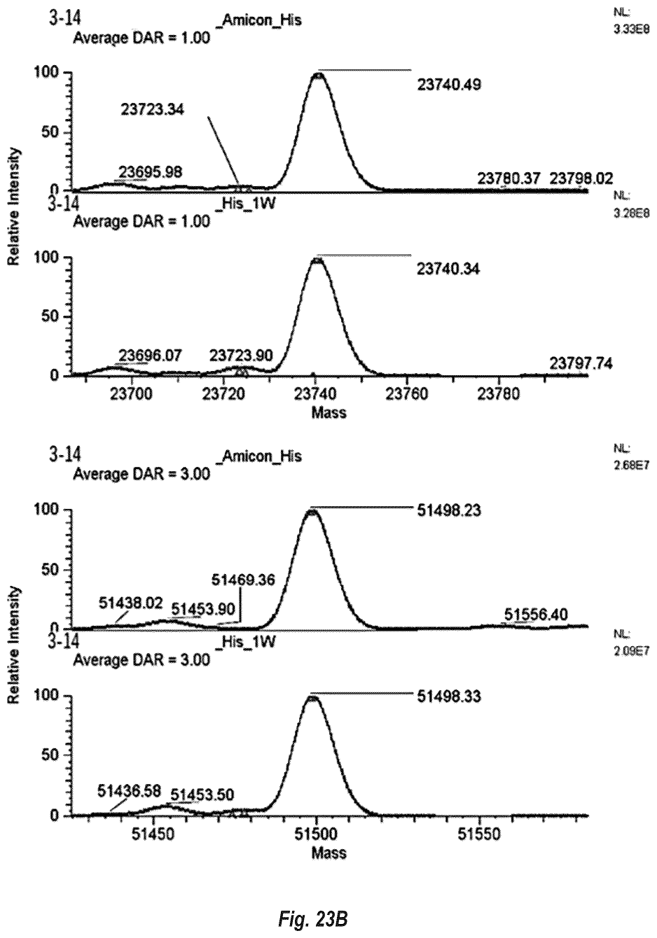

After the conjugation and hydrolysis steps described above (see “DAR8 antibody drug conjugate/conjugator-antibody conjugate preparation”), conjugator-antibody conjugates were incubated in formulation buffer (pH 5.5 20 mM histidine buffer) or glutathione (GSH) buffer (pH 7.4 or 8.0), and the solutions were left at 22 or 37° C. for 1~168 h. Hydrolysis status was monitored via reduced LCMS. Results are presented in Table 6, Table 7, and FIGS. 2A-23B.

Stability with GSH

| C_{ADC} (mM) | GSH (mM) | pH | Time (h) | Temp. (° C.) |
|---|---|---|---|---|
| 0.001-0.03 | 10 | 7.4/8.0 | ≥18 | 22/37 |

TABLE 6
Conjugator-antibody stability in pH 7.4
and 8.0 buffer with and without GSH

| Conjugator-Antibody | Incubation | | Temp. | MS |
|---|---|---|---|---|
| Conjugate No. | Time (h) | pH | (° C.) | DAR |
| 3-3 | 20 | 7.4 | 22 | 8.06 |
| | | | 37 | 7.98 |
| | | 8.0 | 22 | 8.02 |
| 3-4 | 20 | 7.4 | 22 | 8.00 |
| | | | 37 | 8.00 |
| | | 8.0 | 22 | 8.00 |
| 3-5 | 21 | 7.4 | 22 | 8.00 |
| | | | 37 | 8.00 |
| | | 8.0 | 22 | 8.00 |
| 3-6 | 20 | 7.4 | 22 | 8.00 |
| | | | 37 | 8.00 |
| | | 8.0 | 22 | 8.00 |
| 3-7 | 20 | 7.4 | 22 | 8.00 |
| | | | 37 | 8.00 |
| | | 8.0 | 22 | 8.00 |
| 3-8 | 20 | 7.4 | 22 | 7.92 |
| | | | 37 | 7.92 |
| | | 8.0 | 22 | 7.88 |
| 3-9 | 20 | 7.4 | 22 | 8.00 |
| | | | 37 | 8.00 |
| | | 8.0 | 22 | 8.00 |
| 3-10 | 20 | 7.4 | 22 | 8.00 |
| | | | 37 | 8.00 |
| | | 8.0 | 22 | 8.00 |
| 3-11 | 20 | 7.4 | 22 | 8.00 |
| | | | 37 | 8.00 |
| | | 8.0 | 22 | 8.00 |
| 3-12 | 24 | 7.4 | 22 | 8.00 |
| | | | 37 | 8.00 |
| | | 8.0 | 22 | 8.00 |
| 3-13 | 24 | 7.4 | 22 | 7.90 |
| | | | 37 | 7.90 |
| | | 8.0 | 22 | 7.90 |
| 3-14 | 24 | 7.4 | 22 | 7.98 |
| | | | 37 | 8.00 |
| | | 8.0 | 22 | 8.00 |

The results demonstrate that DAR remained at about 8.0 for all conjugator-antibody conjugates when incubated in buffer either with or without GSH for 18 hours.

TABLE 7
Conjugator-antibody stability in formulation buffer (pH 5.5 20 mM histidine buffer)

| | | | | | | Maleimide ring |
|---|---|---|---|---|---|---|
| Conjugator-Antibody | Incubation | | Temp. | MS DAR- | MS DAR- | opening ratio (%) at |
| Conjugate No. | Time (h) | pH | (° C.) | T = 0 h | T = 168 h | T = 168 h |
| 3-3 | 168 | 5.5 | 22 | 7.92 | 7.92 | 96.40 |
| 3-4 | 168 | 5.5 | 22 | 8.00 | 8.00 | 98.86 |
| 3-6 | 168 | 5.5 | 22 | 8.00 | 8.00 | 97.04 |
| 3-7 | 168 | 5.5 | 22 | 8.00 | 8.00 | 97.99 |
| 3-8 | 168 | 5.5 | 22 | 7.76 | 7.80 | 95.50 |
| 3-9 | 240 | 5.5 | 22 | 8.00 | 8.00 | 98.51 |
| 3-10 | 168 | 5.5 | 22 | 8.00 | 8.00 | 98.38 |
| 3-11 | 168 | 5.5 | 22 | 8.02 | 8.00 | 96.99 |
| 3-12 | 168 | 5.5 | 22 | 8.02 | 8.02 | 97.83 |
| 3-13 | 168 | 5.5 | 22 | 7.84 | 7.88 | 98.33 |
| 3-14 | 168 | 5.5 | 22 | 8.00 | 8.00 | 97.15 |

Results presented in Table 7 demonstrate that the disclosed conjugator-antibody conjugates maintain their hydrolyzed form at >96% after 168 hours in acidic formulation buffer.

ADC: Maleimide Hydrolysis after Conjugation

After maleimide hydrolysis, ADCs 4-4 and 4-5 (see Table 4) easily adhered to Slide-A-Lyzer™ dialysis kits or ultrafiltration Amicon® membranes during the buffer exchange process into formulation buffer, which led to low recovery rates (<40%). Without being limited to any mechanism or mode of action, it was suspected that maleimide hydrolysis created an additional 8 negative charges and may have caused changes to the ADCs' physical properties changes such that they became sticky on the purification columns and ultrafiltration Amicon® membranes. ADCs 4-7~4-16 had >80% recovery during purification and no adhesion behavior on Slide-A-Lyzer™ dialysis kits or ultrafiltration Amicon® membranes was observed.

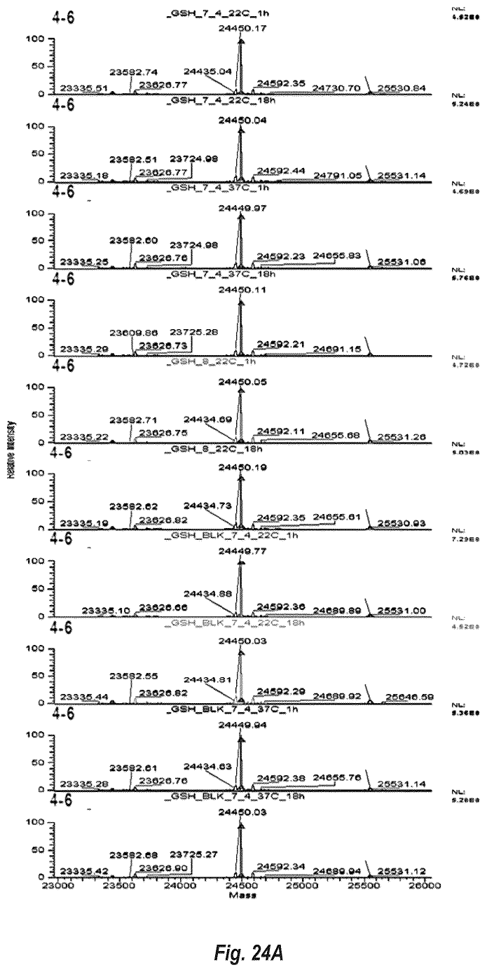

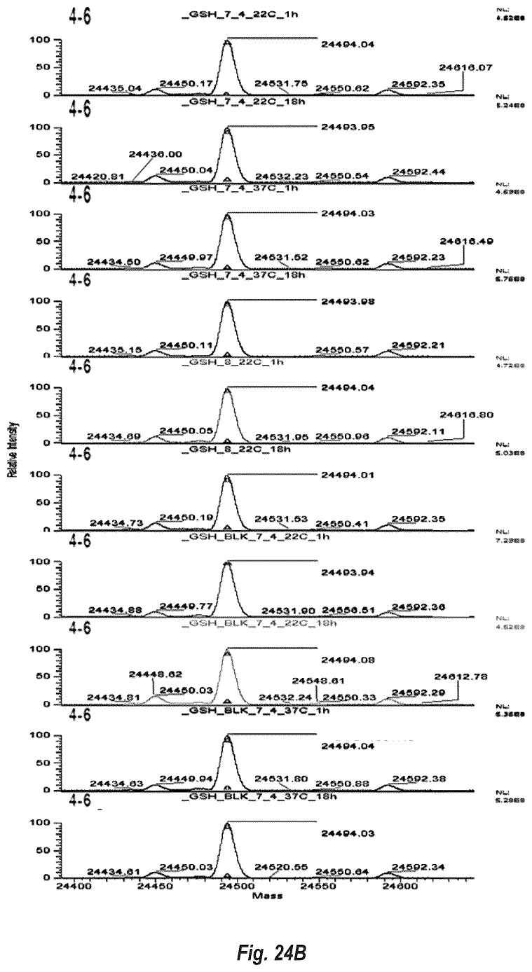

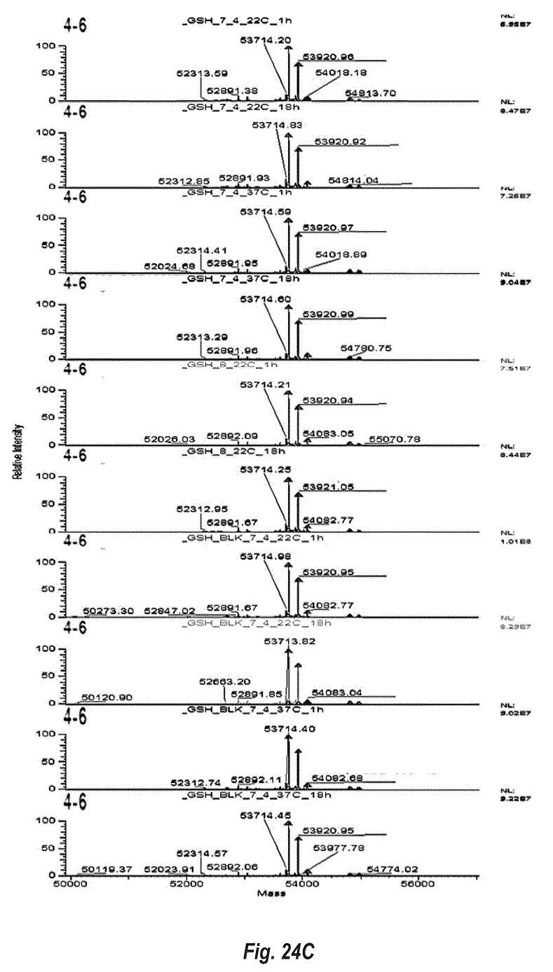

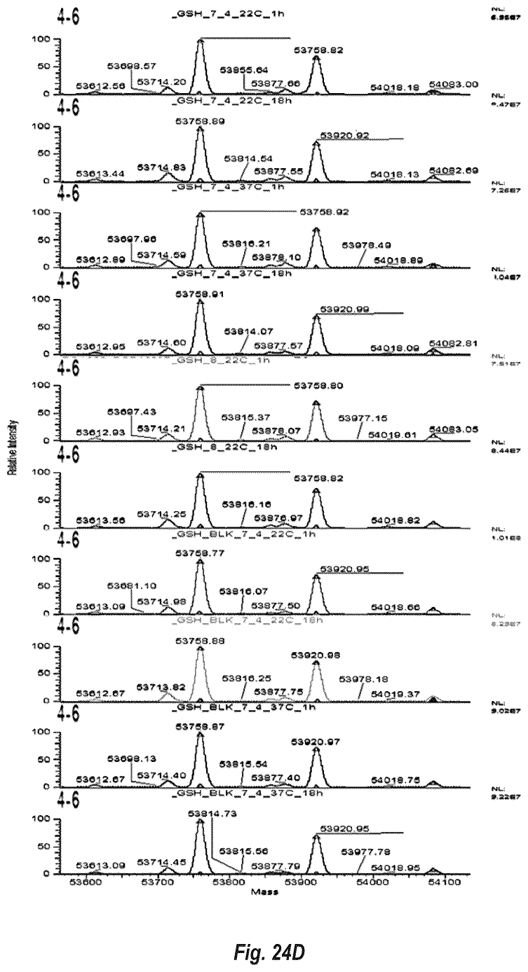

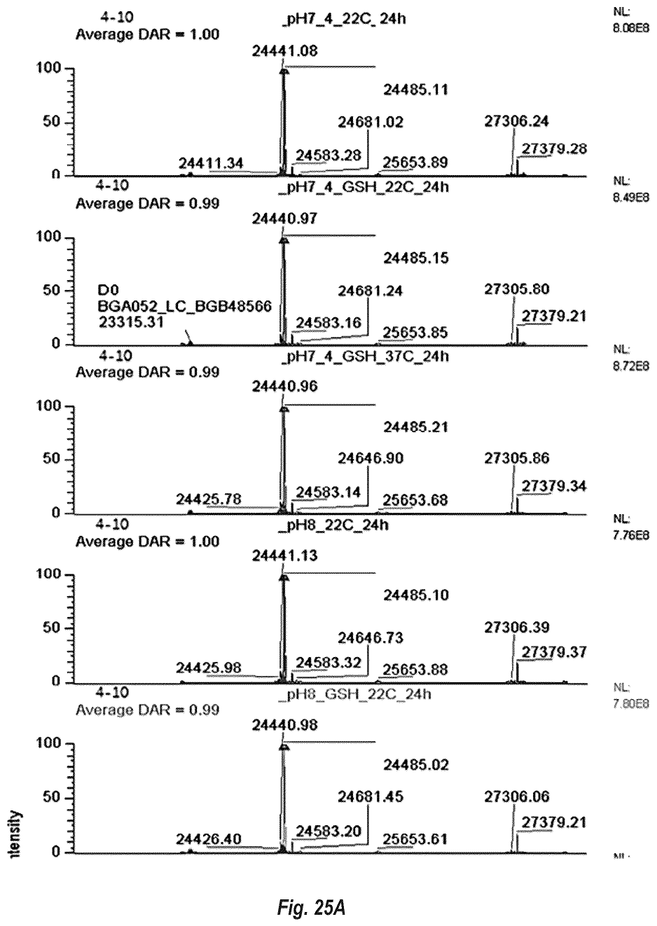

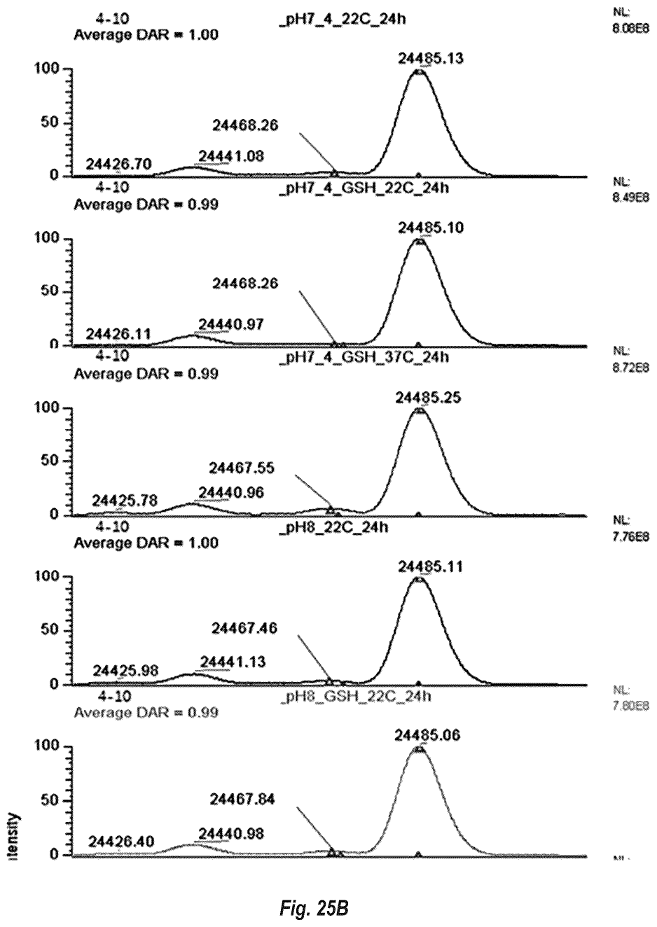

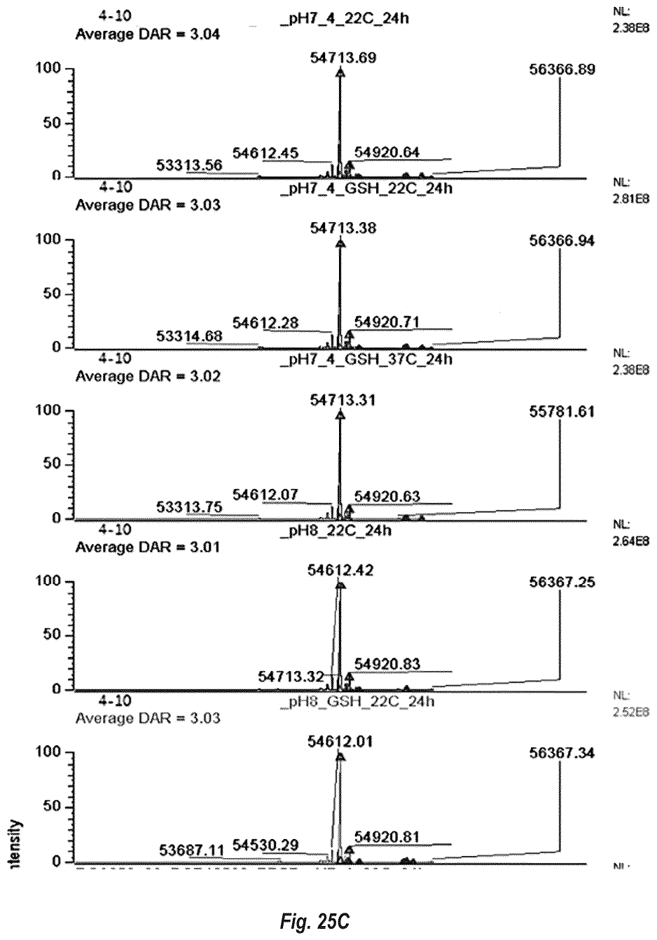

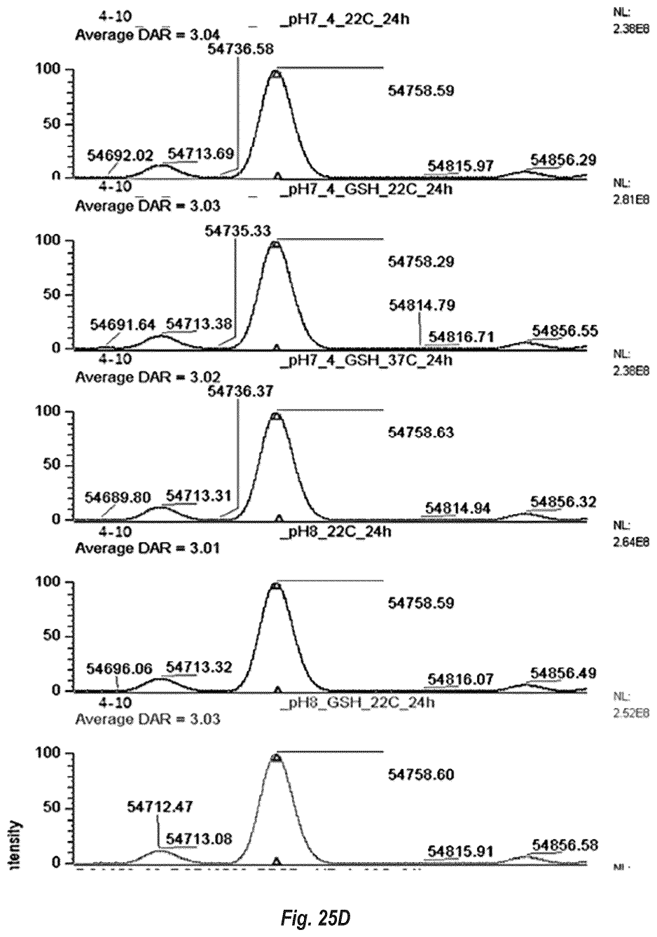

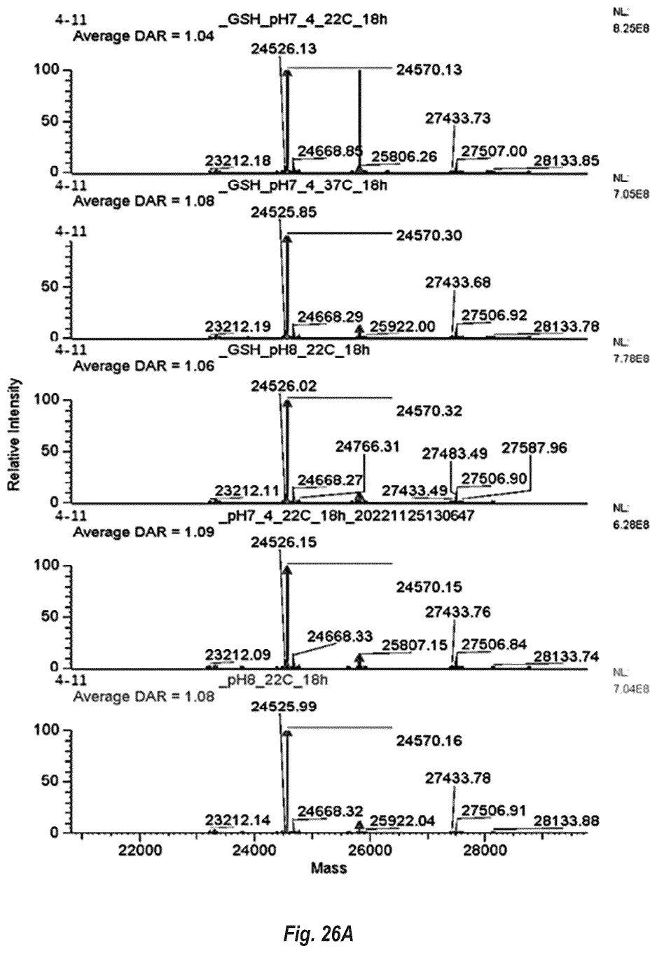

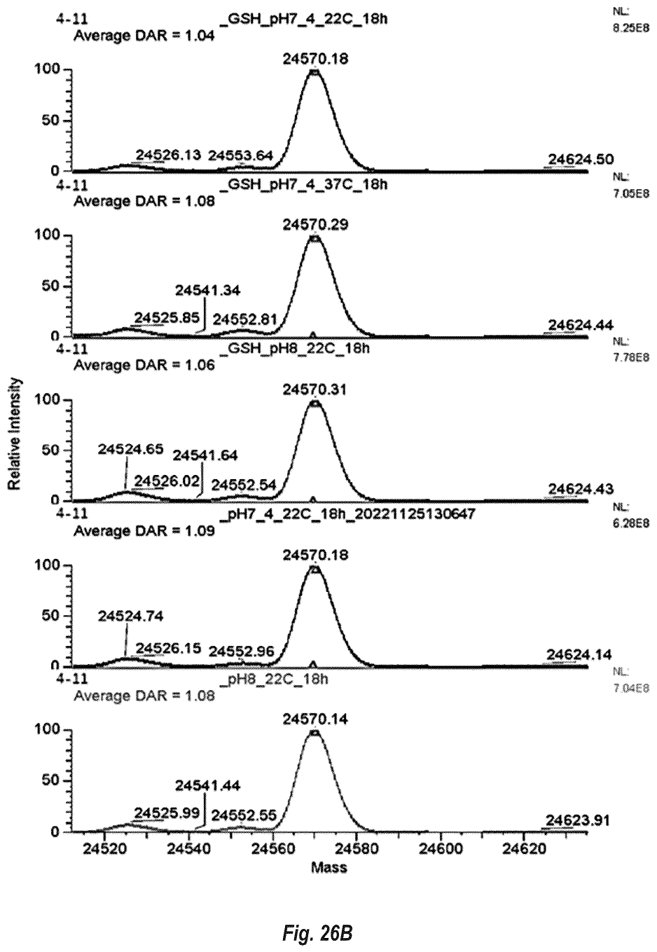

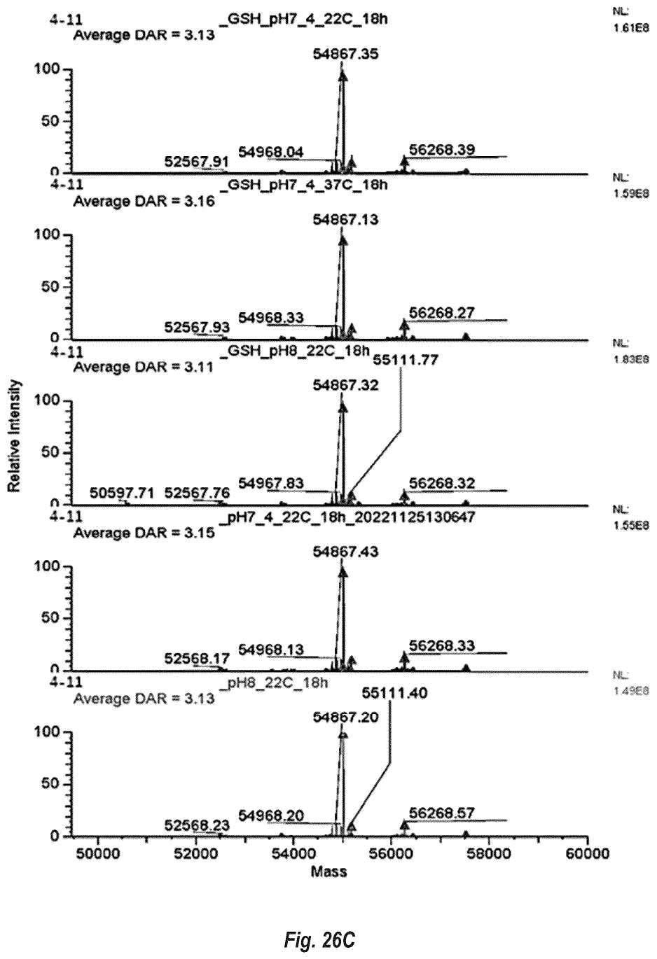

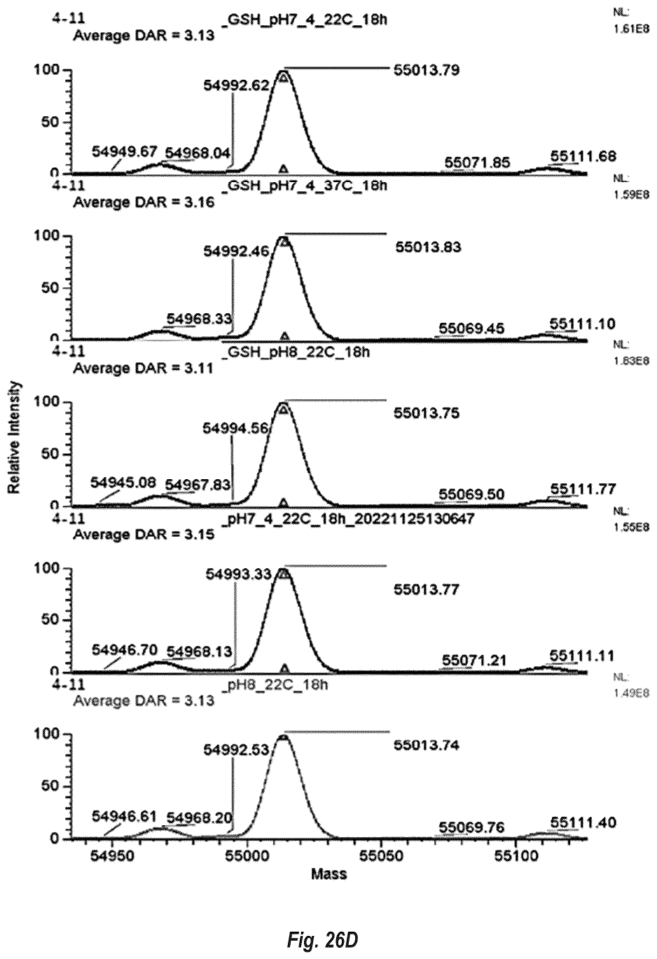

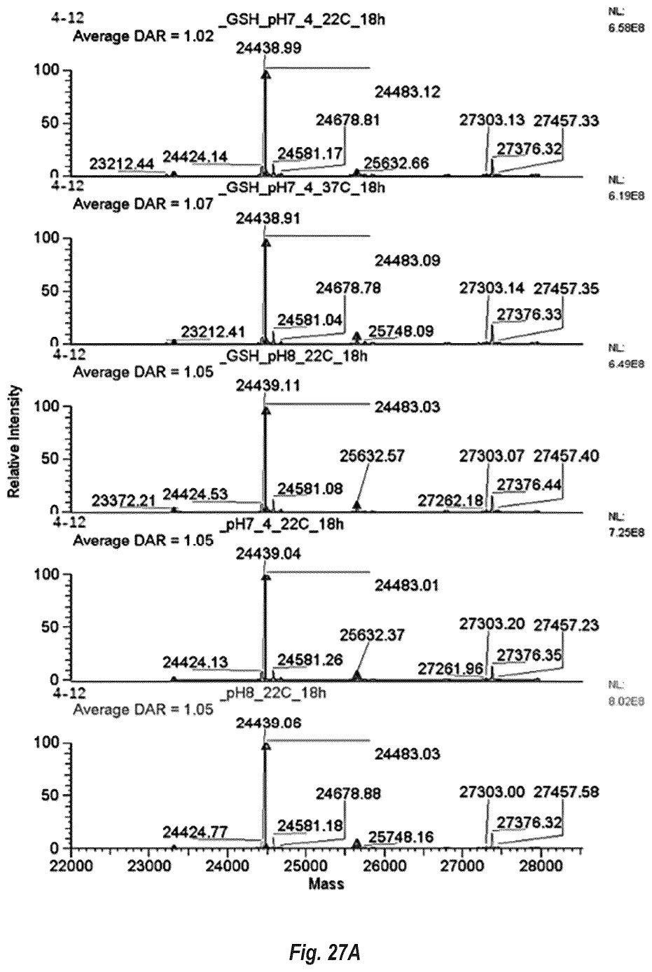

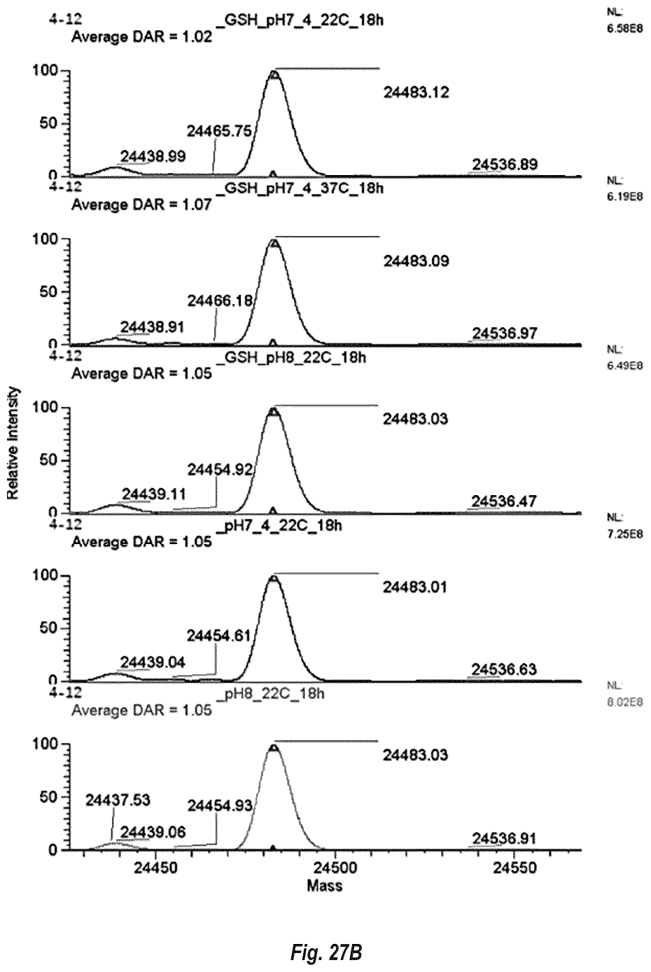

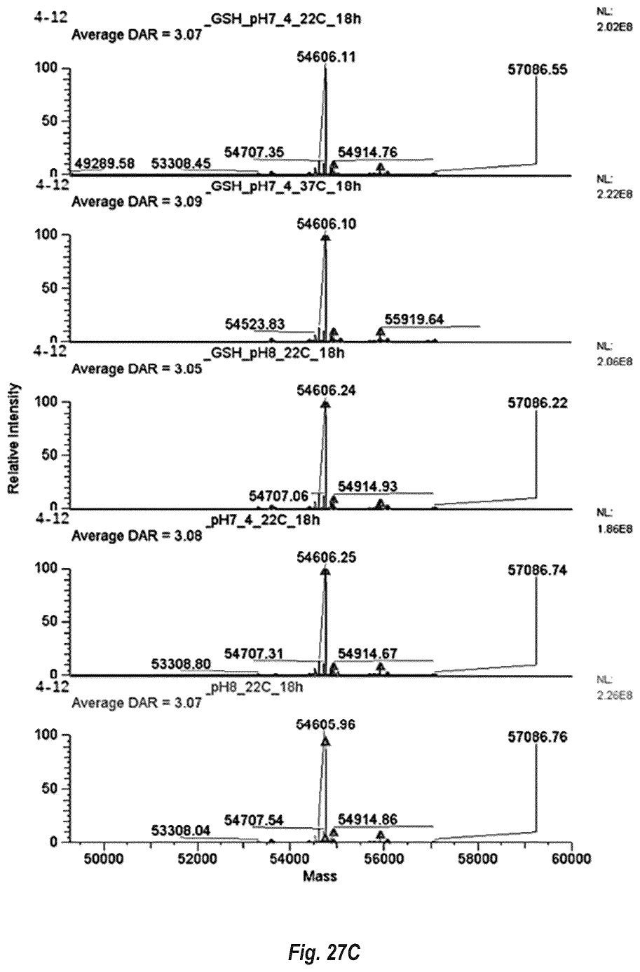

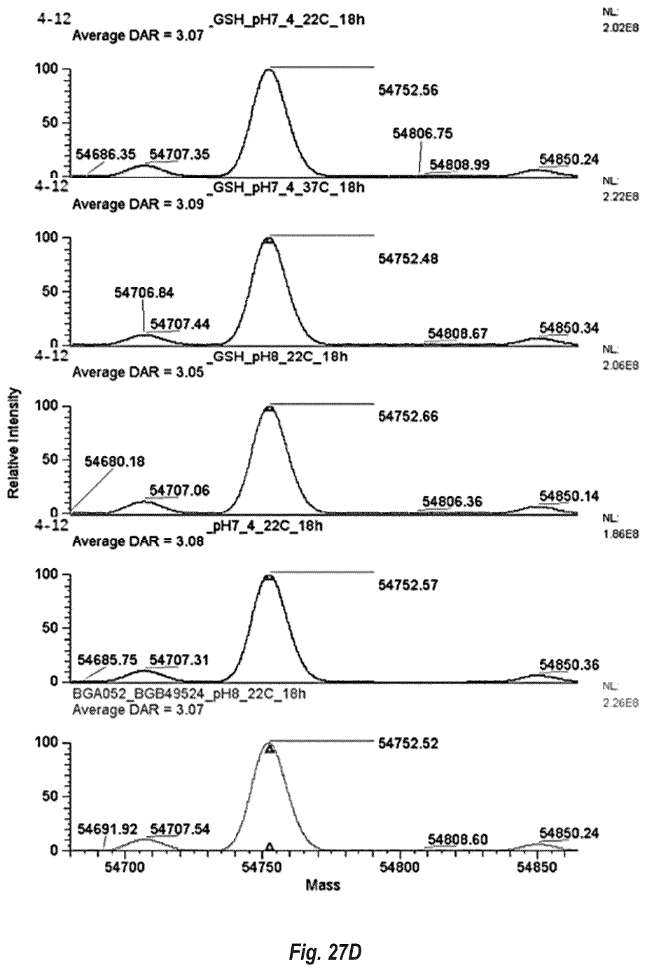

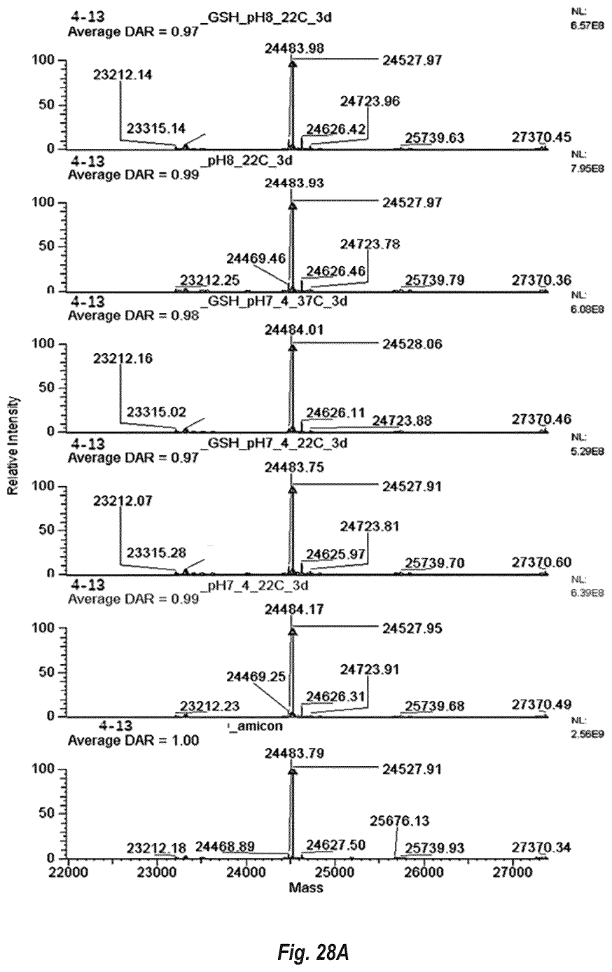

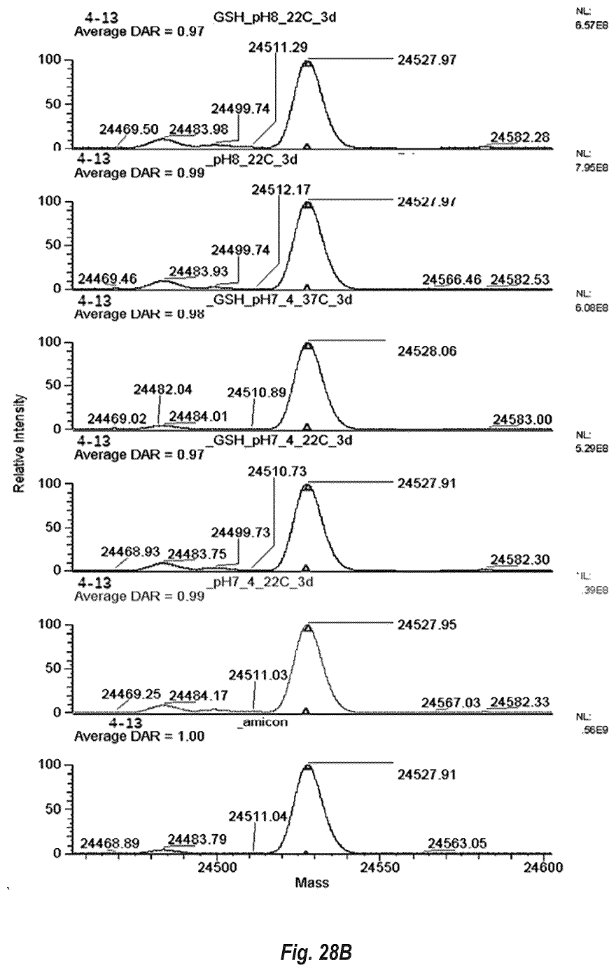

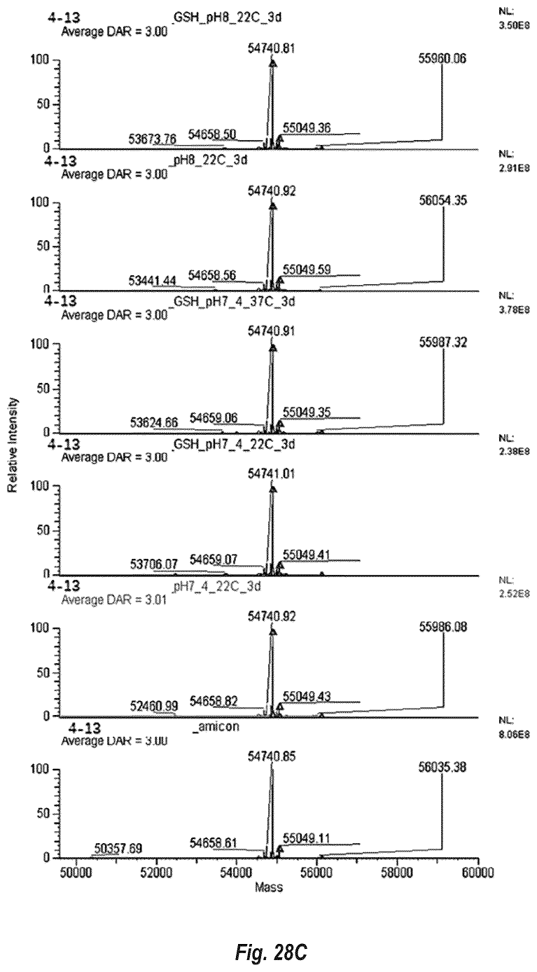

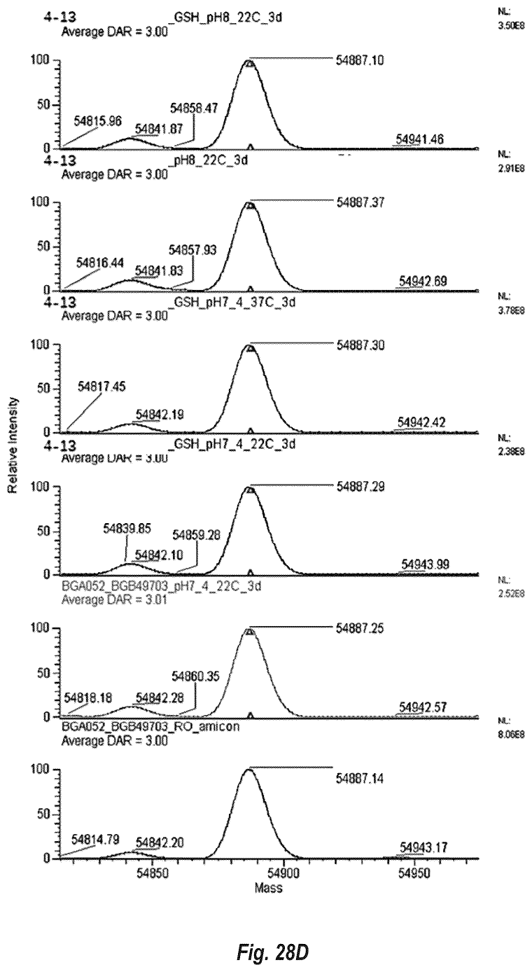

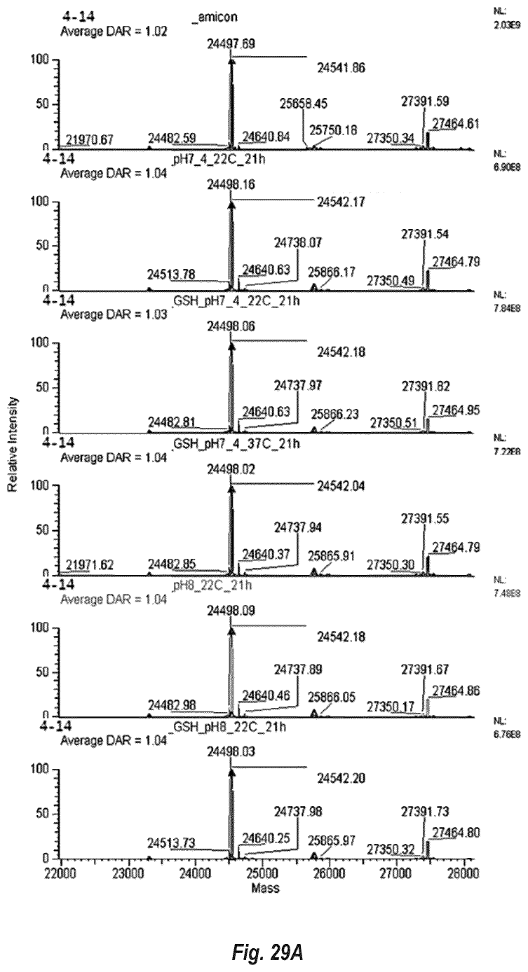

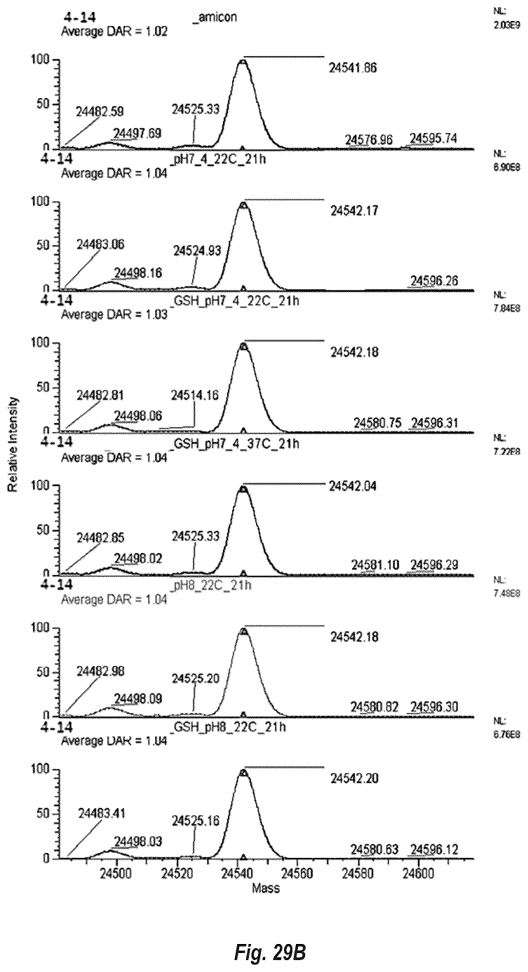

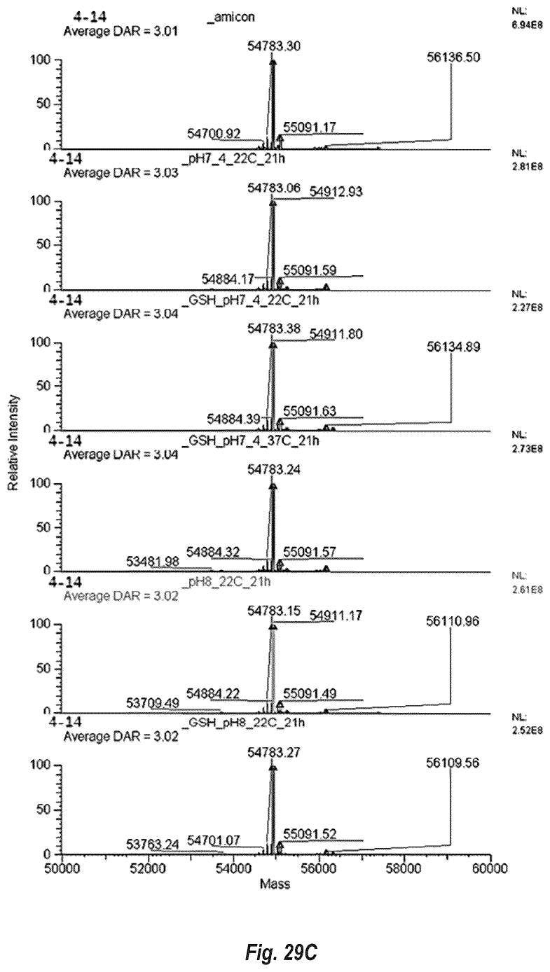

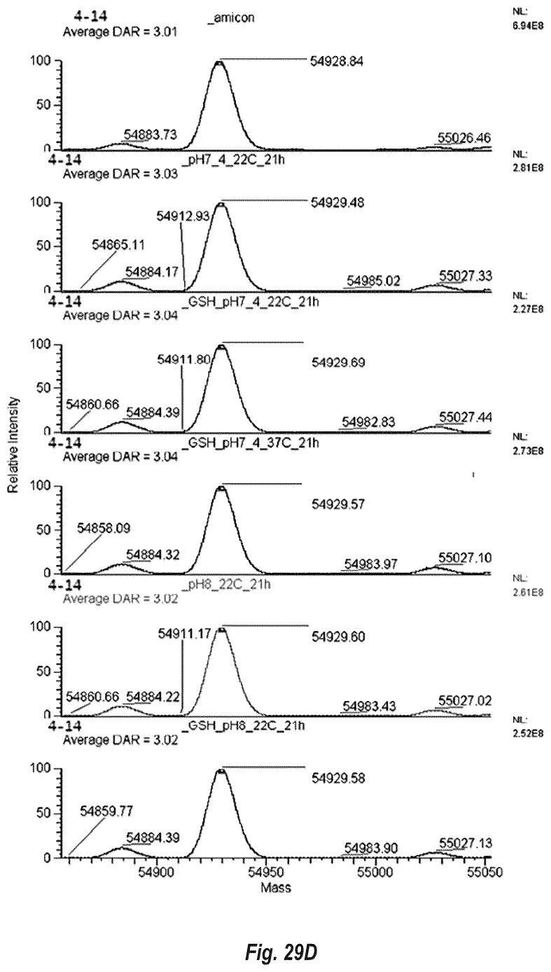

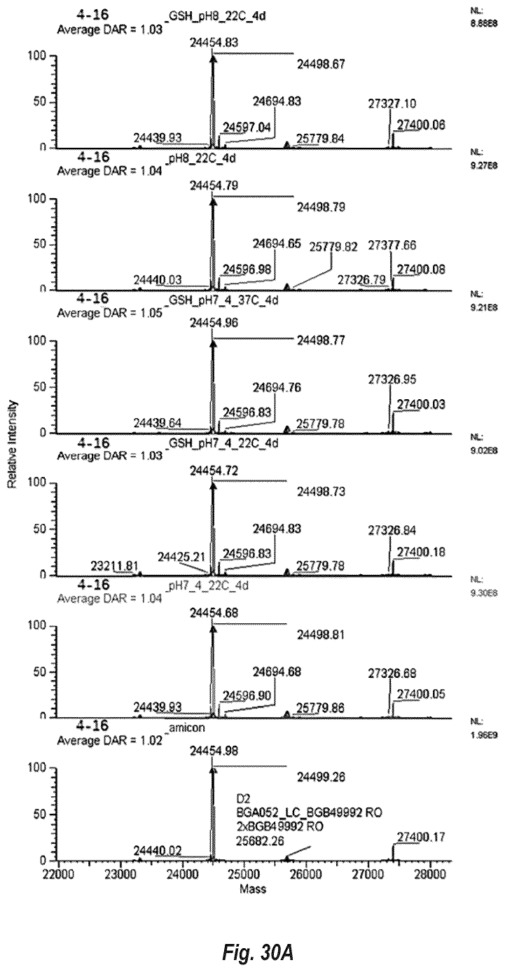

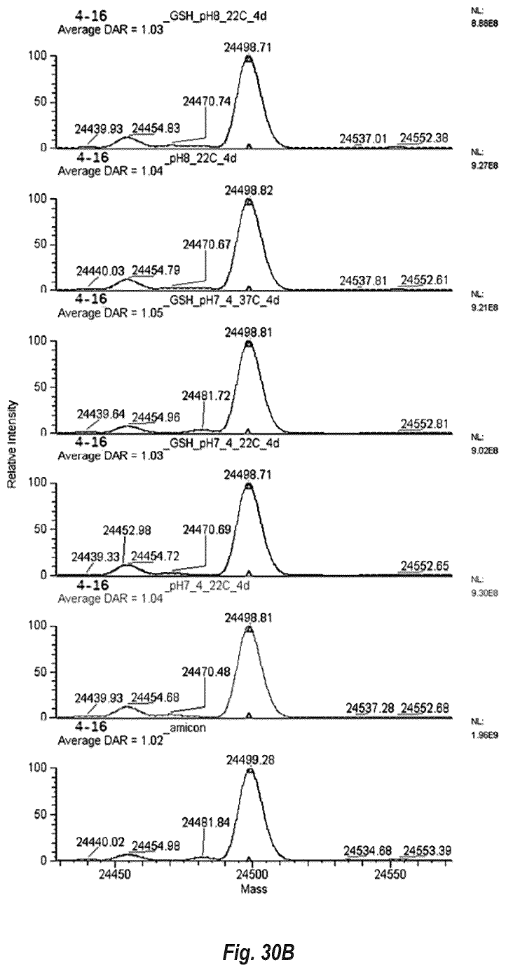

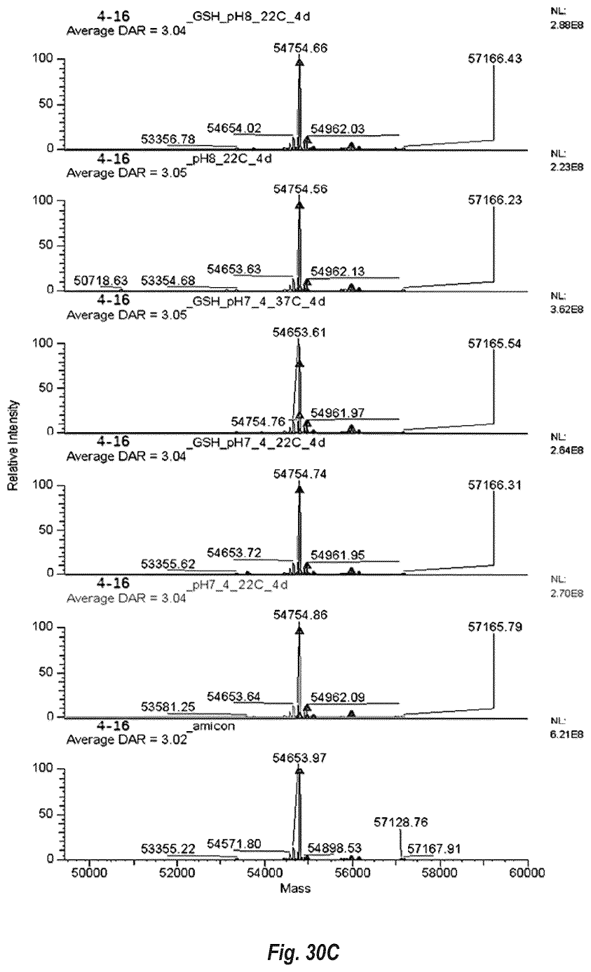

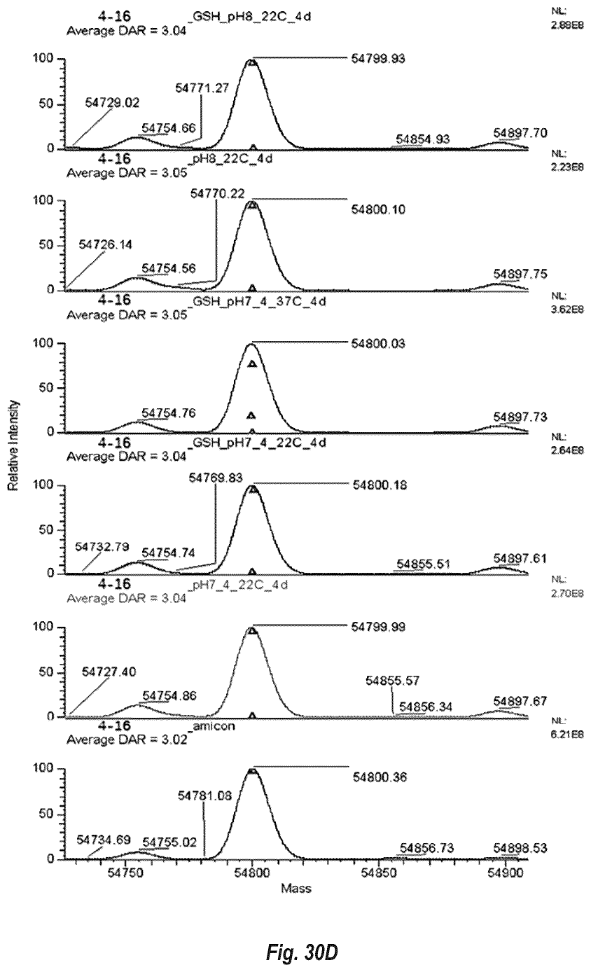

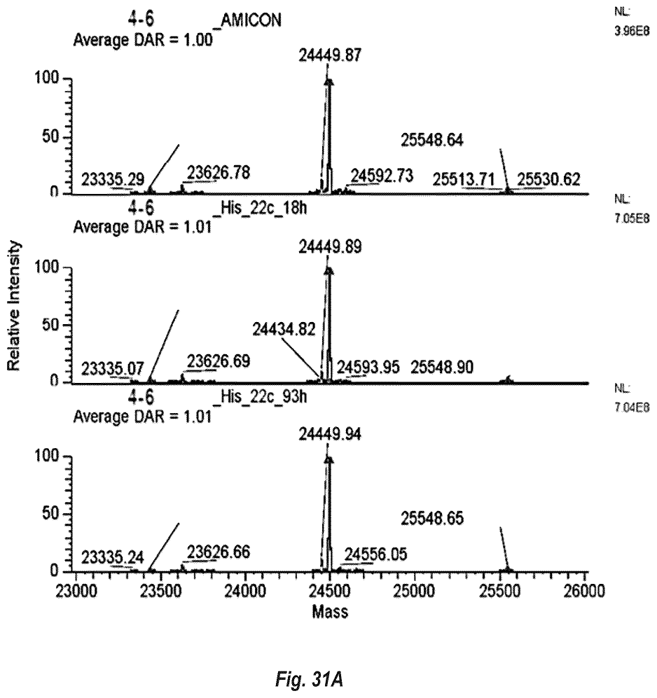

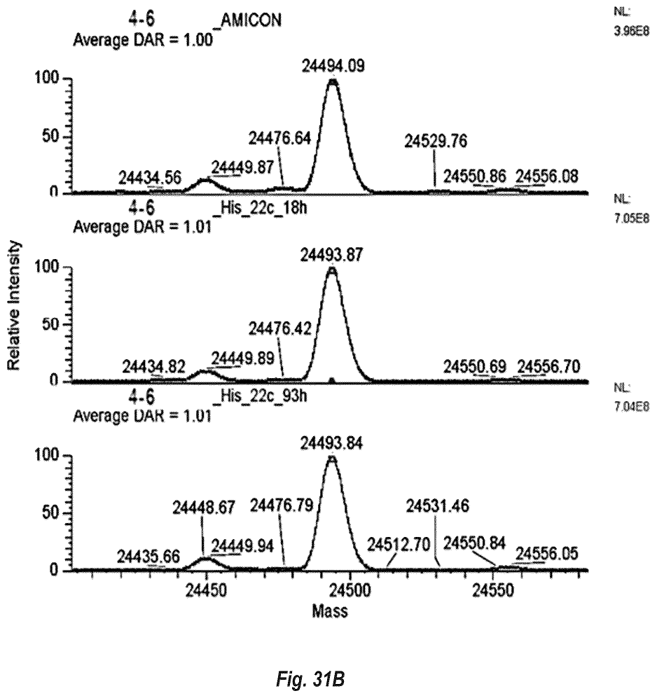

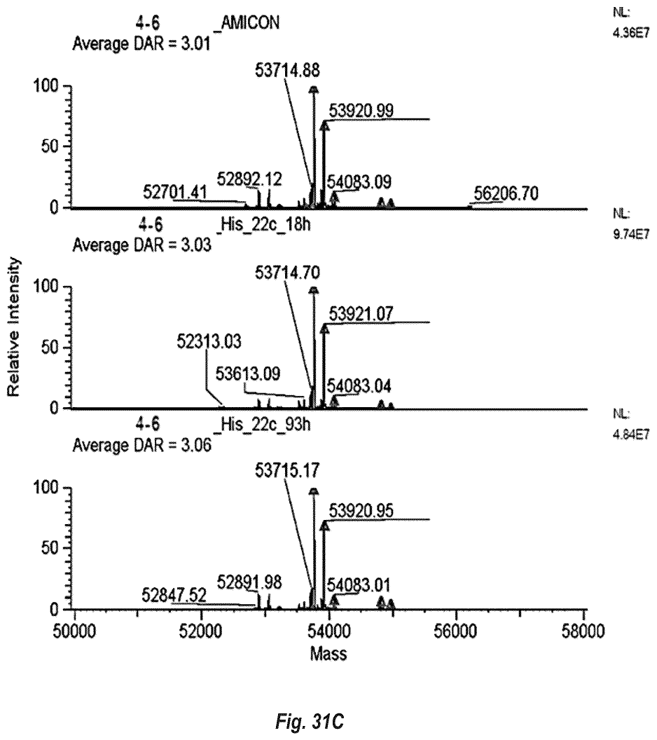

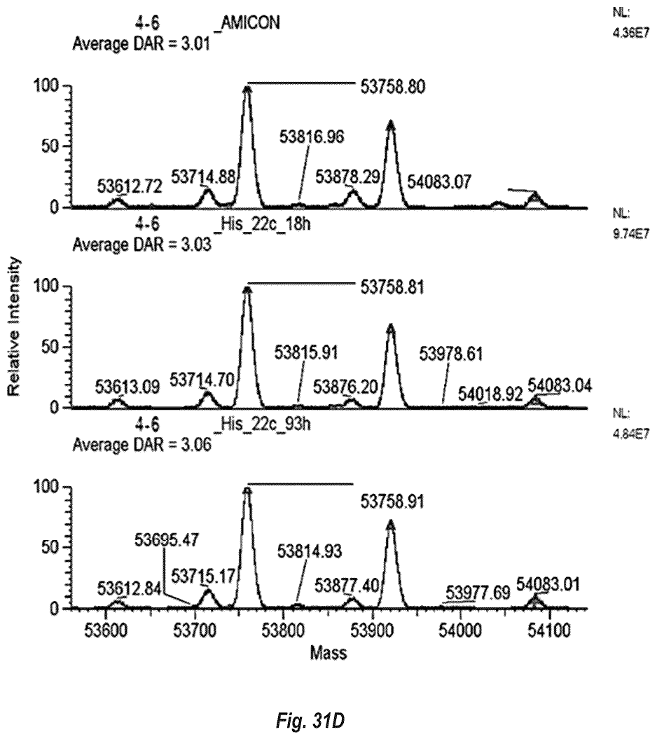

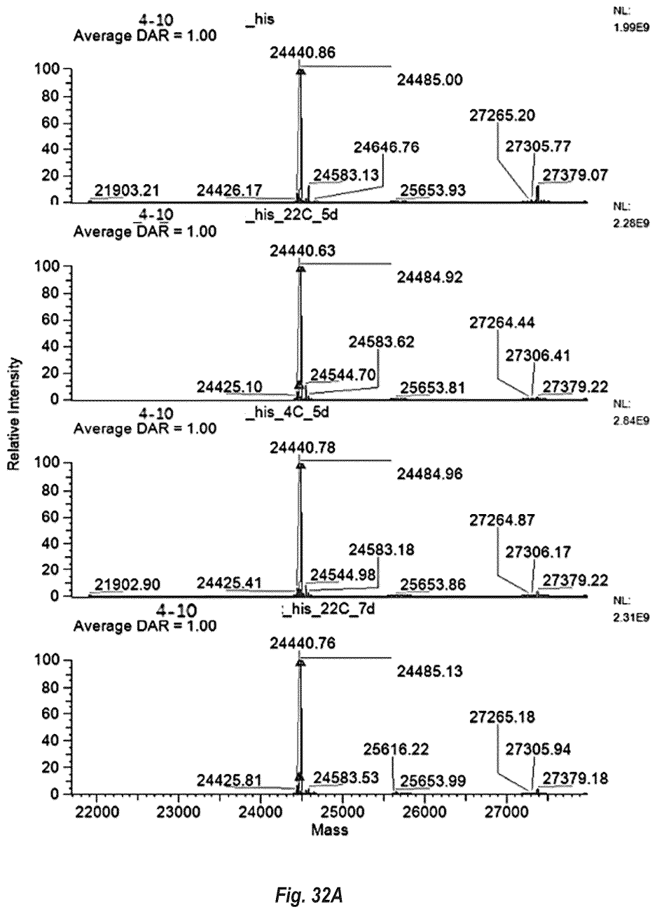

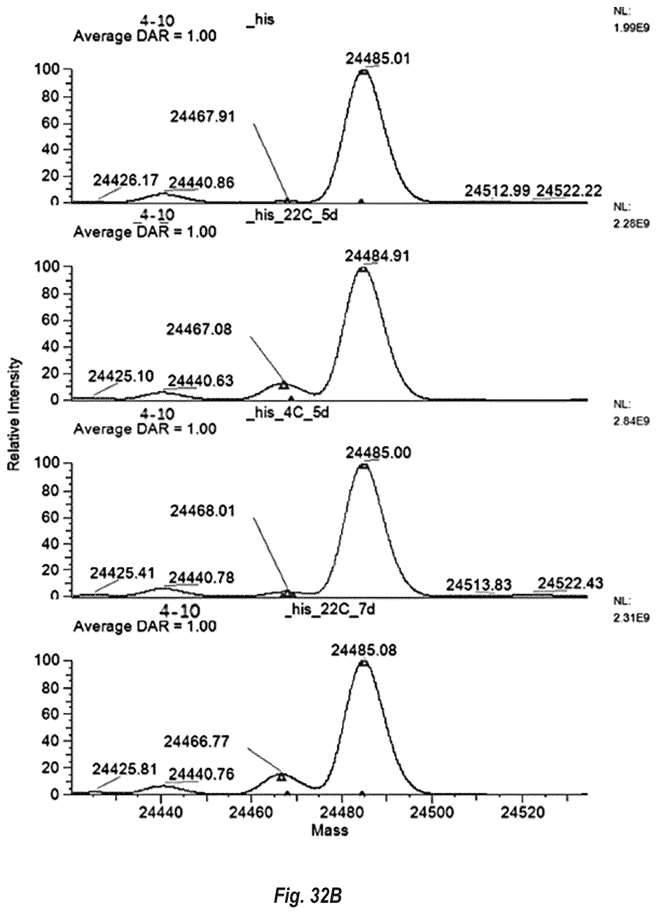

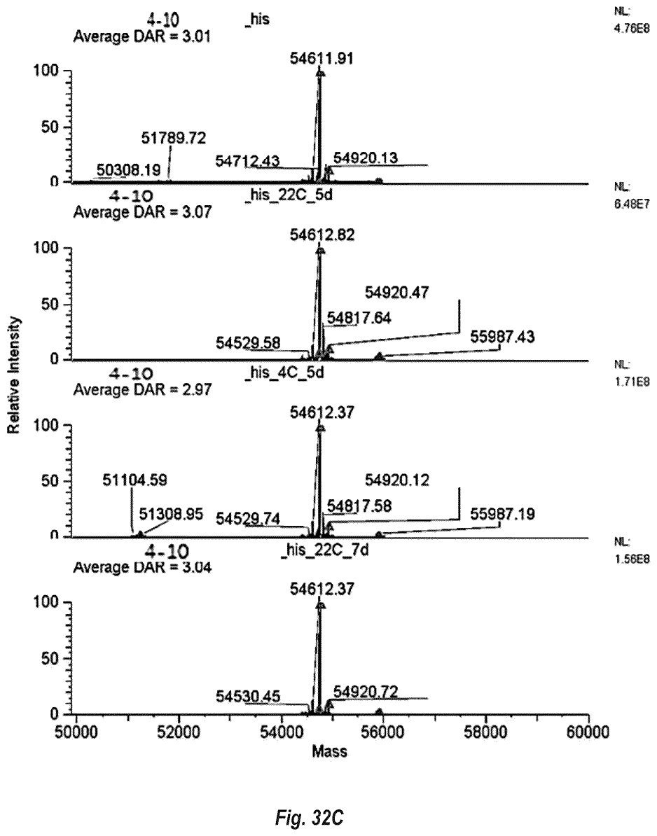

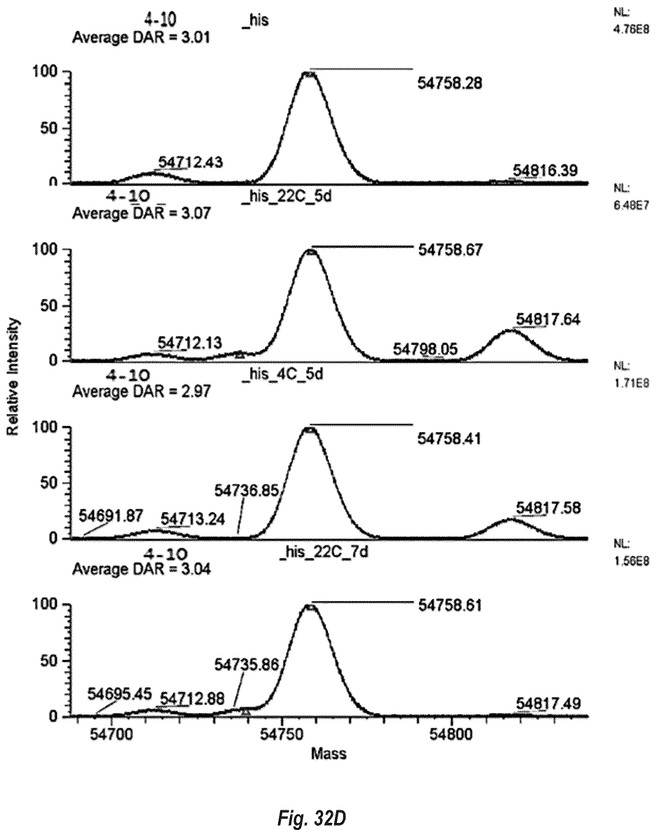

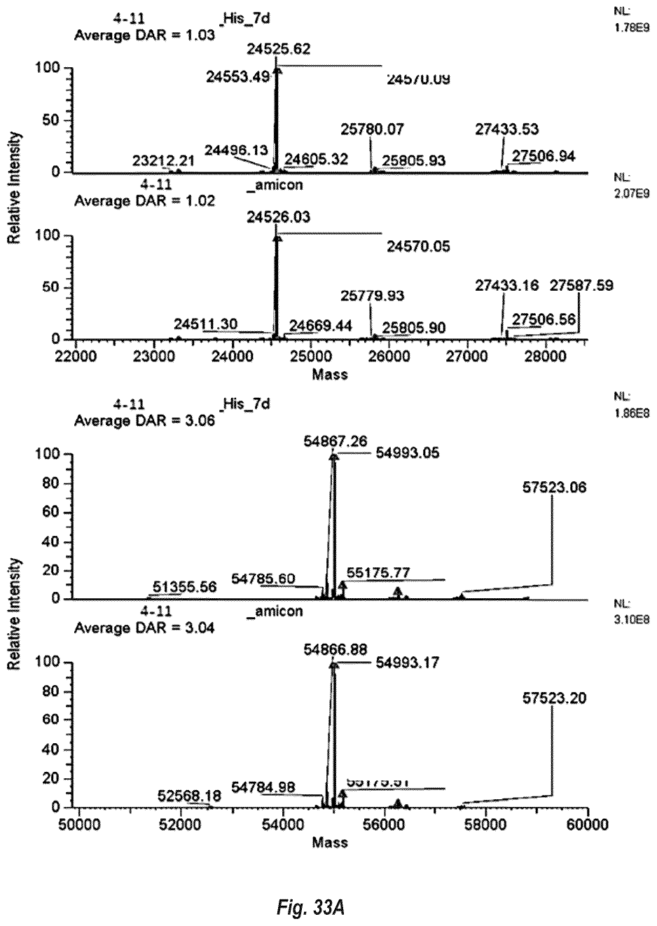

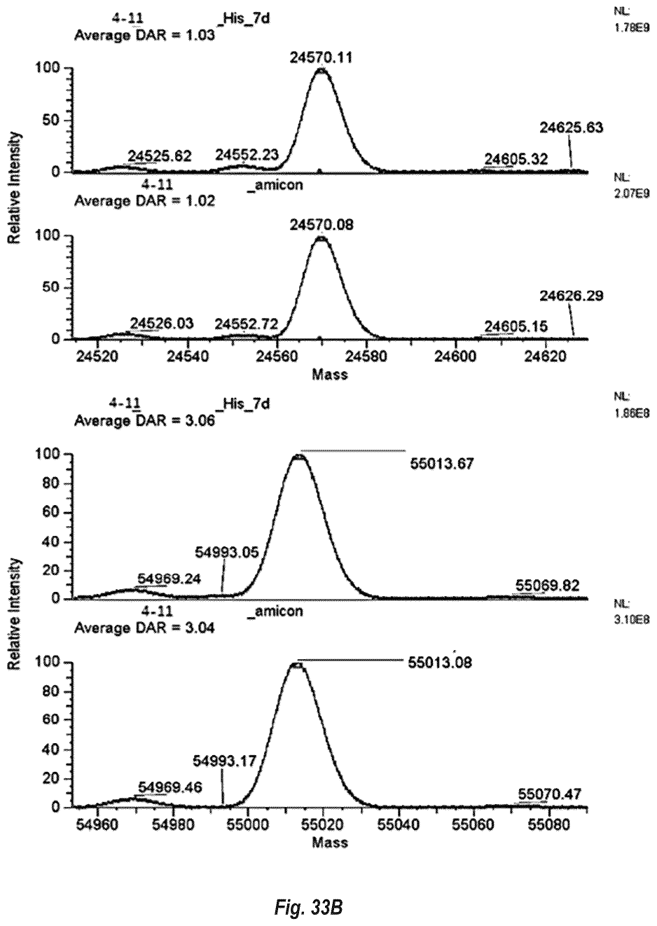

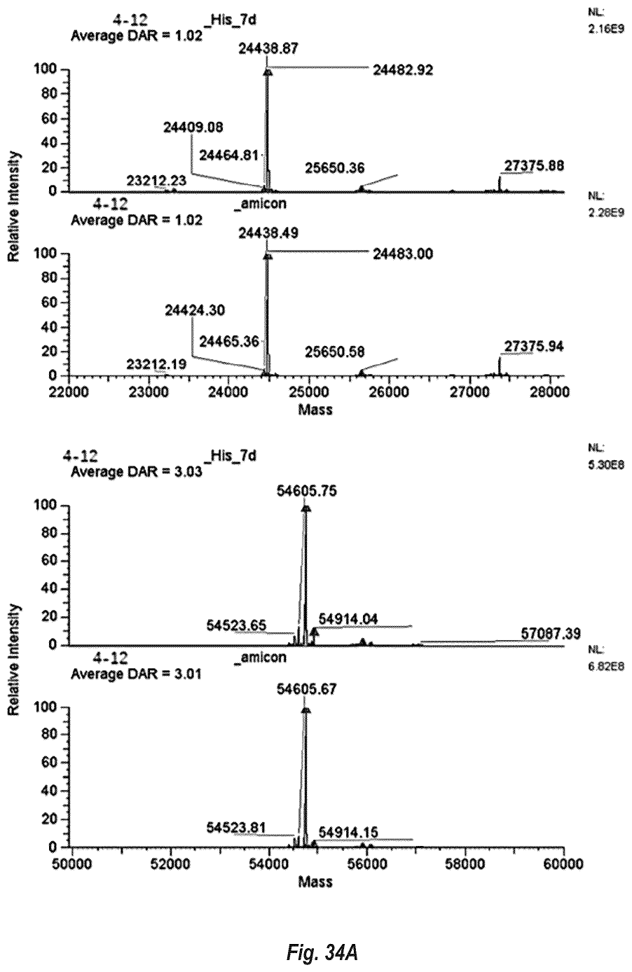

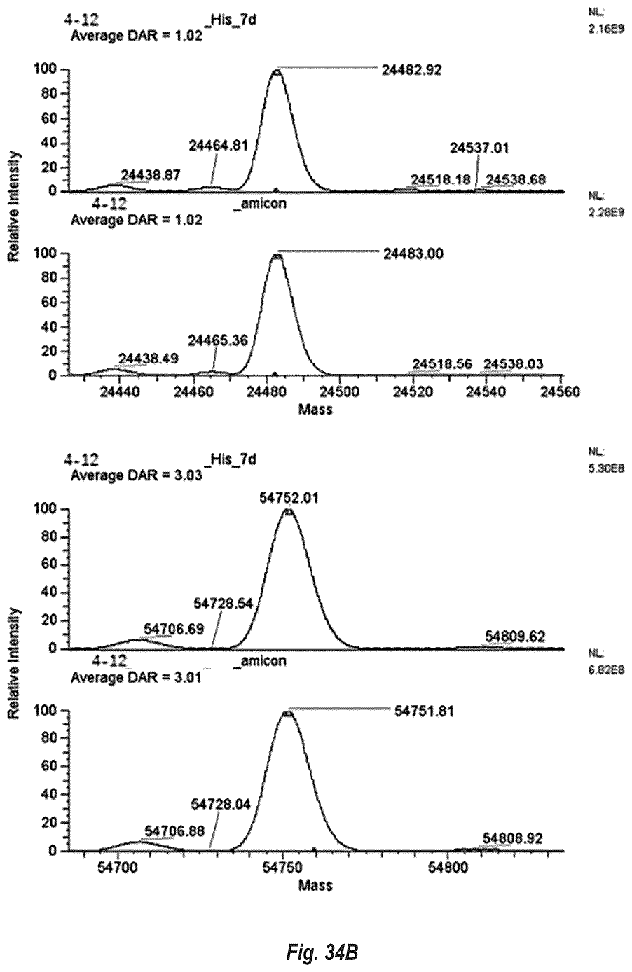

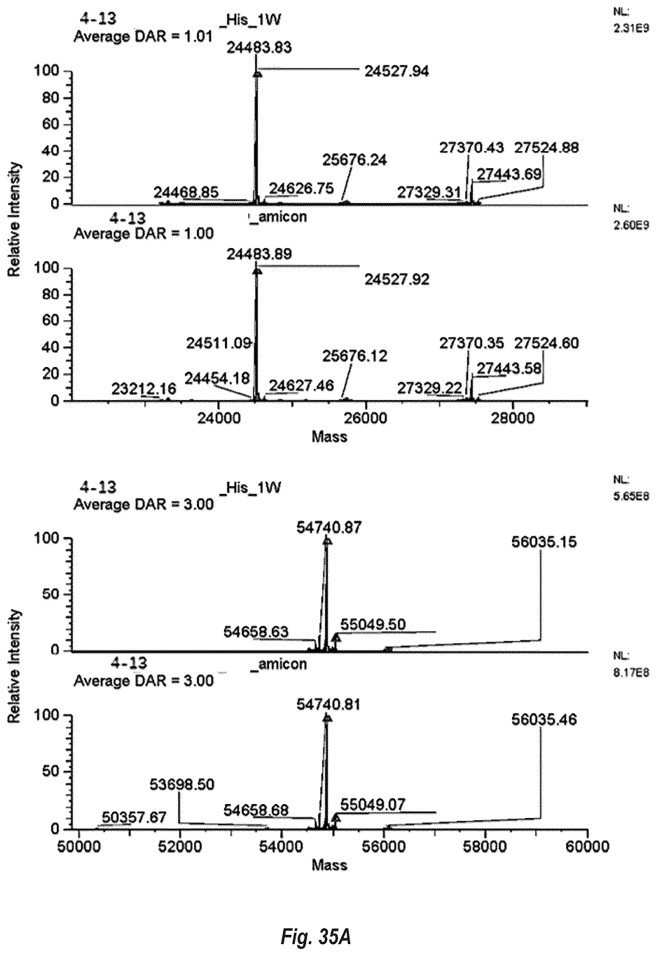

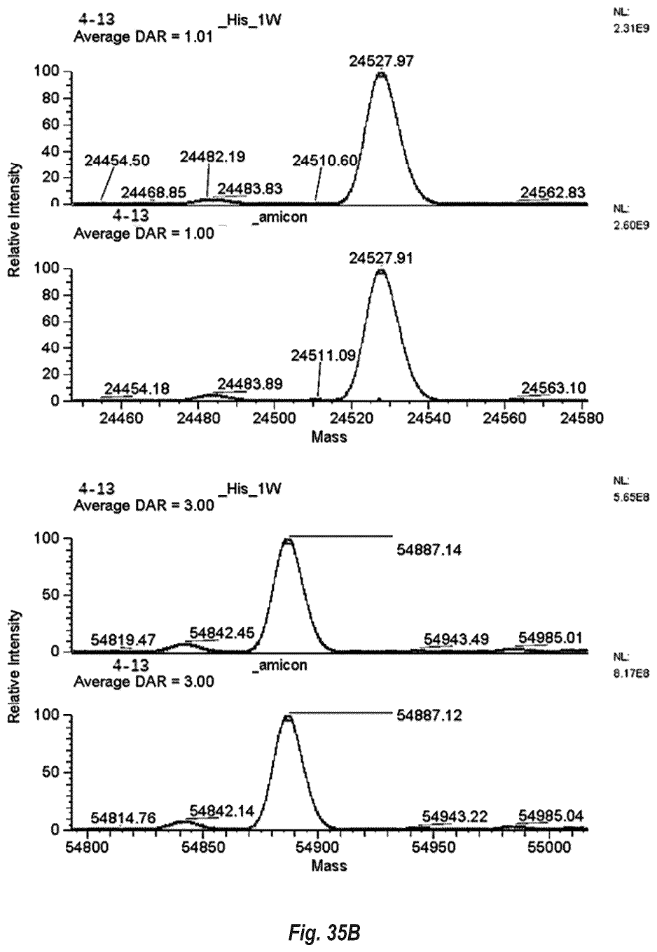

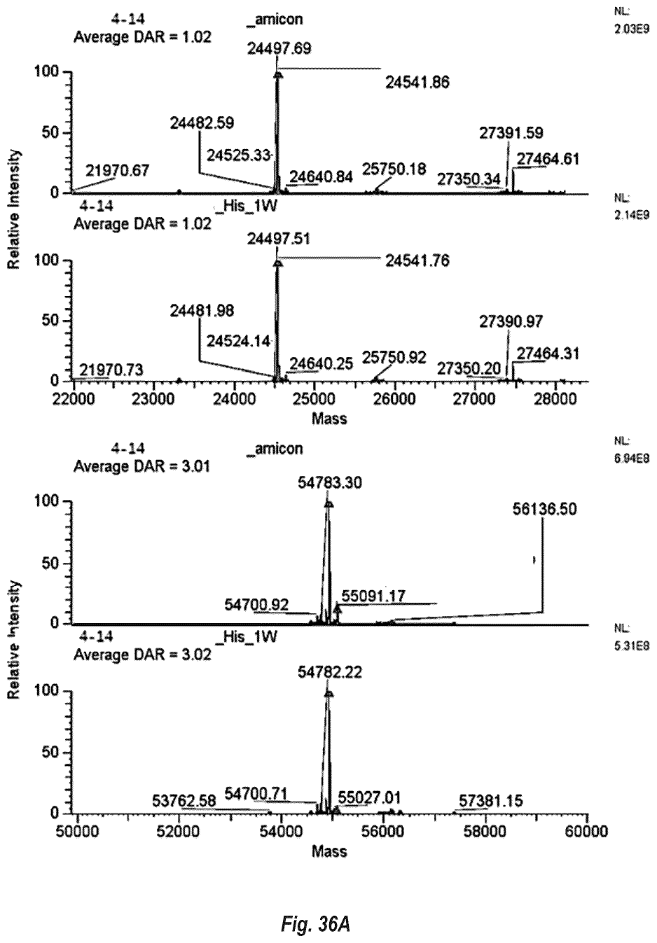

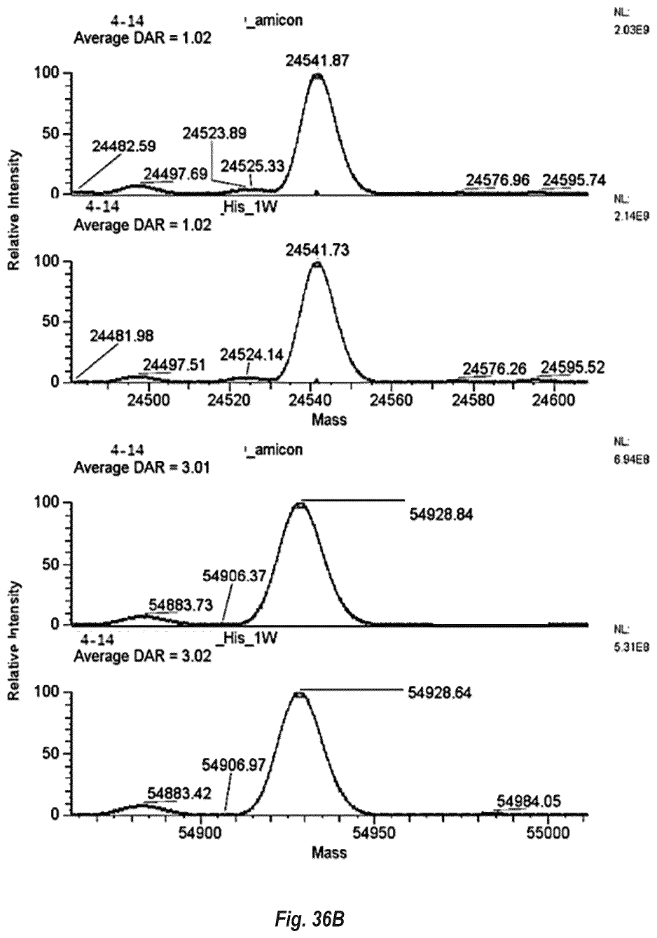

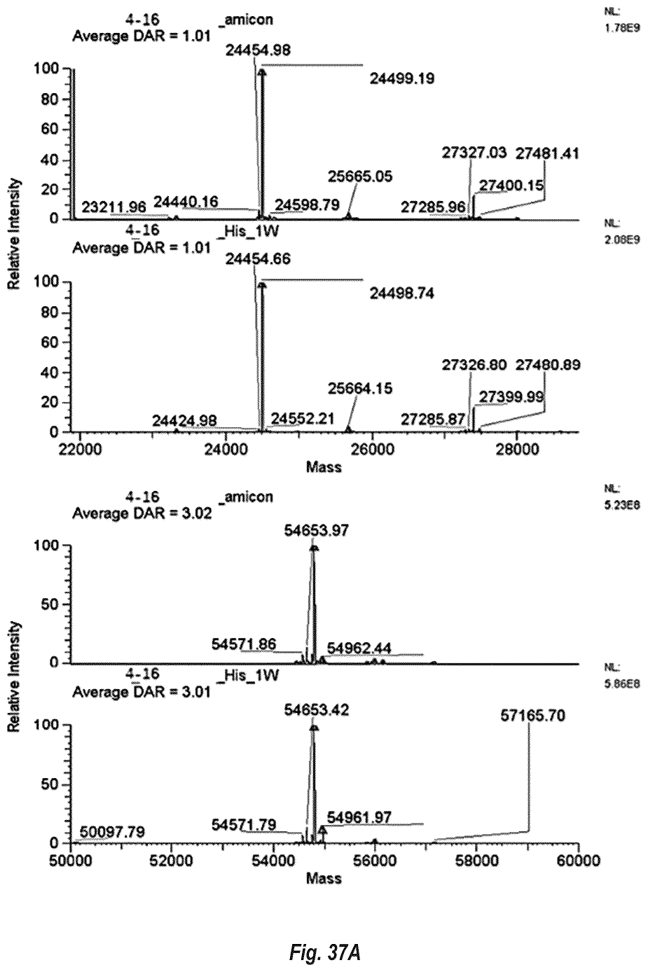

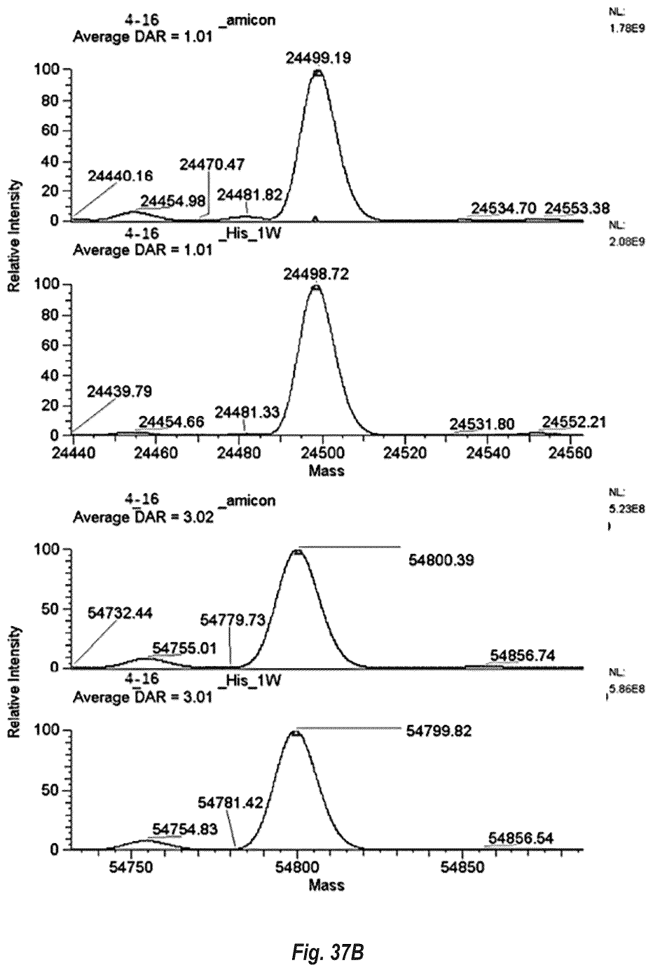

Results of stability assessments of ADCs in GSH solution or formulation buffer are presented in Table 8 and Table 9, respectively, and FIGS. 24A-37B.

TABLE 8
ADC stability in pH 7.4 and 8.0 buffer with and without GSH

| ADC | C_{GSH} (mM) | Time (h) | pH | Temp. (° C.) | MS DAR |
|---|---|---|---|---|---|
| ADC 4-6 | 10 | 18 | 7.4 | 22 | 8.06 |
| | | | | 37 | 8.08 |
| | | | 8.0 | 22 | 8.06 |
| ADC 4-10 | 10 | 18 | 7.4 | 22 | 8.04 |
| | | | | 37 | 8.02 |
| | | | 8.0 | 22 | 8.04 |
| ADC 4-11 | 10 | 18 | 7.4 | 22 | 8.34 |
| | | | | 37 | 8.48 |
| | | | 8.0 | 22 | 8.34 |
| ADC 4-12 | 10 | 18 | 7.4 | 22 | 8.18 |
| | | | | 37 | 8.32 |
| | | | 8.0 | 22 | 8.20 |
| ADC 4-13 | 10 | 72 | 7.4 | 22 | 7.94 |
| | | | | 37 | 7.96 |
| | | | 8.0 | 22 | 7.94 |
| ADC 4-14 | 10 | 72 | 7.4 | 22 | 8.14 |
| | | | | 37 | 8.16 |
| | | | 8.0 | 22 | 8.12 |
| ADC 4-16 | 10 | 96 | 7.4 | 22 | 8.14 |
| | | | | 37 | 8.20 |
| | | | 8.0 | 22 | 8.14 |

The results demonstrate that no deconjugation events were observed for ADC 4-6, 4-10 to 4-14, and 4-16 after incubation in GSH solutions (pH 7.4 or 8.0 for 18 hours).

TABLE 9
ADC stability in formulation buffer (20 mM histidine buffer)

| | | | | | | Maleimide ring |
|---|---|---|---|---|---|---|
| | Time | | Temp. | MS DAR- | MS DAR- | opening ratio (%) at |
| ADC | (h) | pH | (° C.) | T = 0 h | T = 168 h | T = 168 h |
| ADC 4-6 | 93 | 5.5 | 22 | 8.02 | 8.14 | 98.88 |
| ADC 4-10 | 168 | 5.5 | 22 | 8.02 | 8.08 | 95.21 |
| ADC 4-11 | 168 | 5.5 | 22 | 8.12 | 8.18 | 99.93 |
| ADC 4-12 | 168 | 5.5 | 22 | 8.06 | 8.10 | 99.81 |
| ADC 4-13 | 168 | 5.5 | 22 | 8.00 | 8.02 | 99.94 |
| ADC 4-14 | 168 | 5.5 | 22 | 8.06 | 8.08 | 99.96 |
| ADC 4-16 | 168 | 5.5 | 22 | 8.06 | 8.04 | 99.92 |

The results demonstrated that no deconjugation events were observed for ADC 4-6, 4-10 to 4-14, and 4-16 after storage in formulation buffer for one week.

### Cell Lines

NCI-H1650 (ATCC, CRL-5883). NCI-H1650 is a cell line exhibiting epithelial morphology that was isolated in 1987 from the lung tissue of a 27-year-old male smoker with stage 3, bronchoalveolar carcinoma, and NCI-H1650 was purchased from ATCC. The base medium for NCI-H1650 is ATCC-formulated RPMI-1640 Medium, ATCC 30-2001. To make the complete growth medium, fetal bovine serum to a final concentration of 10% (Gibco, 10099-141C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

Capan-1 (ATCC, HTB-79). Capan-1 is a cell line with epithelial morphology that was isolated from the pancreas of a 40-year-old white male with pancreatic adenocarcinoma, and Capan-1 was purchased from ATCC. The base medium for Capan-1 is ATCC-formulated Iscove's Modified Dulbecco's Medium, Catalog No. 30-2005. To make the complete growth medium, fetal bovine serum to a final concentration of 20% (Gibco, 10099-141C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

MDA-MB-453 (SIBS). MDA-MB-453 was derived from an effusion of a 48-year-old female patient with metastatic carcinoma of the breast, involving the nodes, brain, and both pleural and pericardial cavities, and MDA-MB-453 was purchased from SIBS. The base medium for MIDA-MB-453 is RPMI 1640 Medium, HEPES (Gibco, 22400105). To make the complete growth medium, fetal bovine serum to a final concentration of 10% (Gibco, 10099-141C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

TABLE 10
B7H3 expression level

| Cell line | Molecular No. (QSC kit) |
|---|---|
| NCI-H1650 | 505998 |
| Capan-1 | 63062 |
| MDA-MB-453 | 0 |

### ADC Direct Killing in NCI-H1650, Capan-1, and MDA-MB-453 Cancer Cell Lines

ADC direct killing was assessed in NCI-1650, Capan-1, and MDA-MB-453 cancer lines. Cells were seeded (NCI-1650 or MDA-MB-453 (2E3/well) or Capan-1 (4E3/well)) into 3D 96-well plates (Corning: 4520), 80 μl/well, and incubated at 37° C., 5% CO₂, overnight. Fresh growth medium was added containing varying concentrations of ADCs, 40 μl/well, and incubated at 37° C., 5% CO₂, for 6 days. The cell viability was detected by 3D reagent (Promega, G9683), 100 μl/well. The 3D plates were allowed to incubate at room temperature for 30 minutes to stabilize the luminescent signal. The plates were analyzed with a Microplate Reader.

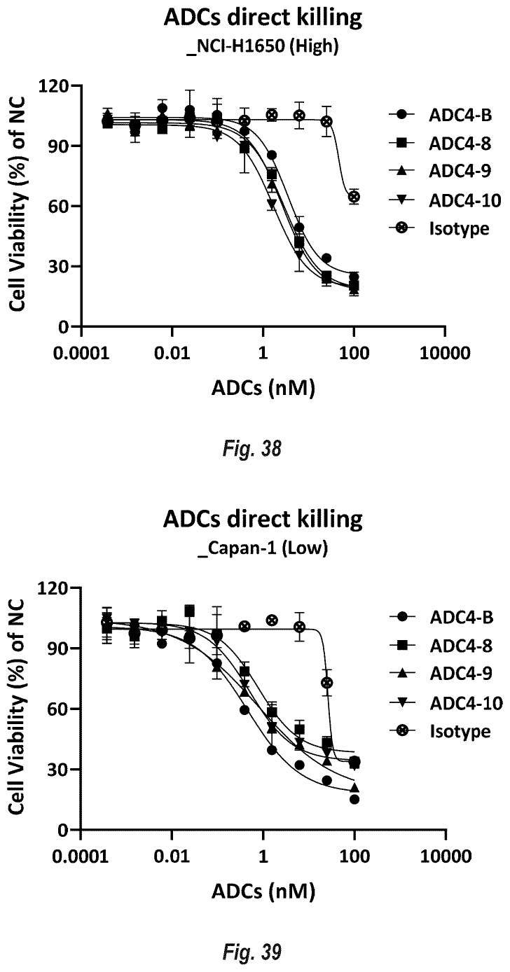

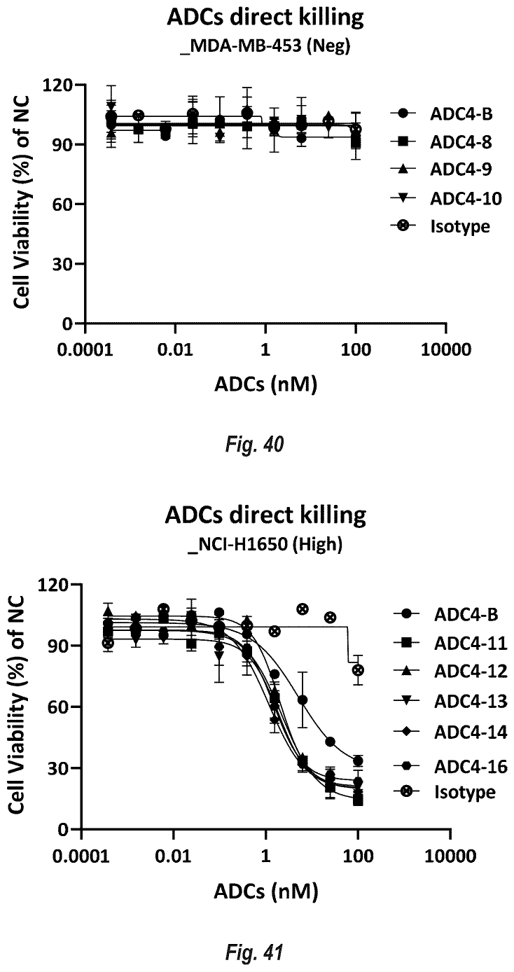

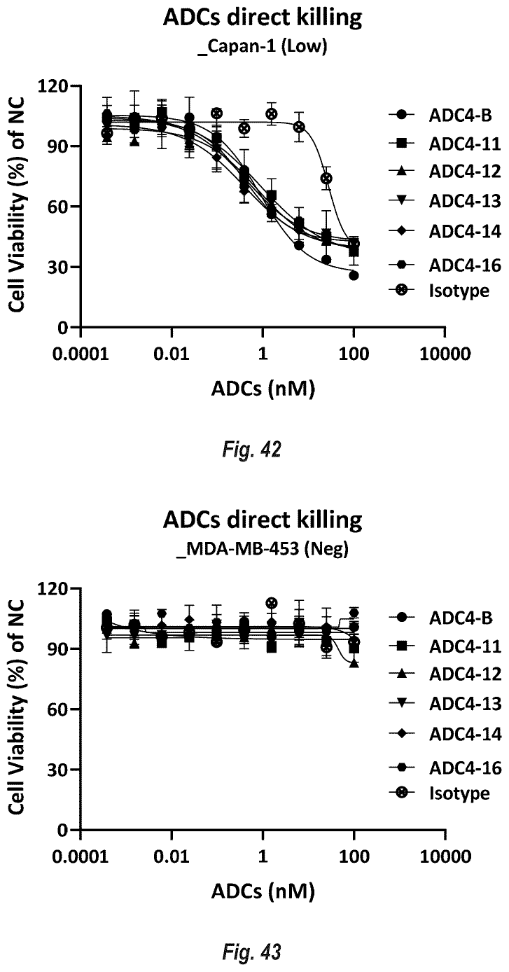

Data are summarized in Tables 11-12 and FIGS. 38-43.

TABLE 11
ADC direct killing potency on B7H3
high- and low-expression cell lines

| | |
|---|---|
| NCI-H1650 (High), 3D | Capan-1 (Low), 3D |

| | Emax | EC50 | Emax | EC50 |
|---|---|---|---|---|
| ADCs | (%) | (nM) | (%) | (nM) |
| ADC4-B | 75.3 | 3.9 | 84.9 | 0.5 |
| ADC4-8 | 79.5 | 2.9 | 66.6 | 1.0 |
| ADC4-9 | 81.4 | 2.8 | 78.8 | 0.8 |
| ADC4-10 | 80.0 | 1.7 | 68.4 | 0.6 |

TABLE 12
ADC direct killing potency on B7H3
high- and low-expression cell lines

| | |
|---|---|
| NCI-H1650 (High), 3D | Capan-1 (Low), 3D |

| | Emax | EC50 | Emax | EC50 |
|---|---|---|---|---|
| ADCs | (%) | (nM) | (%) | (nM) |
| ADC4-B | 66.5 | 4.5 | 74.3 | 1.1 |
| ADC4-11 | 85.6 | 2.1 | 62.4 | 1.0 |
| ADC4-12 | 83.2 | 2.3 | 61.9 | 0.7 |
| ADC4-13 | 79.3 | 1.6 | 58.5 | 0.5 |
| ADC4-14 | 81.6 | 1.3 | 61.3 | 0.4 |
| ADC4-16 | 76.4 | 1.5 | 62.2 | 0.6 |

### ADCs In Vivo Efficacy Study

Female BALB/c Nude mice were subcutaneously implanted with 3× 106 H₁₆₅₀ cells per 200 μL PBS/matrigel in the right flank. After inoculation, tumor volumes were determined twice weekly in two dimensions using a caliper and were expressed in mm³ using the formula V=0.5 (a×b²) where a and b are the long and short diameters of the tumor, respectively. When tumors reached a mean volume of approximately 200 mm³ in size, mice were randomly allocated into 6 groups with 8 animals in each group, and were intravenously treated with vehicle, ADC4-B, ADC4-10, ADC4-12, ADC4-14, or ADC4-16 at 1 or 3 mg/kg QW*2. Partial regression (PR) was defined as tumor volume smaller than 50% of the starting tumor volume on the first day of dosing in three consecutive measurements and complete regression (CR) was defined as tumor volume less than 14 mm³ in three consecutive measurements. Data are presented as mean tumor volume±standard error of the mean (SEM). Tumor growth inhibition (TGI) is calculated using the following formula:

$$\% growth inhibition = 100 \times ( 1 - ( \frac{( treated t ) - ( treated to )}{( placebo t ) - ( placebo to )} ) )$$

$$treated t = treated tumor volume at time t$$

$$treated {t}_{0} = treated tumor volume at time 0$$

$$placebo t = placebo tumor volume at time t$$

$$placebo {t}_{0} = placebo tumor volume at time 0$$

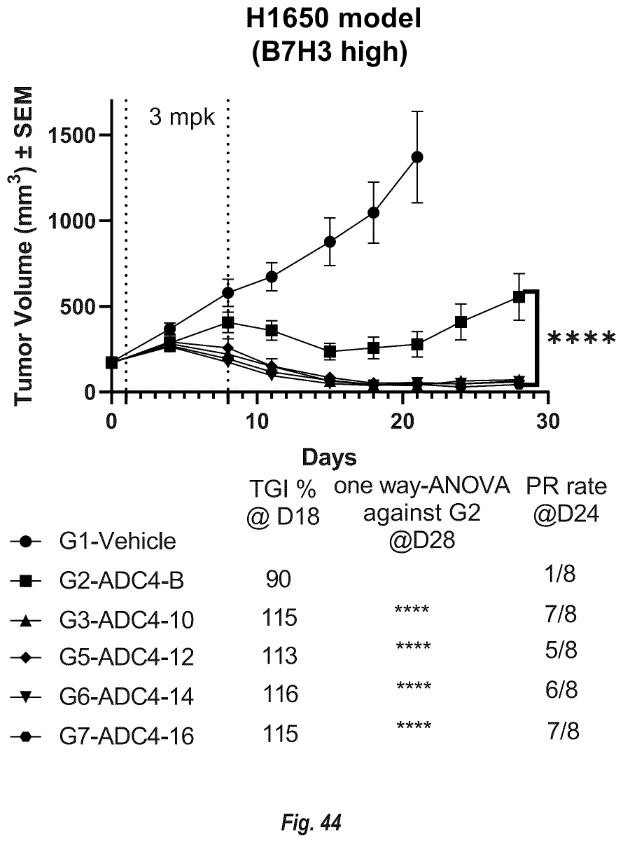

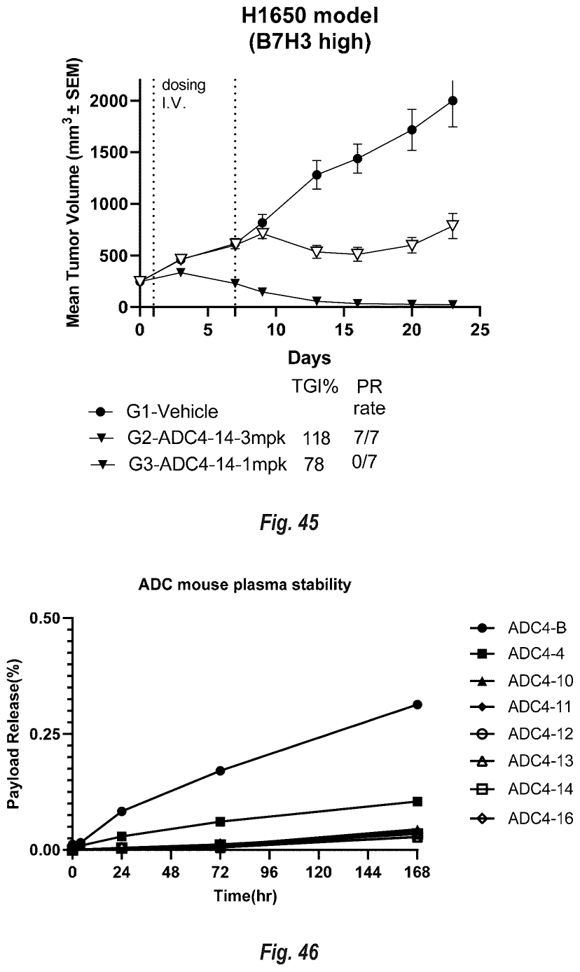

Results are presented in FIGS. 44 and 45. The tested ADCs with open-ring (RO) conjugators (i.e., ADC4-10, ADC 4-12, ADC 4-14, and ADC 4-16) exhibited potent anti-tumor activities in a human lung cancer model, and the activities are superior to a non-RO conjugator ADC (i.e., ACD4-B) (FIG. 44). The RO conjugator ADCs exhibited anti-tumor activity in a dose dependent manner (FIG. 45).

### ADC Plasma Stability

Incubation of ADC with plasma: ADCs were diluted into mouse or human plasma to yield a final solution of 100 g/mL ADC in plasma. The samples were incubated at 37° C. Aliquots (100 μL) are taken at five time points (0, 4, 24, 72, or 168 h). Samples were frozen at −80° C. until analysis.

Plasma payload concentrations were carried out under the following measurement conditions:
Instrument: LC-MS/MS (Triple Quad 6500 plus)
Monitor: MRM
Column: Advanced Materials Technology, HALO AQ-C18 2.7 μm 90 Å, 50*2.1 mm
Column temperature: 40° C.
Mobile phase A: H₂O-0.1% FA
Mobile phase B: ACN-0.1% FA
Gradient program for DXd and Topoli analogues: 2% B-2% B (0 min-0.2 min), 2% B-98% B (0.2 min-1.2 min), 98% B-98% B (1.2 min-2.0 min), 98% B-2% B (2.0 min-2.01 min), 2% B-2% B (2.01 min-4.0 min)
Injected sample amount: 10 μL (DXd or Topoli analogues)

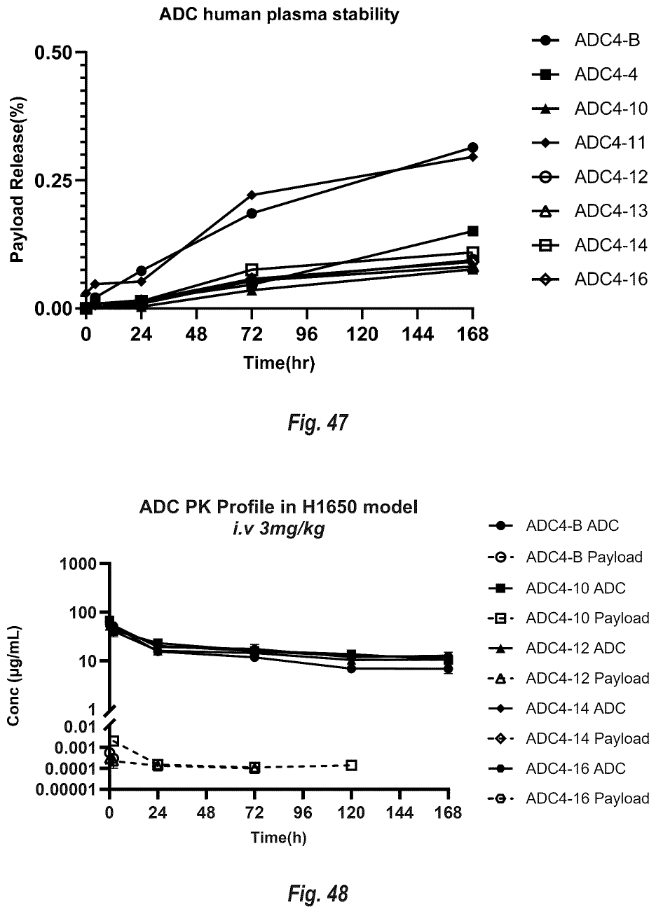

Results are presented in FIGS. 46 and 47. ADCs with a maleimide-hydrolyzed RO conjugator showed low payload release rate in mouse plasma (FIG. 46) and in human plasma (FIG. 47).

### In Mice PK Study

After one dose of intravenously administered ADC to H1650 tumor bearing mice or non-tumor bearing mice, blood samples were collected 0.0833, 2, 24, 72, 120, and 168 h later, followed by centrifugation (4° C., 3000×g, 7 min) to separate plasma. The concentrations of ADCs were measured by in-house developed Meso Scale Discovery (MSD) ligand binding methods. Briefly, a His-tagged B7H3 extracellular domain fusion protein was used as a capture reagent, and biotin-labelled anti-payload Ab was used as the detection reagent for ADCs. Plasma concentration of payload was measured using the same method as described above.

Results are presented in FIG. 48. ADCs with a maleimide-hydrolyzed RO conjugator showed good ADC PK and low payload exposure in an H1650 efficacy model.

Although the foregoing disclosure has been presented in some detail by way of illustration and example for purposes of clarity of understanding, it is apparent to those skilled in the art that certain minor changes and modifications will be practiced. Therefore, the description and examples should not be construed as limiting.

It is to be understood that, if any publication is referred to herein, such reference does not constitute an admission that the publication forms a part of the common general knowledge in the art in any country.

The disclosures of all non-patent publications, patents, patent applications, and published patent applications referred to herein are hereby incorporated herein by reference in their entireties.

What is claimed is:

1. A compound of Formula (I): 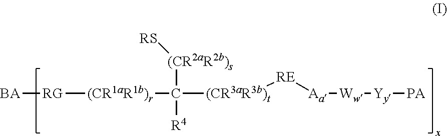
or a pharmaceutically acceptable salt, tautomer, solvate, or stereoisomer thereof, wherein:
BA is a binding agent selected from a humanized, chimeric, or human antibody or an antigen binding fragment thereof;
RG is a reactive group residue;
RS is an open-ring stabilizing group;
RE is an open-ring enhancer;
each of R^{1a} and R^{1b} is, independently, H; substituted or unsubstituted C₁₋₄ alkyl;
substituted or unsubstituted C₃₋₅ cycloalkyl; or R^{1a} and R^{1b} together with the atom to which they are attached form a substituted or unsubstituted C₃₋₅ cycloalkyl;
each of R^{2a} and R^{2b} is, independently, H; substituted or unsubstituted C₁₋₄ alkyl;
substituted or unsubstituted C₃₋₅ cycloalkyl; or R^{2a} and R^{2b} together with the atom to which they are attached form a substituted or unsubstituted C₃₋₅ cycloalkyl;
each of R^{3a} and R^{3b} is, independently, H; substituted or unsubstituted C₁₋₄ alkyl;
substituted or unsubstituted C₃₋₅ cycloalkyl; or R^{3a} and R^{3b} together with the atom to which they are attached form a substituted or unsubstituted C₃₋₅ cycloalkyl;
R⁴ is H; substituted or unsubstituted C₁₋₄ alkyl; or substituted or unsubstituted C₃₋₅ cycloalkyl;
each of r, s, and t is, independently, 0, 1, or 2;
A is a Stretcher unit residue;
subscript a′ is 0 or 1;
W is a Cleavable unit;
subscript w′ is 0 or 1;
Y is a Spacer unit;
subscript y′ is 0 or 1;
PA is a payload residue; and
subscript x is from 1 to 15.

2. The compound of claim 1, wherein each of R^{1a}, R^{1b}, R^{2a}, R^{2b}, R^{3a}, R^{3b}, and R⁴ is, independently, H.

3. The compound of claim 1 or claim 2,
wherein RS is an amino group or —NR^{5a}R^{5b}, and
wherein each of R^{5a} and R^{5b} is, independently, H, or substituted or unsubstituted C₁₋₄ alkyl.

4. The compound of claim 3, wherein RS is an amino group or —N(CH₃)₂.

5. The compound of claim 0, wherein RS is an amino group.

6. The compound of any one of claims 1-5, wherein RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—; and R⁶ is H, or substituted or unsubstituted C₁₋₄ alkyl.

7. The compound of claim 6, wherein R⁶ is H, methyl, ethyl, or isopropyl.

8. The compound of claim 7, wherein RE is —OC(═O)NR⁶—.

9. The compound of claim 8, wherein RE is —OC(═O)NH—.

10. The compound of any one of claims 1-9, wherein RG is

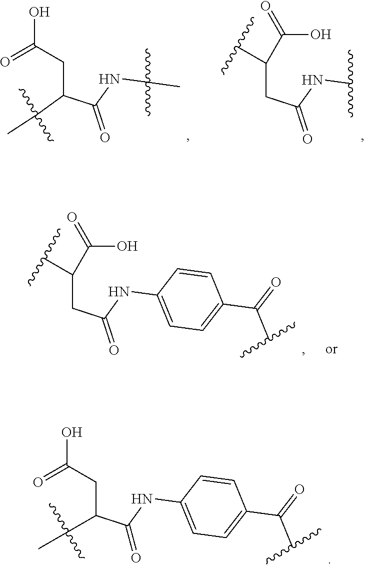 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1]

11. The compound of any one of claims 1-9, wherein RG is

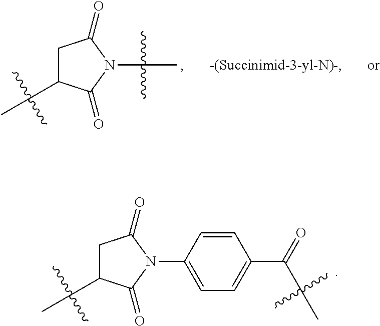 [SMILES: *C1CC(=O)N(*)C1=O ; *C(=O)c1ccc(N2C(=O)CC(*)C2=O)cc1]

12. The compound of any one of claims 1-0, wherein r is 0.

13. The compound of any one of claims 1-0, wherein s is 1.

14. The compound of any one of claims 1-0, wherein t is 1 or 2.

15. The compound of any one of claims 1-0, wherein A
is —(CH₂)ₙ—C(═O)—, —CH₂—C(═O)—NH—(CH₂)ₙ—C(═O)—, —(CH₂CH₂O)ₙ—CH₂CH₂—C(═O)—, —CH [—(CH₂)ₙ—COOH]—C(═O)—, —CH₂—C(═O)—NH—(CH₂)ₙ—C(═O)—NH—(CH₂)ₙ—C(═O)—, or —C(═O)—(CH₂)ₙ—C(═O)—, wherein each n independently represents an integer of 1, 2, 3, 4, or 5.

16. The compound of any one of claims 1-0, wherein W_{w′} is one of the following formulas: 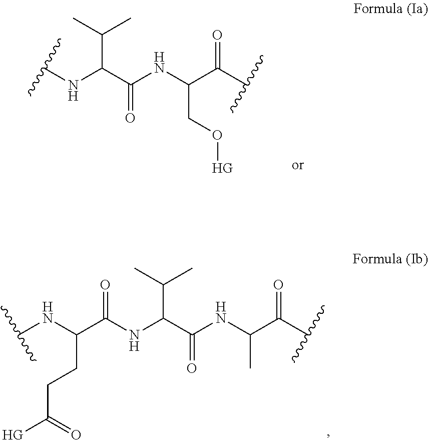
and
wherein HG is a hydrophilic moiety or hydrogen.

17. The compound of claim 16, wherein HG is a saccharide, phosphate ester, sulfate ester, a phosphodiester, or a phosphonate.

18. The compound of claim 17, wherein HG is a saccharide and the saccharide is β-D-galactose, N-acetyl-P-D-galactosamine, N-acetyl-a-D-galactosamine, N-acetyl-P-D-glucosamine, β-D-glucuronic acid, a-L-iduronic acid, a-D-galactose, a-D-glucose, β-D-glucose, a-D-mannose, β-D-mannose, a-L-fucose, β-D-xylose, a neuraminic acid, sulfate, phosphate, carboxyl, amino, or an O-acetyl modification thereof.

19. The compound of claim 16, wherein HG is

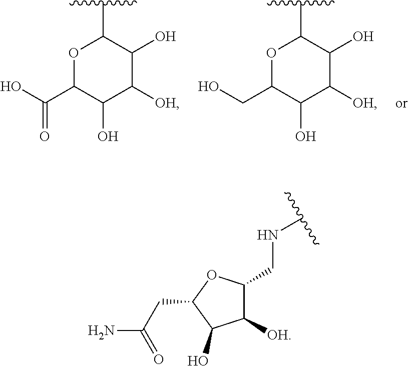 [SMILES: *C1OC(C(=O)O)C(O)C(O)C1O ; *C1OC(CO)C(O)C(O)C1O ; *NC[C@H]1O[C@@H](CC(N)=O)[C@H](O)[C@@H]1O]

20. The compound of any one of claims 1-19, wherein Y_{y′} is a p-aminobenzyl alcohol (PAB) unit.

21. The compound of claim 1, wherein the compound is one of the following formulas:

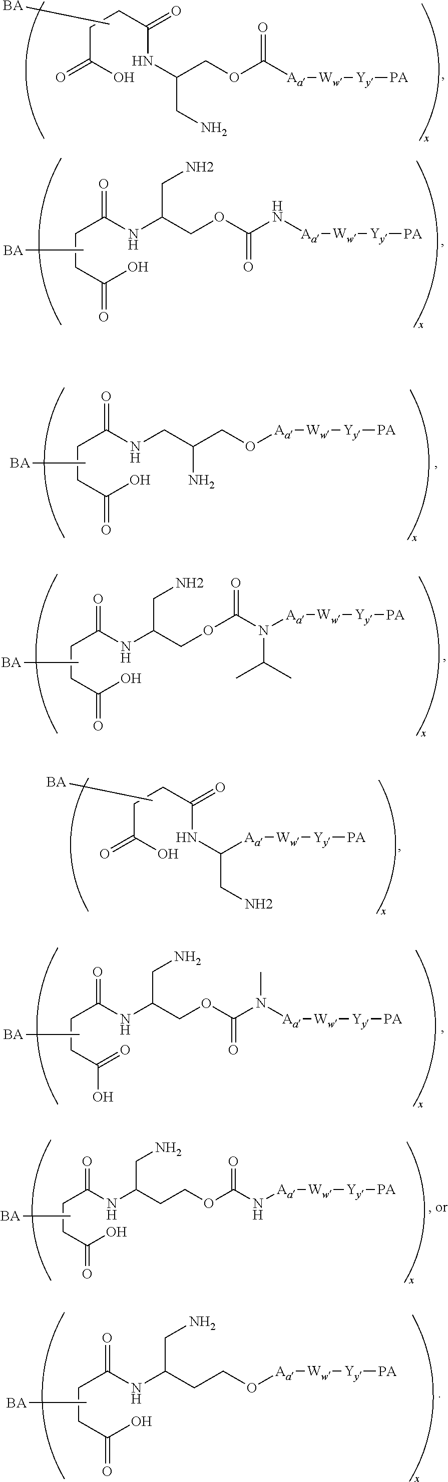

22. The compound of claim 1, wherein the compound is one of the following formulas:

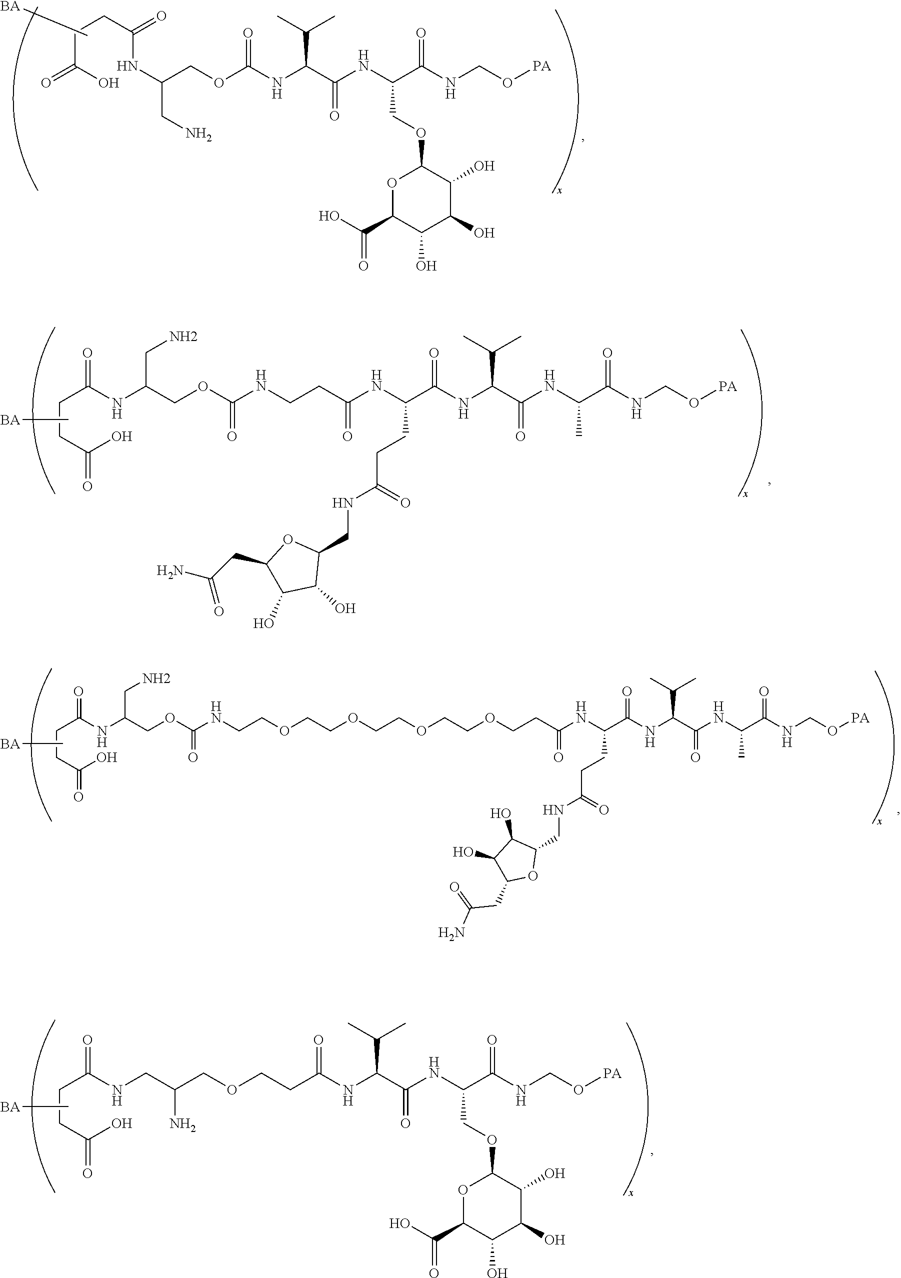

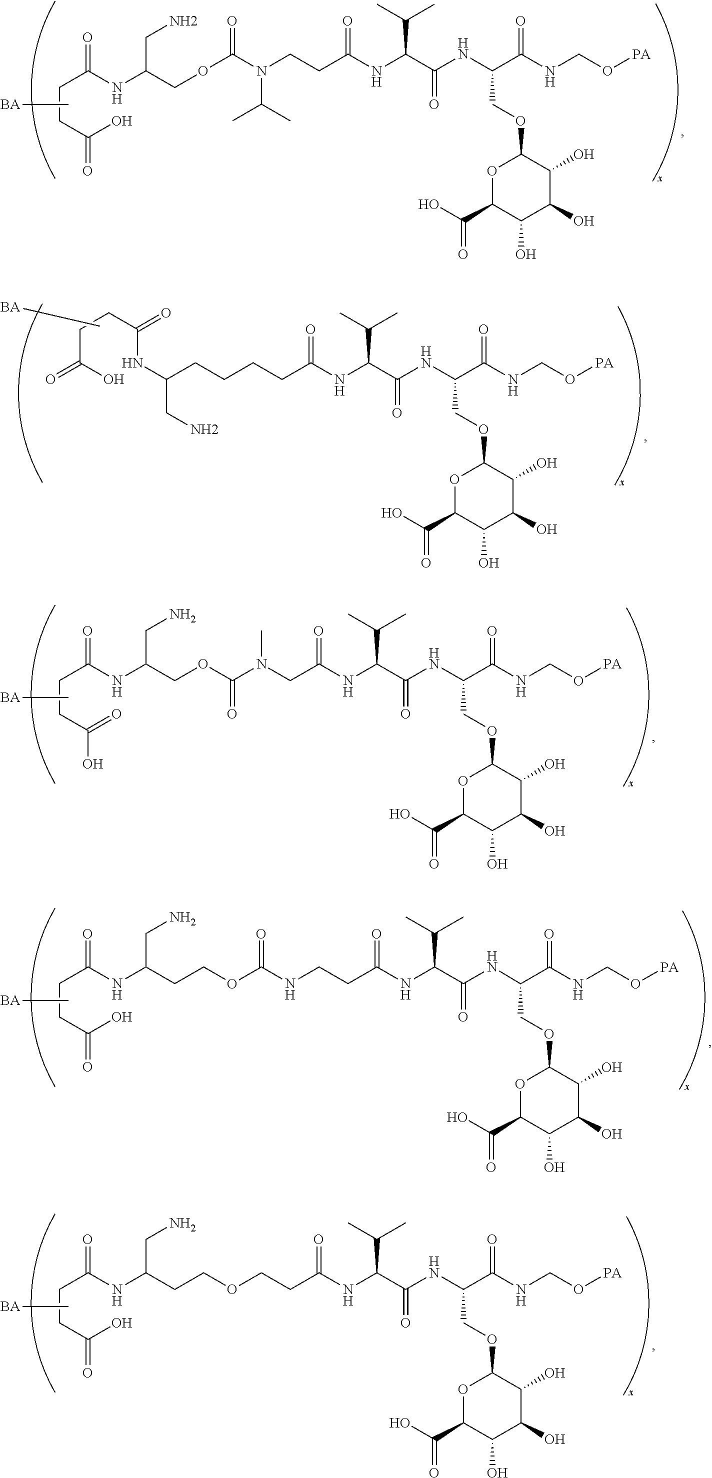

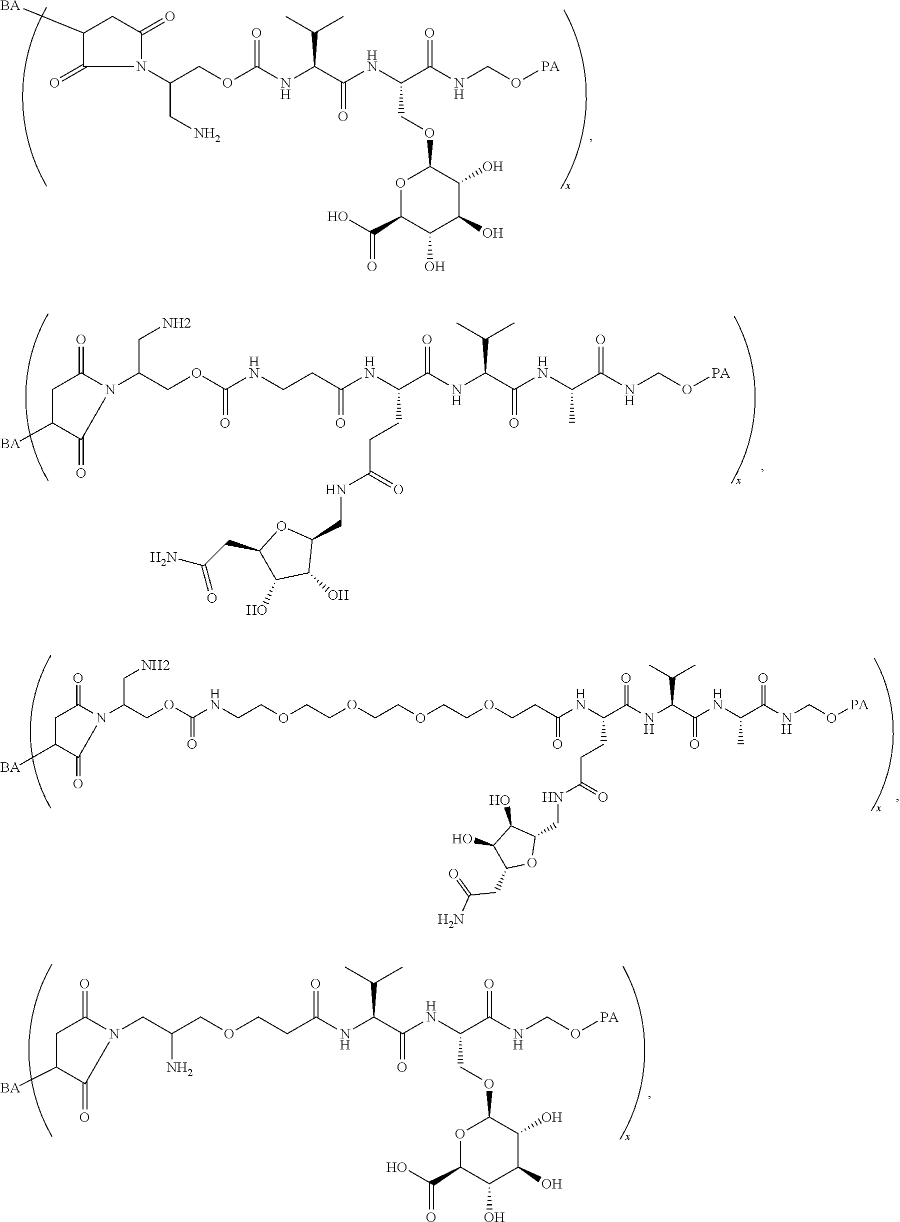

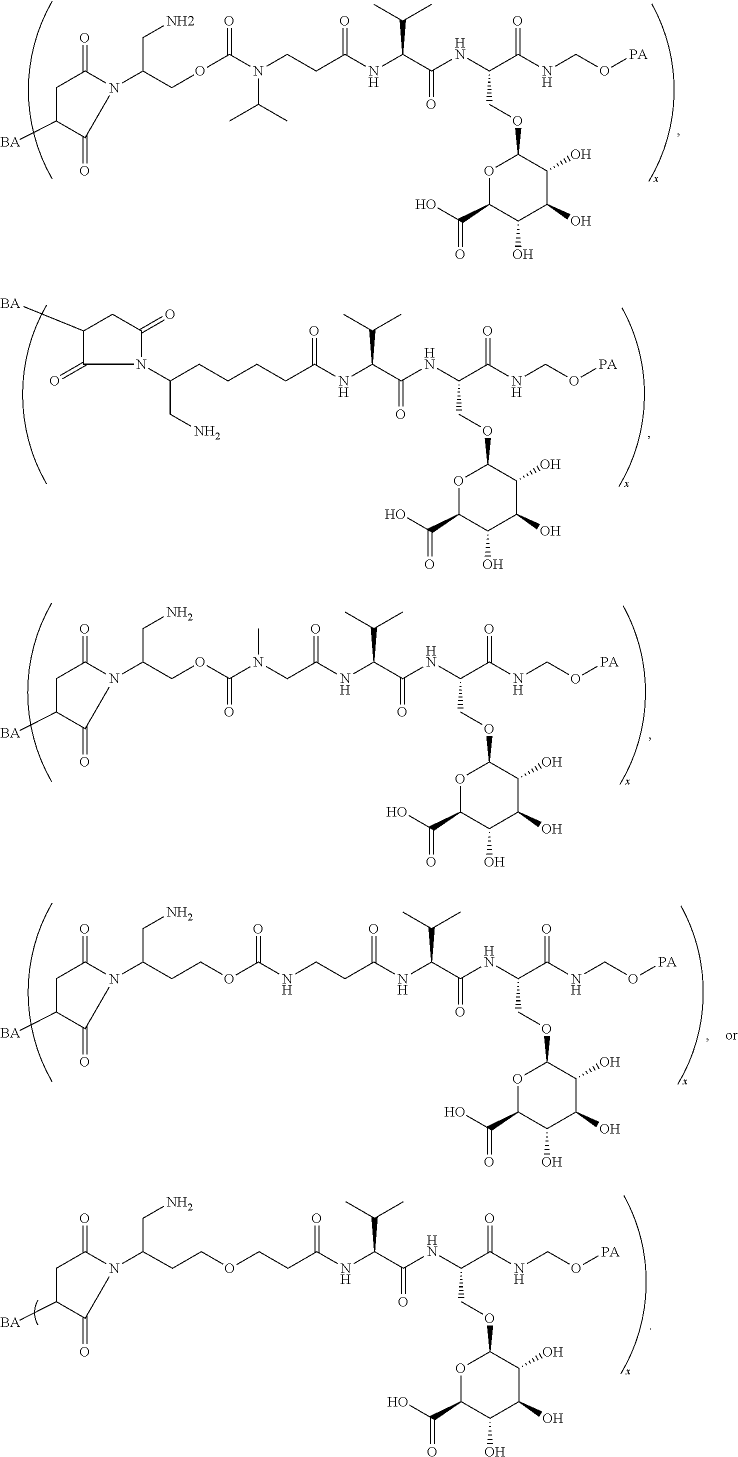

23. The compound of any one of claims 1-22, wherein BA is ifinatamab, cofetuzumab, patritumab, or trastuzumab, or the antigen binding fragment of ifinatamab, cofetuzumab, patritumab, or trastuzumab.

24. The compound of any one of claims 1-22, wherein BA is a humanized, chimeric, or human antibody or the antigen binding fragment thereof which binds to one or more of receptors chosen from HER2, HER3, PTK7, or B7H3.

25. The compound of claim 1, wherein the compound is 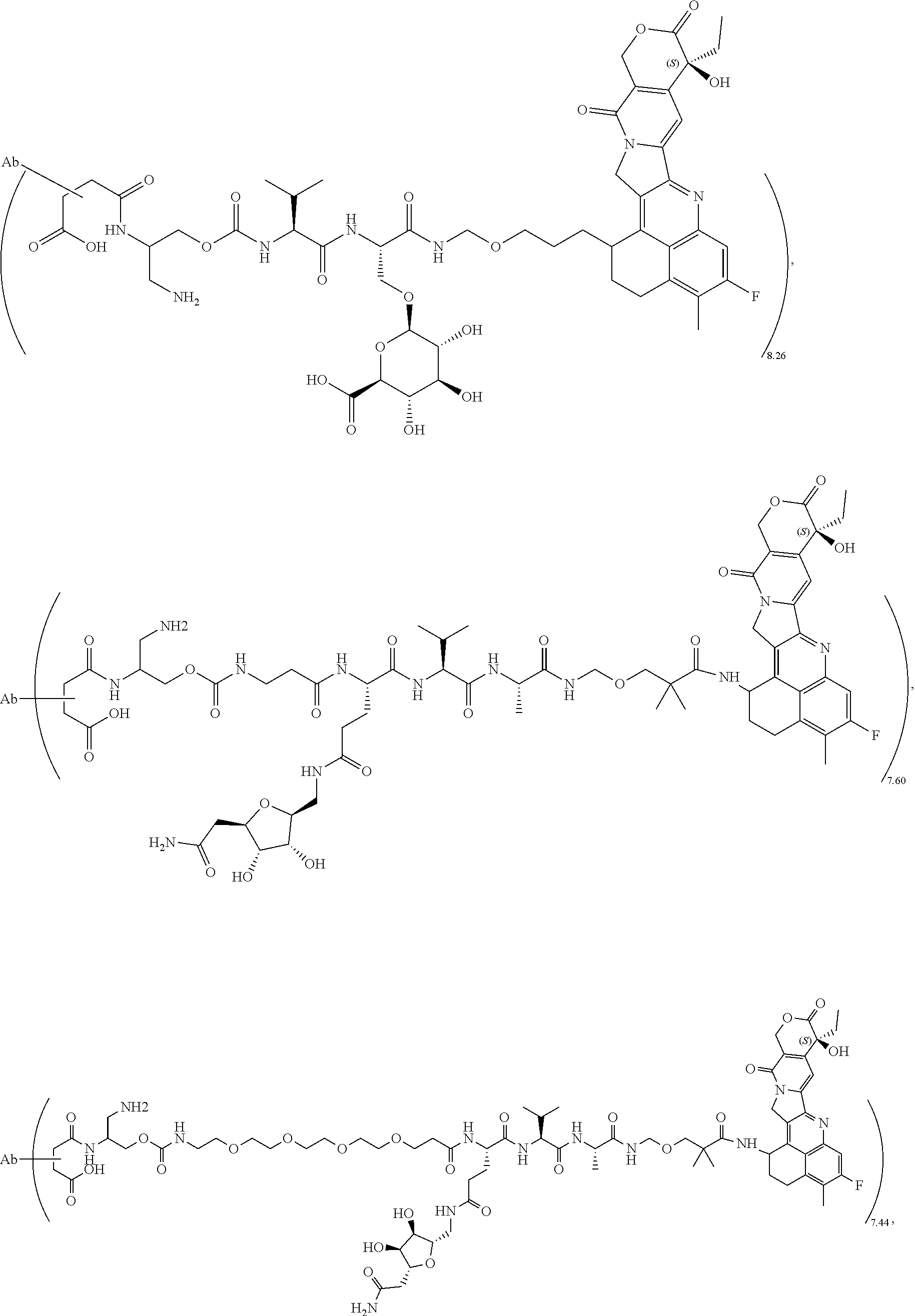 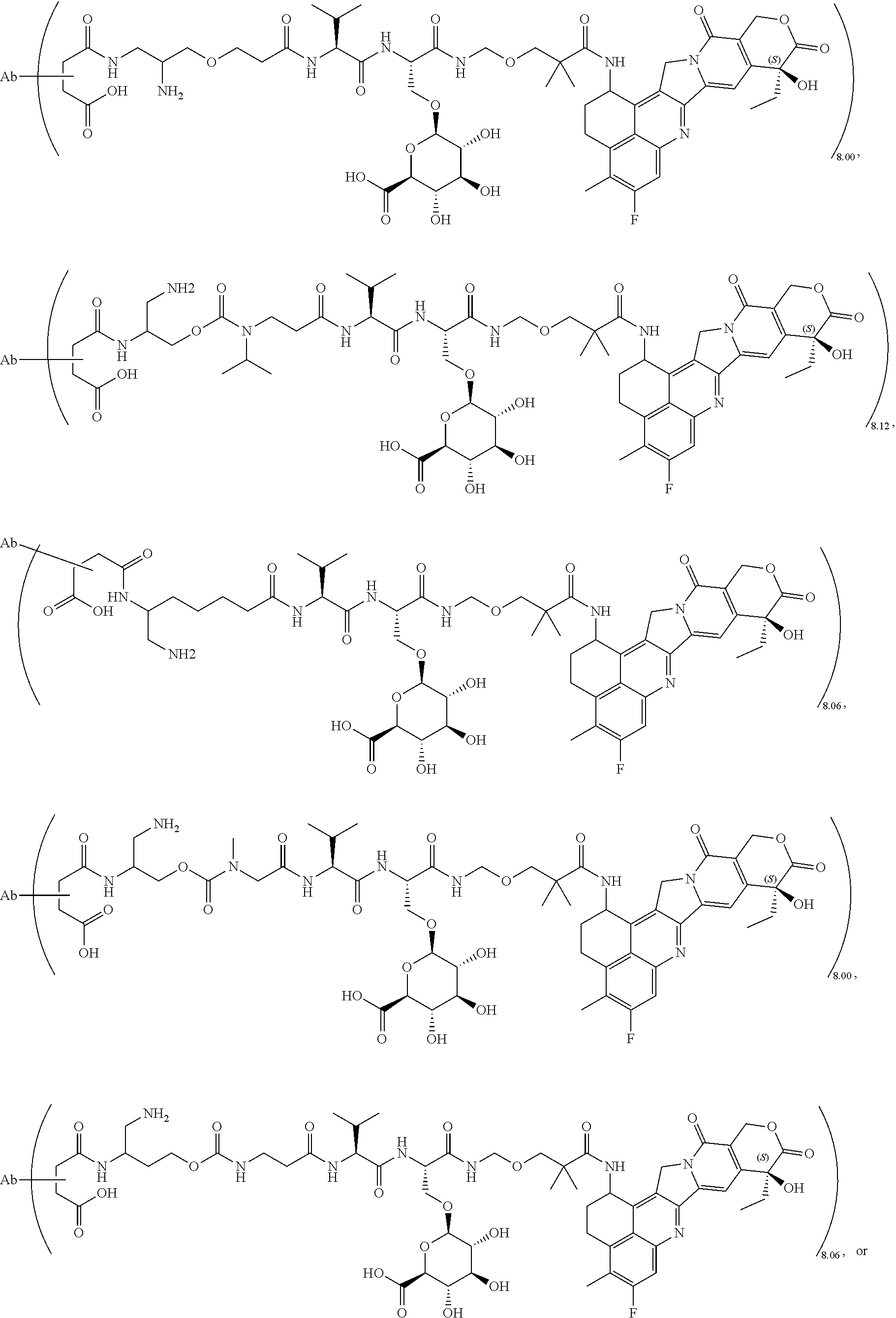 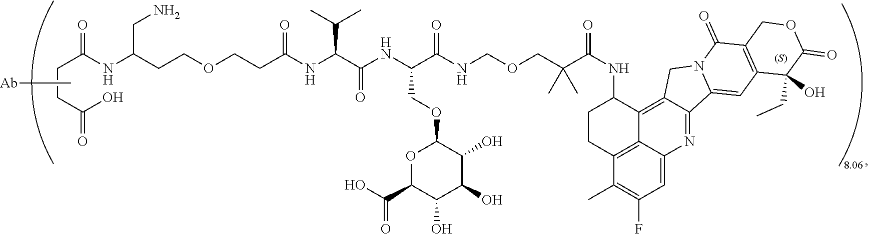
wherein Ab is ifinatamab.

26. The compound of claim 1, wherein the compound is 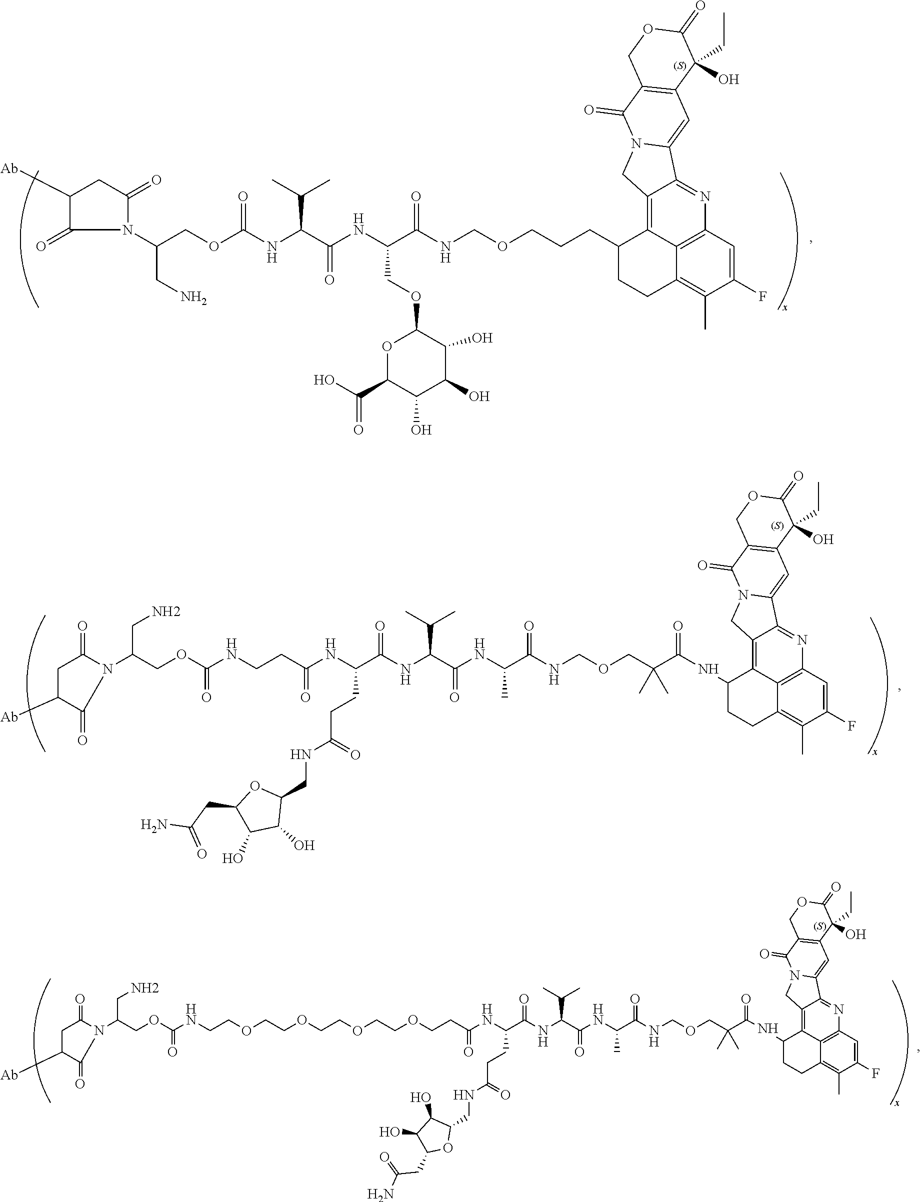 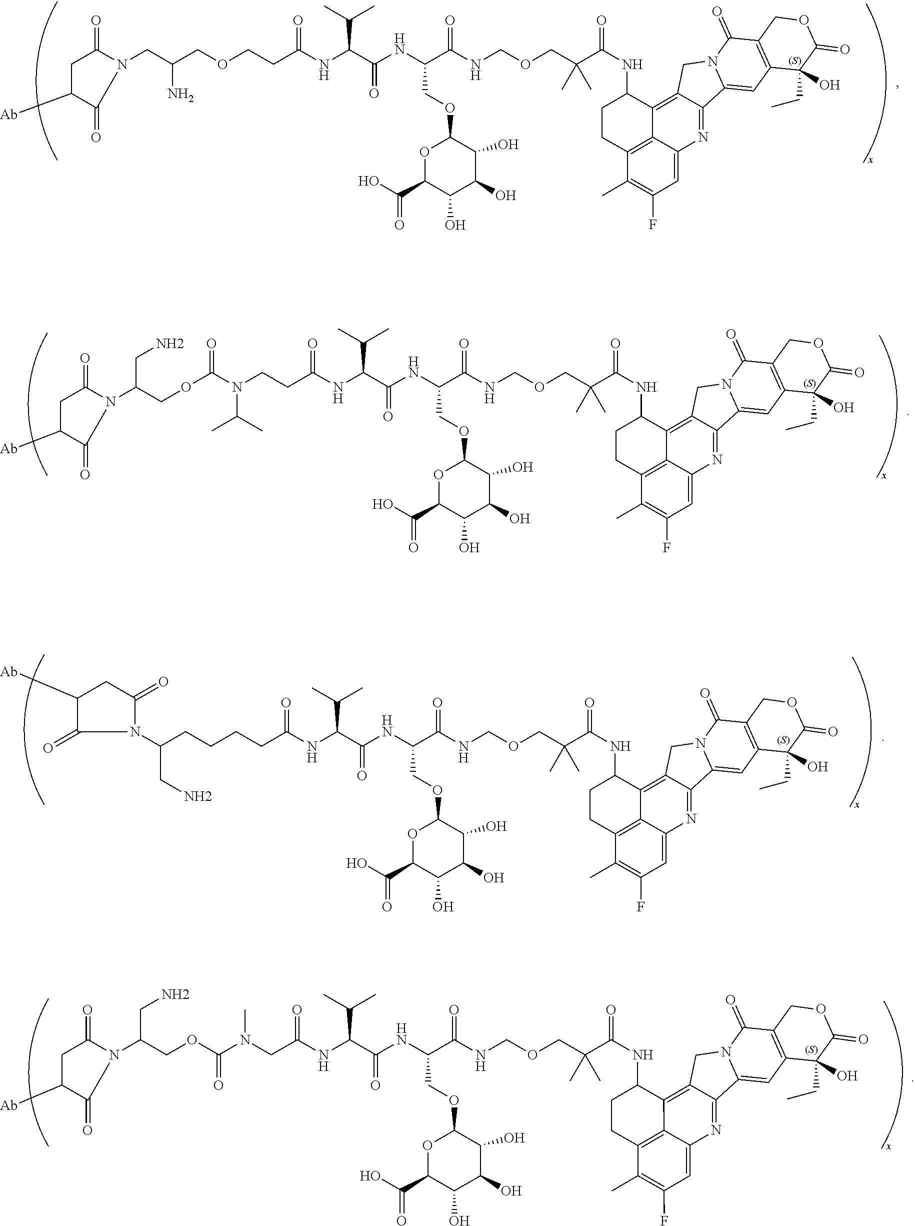 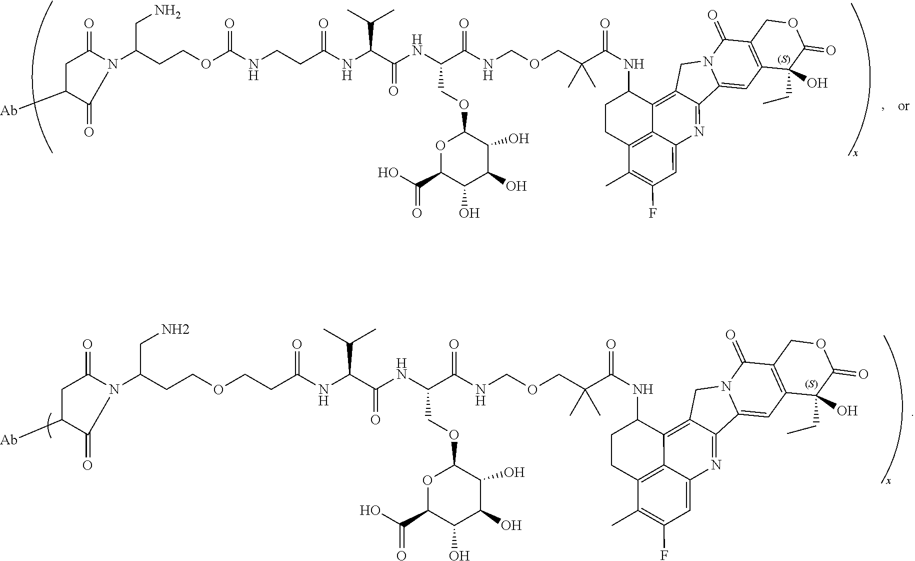
wherein Ab is ifinatamab.

27. A pharmaceutical composition comprising a compound of any one of claims 1-26, or a pharmaceutically acceptable salt, tautomer, solvate, or stereoisomer thereof, and a pharmaceutically acceptable excipient.

28. A compound of Formula (II): 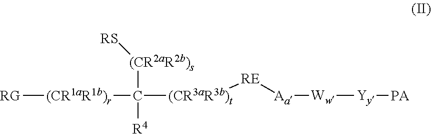
or a pharmaceutically acceptable salt, tautomer, solvate, or stereoisomer, wherein:
RG is a reactive group;
RS is an open-ring stabilizing group;
RE is an open-ring enhancer;
each of R^{1a} and R^{1b} is, independently, H; substituted or unsubstituted C₁₋₄ alkyl; substituted or unsubstituted C₃₋₅ cycloalkyl; or R^{1a} and R^{1b} together with the atom to which they are attached form a substituted or unsubstituted C₃₋₅ cycloalkyl;
each of R^{2a} and R^{2b} is, independently, H; substituted or unsubstituted C₁₋₄ alkyl; substituted or unsubstituted C₃₋₅ cycloalkyl; or R^{2a} and R^{2b} together with the atom to which they are attached form a substituted or unsubstituted C₃₋₅ cycloalkyl;
each of R^{3a} and R^{3b} is, independently, H; substituted or unsubstituted C₁₋₄ alkyl; substituted or unsubstituted C₃₋₅ cycloalkyl; or R^{3a} and R^{3b} together with the atom to which they are attached form a substituted or unsubstituted C₃₋₅ cycloalkyl;
R⁴ is H, substituted or unsubstituted C₁₋₄ alkyl; or substituted or unsubstituted C₃₋₅ cycloalkyl;
each of r, s, and t is, independently, 0, 1, or 2;
A is a Stretcher unit residue;
subscript a′ is 0 or 1;
W is a Cleavable unit;
subscript w′ is 0 or 1;
Y is a Spacer unit;
subscript y′ is 0 or 1; and
PA is a payload residue.

29. The compound of claim 28, wherein each of R^{1a}, R^{1b}, R^{2a}, R^{2b}, R^{3a}, R^{3b}, and R⁴ is, independently, H.

30. The compound of claim 28 or 29,
wherein RS is an amino group or —NR^{5a}R^{5b}, and
wherein each of R^{5a} and R^{5b} is, independently, H, or substituted or unsubstituted C₁₋₄ alkyl.

31. The compound of claim 30, wherein RS is an amino group or —N(CH₃)₂.

32. The compound of claim 31, wherein RS is an amino group.

33. The compound of any one of claims 28-32, wherein RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—; and R⁶ is H, or substituted or unsubstituted C₁₋₄ alkyl.

34. The compound of claim 33, wherein R⁶ is H, methyl, ethyl, or isopropyl.

35. The compound of claim 34, wherein RE is —OC(═O)NR⁶—.

36. The compound of claim 0, wherein RE is —OC(═O)NH—.

37. The compound of any one of claims 28-36, wherein RG is

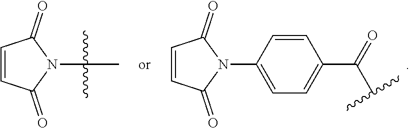 [SMILES: *N1C(=O)C=CC1=O ; *C(=O)c1ccc(N2C(=O)C=CC2=O)cc1]

38. The compound of any one of claims 28-37, wherein r is 0.

39. The compound of any one of claims 28-38, wherein s is 1.

40. The compound of any one of claims 28-39, wherein t is 1 or 2.

41. The compound of any one of claims 28-40, wherein A
is —(CH₂)ₙ—C(═O)—, —CH₂—C(═O)—NH—(CH₂)ₙ—C(═O)—, —(CH₂CH₂O)ₙ—CH₂CH₂—C(═O)—, —CH[—(CH₂)ₙ—COOH]—C(═O)—, —CH₂—C(═O)—NH—(CH₂)ₙ—C(═O)—NH—(CH₂)ₙ—C(═O)—, or —C(═O)—(CH₂)ₙ, —C(═O)—, and wherein each n independently represents an integer of 1, 2, 3, 4, or 5.

42. The compound of any one of claims 28-41, wherein W_{w′} is one of the following formulas: 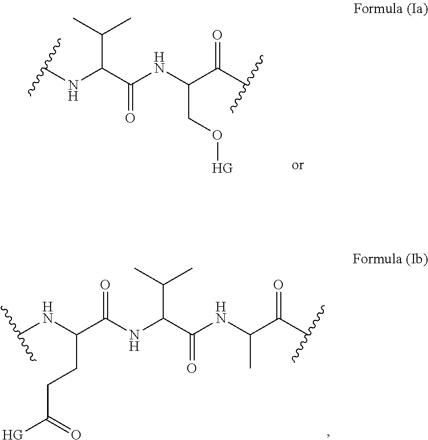
and wherein HG is a hydrophilic moiety or hydrogen.

43. The compound of claim 42, wherein HG is a saccharide, phosphate ester, sulfate ester, a phosphodiester, or a phosphonate.

44. The compound of claim 43, wherein HG is a saccharide and the saccharide is β-D-galactose, N-acetyl-P-D-galactosamine, N-acetyl-a-D-galactosamine, N-acetyl-P-D-glucosamine, β-D-glucuronic acid, a-L-iduronic acid, a-D-galactose, a-D-glucose, β-D-glucose, a-D-mannose, β-D-mannose, a-L-fucose, β-D-xylose, a neuraminic acid, sulfate, phosphate, carboxyl, amino, or an O-acetyl modification thereof.

45. The compound of claim 42, wherein HG is

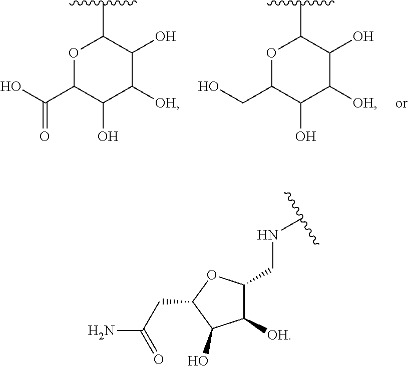 [SMILES: *C1OC(C(=O)O)C(O)C(O)C1O ; *C1OC(CO)C(O)C(O)C1O ; *NC[C@H]1O[C@@H](CC(N)=O)[C@H](O)[C@@H]1O]

46. The compound of any one of claims 28-45, wherein Y_{y′} is a PAB unit.

47. The compound of claim 28, wherein the compound is one of the following formulas:

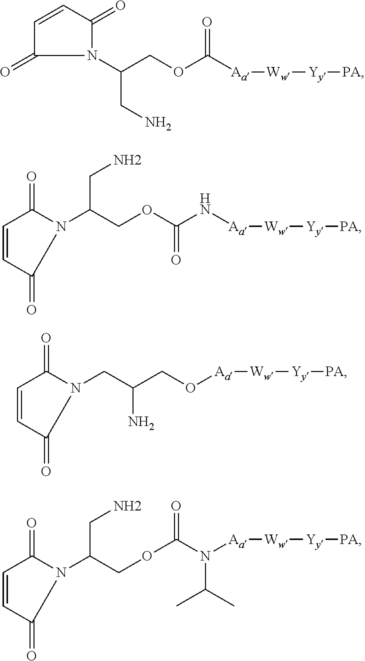

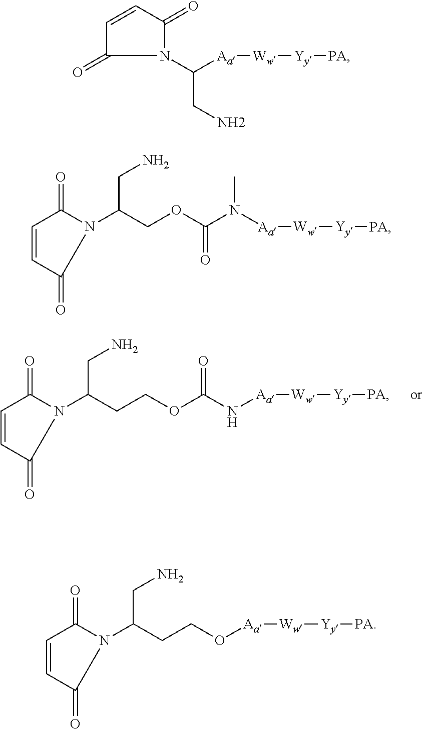

48. The compound of any one of claims 28-47, wherein the compound is one of the following formulas:

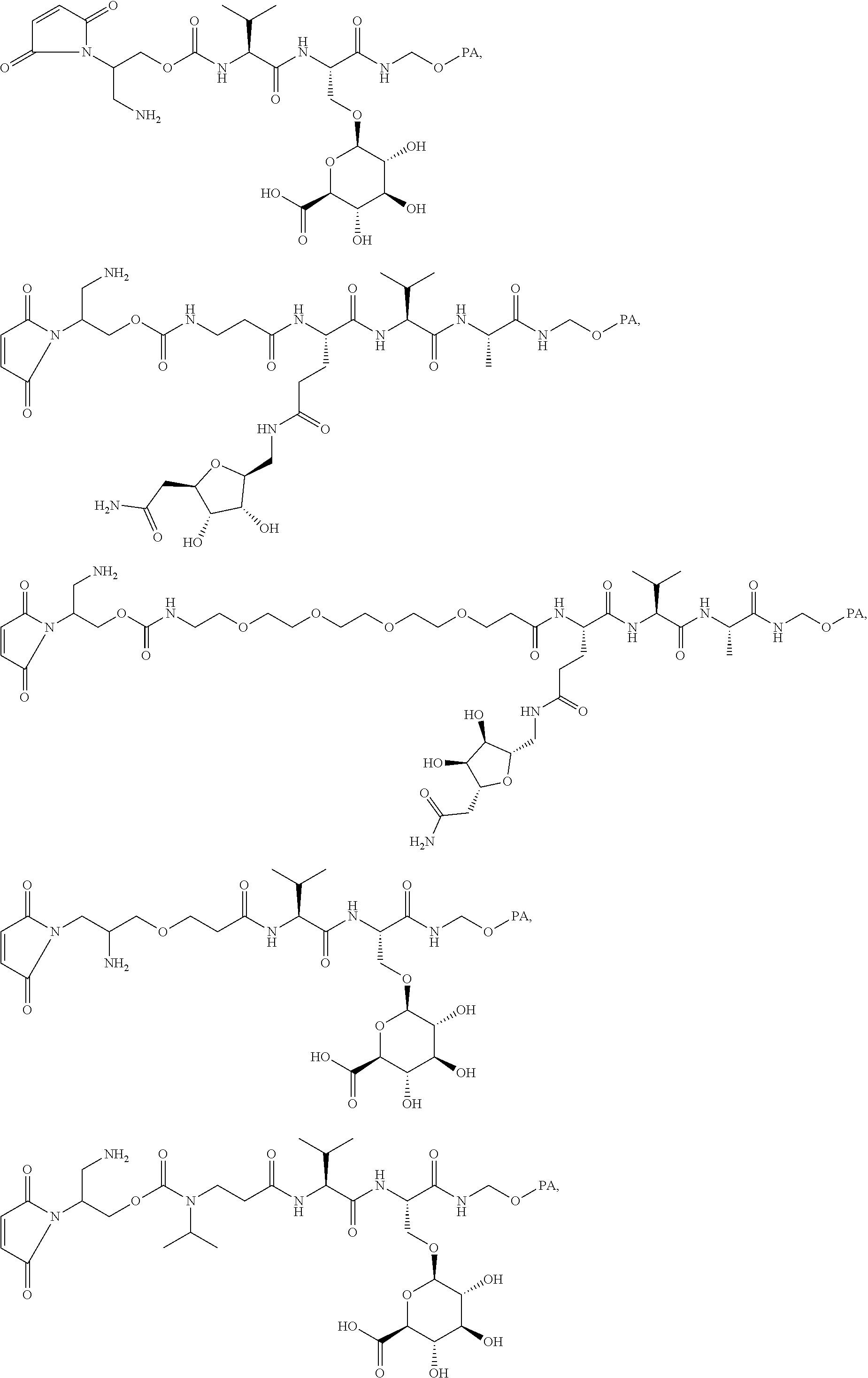

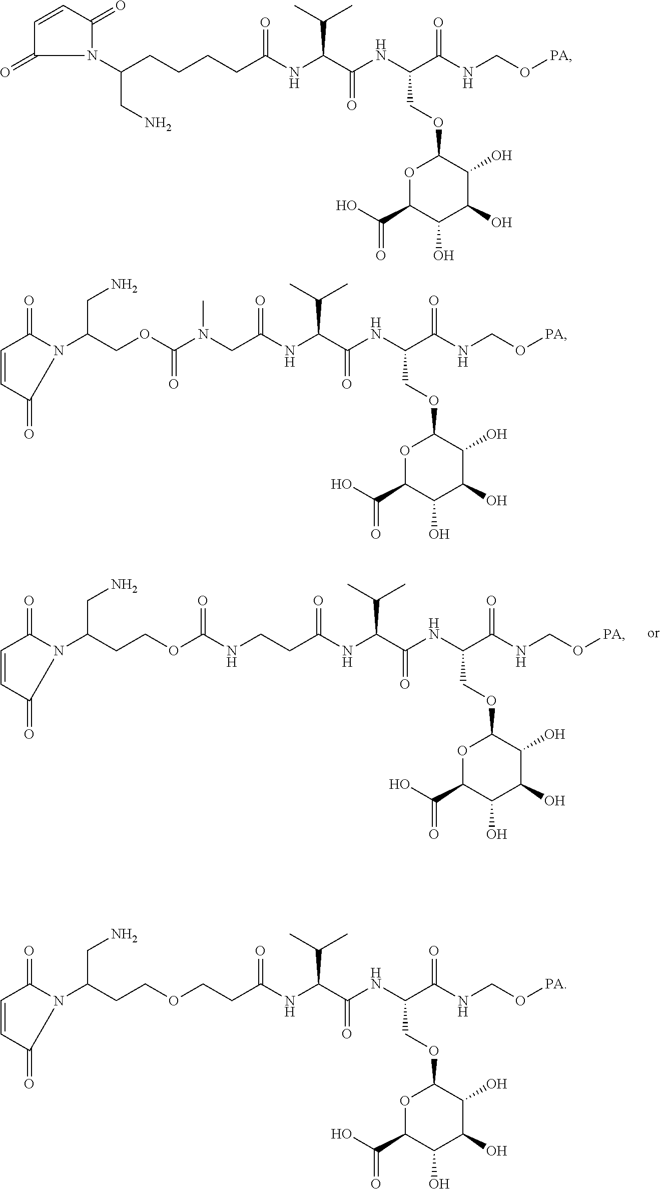

49. The compound of any one of claims 28-48, wherein the compound is

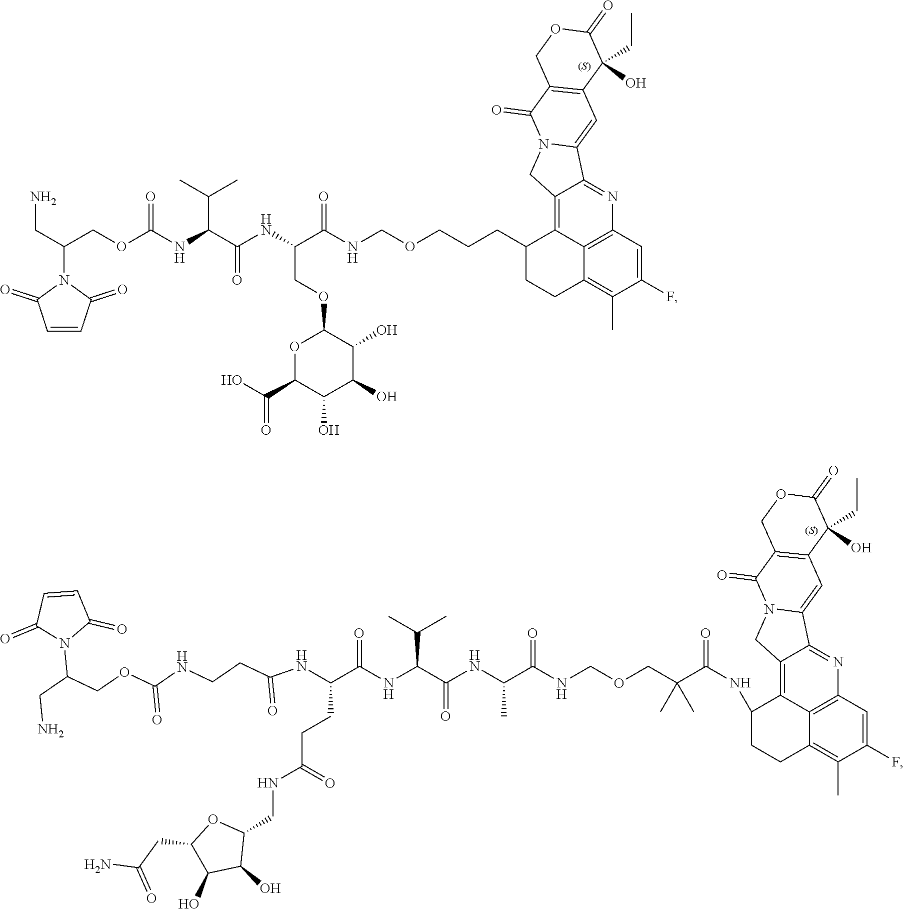

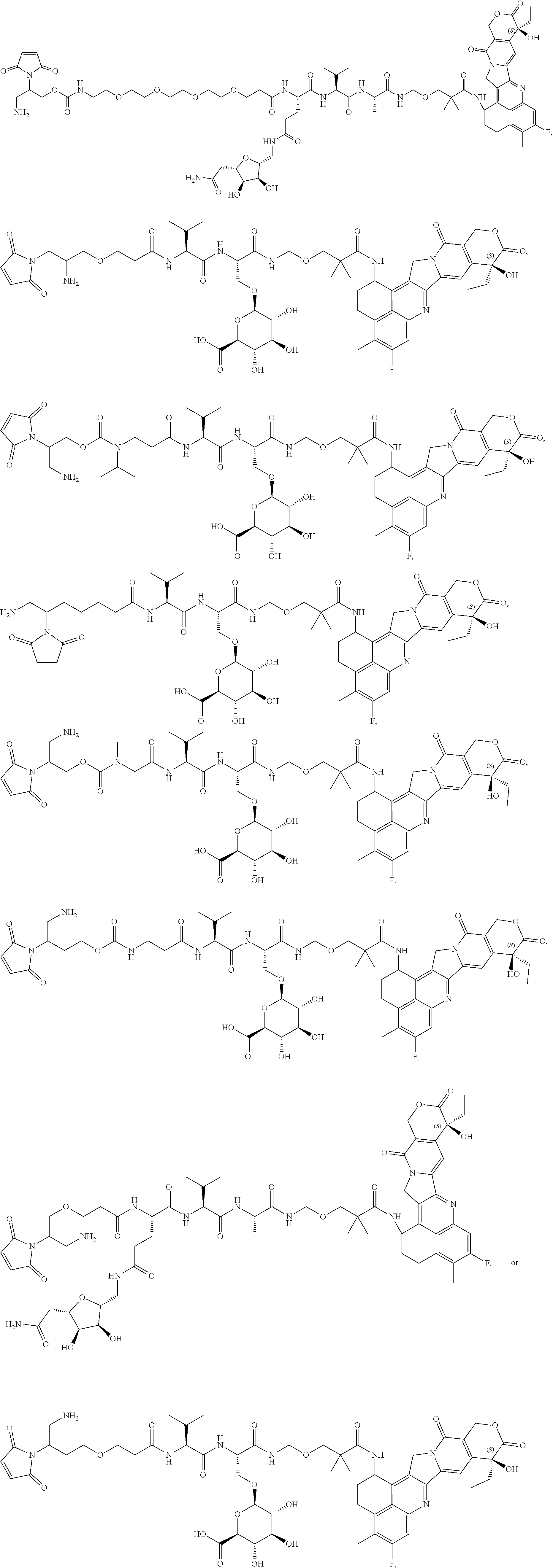

50. The compound of any one of claims 1-26 and 28-48, wherein each PA is independently a cytotoxic agent.

51. The compound of claim 50, wherein PA is independently selected from the group consisting of DXd, 7-Ethyl-10-hydroxy-camptothecin (SN-38), and monomethyl auristatin E (MMAE).

52. The compound of claim 50, wherein PA independently represents a compound of Formula (VI): 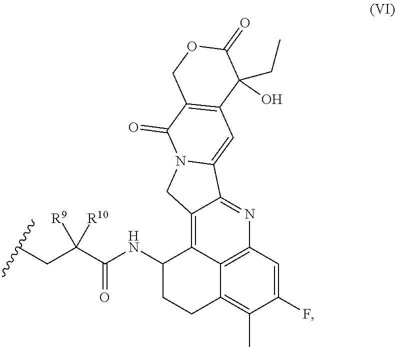
wherein each of R⁹ and R¹⁰ is independently hydrogen, halogen, or substituted or unsubstituted C₁₋₄ alkyl.

53. The compound of claim 50, wherein PA independently represents

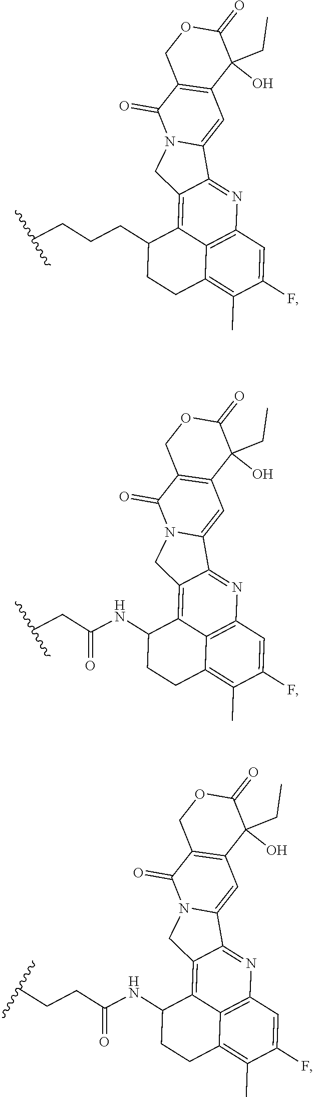 [SMILES: *CCCC1CCc2c(C)c(F)cc3nc4c(c1c23)Cn1c-4cc2c(c1=O)COC(=O)C2(O)CC ; *CC(=O)NC1CCc2c(C)c(F)cc3nc4c(c1c23)Cn1c-4cc2c(c1=O)COC(=O)C2(O)CC ; *CCC(=O)NC1CCc2c(C)c(F)cc3nc4c(c1c23)Cn1c-4cc2c(c1=O)COC(=O)C2(O)CC]

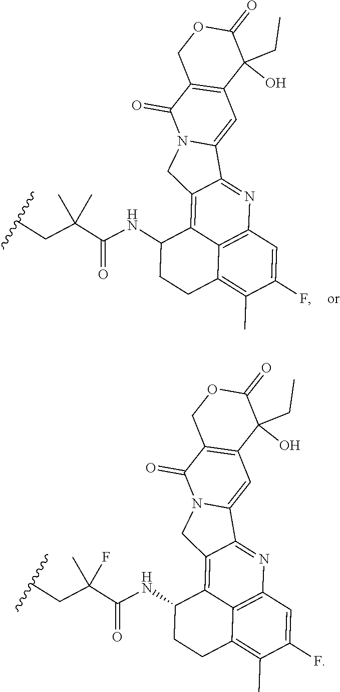

54. A compound of Formula (III): 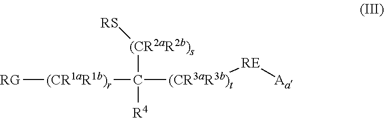
or a pharmaceutically acceptable salt, tautomer, solvate, or stereoisomer thereof,
wherein:
RG is a reactive group;
RS is an open-ring stabilizing group;
RE is an open-ring enhancer;
each of R^{1a} and R^{1b} is, independently, H; substituted or unsubstituted C₁₋₄ alkyl; substituted or unsubstituted C₃₋₅ cycloalkyl; or R^{1a} and R^{1b} together with the atom to which they are attached form a substituted or unsubstituted C₃₋₅ cycloalkyl;
each of R^{2a} and R^{2b} is, independently, H; substituted or unsubstituted C₁₋₄ alkyl; substituted or unsubstituted C₃₋₅ cycloalkyl; or R^{2a} and R^{2b} together with the atom to which they are attached form a substituted or unsubstituted C₃₋₅ cycloalkyl;
each of R^{3a} and R^{3b} is, independently, H; substituted or unsubstituted C₁₋₄ alkyl; substituted or unsubstituted C₃₋₅ cycloalkyl; or R^{3a} and R^{3b} together with the atom to which they are attached form a substituted or unsubstituted C₃₋₅ cycloalkyl;
R⁴ is H; substituted or unsubstituted C₁₋₄ alkyl; or substituted or unsubstituted C₃₋₅ cycloalkyl;
each of r, s, and t is, independently, 0, 1, or 2;
A is a Stretcher unit; and
subscript a′ is 0 or 1.

55. The compound of claim 54, wherein RG is

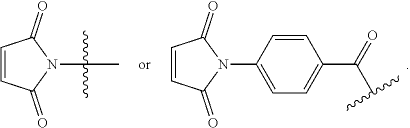 [SMILES: *N1C(=O)C=CC1=O ; *C(=O)c1ccc(N2C(=O)C=CC2=O)cc1]

56. The compound of any claim 54 or 55, wherein each of R^{1a}, R^{1b}, R^{2a}, R^{2b}, R^{3a}, R^{3b}, and R⁴ is, independently, H.

57. The compound of any one of claims 54-56, wherein RS is an amino group or —NR^{5a}R^{5b} and wherein each of R^{5a} and R^{5b} is, independently, H, or substituted or unsubstituted C₁₋₄ alkyl.

58. The compound of claim 57, wherein RS is an amino group or —N(CH₃)₂.

59. The compound of claim 58, wherein RS is an amino group.

60. The compound of any one of claims 54-59, wherein RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—; and R⁶ is H, or substituted or unsubstituted C₁₋₄ alkyl.

61. The compound of claim 60, wherein R⁶ is H, methyl, ethyl, or isopropyl.

62. The compound of claim 61, wherein RE is —OC(═O)NR⁶—.

63. The compound of claim 62, wherein RE is —OC(═O)NH—.

64. The compound of any one of claims 54-63, wherein r is 0.

65. The compound of any one of claims 54-64, wherein s is 1.

66. The compound of any one of claims 54-65, wherein t is 1 or 2.

67. The compound of any one of claims 54-66, wherein
A is a bond, —(CH₂)ₙ—C(═O)R⁷, —CH₂—C(═O)—NH—(CH₂)ₙ—C(═O)R⁷, —(CH₂CH₂O)ₙ, —CH₂CH₂—C(═O)R⁷, —CH[—(CH₂)ₙ—COOH]—C(═O)R⁷, —CH₂—C(═O)—NH—(CH₂)ₙ, —C(═O)—NH—(CH₂)ₙ—C(═O)R⁷, or —C(═O)—(CH₂)ₙ—C(═O)R⁷,
each n independently represents an integer of 1, 2, 3, 4, or 5;
R⁷ is OH or NR^{8a}R^{8b}, and
each of R^{8a} and R^{8b} is, independently, H; substituted or unsubstituted C₁₋₄ alkyl;
substituted or unsubstituted C₃₋₅ cycloalkyl; or R^{8a} and R^{8b} together with the atom to which they are attached form a substituted or unsubstituted C₃₋₅ cycloalkyl.

68. The compound of claim 0, wherein R⁷ is OH, NH₂, NHCH₃, or N(CH₃)₂.

69. The compound of claim 54, wherein the compound is 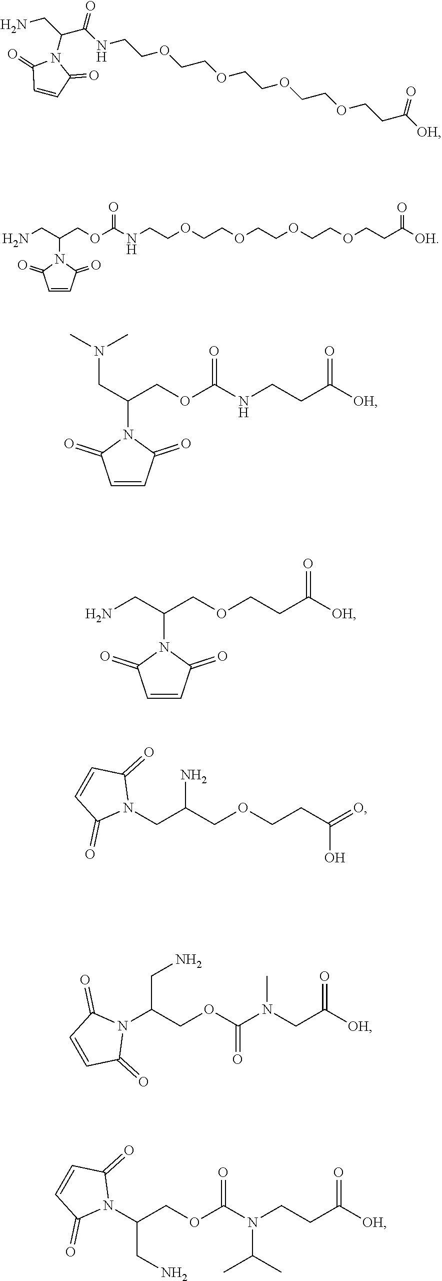 [chemical structure]
